(19) United States
(12) Patent Application Publication
(10) Pub. No.: US 2016/0122760 A1
(43) Pub. Date: May 5, 2016

(54) COMPOSITIONS AND METHODS FOR MODULATING FOXP3 EXPRESSION

(71) Applicant: RANA THERAPEUTICS, INC., Cambridge, MA (US)

(72) Inventors: James Barsoum, Lexington, MA (US); Mariano Severgnini, Newton, MA (US); James McSwiggen, Arlington, MA (US)

(73) Assignee: RaNA Therapeutics, Inc., Cambridge, MA (US)

(21) Appl. No.: 14/896,537

(22) PCT Filed: Jun. 6, 2014
(86) PCT No.: PCT/US14/41345

§ 371 Date: Dec. 7, 2015

Related U.S. Application Data
(60) Provisional application No. 61/832,677, filed on Jun. 7, 2013.

(51) Int. Cl. C12N 15/113

(52) U.S. Cl. CPC C12N 15/113; C12N 2310/3231; C12N 2310/321; C12N 2310/11; C12N 2310/315; C12N 2310/351

(57) ABSTRACT

Aspects of the invention provide single stranded oligonucleotides for activating or enhancing expression of FOXP3. Further aspects provide compositions and kits comprising single stranded oligonucleotides for activating or enhancing expression of FOXP3. Methods for modulating expression of FOXP3 using the single stranded oligonucleotides are also provided. Further aspects of the invention provide methods for selecting a candidate oligonucleotide for activating or enhancing expression of FOXP3.

### CROSS-REFERENCE TO RELATED APPLICATIONS

This application claims the benefit under 35 U.S.C. §119(e) of U.S. Provisional Application No. 61/832,677, entitled “COMPOSITIONS AND METHODS FOR MODULATING FOXP3 EXPRESSION”, filed Jun. 7, 2013, the contents of which are incorporated herein by reference in its entirety.

### FIELD OF THE INVENTION

The invention relates to oligonucleotide based compositions, as well as methods of using oligonucleotide based compositions for treating disease.

### BACKGROUND OF THE INVENTION

FOXP3 (forkhead box P3), a member of the FOX protein family, is a master regulator transcription factor that drives the differentiation and activity of immune suppressive regulatory T cells (Tregs). Tregs are Foxp3⁺CD4⁺CD25⁺ T lymphocytes which have immune suppressive activity and can establish a toleragenic response. It has been shown previously that administration of Foxp3⁺ Treg cells leads to marked reductions in inflammatory/autoimmune disease severity in animal models of type 1 diabetes, multiple sclerosis, asthma, inflammatory bowel disease, and thyroiditis. Expression of FOXP3 decreases effector T cell proliferation and activity. Additionally, Foxp3+ T cells can control a Th1 response, Th17 response, suppress antibody production, CD8+ cytotoxic T cell activity and antigen presentation.

Alterations in the number or function of Tregs, such as those that express Foxp3, are associated with several disease states. For example, patients with autoimmune diseases such as systemic lupus erythematosus (SLE) have been found to have defective regulatory function of Tregs. The FOXP3 gene has also been shown to be mutated in patients with IPEX (Immunodysregulation, Polyendocrinopathy, and Enteropathy, X-linked) syndrome. IPEX syndrome is characterized by the development of multiple autoimmune disorders, such as enteropathy, dermatitis, and Type 1 diabetes, in affected patients.

### SUMMARY OF THE INVENTION

Aspects of the invention disclosed herein provide methods and compositions that are useful for upregulating FOXP3 in cells. In some embodiments, single stranded oligonucleotides are provided that target a PRC2-associated region of an FOXP3 gene (e.g., human FOXP3) and thereby cause upregulation of the gene. In some embodiments, single stranded oligonucleotides are provided that target a PRC2-associated region of the gene encoding FOXP3. In some embodiments, these single stranded oligonucleotides activate or enhance expression of FOXP3 by relieving or preventing PRC2 mediated repression of FOXP3. Aspects of the invention disclosed herein provide methods and compositions that are useful for upregulating FOXP3 for the treatment and/or prevention of diseases or disorders associated with aberrant immune cell (e.g., T cell) activation, e.g., autoimmune or inflammatory diseases or disorders.

Further aspects of the invention provide methods for selecting oligonucleotides for activating or enhancing expression of FOXP3. In some embodiments, methods are provided for selecting a set of oligonucleotides that is enriched in candidates (e.g., compared with a random selection of oligonucleotides) for activating or enhancing expression of FOXP3. Accordingly, the methods may be used to establish sets of clinical candidates that are enriched in oligonucleotides that activate or enhance expression of FOXP3. Such libraries may be utilized, for example, to identify lead oligonucleotides for developing therapeutics to treat FOXP3. Furthermore, in some embodiments, oligonucleotide chemistries are provided that are useful for controlling the pharmacokinetics, biodistribution, bioavailability and/or efficacy of the single stranded oligonucleotides for activating expression of FOXP3.

According to some aspects of the invention single stranded oligonucleotides are provided that have a region of complementarity that is complementary with (e.g., at least 8 consecutive nucleotides of) a PRC2-associated region of a FOXP3 gene, e.g., a PRC2-associated region of the nucleotide sequence set forth as SEQ ID NO: 1, 2, 5, 6, 7, 46, or 47. In some embodiments, the oligonucleotide has at least one of the following features: a) a sequence that is 5′X-Y-Z, in which X is any nucleotide and in which X is at the 5′ end of the oligonucleotide, Y is a nucleotide sequence of 6 nucleotides in length that is not a human seed sequence of a microRNA, and Z is a nucleotide sequence of 1 to 23 nucleotides in length; b) a sequence that does not comprise three or more consecutive guanosine nucleotides; c) a sequence that has less than a threshold level of sequence identity with every sequence of nucleotides, of equivalent length to the second nucleotide sequence, that are between 50 kilobases upstream of a 5′-end of an off-target gene and 50 kilobases downstream of a 3′-end of the off-target gene; d) a sequence that is complementary to a PRC2-associated region that encodes an RNA that forms a secondary structure comprising at least two single stranded loops; and e) a sequence that has greater than 60% G-C content. In some embodiments, the single stranded oligonucleotide has at least two of features a), b), c), d), and e), each independently selected. In some embodiments, the single stranded oligonucleotide has at least three of features a), b), c), d), and e), each independently selected. In some embodiments, the single stranded oligonucleotide has at least four of features a), b), c), d), and e), each independently selected. In some embodiments, the single stranded oligonucleotide has each of features a), b), c), d), and e). In certain embodiments, the oligonucleotide has the sequence 5′X-Y-Z, in which the oligonucleotide is 8-50 nucleotides in length.

According to some aspects of the invention, single stranded oligonucleotides are provided that have a sequence X-Y-Z, in which X is any nucleotide, Y is a nucleotide sequence of 6 nucleotides in length that is not a seed sequence of a human microRNA, and Z is a nucleotide sequence of 1 to 23 nucleotides in length, in which the single stranded oligonucleotide is complementary with a PRC2-associated region of a FOXP3 gene, e.g., a PRC2-associated region of the nucleotide sequence set forth as SEQ ID NO: 1, 2, 5, 6, 7, 46, or 47. In some aspects of the invention, single stranded oligonucleotides are provided that have a sequence 5′-X-Y-Z, in which X is any nucleotide, Y is a nucleotide sequence of 6 nucleotides in length that is not a seed sequence of a human microRNA, and Z is a nucleotide sequence of 1 to 23 nucleotides in length, in which the single stranded oligonucleotide is complementary with at least 8 consecutive nucleotides of a PRC2-associated region of FOXP3 gene, e.g., a PRC2-associated region of the nucleotide sequence set forth as SEQ ID NO: 1, 2, 5, 6, 7, 46, or 47. In some embodiments, Y is a sequence selected from Table 1. In some embodiments, the PRC2-associated region is a sequence listed in any one of SEQ ID NOS: 8-45 or 48-59.

In some embodiments, the single stranded oligonucleotide comprises a nucleotide sequence as set forth in any one of SEQ ID NOS: 60-45713, or a fragment thereof that is at least 8 nucleotides. In some embodiments, the single stranded oligonucleotide comprises a nucleotide sequence as set forth in any one of SEQ ID NOS: 60-45713, in which the 5′ end of the nucleotide sequence provided is the 5′ end of the oligonucleotide. In some embodiments, the region of complementarity (e.g., the at least 8 consecutive nucleotides) is also present within the nucleotide sequence set forth as SEQ ID NO: 3 or 4.

In some embodiments, the PRC2-associated region is a sequence listed in any one of SEQ ID NOS: 8-45. In some embodiments, the single stranded oligonucleotide comprises a nucleotide sequence as set forth in any one of SEQ ID NOS: 16426-45713 or a fragment thereof that is at least 8 nucleotides. In some embodiments, the single stranded oligonucleotide comprises a nucleotide sequence as set forth in any one of SEQ ID NOS: 16426-45713, wherein the 5′ end of the nucleotide sequence provided in any one of SEQ ID NOS: 16426-45713 is the 5′ end of the oligonucleotide. In some embodiments, the at least 8 consecutive nucleotides are also present within the nucleotide sequence set forth as SEQ ID NO: 4.

In some embodiments, the PRC2-associated region is a sequence listed in any one of SEQ ID NOS: 48-59. In some embodiments, the single stranded oligonucleotide comprises a nucleotide sequence as set forth in any one of SEQ ID NOS: 60-16461 or a fragment thereof that is at least 8 nucleotides. In some embodiments, the single stranded oligonucleotide comprises a nucleotide sequence as set forth in any one of SEQ ID NOS: 60-16461, wherein the 5′ end of the nucleotide sequence provided in any one of SEQ ID NOS: 60-16461 is the 5′ end of the oligonucleotide. In some embodiments, the at least 8 consecutive nucleotides are also present within the nucleotide sequence set forth as SEQ ID NO: 3.

In some embodiments, a single stranded oligonucleotide comprises a nucleotide sequence as set forth in any one of SEQ ID NOS: 60-16461. In some embodiments, the oligonucleotide is up to 50 nucleotides in length. In some embodiments, a single stranded oligonucleotide comprises a fragment of at least 8 nucleotides of a nucleotide sequence as set forth in any one of SEQ ID NOS: 60-16461.

In some embodiments, a single stranded oligonucleotide comprises a nucleotide sequence as set forth in Table 4. In some embodiments, the single stranded oligonucleotide comprises a fragment of at least 8 nucleotides of a nucleotide sequence as set forth in Table 4. In some embodiments, a single stranded oligonucleotide consists of a nucleotide sequence as set forth in Table 4.

In some embodiments, a single stranded oligonucleotide, when delivered to a cell, is capable of increasing the level of CTLA4, GITR, and/or IL-10 expression in the cell (e.g., results in a level of expression of CTLA4, GITR, and/or IL-10 that is at least 30% greater than a level of expression of CTLA4, GITR, and/or IL-10 in a control cell). In some embodiments, the cell is a T cell. In some embodiments, the single stranded oligonucleotide, when delivered to a population of T cells, is capable of increasing the number of CD4+CD25+FOXP3+ T cells in the population of T cells (e.g., results in a number of CD4+CD25+FOXP3+ T cells in the population that is at least 30% greater than a number of CD4+CD25+FOXP3+ T cells in a control population). In some embodiments, the single stranded oligonucleotide does not comprise three or more consecutive guanosine nucleotides. In some embodiments, the single stranded oligonucleotide does not comprise four or more consecutive guanosine nucleotides.

In some embodiments, the single stranded oligonucleotide is 8 to 30 nucleotides in length. In some embodiments, the single stranded oligonucleotide is up to 50 nucleotides in length. In some embodiments, the single stranded oligonucleotide is 8 to 10 nucleotides in length and all but 1, 2, or 3 of the nucleotides of the complementary sequence of the PRC2-associated region are cytosine or guanosine nucleotides.

In some embodiments, the single stranded oligonucleotide is complementary with at least 8 consecutive nucleotides of a PRC2-associated region of a FOXP3 gene, e.g., a PRC2-associated region of a nucleotide sequence set forth as SEQ ID NO: 1, 2, 5, 6, 7, 46, or 47, in which the nucleotide sequence of the single stranded oligonucleotide comprises one or more of a nucleotide sequence selected from the group consisting of

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, wherein “X” denotes a nucleotide analogue, (X) denotes an optional nucleotide analogue, and “x” denotes a DNA or RNA nucleotide unit.

In some embodiments, at least one nucleotide of the oligonucleotide is a nucleotide analogue. In some embodiments, the at least one nucleotide analogue results in an increase in Tm of the oligonucleotide in a range of 1 to 5° C. compared with an oligonucleotide that does not have the at least one nucleotide analogue.

In some embodiments, at least one nucleotide of the oligonucleotide comprises a 2′ O-methyl. In some embodiments, each nucleotide of the oligonucleotide comprises a 2′ O-methyl. In some embodiments, the oligonucleotide comprises at least one ribonucleotide, at least one deoxyribonucleotide, or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide.

In some embodiments, the nucleotides of the oligonucleotide comprise alternating deoxyribonucleotides and 2′-fluoro-deoxyribonucleotides. In some embodiments, the nucleotides of the oligonucleotide comprise alternating deoxyribonucleotides and 2′-O-methyl nucleotides. In some embodiments, the nucleotides of the oligonucleotide comprise alternating deoxyribonucleotides and ENA nucleotide analogues. In some embodiments, the nucleotides of the oligonucleotide comprise alternating deoxyribonucleotides and LNA nucleotides. In some embodiments, the 5′ nucleotide of the oligonucleotide is a deoxyribonucleotide. In some embodiments, the nucleotides of the oligonucleotide comprise alternating LNA nucleotides and 2′-O-methyl nucleotides. In some embodiments, the 5′ nucleotide of the oligonucleotide is a LNA nucleotide. In some embodiments, the nucleotides of the oligonucleotide comprise deoxyribonucleotides flanked by at least one LNA nucleotide on each of the 5′ and 3′ ends of the deoxyribonucleotides.

In some embodiments, the single stranded oligonucleotide comprises modified internucleotide linkages (e.g., phosphorothioate internucleotide linkages or other linkages) between at least two, at least three, at least four, at least five or more nucleotides. In some embodiments, the single stranded oligonucleotide comprises modified internucleotide linkages (e.g., phosphorothioate internucleotide linkages or other linkages) between all nucleotides.

In some embodiments, the nucleotide at the 3′ position of the oligonucleotide has a 3′ hydroxyl group. In some embodiments, the nucleotide at the 3′ position of the oligonucleotide has a 3′ thiophosphate. In some embodiments, the single stranded oligonucleotide has a biotin moiety or other moiety conjugated to its 5′ or 3′ nucleotide. In some embodiments, the single stranded oligonucleotide has cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

According to some aspects of the invention compositions are provided that comprise any of the oligonucleotides disclosed herein, and a carrier. In some embodiments, compositions are provided that comprise any of the oligonucleotides in a buffered solution. In some embodiments, the oligonucleotide is conjugated to the carrier. In some embodiments, the carrier is a peptide. In some embodiments, the carrier is a steroid. According to some aspects of the invention pharmaceutical compositions are provided that comprise any of the oligonucleotides disclosed herein, and a pharmaceutically acceptable carrier.

According to other aspects of the invention, kits are provided that comprise a container housing any of the compositions disclosed herein.

According to some aspects of the invention, methods of increasing expression of FOXP3 in a cell are provided. In some embodiments, the methods involve delivering any one or more of the single stranded oligonucleotides disclosed herein into the cell. In some embodiments, delivery of the single stranded oligonucleotide into the cell results in a level of expression of FOXP3 that is greater (e.g., at least 50% greater) than a level of expression of FOXP3 in a control cell that does not comprise the single stranded oligonucleotide. In some embodiments, delivery of the single stranded oligonucleotide into the cell results in an increased level of CTLA4, GITR, and/or IL-10 expression compared to an appropriate control cell that does not comprise the singled stranded oligonucleotide. In some embodiments, delivery of the single stranded oligonucleotide into the cell results in a level of expression of CTLA4, GITR, and/or IL-10 that is greater than (e.g., at least 30% greater than) a level of expression of CTLA4, GITR, and/or IL-10 in a control cell that does not comprise the single stranded oligonucleotide. In some embodiments, the cell is a T cell.

According to some aspects of the invention, methods of increasing levels of FOXP3 in a subject are provided. According to some aspects of the invention, methods of treating a condition or disease (e.g., a disease or disorder associated with aberrant immune cell activation such as an autoimmune or inflammatory disease or disorder) associated with decreased levels of FOXP3 in a subject are provided. In some embodiments, the methods involve administering any one or more of the single stranded oligonucleotides disclosed herein to the subject. In some embodiments, administration of the single stranded oligonucleotide to the subject results in an increased level of CTLA4, GITR, and/or IL-10 expression the subject compared to an appropriate control subject who has not been administered the single stranded oligonucleotide. In some embodiments, administration of the single stranded oligonucleotide to the subject results in a level of expression of CTLA4, GITR, and/or IL-10 that is greater than (e.g., at least 30% greater than) a level of CTLA4, GITR, and/or IL-10 in the appropriate control subject who has not been administered the single stranded oligonucleotide. In some embodiments, administration of the single stranded oligonucleotide to the subject results in an increased level of CTLA4, GITR, and/or IL-10 in a T cell of the subject compared to a T cell in the control subject who has not been administered the single stranded oligonucleotide. In some embodiments, administration of the single stranded oligonucleotide to the subject results in a level of expression of CTLA4, GITR, and/or IL-10 in the T cell of the subject that is greater than (e.g., at least 30% greater than) a level of CTLA4, GITR, and/or IL-10 in the T cell in the control subject who has not been administered the single stranded oligonucleotide. In some embodiments, administration of the of the single stranded oligonucleotide to the subject results in an increased number of CD4+CD25+FOXP3+ T cells in the subject compared to a control subject who has not been administered the single stranded oligonucleotide. In some embodiments, administration of the single stranded oligonucleotide to the subject results in a number of CD4+CD25+FOXP3+ T cells in the subject that is greater than (e.g., at least 30% greater than) a number of CD4+CD25+FOXP3+ T cells in the control subject who has not been administered the single stranded oligonucleotide.

Other aspects of the invention relate to a method of increasing expression of FOXP3 in a cell, activating T cells, and/or treating a condition or disease (e.g., a disease or disorder associated with aberrant immune cell activation such as an autoimmune or inflammatory disease or disorder) associated with decreased levels of FOXP3 by inhibiting or decreasing expression of EZH1 and/or EZH2 or another component of PRC2, e.g., Suz12, EED1, or RbAp48. In some embodiments, the method comprises delivering an oligonucleotide having a region of complementarity that is complementary with at least 8 consecutive nucleotides of an EZH1 mRNA or EZH2 mRNA to the cell. In some embodiments, the method comprises delivering to the cell a first oligonucleotide having a region of complementarity that is complementary with at least 8 consecutive nucleotides of an EZH1 mRNA and a second oligonucleotide having a region of complementarity that is complementary with at least 8 consecutive nucleotides of an EZH2 mRNA.

In some embodiments, the oligonucleotide is 8 to 30 nucleotides in length. In some embodiments, at least one nucleotide of the oligonucleotide is a nucleotide analogue.

In some embodiments, the oligonucleotide comprises a gapmer. In some embodiments, the gapmer comprises a central region of at least 4 DNA nucleotides flanked one both sides by at least two nucleotide analogues. In some embodiments, the at least two nucleotide analogues comprise at least one LNA or at least one 2′-O modified ribonucleotide.

In some embodiments, the oligonucleotide comprises at least 8 nucleotides of a nucleotide sequence as set forth in Table 8. In some embodiments, the oligonucleotide comprises a nucleotide sequence as set forth in Table 8. In some embodiments, the oligonucleotide consists of a nucleotide sequence as set forth in Table 8. In some embodiments, the oligonucleotide (e.g., single stranded oligonucleotide) comprises a sequence as set forth in any one of SEQ ID NO: 45714-45717 or a complement of anyone of them.

In some embodiments, at least one nucleotide of the oligonucleotide comprises a 2′ O-methyl. In some embodiments, each nucleotide of the oligonucleotide comprises a 2′ O-methyl. In some embodiments, the oligonucleotide comprises at least one ribonucleotide, at least one deoxyribonucleotide, or at least one bridged nucleotide. In some embodiments, the bridged nucleotide is a LNA nucleotide, a cEt nucleotide or a ENA modified nucleotide. In some embodiments, each nucleotide of the oligonucleotide is a LNA nucleotide.

In some embodiments, the nucleotides of the oligonucleotide comprise alternating deoxyribonucleotides and 2′-fluoro-deoxyribonucleotides. In some embodiments, the nucleotides of the oligonucleotide comprise alternating deoxyribonucleotides and 2′-O-methyl nucleotides. In some embodiments, the nucleotides of the oligonucleotide comprise alternating deoxyribonucleotides and ENA nucleotide analogues. In some embodiments, the nucleotides of the oligonucleotide comprise alternating deoxyribonucleotides and LNA nucleotides. In some embodiments, the 5′ nucleotide of the oligonucleotide is a deoxyribonucleotide. In some embodiments, the nucleotides of the oligonucleotide comprise alternating LNA nucleotides and 2′-O-methyl nucleotides. In some embodiments, the 5′ nucleotide of the oligonucleotide is a LNA nucleotide. In some embodiments, the nucleotides of the oligonucleotide comprise deoxyribonucleotides flanked by at least one LNA nucleotide on each of the 5′ and 3′ ends of the deoxyribonucleotides.

In some embodiments, the single stranded oligonucleotide comprises modified internucleotide linkages (e.g., phosphorothioate internucleotide linkages or other linkages) between at least two, at least three, at least four, at least five or more nucleotides. In some embodiments, the single stranded oligonucleotide comprises modified internucleotide linkages (e.g., phosphorothioate internucleotide linkages or other linkages) between all nucleotides.

In some embodiments, the nucleotide at the 3′ position of the oligonucleotide has a 3′ hydroxyl group. In some embodiments, the nucleotide at the 3′ position of the oligonucleotide has a 3′ thiophosphate. In some embodiments, the single stranded oligonucleotide has a biotin moiety or other moiety conjugated to its 5′ or 3′ nucleotide. In some embodiments, the single stranded oligonucleotide has cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Other aspects of the invention relate to a single stranded oligonucleotide having a region of complementarity that is complementary with at least 8 consecutive nucleotides of an EZH1 mRNA or EZH2 mRNA. In some embodiments, a single stranded oligonucleotide comprises a nucleotide sequence as set forth in Table 8. In some embodiments, the single stranded oligonucleotide comprises a fragment of at least 8 nucleotides of a nucleotide sequence as set forth in Table 8. In some embodiments, a single stranded oligonucleotide consists of a nucleotide sequence as set forth in Table 8.

In some embodiments, the oligonucleotide is 8 to 30 nucleotides in length. In some embodiments, at least one nucleotide of the oligonucleotide is a nucleotide analogue.

In some embodiments, the oligonucleotide comprises a gapmer. In some embodiments, the gapmer comprises a central region of at least 4 DNA nucleotides flanked one both sides by at least two nucleotide analogues. In some embodiments, the at least two nucleotide analogues comprise at least one LNA or at least one 2′-O modified ribonucleotide.

### BRIEF DESCRIPTION OF DRAWINGS

FIG. 1 is a series of four graphs showing the levels of CD69, CD62L, CDKN1A, and IL-2RA expression in human T cells that were activated with PMA and Ionomycin.

FIG. 2 is a diagram showing the location along the human FOXP3 gene where the FOXP3 oligos in Table 4 bind.

FIG. 3 is a series of two graphs showing the levels of CD62L and CD69 mRNA in human T cells activated with different concentrations of PMA and Ionomycin (1× or 2×), compared to T cells treated with DMSO, or untreated.

FIG. 4 is a series of two graphs showing the levels of CD62L and CD25 mRNA in cells activated with dynabeads at a ratio of 2:1 beads to cells (left bars) or 1:1 beads to cells (right bars).

FIG. 5 is a graph showing the downregulation of GAPDH mRNA with GAPDH gapmers at concentrations 0, 0.032, 0.16, 0.8, 4 and 20 uM delivered gymnotically to activated human T cells.

FIG. 6 is a graph showing FOXP3 mRNA levels at 48 hours in PMA/Iono activated human T cells treated with 10 uM FOXP3 oligos. Bars with stars indicate oligo treatments where stable housekeeper gene Ct values were observed.

FIG. 7 is a graph showing FOXP3 mRNA levels at 96 hours in dynabead activated human T cells treated with 10 uM FOXP3 oligos.

FIG. 8 is a graph showing GAPDH mRNA levels at 96 hours in dynabead activated human T cells treated with FOXP3 oligos. Black colored bars indicate oligos where housekeeper gene varied more than 1.5Cts from negative control.

FIG. 9 is a graph showing CTLA4 mRNA levels at 96 hours in dynabead activated human T cells treated with FOXP3 oligos.

FIG. 10 is a graph showing GITR mRNA levels at 96 hours in dynabead activated human T cells treated with FOXP3 oligos.

FIG. 11 is a graph showing FOXP3 mRNA levels at 96 hours in dynabead activated human T cells treated with FOXP3 oligos.

FIG. 12 is a graph showing GAPDH mRNA levels at 96 hours in dynabead activated human T cells treated with FOXP3 oligos. Black colored bars indicate oligos where housekeeper gene varied more than 1.5Cts from negative control.

FIG. 13 is a graph showing FoxP3 fluorescent intensity at 96 hours in dynabead activated in CD4+CD25+FoxP3+ human T cells treated with FOXP3 oligos.

FIG. 14 is a diagram showing flow cytometry results in activated human T cells treated with a negative control oligo (293) and an exemplary FOXP3 oligo (FOXP3-35).

FIG. 15 is a graph showing the percentage of CD4+CD25+FoxP3+ cells at 96 hours in dynabead activated in human T cells treated with FOXP3 oligos.

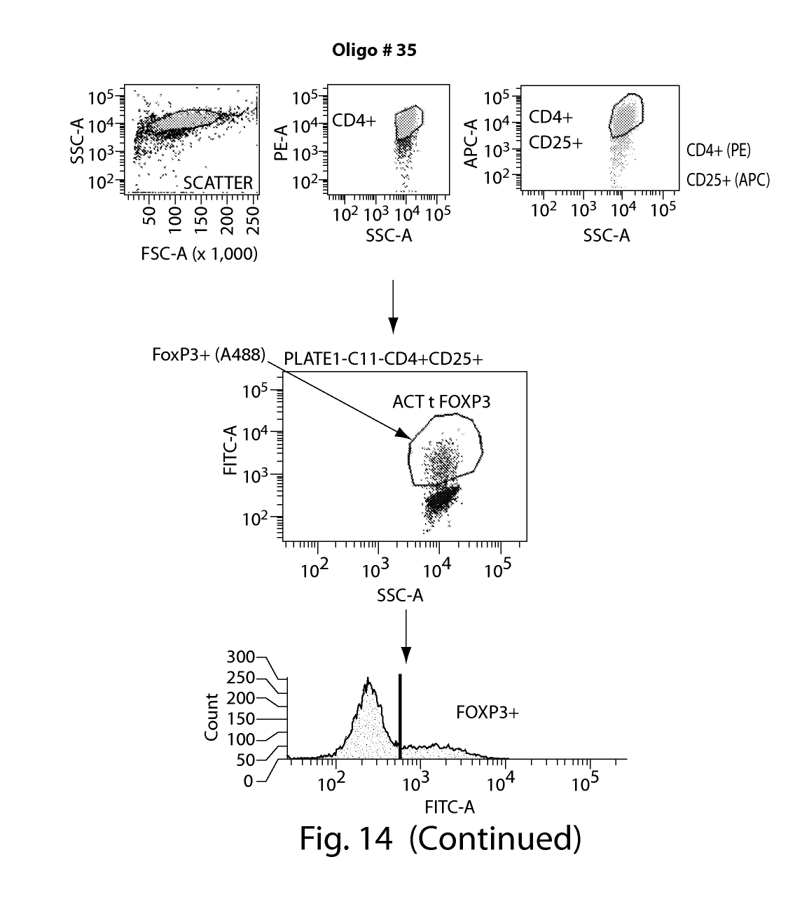

FIG. 16 is a graph showing IL-10 protein levels at 96 hours in dynabead activated in CD4+CD25+FoxP3+ human T cells treated with FOXP3 oligos.

FIG. 17 is a graph showing MALAT-1 mRNA levels in sorted CD4+ cells from whole blood collected from mice treated with MALAT-1 gapmer oligos.

FIG. 18 is a graph showing MALAT-1 mRNA levels in liver collected from mice treated with MALAT-1 gapmer oligos.

FIG. 19 is a graph showing EZH1 mRNA levels at 3 or 5 days in activated human T cells treated with EZH1 gapmers, EZH2 gapmers, or combinations of EZH1 and EZH2 gapmers. The left bar in each pair of bars is 3 days. The right bar in each pair of bars is 5 days.

FIG. 20 is a graph showing EZH2 mRNA levels at 3 or 5 days in activated human T cells treated with EZH1 gapmers, EZH2 gapmers, or combinations of EZH1 and EZH2 gapmers. The left bar in each pair of bars is 3 days. The right bar in each pair of bars is 5 days.

FIG. 21 is a graph showing FOXP3 mRNA levels at 3 or 5 days in activated human T cells treated with EZH1 gapmers, EZH2 gapmers, or combinations of EZH1 and EZH2 gapmers. The left bar in each pair of bars is 3 days. The right bar in each pair of bars is 5 days.

FIG. 22 is a heatmap showing mRNA expression of T cell genes after EZH1/2 knockdown.

FIG. 23 is a series of graphs showing flow cytometry data of FOXP3 protein levels in activated human T cells treated with EZH1 gapmers, EZH2 gapmers, or combinations of EZH1 and EZH2 gapmers.

### BRIEF DESCRIPTION OF CERTAIN TABLES

Table 1: Hexamers that are not seed sequences of human miRNAs

Table 2: Experimental evaluation of single stranded oligonucleotides. SEQ ID (column 1) refers to the SEQ ID NO: that corresponds to the base sequence of the oligonucleotide. The formatted sequence, including any modifications, for each oligonucleotide is provided in Table 4. Oligo name (column 2) refers to the name for a given oligonucleotide and also refers to the same formatted oligonucleotide in Table 4. RQ (column 3) and AVG RQ SD (column 4) shows the expression level of the “probe” gene in a well containing oligo relative to a control well (carrier alone or a universal negative control oligo 293) and the standard deviation for the triplicate replicates of the experiment. Target (column 5) refers to the gene that is targeted by the oligonucleotide. Probe (column 6) refers to the gene whose expression was measured in a given assay. For example, Target FOXP3 and Probe GITR refers to an experiment where are oligo that targets FOXP3 was added to a well and the level of GITR was measured by qRT-PCR. The RQ and AVG RQ SD for that experiment would be the RQ and AVG RQ SD for GITR. [Oligo] is shown in nanomolar for in vitro experiments and in milligrams per kilogram of body weight for in vivo experiments.

Table 3: A listing of oligonucleotide modifications

Table 4: Formatted oligonucleotide sequences made for testing showing nucleotide modifications. The table shows the sequence of the modified nucleotides, where lnaX represents an LNA nucleotide with 3′ phosphorothioate linkage, omeX is a 2′-O-methyl nucleotide, dX is a deoxy nucleotide. An s at the end of a nucleotide code indicates that the nucleotide had a 3′ phosphorothioate linkage. The “-Sup” at the end of the sequence marks the fact that the 3′ end lacks either a phosphate or thiophosphate on the 3′ linkage. The Formatted Sequence column shows the sequence of the oligonucleotide, including modified nucleotides, for the oligonucleotides tested in Table 2.

### DETAILED DESCRIPTION OF CERTAIN EMBODIMENTS OF THE INVENTION

Aspects of the invention provided herein relate to the discovery of polycomb repressive complex 2 (PRC2)-interacting RNAs. Polycomb repressive complex 2 (PRC2) is a histone methyltransferase and a known epigenetic regulator involved in silencing of genomic regions through methylation of histone H3. Among other functions, PRC2 interacts with long noncoding RNAs (lncRNAs), such as RepA, Xist, and Tsix, to catalyze trimethylation of histone H3-lysine27. PRC2 contains four subunits, Eed, Suz12, RbAp48, and Ezh2.

Aspects of the invention relate to the recognition that single stranded oligonucleotides that bind to PRC2-associated regions of RNAs (e.g., lncRNAs) that are expressed from within a genomic region that encompasses or that is in functional proximity to the FOXP3 gene can induce or enhance expression of FOXP3. In some embodiments, this upregulation is believed to result from inhibition of PRC2 mediated repression of FOXP3. FOXP3 is a master regulator transcription factor that drives T cell differentiation and activity of T regulatory cells (Tregs). Tregs have immune suppressive activity and can help to promote a toleragenic response. Tregs have been shown to be helpful in shutting down T cell-mediated immunity toward the end of an immune reaction and in suppressing self-reactive T cells that have escaped the process of negative selection in the thymus. Activated T cells are important for immunoprotection of a host from pathogens and tumor cells. However, inappropriately activated or self-reactive T cells may have deleterious effects, e.g., by causing uncontrolled immune responses or a self-targeting autoimmune response. It is contemplated herein that upregulation of FOXP3 may be used to drive T cell differentiation and/or activity toward a T regulatory state. This may be useful, e.g., to drive activated T cells to differentiate into Tregs or to suppress activated T cell activity. Accordingly, aspects of the invention relate to compositions and methods for upregulating FOXP3.

As used herein, the term “PRC2-associated region” refers to a region of a nucleic acid that comprises or encodes a sequence of nucleotides that interact directly or indirectly with a component of PRC2. A PRC2-associated region may be present in a RNA (e.g., a long non-coding RNA (lncRNA)) that interacts with a PRC2. A PRC2-associated region may be present in a DNA that encodes an RNA that interacts with PRC2. In some cases, the PRC2-associated region is equivalently referred to as a PRC2-interacting region.

In some embodiments, a PRC2-associated region is a region of an RNA that crosslinks to a component of PRC2 in response to in situ ultraviolet irradiation of a cell that expresses the RNA, or a region of genomic DNA that encodes that RNA region. In some embodiments, a PRC2-associated region is a region of an RNA that immunoprecipitates with an antibody that targets a component of PRC2, or a region of genomic DNA that encodes that RNA region. In some embodiments, a PRC2-associated region is a region of an RNA that immunoprecipitates with an antibody that binds specifically to SUZ12, EED, EZH2 or RBBP4 (which as noted above are components of PRC2), or a region of genomic DNA that encodes that RNA region.

In some embodiments, a PRC2-associated region is a region of an RNA that is protected from nucleases (e.g., RNases) in an RNA-immunoprecipitation assay that employs an antibody that targets a component of PRC2, or a region of genomic DNA that encodes that protected RNA region. In some embodiments, a PRC2-associated region is a region of an RNA that is protected from nucleases (e.g., RNases) in an RNA-immunoprecipitation assay that employs an antibody that targets SUZ12, EED, EZH2 or RBBP4, or a region of genomic DNA that encodes that protected RNA region.

In some embodiments, a PRC2-associated region is a region of an RNA within which occur a relatively high frequency of sequence reads in a sequencing reaction of products of an RNA-immunoprecipitation assay that employs an antibody that targets a component of PRC2, or a region of genomic DNA that encodes that RNA region. In some embodiments, a PRC2-associated region is a region of an RNA within which occur a relatively high frequency of sequence reads in a sequencing reaction of products of an RNA-immunoprecipitation assay that employs an antibody that binds specifically to SUZ12, EED, EZH2 or RBBP4, or a region of genomic DNA that encodes that protected RNA region. In such embodiments, the PRC2-associated region may be referred to as a “peak.”

In some embodiments, a PRC2-associated region comprises a sequence of 40 to 60 nucleotides that interact with PRC2 complex. In some embodiments, a PRC2-associated region comprises a sequence of 40 to 60 nucleotides that encode an RNA that interacts with PRC2. In some embodiments, a PRC2-associated region comprises a sequence of up to 5 kb in length that comprises a sequence (e.g., of 40 to 60 nucleotides) that interacts with PRC2. In some embodiments, a PRC2-associated region comprises a sequence of up to 5 kb in length within which an RNA is encoded that has a sequence (e.g., of 40 to 60 nucleotides) that is known to interact with PRC2. In some embodiments, a PRC2-associated region comprises a sequence of about 4 kb in length that comprise a sequence (e.g., of 40 to 60 nucleotides) that interacts with PRC2. In some embodiments, a PRC2-associated region comprises a sequence of about 4 kb in length within which an RNA is encoded that includes a sequence (e.g., of 40 to 60 nucleotides) that is known to interact with PRC2. In some embodiments, a PRC2-associated region has a sequence as set forth in any one of SEQ ID NOS: SEQ ID NOS: 8-45 or 48-59.

In some embodiments, single stranded oligonucleotides are provided that specifically bind to, or are complementary to, a PRC2-associated region in a genomic region that encompasses or that is in proximity to the FOXP3 gene. In some embodiments, single stranded oligonucleotides are provided that specifically bind to, or are complementary to, a PRC2-associated region that has a sequence as set forth in any one of SEQ ID NOS: 8-45 or 48-59. In some embodiments, single stranded oligonucleotides are provided that specifically bind to, or are complementary to, a PRC2-associated region that has a sequence as set forth in any one of SEQ ID NOS: 8-45 or 48-59 combined with up to 2 kb, up to 5 kb, or up to 10 kb of flanking sequences from a corresponding genomic region to which these SEQ IDs map (e.g., in a human genome). In some embodiments, single stranded oligonucleotides have a sequence as set forth in any one of SEQ ID NOS: 60-45713. In some embodiments, a single stranded oligonucleotide has a sequence as set forth in Table 4.

Without being bound by a theory of invention, these oligonucleotides are able to interfere with the binding of and function of PRC2, by preventing recruitment of PRC2 to a specific chromosomal locus. For example, a single administration of single stranded oligonucleotides designed to specifically bind a PRC2-associated region lncRNA can stably displace not only the lncRNA, but also the PRC2 that binds to the lncRNA, from binding chromatin. After displacement, the full complement of PRC2 is not recovered for up to 24 hours. Further, lncRNA can recruit PRC2 in a cis fashion, repressing gene expression at or near the specific chromosomal locus from which the lncRNA was transcribed.

Methods of modulating gene expression are provided, in some embodiments, that may be carried out in vitro, ex vivo, or in vivo. It is understood that any reference to uses of compounds throughout the description contemplates use of the compound in preparation of a pharmaceutical composition or medicament for use in the treatment of condition or a disease (e.g., a disease or disorder associated with aberrant immune cell activation such as an autoimmune or inflammatory disease or disorder) associated with decreased levels or activity of FOXP3. Thus, as one nonlimiting example, this aspect of the invention includes use of such single stranded oligonucleotides in the preparation of a medicament for use in the treatment of disease, wherein the treatment involves upregulating expression of FOXP3.

In further aspects of the invention, methods are provided for selecting a candidate oligonucleotide for activating expression of FOXP3. The methods generally involve selecting as a candidate oligonucleotide, a single stranded oligonucleotide comprising a nucleotide sequence that is complementary to a PRC2-associated region (e.g., a nucleotide sequence as set forth in any one of SEQ ID NOS: 8-45 or 48-59). In some embodiments, sets of oligonucleotides may be selected that are enriched (e.g., compared with a random selection of oligonucleotides) in oligonucleotides that activate expression of FOXP3.

### Single Stranded Oligonucleotides for Modulating Expression of FOXP3

In one aspect of the invention, single stranded oligonucleotides complementary to the PRC2-associated regions are provided for modulating expression of FOXP3 in a cell. In some embodiments, expression of FOXP3 is upregulated or increased. In some embodiments, single stranded oligonucleotides complementary to these PRC2-associated regions inhibit the interaction of PRC2 with long RNA transcripts such that gene expression is upregulated or increased. In some embodiments, single stranded oligonucleotides complementary to these PRC2-associated regions inhibit the interaction of PRC2 with long RNA transcripts, resulting in reduced methylation of histone H3 and reduced gene inactivation, such that gene expression is upregulated or increased. In some embodiments, this interaction may be disrupted or inhibited due to a change in the structure of the long RNA that prevents or reduces binding to PRC2. The oligonucleotide may be selected using any of the methods disclosed herein for selecting a candidate oligonucleotide for activating expression of FOXP3.

The single stranded oligonucleotide may comprise a region of complementarity that is complementary with a PRC2-associated region of a nucleotide sequence set forth in any one of SEQ ID NOS: 1-7, 46, or 47. The region of complementarity of the single stranded oligonucleotide may be complementary with at least 6, e.g., at least 7, at least 8, at least 9, at least 10, at least 15 or more consecutive nucleotides of the PRC2-associated region.

The PRC2-associated region of a FOXP3 gene may map to a position in a chromosome between 50 kilobases upstream of a 5′-end of the FOXP3 gene and 50 kilobases downstream of a 3′-end of the FOXP3 gene. For example, the PRC2 associated region of a FOXP3 gene may have a sequence that maps to a position in chromosome X of a human genome within the coordinates chrX:49,057,795-49,164,962, based on the February 2009 UCSC genome assembly (GRCh37/hg19). The PRC2-associated region may map to a position in a chromosome between 25 kilobases upstream of a 5′-end of the FOXP3 gene and 25 kilobases downstream of a 3′-end of the FOXP3 gene. The PRC2-associated region may map to a position in a chromosome between 12 kilobases upstream of a 5′-end of the FOXP3 gene and 12 kilobases downstream of a 3′-end of the FOXP3 gene. The PRC2-associated region may map to a position in a chromosome between 5 kilobases upstream of a 5′-end of the FOXP3 gene and 5 kilobases downstream of a 3′-end of the FOXP3 gene.

The genomic position of the selected PRC2-associated region relative to the FOXP3 gene may vary. For example, the PRC2-associated region may be upstream of the 5′ end of the FOXP3 gene. The PRC2-associated region may be downstream of the 3′ end of the FOXP3 gene. The PRC2-associated region may be within an intron of the FOXP3 gene. The PRC2-associated region may be within an exon of the FOXP3 gene. The PRC2-associated region may traverse an intron-exon junction, a 5′-UTR-exon junction or a 3′-UTR-exon junction of the FOXP3 gene.

The single stranded oligonucleotide may comprise a sequence having the formula X-Y-Z, in which X is any nucleotide, Y is a nucleotide sequence of 6 nucleotides in length that is not a human seed sequence of a microRNA, and Z is a nucleotide sequence of varying length. In some embodiments X is the 5′ nucleotide of the oligonucleotide. In some embodiments, when X is anchored at the 5′ end of the oligonucleotide, the oligonucleotide does not have any nucleotides or nucleotide analogs linked 5′ to X. In some embodiments, other compounds such as peptides or sterols may be linked at the 5′ end in this embodiment as long as they are not nucleotides or nucleotide analogs. In some embodiments, the single stranded oligonucleotide has a sequence 5′X-Y-Z and is 8-50 nucleotides in length. Oligonucleotides that have these sequence characteristics are predicted to avoid the miRNA pathway. Therefore, in some embodiments, oligonucleotides having these sequence characteristics are unlikely to have an unintended consequence of functioning in a cell as a miRNA molecule. The Y sequence may be a nucleotide sequence of 6 nucleotides in length set forth in Table 1.

The single stranded oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches.

The single stranded oligonucleotide may have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than FOXP3. In a similar embodiment, an oligonucleotide may be designed to ensure that it does not have a sequence that maps to any other known PRC2-associated region, particularly PRC2-associated regions that are functionally related to any other known gene (e.g., any other known protein coding gene). In either case, the oligonucleotide is expected to have a reduced likelihood of having off-target effects. The threshold level of sequence identity may be 50%, 60%, 70%, 80%, 85%, 90%, 95%, 99% or 100% sequence identity.

The single stranded oligonucleotide may have a sequence that is complementary to a PRC2-associated region that encodes an RNA that forms a secondary structure comprising at least two single stranded loops. In has been discovered that, in some embodiments, oligonucleotides that are complementary to a PRC2-associated region that encodes an RNA that forms a secondary structure comprising one or more single stranded loops (e.g., at least two single stranded loops) have a greater likelihood of being active (e.g., of being capable of activating or enhancing expression of a target gene) than a randomly selected oligonucleotide. In some cases, the secondary structure may comprise a double stranded stem between the at least two single stranded loops. Accordingly, the region of complementarity between the oligonucleotide and the PRC2-associated region may be at a location of the PRC2 associated region that encodes at least a portion of at least one of the loops. In some cases, the region of complementarity between the oligonucleotide and the PRC2-associated region may be at a location of the PRC2-associated region that encodes at least a portion of at least two of the loops. In some cases, the region of complementarity between the oligonucleotide and the PRC2-associated region may be at a location of the PRC2 associated region that encodes at least a portion of the double stranded stem. In some embodiments, a PRC2-associated region (e.g., of an lncRNA) is identified (e.g., using RIP-Seq methodology or information derived therefrom [see, e.g., Zhao et al. Genome-wide identification of Polycomb-associated RNAs by RIP-seq. Mol Cell. 2010 Dec. 22; 40(6): 939-953]). In some embodiments, the predicted secondary structure RNA (e.g., lncRNA) containing the PRC2-associated region is determined using RNA secondary structure prediction algorithms, e.g., RNAfold, mfold. In some embodiments, oligonucleotides are designed to target a region of the RNA that forms a secondary structure comprising one or more single stranded loop (e.g., at least two single stranded loops) structures which may comprise a double stranded stem between the at least two single stranded loops.

The single stranded oligonucleotide may have a sequence that is has greater than 30% G-C content, greater than 40% G-C content, greater than 50% G-C content, greater than 60% G-C content, greater than 70% G-C content, or greater than 80% G-C content. The single stranded oligonucleotide may have a sequence that has up to 100% G-C content, up to 95% G-C content, up to 90% G-C content, or up to 80% G-C content. In some embodiments in which the oligonucleotide is 8 to 10 nucleotides in length, all but 1, 2, 3, 4, or 5 of the nucleotides of the complementary sequence of the PRC2-associated region are cytosine or guanosine nucleotides. In some embodiments, the sequence of the PRC2-associated region to which the single stranded oligonucleotide is complementary comprises no more than 3 nucleotides selected from adenine and uracil.

The single stranded oligonucleotide may be complementary to a chromosome of a different species (e.g., a mouse, rat, rabbit, goat, monkey, etc.) at a position that encompasses or that is in proximity to that species' homolog of FOXP3. The single stranded oligonucleotide may be complementary to a human genomic region encompassing or in proximity to the FOXP3 gene and also be complementary to a mouse genomic region encompassing or in proximity to the mouse homolog of FOXP3. For example, the single stranded oligonucleotide may be complementary to a sequence as set forth in SEQ ID NO: 1, 2, 5, 6, 7, 46, or 47, which is a human genomic region encompassing or in proximity to the FOXP3 gene, and also be complementary to a sequence as set forth in SEQ ID NO: 3 or 4, which is a mouse genomic region encompassing or in proximity to the mouse homolog of the FOXP3 gene. Oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of the single stranded oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a PRC2-associated region. In some embodiments, the region of complementarity is complementary with at least 8 consecutive nucleotides of a PRC2-associated region. In some embodiments the sequence of the single stranded oligonucleotide is based on an RNA sequence that binds to PRC2, or a portion thereof, said portion having a length of from 5 to 40 contiguous base pairs, or about 8 to 40 bases, or about 5 to 15, or about 5 to 30, or about 5 to 40 bases, or about 5 to 50 bases.

Complementary, as the term is used in the art, refers to the capacity for precise pairing between two nucleotides. For example, if a nucleotide at a certain position of an oligonucleotide is capable of hydrogen bonding with a nucleotide at the same position of PRC2-associated region, then the single stranded nucleotide and PRC2-associated region are considered to be complementary to each other at that position. The single stranded nucleotide and PRC2-associated region are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the single stranded nucleotide and PRC2-associated region. For example, if a base at one position of a single stranded nucleotide is capable of hydrogen bonding with a base at the corresponding position of a PRC2-associated region, then the bases are considered to be complementary to each other at that position. 100% complementarity is not required.

The single stranded oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a PRC2-associated region. In some embodiments the single stranded oligonucleotide may contain 1, 2 or 3 base mismatches compared to the portion of the consecutive nucleotides of a PRC2-associated region. In some embodiments the single stranded oligonucleotide may have up to 3 mismatches over 15 bases, or up to 2 mismatches over 10 bases.

It is understood in the art that a complementary nucleotide sequence need not be 100% complementary to that of its target to be specifically hybridizable. In some embodiments, a complementary nucleic acid sequence for purposes of the present disclosure is specifically hybridizable when binding of the sequence to the target molecule (e.g., lncRNA) interferes with the normal function of the target (e.g., lncRNA) to cause a loss of activity (e.g., inhibiting PRC2-associated repression with consequent up-regulation of gene expression) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the single stranded oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. In a preferred embodiment, the oligonucleotide is 8 to 30 nucleotides in length.

In some embodiments, the PRC2-associated region occurs on the same DNA strand as a gene sequence (sense). In some embodiments, the PRC2-associated region occurs on the opposite DNA strand as a gene sequence (anti-sense). Oligonucleotides complementary to a PRC2-associated region can bind either sense or anti-sense sequences. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, GC content of the single stranded oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

In some embodiments, the single stranded oligonucleotide specifically binds to, or is complementary to an RNA that is encoded in a genome (e.g., a human genome) as a single contiguous transcript (e.g., a non-spliced RNA). In some embodiments, the single stranded oligonucleotide specifically binds to, or is complementary to an RNA that is encoded in a genome (e.g., a human genome), in which the distance in the genome between the 5′ end of the coding region of the RNA and the 3′ end of the coding region of the RNA is less than 1 kb, less than 2 kb, less than 3 kb, less than 4 kb, less than 5 kb, less than 7 kb, less than 8 kb, less than 9 kb, less than 10 kb, or less than 20 kb.

It is to be understood that any oligonucleotide provided herein can be excluded.

In some embodiments, it has been found that single stranded oligonucleotides disclosed herein may increase expression of mRNA corresponding to a target gene by at least about 50% (i.e. 150% of normal or 1.5 fold), or by about 2 fold to about 5 fold. In some embodiments, expression may be increased by at least about 15 fold, 20 fold, 30 fold, 40 fold, 50 fold or 100 fold, or any range between any of the foregoing numbers. It has also been found that increased mRNA expression has been shown to correlate to increased protein expression.

In some embodiments, it has been found that single stranded oligonucleotides disclosed herein may increase expression of mRNA or protein corresponding to CTLA4, GITR, and/or IL-10 by at least about 30% (i.e. 130% of normal or 1.3 fold), or by about 1.5 fold, or by about 2 fold to about 5 fold. In some embodiments, expression may be increased by at least about 5 fold, 10 fold, 15 fold, 20 fold, 30 fold, 40 fold, 50 fold or 100 fold, or any range between any of the foregoing numbers. For example, mRNA or protein corresponding to CTLA4, GITR, and/or IL-10 may be increased by an amount in a range of 1.3 fold to 2 fold, 1.3 fold to 5 fold, 1.3 fold to 10 fold, 1.3 fold to 20 fold, 1.3 fold to 50 fold, 1.3 fold to 100 fold, 2 fold to 5 fold, 2 fold to 10 fold, 2 fold to 20 fold, 2 fold to 10 fold. 2 fold to 20 fold, 2 fold to 50 fold, or 2 fold to 100 fold. Exemplary human mRNA and protein sequence identifiers for CTLA4, GITR, and IL-10 are provided below. These sequence identifiers can be used to identify exemplary mRNA and protein sequences for CTLA4, GITR, and IL-10 by using the NCBI Gene search as of the filing of the instant application.

CTLA4: NM_001037631.2, NM_005214.4, NP_001032720.1, NP_005205.2

GITR (also called TNFRSF18): NM_004195.2, NM_148901.1, NM_148902.1, NP_004186.1, NP_683699.1, NP_683700.1

IL-10: NM_000572.2, NP_000563.1

In some embodiments, it has been found that single stranded oligonucleotides disclosed herein may increase the number of CD4+CD25+FOXP3+ T cells by at least about 30% (i.e. 130% of normal or 1.3 fold), or by about 1.5 fold, or by about 2 fold to about 5 fold. In some embodiments, the number may be increased by at least about 5 fold, 10 fold, 15 fold, 20 fold, 30 fold, 40 fold, 50 fold or 100 fold, or any range between any of the foregoing numbers. For example, numbers of CD4+CD25+FOXP3+ T cells may be increased in a population of T cells by an amount in a range of 1.3 fold to 2 fold, 1.3 fold to 5 fold, 1.3 fold to 10 fold, 1.3 fold to 20 fold, 1.3 fold to 50 fold, 1.3 fold to 100 fold, 2 fold to 5 fold, 2 fold to 10 fold, 2 fold to 20 fold, 2 fold to 10 fold. 2 fold to 20 fold, 2 fold to 50 fold, or 2 fold to 100 fold.

In some or any of the embodiments of the oligonucleotides described herein, or processes for designing or synthesizing them, the oligonucleotides will upregulate gene expression and may specifically bind or specifically hybridize or be complementary to the PRC2 binding RNA that is transcribed from the same strand as a protein coding reference gene. The oligonucleotide may bind to a region of the PRC2 binding RNA that originates within or overlaps an intron, exon, intron exon junction, 5′ UTR, 3′ UTR, a translation initiation region, or a translation termination region of a protein coding sense strand of a reference gene (refGene).

In some or any of the embodiments of oligonucleotides described herein, or processes for designing or synthesizing them, the oligonucleotides will upregulate gene expression and may specifically bind or specifically hybridize or be complementary to a PRC2 binding RNA that transcribed from the opposite strand (the antisense strand) of a protein coding reference gene. The oligonucleotide may bind to a region of the PRC2 binding RNA that originates within or overlaps an intron, exon, intron exon junction, 5′ UTR, 3′ UTR, a translation initiation region, or a translation termination region of a protein coding antisense strand of a reference gene

The oligonucleotides described herein may be modified, e.g., comprise a modified sugar moiety, a modified internucleoside linkage, a modified nucleotide and/or combinations thereof. In addition, the oligonucleotides can exhibit one or more of the following properties: do not induce substantial cleavage or degradation of the target RNA; do not cause substantially complete cleavage or degradation of the target RNA; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; may have improved endosomal exit; do interfere with interaction of lncRNA with PRC2, preferably the Ezh2 subunit but optionally the Suz12, Eed, RbAp46/48 subunits or accessory factors such as Jarid2; do decrease histone H3 lysine27 methylation and/or do upregulate gene expression.

Oligonucleotides that are designed to interact with RNA to modulate gene expression are a distinct subset of base sequences from those that are designed to bind a DNA target (e.g., are complementary to the underlying genomic DNA sequence from which the RNA is transcribed).

Any of the oligonucleotides disclosed herein may be linked to one or more other oligonucleotides disclosed herein by a linker, e.g., a cleavable linker.

### Method for Selecting Candidate Oligonucleotides for Activating Expression of FOXP3

Methods are provided herein for selecting a candidate oligonucleotide for activating or enhancing expression of FOXP3. The target selection methods may generally involve steps for selecting single stranded oligonucleotides having any of the structural and functional characteristics disclosed herein. Typically, the methods involve one or more steps aimed at identifying oligonucleotides that target a PRC2-associated region that is functionally related to FOXP3, for example a PRC2-associated region of a lncRNA that regulates expression of FOXP3 by facilitating (e.g., in a cis-regulatory manner) the recruitment of PRC2 to the FOXP3 gene. Such oligonucleotides are expected to be candidates for activating expression of FOXP3 because of their ability to hybridize with the PRC2-associated region of a nucleic acid (e.g., a lncRNA). In some embodiments, this hybridization event is understood to disrupt interaction of PRC2 with the nucleic acid (e.g., a lncRNA) and as a result disrupt recruitment of PRC2 and its associated co-repressors (e.g., chromatin remodeling factors) to the FOXP3 gene locus.

Methods of selecting a candidate oligonucleotide may involve selecting a PRC2-associated region (e.g., a nucleotide sequence as set forth in any one of SEQ ID NOS: 8-45 or 48-59) that maps to a chromosomal position encompassing or in proximity to the FOXP3 gene (e.g., a chromosomal position having a sequence as set forth in any one of SEQ ID NOS: 1-7, 46, or 47). The PRC2-associated region may map to the strand of the chromosome comprising the sense strand of the FOXP3 gene, in which case the candidate oligonucleotide is complementary to the sense strand of the FOXP3 gene (i.e., is antisense to the FOXP3 gene). Alternatively, the PRC2-associated region may map to the strand of the first chromosome comprising the antisense strand of the FOXP3 gene, in which case the oligonucleotide is complementary to the antisense strand (the template strand) of the FOXP3 gene (i.e., is sense to the FOXP3 gene).

Methods for selecting a set of candidate oligonucleotides that is enriched in oligonucleotides that activate expression of FOXP3 may involve selecting one or more PRC2-associated regions that map to a chromosomal position that encompasses or that is in proximity to the FOXP3 gene and selecting a set of oligonucleotides, in which each oligonucleotide in the set comprises a nucleotide sequence that is complementary with the one or more PRC2-associated regions. As used herein, the phrase, “a set of oligonucleotides that is enriched in oligonucleotides that activate expression of” refers to a set of oligonucleotides that has a greater number of oligonucleotides that activate expression of a target gene (e.g., FOXP3) compared with a random selection of oligonucleotides of the same physicochemical properties (e.g., the same GC content, Tₘ, length etc.) as the enriched set.

Where the design and/or synthesis of a single stranded oligonucleotide involves design and/or synthesis of a sequence that is complementary to a nucleic acid or PRC2-associated region described by such sequence information, the skilled person is readily able to determine the complementary sequence, e.g., through understanding of Watson Crick base pairing rules which form part of the common general knowledge in the field.

In some embodiments design and/or synthesis of a single stranded oligonucleotide involves manufacture of an oligonucleotide from starting materials by techniques known to those of skill in the art, where the synthesis may be based on a sequence of a PRC2-associated region, or portion thereof.

Methods of design and/or synthesis of a single stranded oligonucleotide may involve one or more of the steps of:

Identifying and/or selecting PRC2-associated region;

Designing a nucleic acid sequence having a desired degree of sequence identity or complementarity to a PRC2-associated region or a portion thereof;

Synthesizing a single stranded oligonucleotide to the designed sequence;

Purifying the synthesized single stranded oligonucleotide; and

Optionally mixing the synthesized single stranded oligonucleotide with at least one pharmaceutically acceptable diluent, carrier or excipient to form a pharmaceutical composition or medicament.

Single stranded oligonucleotides so designed and/or synthesized may be useful in method of modulating gene expression as described herein.

Preferably, single stranded oligonucleotides of the invention are synthesized chemically. Oligonucleotides used to practice this invention can be synthesized in vitro by well-known chemical synthesis techniques.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O—NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

It is understood that any of the modified chemistries or formats of single stranded oligonucleotides described herein can be combined with each other, and that one, two, three, four, five, or more different types of modifications can be included within the same molecule.

In some embodiments, the method may further comprise the steps of amplifying the synthesized single stranded oligonucleotide, and/or purifying the single stranded oligonucleotide (or amplified single stranded oligonucleotide), and/or sequencing the single stranded oligonucleotide so obtained.

As such, the process of preparing a single stranded oligonucleotide may be a process that is for use in the manufacture of a pharmaceutical composition or medicament for use in the treatment of disease, optionally wherein the treatment involves modulating expression of a gene associated with a PRC2-associated region.

In the methods described above a PRC2-associated region may be, or have been, identified, or obtained, by a method that involves identifying RNA that binds to PRC2.

Such methods may involve the following steps: providing a sample containing nuclear ribonucleic acids, contacting the sample with an agent that binds specifically to PRC2 or a subunit thereof, allowing complexes to form between the agent and protein in the sample, partitioning the complexes, synthesizing nucleic acid that is complementary to nucleic acid present in the complexes.

Where the single stranded oligonucleotide is based on a PRC2-associated region, or a portion of such a sequence, it may be based on information about that sequence, e.g., sequence information available in written or electronic form, which may include sequence information contained in publicly available scientific publications or sequence databases.

### Nucleotide Analogues

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the single stranded oligonucleotide has one or more nucleotide analogues. For example, the single stranded oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The single stranded oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Preferably the single stranded oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the single stranded oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric single stranded oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the single stranded oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone, CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-O,4′-C-ethylene-bridged nucleic acids.

Examples of LNAs are described in WO/2008/043753 and include compounds of the following general formula.

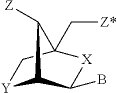

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligonucleotides described herein comprises at least one LNA unit according any of the formulas

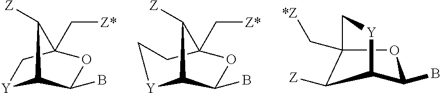

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown in scheme 2:

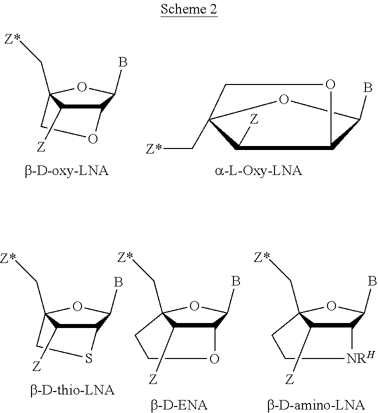

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃OCH₃, OCH₃O(CH₂)nCH₃, O(CH₂)nNH₂ or O(CH₂)nCH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Single stranded oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Single stranded oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the single stranded oligonucleotides are chemically linked to one or more moieties or conjugates that enhance the activity, cellular distribution, or cellular uptake of the oligonucleotide. For example, one or more single stranded oligonucleotides, of the same or different types, can be conjugated to each other; or single stranded oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, single stranded oligonucleotide modification include modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of the single stranded oligonucleotide. In some embodiments, the single stranded oligonucleotide comprises a biotin moiety conjugated to the 5′ nucleotide.

In some embodiments, the single stranded oligonucleotide comprises locked nucleic acids (LNA), ENA modified nucleotides, 2′-O-methyl nucleotides, or 2′-fluoro-deoxyribonucleotides. In some embodiments, the single stranded oligonucleotide comprises alternating deoxyribonucleotides and 2′-fluoro-deoxyribonucleotides. In some embodiments, the single stranded oligonucleotide comprises alternating deoxyribonucleotides and 2′-O-methyl nucleotides. In some embodiments, the single stranded oligonucleotide comprises alternating deoxyribonucleotides and ENA modified nucleotides. In some embodiments, the single stranded oligonucleotide comprises alternating deoxyribonucleotides and locked nucleic acid nucleotides. In some embodiments, the single stranded oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the single stranded oligonucleotide comprises phosphorothioate internucleotide linkages. In some embodiments, the single stranded oligonucleotide comprises phosphorothioate internucleotide linkages between at least two nucleotides. In some embodiments, the single stranded oligonucleotide comprises phosphorothioate internucleotide linkages between all nucleotides.

It should be appreciated that the single stranded oligonucleotide can have any combination of modifications as described herein.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

Aspects of the disclosure relate to methods for inducing FOXP3 expression, activating T cells, and/or treating a condition or disease (e.g., a disease or disorder associated with aberrant immune cell activation such as an autoimmune or inflammatory disease or disorder) associated with decreased levels of FOXP3 that involve inhibiting expression or activity of EZH1 and/or EZH2 or another component of PRC2, e.g., Suz12, EED1 or RbAp48. For example, expression of EZH1 and/or EZH2 may inhibited through the using any of oligonucleotides (e.g., single stranded oligonucleotides) disclosed herein. In some embodiments, expression or activity may be inhibited through the use of a gapmer, siRNA, miRNA or other oligonucleotide that inhibits expression of a target mRNA.

Exemplary human mRNA and protein sequence identifiers for EZH1, EZH2, Suz12, EED1 and RbAp48 are provided below. These sequence identifiers can be used to identify exemplary mRNA and protein sequences by using the NCBI Gene search as of the filing of the instant application.

EZH1: NM_001991.3, NP_001982.2

EZH2: NM_001203247.1, NM_001203248.1, NM_001203249.1, NM_004456.4, NP_004447.2, NM_152998.2, NP_001190177.1, NP_001190176.1, NP_001190178.1, NP_694543.1

Suz12: NM_015355.2, NP_056170.2

EED1: NM_003797.3, NM_152991.2, NP_003788.2, NP_694536.1

RbAp48: NM_001135255.1, NM_001135256.1, NM_005610.2, NP_001128727.1, NP_001128728.1, NP_005601.1.

Accordingly, in some embodiments, gapmer oligonucleotides are provided herein. In some embodiments, a gapmer oligonucleotide has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAseH. Without wishing to be bound by theory, it is thought that the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., 1-6 modified nucleotides. Exemplary modified oligonucleotides include, but are not limited to, 2′ MOE or 2′OMe or Locked Nucleic Acid bases (LNA). The flanks X and Z may be have a of length 1-20 nucleotides, preferably 1-8 nucleotides and even more preferred 1-5 nucleotides. The flanks X and Z may be of similar length or of dissimilar lengths. The gap-segment Y may be a nucleotide sequence of length 5-20 nucleotides, preferably 6-12 nucleotides and even more preferred 6-10 nucleotides. In some aspects, the gap region of the gapmer oligonucleotides of the invention may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

In some embodiments, oligonucleotides provided herein may be in the form of small interfering RNAs (siRNA), also known as short interfering RNA or silencing RNA. SiRNA, is a class of double-stranded RNA molecules, typically about 18-23 or 20-25 base pairs in length that target nucleic acids (e.g., mRNAs) for degradation via the RNA interference (RNAi) pathway in cells. Specificity of siRNA molecules may be determined by the binding of the antisense strand of the molecule to its target RNA. Effective siRNA molecules are generally less than 30 to 35 base pairs in length to prevent the triggering of non-specific RNA interference pathways in the cell via the interferon response, although longer siRNA can also be effective. The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

Double-stranded siRNA may comprise RNA strands that are the same length or different lengths. Double-stranded siRNA molecules can also be assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi. Small hairpin RNA (shRNA) molecules thus are also contemplated herein. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form a dsRNA molecule (optionally with additional processing steps that may result in addition or removal of one, two, three or more nucleotides from the 3′ end and/or the 5′ end of either or both strands). A spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). A spacer sequence is may be an unrelated nucleotide sequence that is situated between two complementary nucleotide sequence regions which, when annealed into a double-stranded nucleic acid, comprise a shRNA.

The overall length of the siRNA molecules can vary from about 14 to about 200 nucleotides, e.g., about 14-100, 14-50, 14-30 or 18-23 nucleotides, depending on the type of siRNA molecule being designed. Generally between about 14 and about 50 of these nucleotides are complementary to the RNA target sequence, i.e. constitute the specific antisense sequence of the siRNA molecule. For example, when the siRNA is a double- or single-stranded siRNA, the length can vary from about 14 to about 50 nucleotides, whereas when the siRNA is a shRNA or circular molecule, the length can vary from about 40 nucleotides to about 200 nucleotides.

An siRNA molecule may comprise a 3′ overhang at one end of the molecule. The other end may be blunt-ended or have also an overhang (5′ or 3′). When the siRNA molecule comprises an overhang at both ends of the molecule, the length of the overhangs may be the same or different. In one embodiment, the siRNA molecule of the present invention comprises 3′ overhangs of about 1 to about 3 nucleotides on both ends of the molecule.

In some embodiments, an oligonucleotide may be a microRNA (miRNA). MicroRNAs (referred to as “miRNAs”) are small non-coding RNAs, belonging to a class of regulatory molecules found in plants and animals that control gene expression by binding to complementary sites on a target RNA transcript. miRNAs are generated from large RNA precursors (termed pri-miRNAs) that are processed in the nucleus into approximately 70 nucleotide pre-miRNAs, which fold into imperfect stem-loop structures (Lee, Y., et al., Nature (2003) 425(6956):415-9). The pre-miRNAs undergo an additional processing step within the cytoplasm where mature miRNAs of 18-25 nucleotides in length are excised from one side of the pre-miRNA hairpin by an RNase III enzyme, Dicer (Hutvagner, G., et al., Science (2001) 12:12 and Grishok, A., et al., Cell (2001) 106(1):23-34).

As used herein, miRNAs including pri-miRNA, pre-miRNA, mature miRNA or fragments of variants thereof that retain the biological activity of mature miRNA. In one embodiment, the size range of the miRNA can be from 21 nucleotides to 170 nucleotides, although miRNAs of up to 2000 nucleotides can be utilized. In a preferred embodiment the size range of the miRNA is from 70 to 170 nucleotides in length. In another preferred embodiment, mature miRNAs of from 21 to 25 nucleotides in length can be used.

In some embodiments, the miRNA may be a miR-30 precursor. As used herein, an “miR-30 precursor”, also called an miR-30 hairpin, is a precursor of the human microRNA miR-30, as it is understood in the literature (e.g., Zeng and Cullen, 2003; Zeng and Cullen, 2005; Zeng et al., 2005; United States Patent Application Publication No. US 2004/005341), where the precursor could be modified from the wild-type miR-30 precursor in any manner described or implied by that literature, while retaining the ability to be processed into an miRNA. In some embodiments, a miR-30 precursor is at least 80 nucleotides long and comprises a stem-loop structure. In some embodiments, the miR-30 precursor further comprises a first miRNA sequence of 20-22 nucleotides on the stem of the stem-loop structure complementary to a portion of a first target sequence (e.g., a sequence within a euchromatic region of a target gene disclosed herein).

A miRNA may be isolated from a variety of sources or may be synthesized according to methods well known in the art (see, e.g., Current Protocols in Molecular Biology, Wiley Online Library; U.S. Pat. No. 8,354,384; and Wahid et al. MicroRNAs: synthesis, mechanism, function, and recent clinical trials. Biochim Biophys Acta. 2010; 1803(11):1231-43). In some embodiments, a miRNA is expressed from a vector as known in the art or described herein. In some embodiments, the vector may include a sequence encoding a mature miRNA. In some embodiments, the vector may include a sequence encoding a pre-miRNA such that the pre-miRNA is expressed and processed in a cell into a mature miRNA. In some embodiments, the vector may include a sequence encoding a pri-miRNA. In this embodiment, the primary transcript is first processed to produce the stem-loop precursor miRNA molecule. The stem-loop precursor is then processed to produce the mature microRNA.

### Methods for Modulating Gene Expression

In one aspect, the invention relates to methods for modulating gene expression in a cell (e.g., a cell for which FOXP3 levels are reduced) for research purposes (e.g., to study the function of the gene in the cell). In another aspect, the invention relates to methods for modulating gene expression in a cell (e.g., a cell for which FOXP3 levels are reduced) for gene or epigenetic therapy. The cells can be in vitro, ex vivo, or in vivo (e.g., in a subject who has a disease or condition resulting from reduced expression or activity of FOXP3). In some embodiments, methods for modulating gene expression in a cell comprise delivering a single stranded oligonucleotide as described herein. In some embodiments, delivery of the single stranded oligonucleotide to the cell results in a level of expression of gene that is at least 5%, 10%, 20%, 30%, 40%, 50%, 60%, 70%, 80%, 90%, 100%, 200% or more greater than a level of expression of gene in a control cell to which the single stranded oligonucleotide has not been delivered. In certain embodiments, delivery of the single stranded oligonucleotide to the cell results in a level of expression of gene that is at least 50% greater than a level of expression of gene in a control cell to which the single stranded oligonucleotide has not been delivered.

In another aspect of the invention, methods comprise administering to a subject (e.g. a human) a composition comprising a single stranded oligonucleotide as described herein to increase protein levels in the subject. In some embodiments, the increase in protein levels is at least 5%, 10%, 20%, 30%, 40%, 50%, 60%, 70%, 80%, 90%, 100%, 200%, or more, higher than the amount of a protein in the subject before administering.

As another example, to increase expression of FOXP3 in a cell, the methods include introducing into the cell a single stranded oligonucleotide that is sufficiently complementary to a PRC2-associated region (e.g., of a long non-coding RNA) that maps to a genomic position encompassing or in proximity to the FOXP3 gene.

In another aspect of the invention provides methods of treating a condition (e.g., a disease or disorder associated with aberrant immune cell activation such as an autoimmune disease or disorder) associated with decreased levels of expression of FOXP3 in a subject, the method comprising administering a single stranded oligonucleotide as described herein.

A subject can include a non-human mammal, e.g. mouse, rat, guinea pig, rabbit, cat, dog, goat, cow, or horse. In preferred embodiments, a subject is a human. Single stranded oligonucleotides have been employed as therapeutic moieties in the treatment of disease states in animals, including humans. Single stranded oligonucleotides can be useful therapeutic modalities that can be configured to be useful in treatment regimes for the treatment of cells, tissues and animals, especially humans.

For therapeutics, an animal, preferably a human, suspected of having a disease or disorder associated with aberrant immune cell activation such as an autoimmune disease or disorder is treated for the disease or disorder by administering single stranded oligonucleotide in accordance with this invention. For example, in one non-limiting embodiment, the methods comprise the step of administering to an animal in need of treatment, a therapeutically effective amount of a single stranded oligonucleotide as described herein.

Examples of autoimmune diseases and disorders that may be treated according to the methods disclosed herein include, but are not limited to, Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, inflammatory bowel disease (e.g., Crohn's disease or Ulcerative colitis), Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, IPEX (Immunodysregulation, Polyendocrinopathy, and Enteropathy, X-linked) syndrome, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), systemic lupus erythematosus (SLE), chronic Lyme disease, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Undifferentiated connective tissue disease (UCTD), Uveitis, Vasculitis, Vesiculobullous dermatosis, Vitiligo, and Wegener's granulomatosis (also called Granulomatosis with Polyangiitis (GPA)). In some embodiments, the autoimmune disease or disorder is inflammatory bowel disease (e.g., Crohn's disease or Ulcerative colitis), IPEX syndrome, Multiple sclerosis, Psoriasis, Rheumatoid arthritis, SLE or Type 1 diabetes.

Examples of inflammatory diseases or disorders that may be treated according to the methods disclosed herein include, but are not limited to, Acne Vulgaris, Appendicitis, Arthritis, Asthma, Atherosclerosis, Allergies (Type 1 Hypersensitivity), Bursitis, Colitis, Chronic Prostatitis, Cystitis, Dermatitis, Glomerulonephritis, Inflammatory Bowel Disease, Inflammatory Myopathy (e.g., Polymyositis, Dermatomyositis, or Inclusion-body Myositis), Inflammatory Lung Disease, Interstitial Cystitis, Meningitis, Pelvic Inflammatory Disease, Phlebitis, Psoriasis, Reperfusion Injury, Rheumatoid Arthritis, Sarcoidosis, Tendonitis, Tonsilitis, Transplant Rejection, and Vasculitis. In some embodiments, the inflammatory disease or disorder is asthma.

### Formulation, Delivery, and Dosing

The oligonucleotides described herein can be formulated for administration to a subject for treating a condition (e.g., a disease or disorder associated with aberrant immune cell activation such as an autoimmune or inflammatory disease or disorder) associated with decreased levels of FOXP3. It should be understood that the formulations, compositions and methods can be practiced with any of the oligonucleotides disclosed herein.

The formulations may conveniently be presented in unit dosage form and may be prepared by any methods well known in the art of pharmacy. The amount of active ingredient (e.g., an oligonucleotide or compound of the invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor regression.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated single stranded oligonucleotide composition can assume a variety of states. In some examples, the composition is at least partially crystalline, uniformly crystalline, and/or anhydrous (e.g., less than 80, 50, 30, 20, or 10% water). In another example, the single stranded oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the single stranded oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A single stranded oligonucleotide preparation can be formulated or administered (together or separately) in combination with another agent, e.g., another therapeutic agent or an agent that stabilizes a single stranded oligonucleotide, e.g., a protein that complexes with single stranded oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the single stranded oligonucleotide preparation includes another single stranded oligonucleotide, e.g., a second single stranded oligonucleotide that modulates expression of a second gene or a second single stranded oligonucleotide that modulates expression of the first gene. Still other preparation can include at least 3, 5, ten, twenty, fifty, or a hundred or more different single stranded oligonucleotide species. Such single stranded oligonucleotides can mediated gene expression with respect to a similar number of different genes. In one embodiment, the single stranded oligonucleotide preparation includes at least a second therapeutic agent (e.g., an agent other than an oligonucleotide).

### Route of Delivery

A composition that includes a single stranded oligonucleotide can be delivered to a subject by a variety of routes. Exemplary routes include: intravenous, intradermal, topical, rectal, parenteral, anal, intravaginal, intranasal, pulmonary, ocular, subcutaneous, intramuscular, intraperitoneal, and intra-articular (e.g., injection into a joint for, e.g., rheumatoid arthritis) administration. The term “therapeutically effective amount” is the amount of oligonucleotide present in the composition that is needed to provide the desired level of FOXP3 expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The single stranded oligonucleotide molecules of the invention can be incorporated into pharmaceutical compositions suitable for administration. Such compositions typically include one or more species of single stranded oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the single stranded oligonucleotide in aerosol form. The vascular endothelial cells could be targeted by coating a balloon catheter with the single stranded oligonucleotide and mechanically introducing the oligonucleotide.

In some embodiments, a T cell or population of T cells may be obtained from a subject, e.g., a human subject, and contacted with a single-stranded oligonucleotide as described herein. In some embodiments, the T cell or population of T cells contacted with a single-stranded oligonucleotide as described herein are readminstered to the subject. In some embodiments, the T cell or population of T cells contacted with a single-stranded oligonucleotide as described herein are cultured for a time period (e.g., 1 hour, 2 hours, 3 hours, 4 hours, or more; 1 day, 2 days, 3 days, 4 days, 5 days, 6 days, 7 days or more) before being readministered to the subject.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of single stranded oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the single stranded oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The single stranded oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably single stranded oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. A single stranded oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar single stranded oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to a single stranded oligonucleotide, e.g., a device can release insulin.

In one embodiment, unit doses or measured doses of a composition that includes single stranded oligonucleotide are dispensed by an implanted device. The device can include a sensor that monitors a parameter within a subject. For example, the device can include pump, e.g., and, optionally, associated electronics.

Tissue, e.g., cells or organs can be treated with a single stranded oligonucleotide, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, the single stranded oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

In one embodiment, a contraceptive device is coated with or contains a single stranded oligonucleotide. Exemplary devices include condoms, diaphragms, IUD (implantable uterine devices, sponges, vaginal sheaths, and birth control devices.

### Dosage

In one aspect, the invention features a method of administering a single stranded oligonucleotide (e.g., as a compound or as a component of a composition) to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with FOXP3. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of a single stranded oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the oligonucleotide pharmaceutical composition includes a plurality of single stranded oligonucleotide species. In another embodiment, the single stranded oligonucleotide species has sequences that are non-overlapping and non-adjacent to another species with respect to a naturally occurring target sequence (e.g., a PRC2-associated region). In another embodiment, the plurality of single stranded oligonucleotide species is specific for different PRC2-associated regions. In another embodiment, the single stranded oligonucleotide is allele specific.

In some cases, a patient is treated with a single stranded oligonucleotide in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of the single stranded oligonucleotide composition is an amount sufficient to be effective in treating or preventing a disorder or to regulate a physiological condition in humans. The concentration or amount of single stranded oligonucleotide administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a single stranded oligonucleotide can include a single treatment or, preferably, can include a series of treatments. It will also be appreciated that the effective dosage of a single stranded oligonucleotide used for treatment may increase or decrease over the course of a particular treatment. For example, the subject can be monitored after administering a single stranded oligonucleotide composition. Based on information from the monitoring, an additional amount of the single stranded oligonucleotide composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of FOXP3 expression levels in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human FOXP3. In another embodiment, the composition for testing includes a single stranded oligonucleotide that is complementary, at least in an internal region, to a sequence that is conserved between FOXP3 in the animal model and the FOXP3 in a human.

In one embodiment, the administration of the single stranded oligonucleotide composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising a single stranded oligonucleotide. In some embodiments, the composition is a pharmaceutical composition comprising a single stranded oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for single stranded oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

The invention is further described in the following examples, which do not limit the scope of the invention described in the claims.

### Example 1

### Materials and Methods:

### Real Time (Quantitative) PCR

RNA was harvested from the cells and converted to cDNA as described below. Real time PCR (also referred to herein as quantitative PCR or qPCR or qRTPCR) was performed in a 96 well format with a final volume of 20 ul/well. Each well included 2.5 ul of cDNA, 5.5 ul of water, 1 ul of target gene probe, 1 ul of housekeeper probe and 10 ul of TaqMan® Fast Advanced master mix (Life Technologies™, Invitrogen).

Target gene probes for qPCR for detection of human specific FoxP3, IL2RA, CD69, CD62L, CDKN1A, TNFRSF18 (GITR) and B-Actin (Life Technologies) were used to detect mRNA levels. The target gene probes were labeled with Fam™ and the housekeeper probe was labeled with VIC® (Life Technologies™, Invitrogen).

An Applied Biosystems® StepOne™ Plus Real Time PCR System machine was used to read the samples and determine levels of mRNA. The following cycle program was used:

Step 1: 1 Cycle of 95 degrees Celsius for 20 seconds

Step 2: 40 cycles of 95 degrees Celsius for 3 seconds

Step 3: 40 cycles of 60 degrees Celsius for 20 seconds

A baseline level of mRNA expression for each target gene was determined. Baseline levels were also determined for mRNA of various housekeeping genes which are constitutively expressed. A “control” housekeeping gene with approximately the same level of baseline expression as the target gene was chosen for comparison purposes. Quantitative PCR data presented in Table 2 is represented as RQ (relative quantification): Target dCT=Target Ct−Housekeeper Ct; ddCT=Target dCT−Negative control dCT (unc-293 m01, which is a universal negative control oligonucleotide); RQ=Log 2−ddCT.

### Oligonucleotide Design

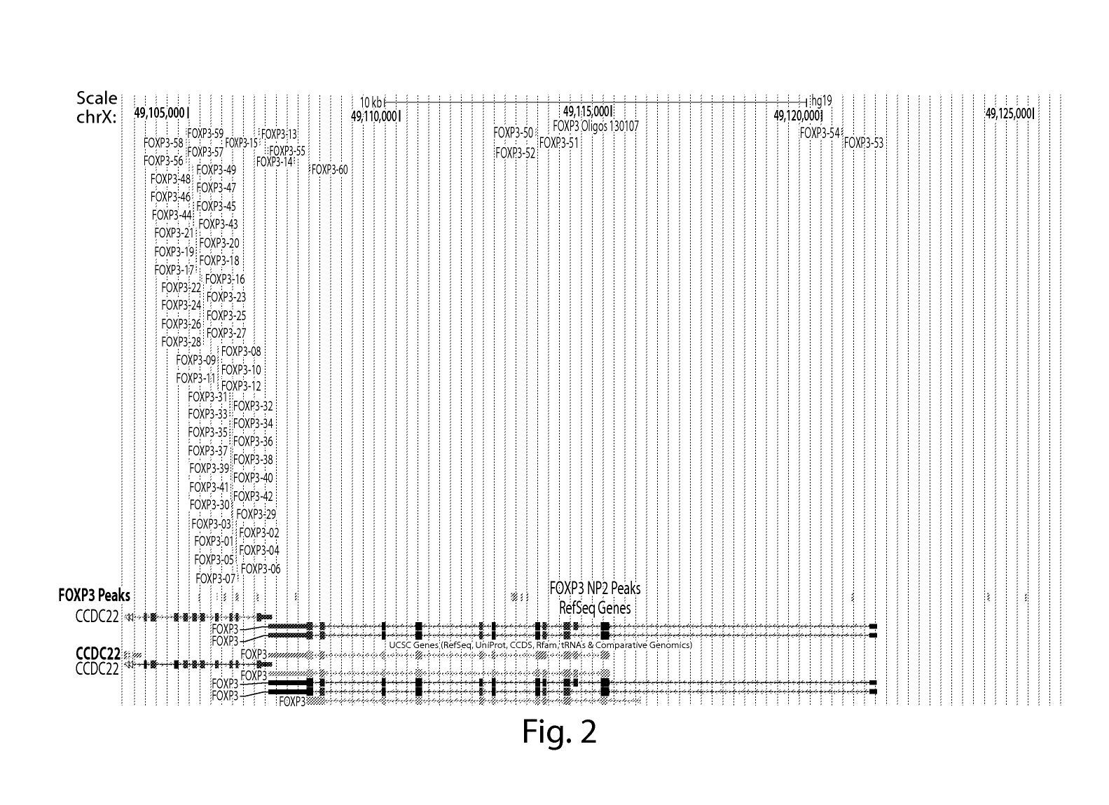

Oligonucleotides were designed within PRC2-interacting regions in order to upregulate FOXP3 (see, e.g., FIG. 2). The sequence and structure of each oligonucleotide is shown in Table 4. Table 3 provides a description of the nucleotide analogs, modifications and intranucleotide linkages used for certain oligonucleotides tested and described in Table 2 or Table 4.

### T Cell Isolation and Culture

Human T cells were obtained from one healthy male and one healthy female donor, both donors were of similar age and health status. For each experiment, T cells were freshly isolated from the donors and sorted for CD4-positivity using fluorescent activated cell sorting (FACS). The human T cells (Stem Cells Technologies) were cultured in RPMI 1640/10% fetal bovine serum in the presence of Anti-Anti (Antibiotic-Antimycotic, Life Technologies™, Invitrogen). Cells were thawed and cultured for one day before stimulation.

### T Cell Stimulation

T-Cells stimulation was performed by using 5 ng/ml of Phorbol 12-myristate 13-acetate (PMA) and 1 uM of Ionomycin (Sigma) for 5 hours. For 2× concentrations doses of PMA and Ionomcin were double. The initial T-cell stimulation conditions were determined using T cells from the healthy male donor. The screening of the oligonucleotides (described below) was carried out with T cells from the healthy female donor (referred to as huTcell+ in Table 2).

In Vitro Transfection of Cells with Oligonucleotides

T-cells were stimulated by PMA/Ionomycin for 5 hours as described above. After stimulation, cells were plated into a 96 well V-bottom plate format with approximately 100,000 cells/well. Each well contained the adequate oligonucleotide amount to produce a final concentration of 10 uM of oligonucleotide. Final volume per well was 100 ul. The same method was used for dose response experiments. The oligonucleotides were delivered gymnotically. The term gymnotic (or gymnotically), with reference to delivery, refers to unassisted uptake of agents into cells without use of transfection reagents or delivery to a subject without transfection reagents. After 48 hours cells were spun down at 2000 RPM, 4 C for 5 minutes, washed with ice cold PBS (Life Technologies) once and a cell lysate was generated using a Cell-to-Ct kit (Life Technologies). The amount of buffer used was 35 ul/well. cDNA was generated utilizing 15 ul of lysate for a total of 50 ul of reaction volume. Quantitative RT-PCR was then carried out as outlined above.

### Results:

### Use of PMA and Ionomycin to Stimulate T Cells

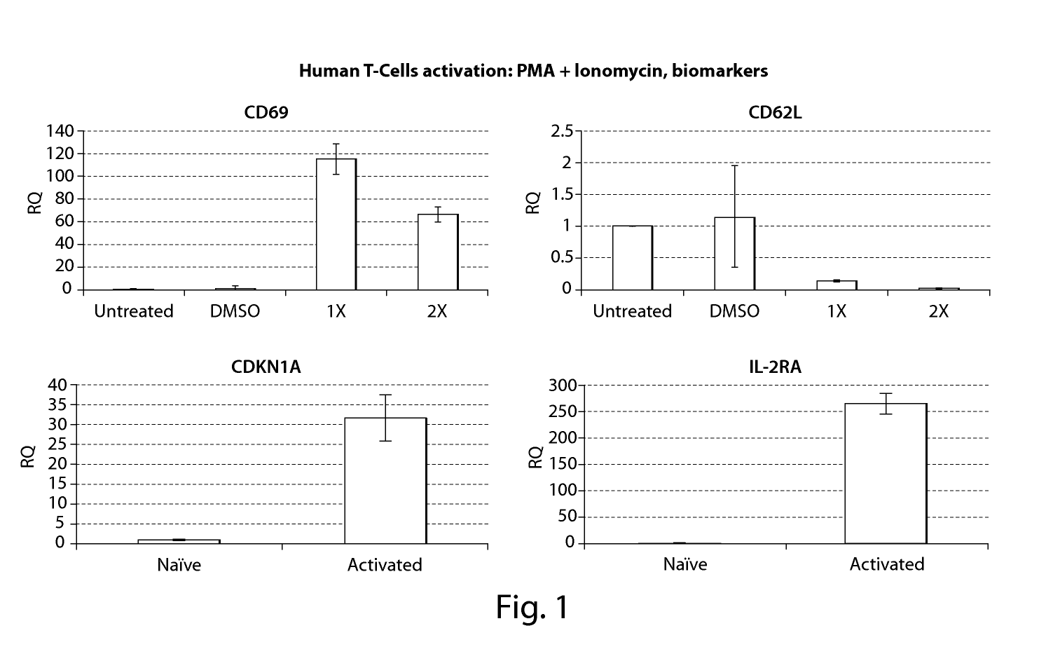

T cells were stimulated with either (a) 5 ng/ml PMA and 1 uM Ionomycin, (b) 10 ng/ml PMA and 2 uM Ionomycin, (c) DMSO alone, or (d) no treatment. Biomarkers for activation and proliferation of the T cells were evaluated to determine whether PMA and Ionomycin treatment successfully stimulated the T cells. CDKN1A is a housekeeper gene that is upregulated upon or during active cell proliferation. CD69 and IL-2RA are known to be upregulated in activated T cells. CD69, CDKN1A, and IL-2RA were found to be upregulated upon stimulation with PMA and Ionomycin (FIG. 1). CD62L, a biomarker for naïve T cells, was found to be downregulated upon stimulation with PMA and Ionomycin (FIG. 1). These results show that PMA and Inomycin were able to stimulate T cells.

### In Vitro Delivery of Single Stranded Oligonucleotides Upregulated FOXP3 Expression

Oligonucleotides were designed as candidates for upregulating FOXP3 expression. Single stranded oligonucleotides were designed to be complementary to a PRC2-interacting region within a sequence as set forth in SEQ ID NO: 1-7, 46, or 47. Multiple oligonucleotides were tested in at least duplicate. The sequence and structural features of the oligonucleotides are set forth in Table 4. Briefly, T-cells were stimulated as described above and then gymnotically transfected in vitro with each of the oligonucleotides as described above. The unc-293 m01 oligo is a universal negative control oligo. “Cntl un” refers to a well that contained no oligonucleotide, which also served as a negative control. FOXP3 expression in stimulated T cells following treatment was evaluated by qRT-PCR. Oligonucleotides that upregulated FOXP3 expression were identified. A subset of the oligonucleotides that upregulated FOXP3 were further tested for expression of two T cell biomarkers, GITR (also called TNFRSF18) and IL2RA. The levels of these two biomarkers were measured by qRT-PCR. GITR is a biomarker for Tregs and thus increased expression of this biomarker may indicate that the activated T cells are switching to a T-regulatory state. IL2RA is biomarker for activated T cells and thus a decrease in IL2RA may indicate a decrease in the T cell activation state (e.g., switching to a T-regulatory state). Further details regarding FOXP3 and T cell biomarker expression are outlined in Table 2.

### Example 2

### Methods:

### T Cell Activation by PMA/Ionomycin

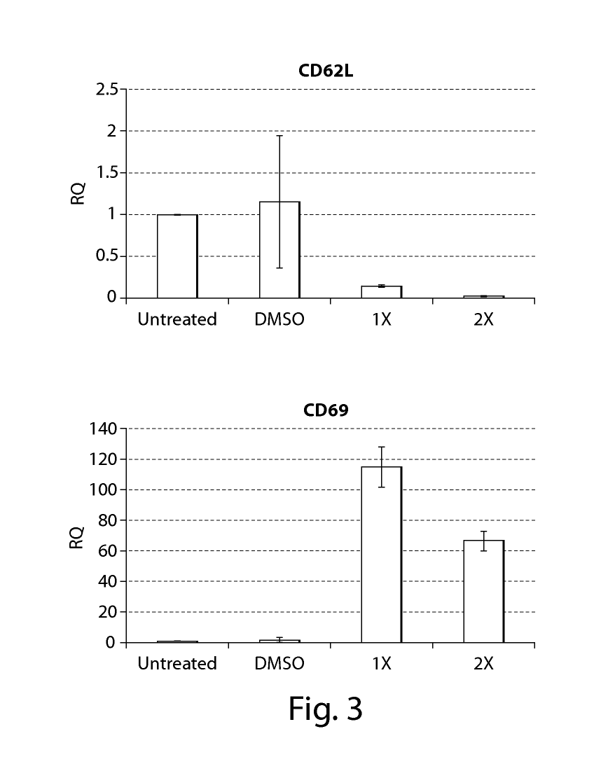

Cells were incubated for 5 hours with two different concentrations of PMA/Ionomycin (1× and 2×). 1× and 2× concentrations of PMA/Ionomycin were the same as defined in Example 1. Naïve T cells were used as control (untreated). CD62L (Naïve cell marker) and CD69 (activated cell marker) mRNA levels were measured (FIG. 3).

### T Cell Activation by Dynabeads

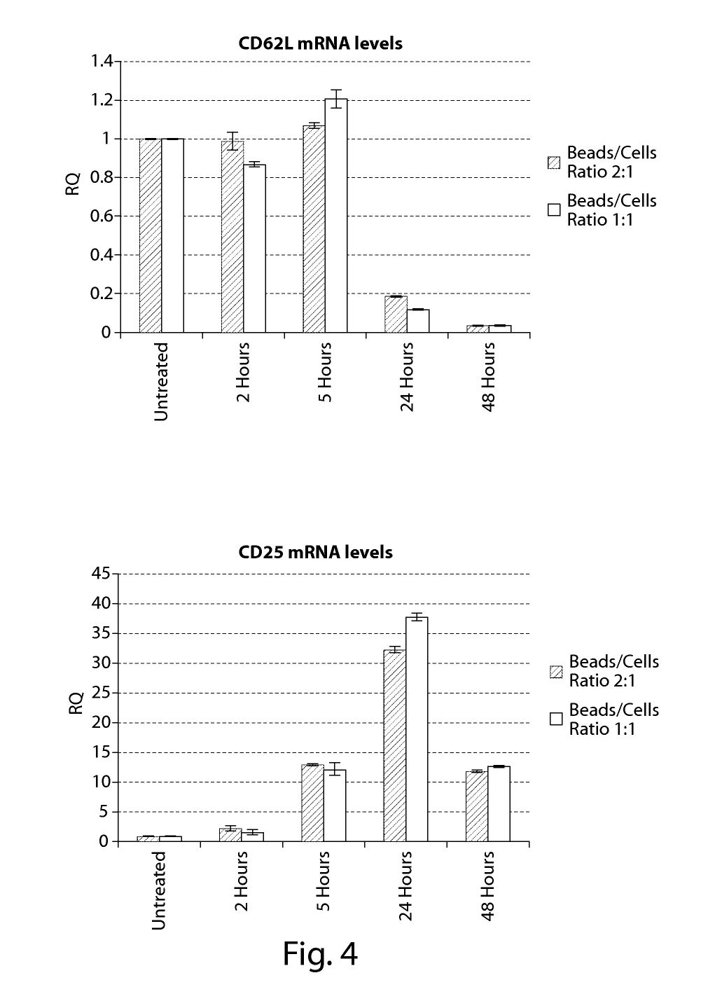

Human T cells were incubated with different ratios of Dynabeads (2:1 or 1:1 beads/cells ratios) for 2, 5, 24 and 48 hours. Naïve T cells were used as control (untreated). CD62L (naïve T cell marker) and CD25 (activated T cell marker) mRNA levels were measured to test T cell activation (FIG. 4).

### Screening

Cells were activated with PMA/Ionomycin or Dynabeads (anti-CD3/CD28) for 5 hours prior to adding oligos. GAPDH gapmers were used to determine an optimized transfection method. The FOXP3 oligos in Table 4 were then screened by gymnotic delivery (unassisted uptake of oligonucleotides into cells without use of transfection reagents) to cells, at 10 μM for 24, 48 and 96 hours. Two separate experiments were performed with two biological replicates each. Foxp3 mRNA levels were measured by qPCR and Foxp3 protein levels were measured by flow cytometry. GITR and CTLA4 (Treg biomarkers) mRNA levels were also measured after Foxp3 oligo treatment. Anti-inflammatory cytokine IL-10 was measured in cells supernatants derived from triple positive cells: Foxp3+, CTLA4+ and GITR+. Certain FOXP3 oligos from Table were selected as having desired properties by the following criteria: Foxp3 mRNA and protein levels, presence of Tregs biomarkers.

### Results:

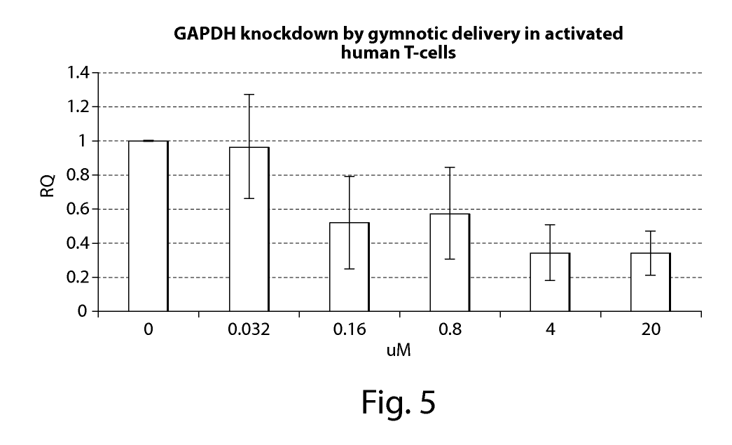

Human T cells were activated using PMA/Ionomycin. Activation of T cells was confirmed by measuring CD62L and CD69 mRNA levels (FIG. 3). GAPDH gapmer oligos were used to show that oligos could be delivered to activated human T cells. GAPDH gapmers showed up to 70% mRNA knockdown in PMA/Iono activated T cells at 4 and 20 μM after 48 hours of oligo treatment (FIG. 5).

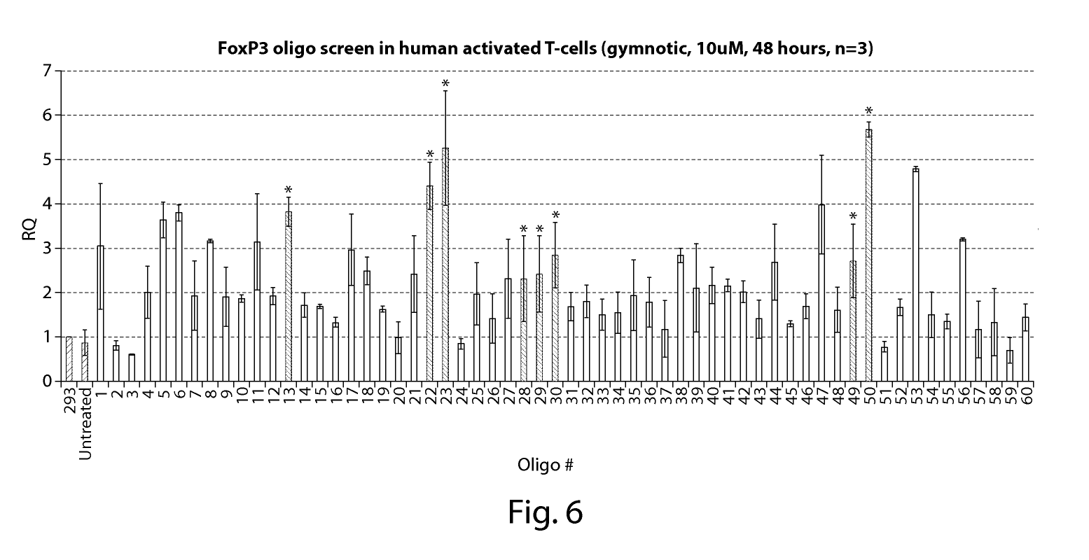

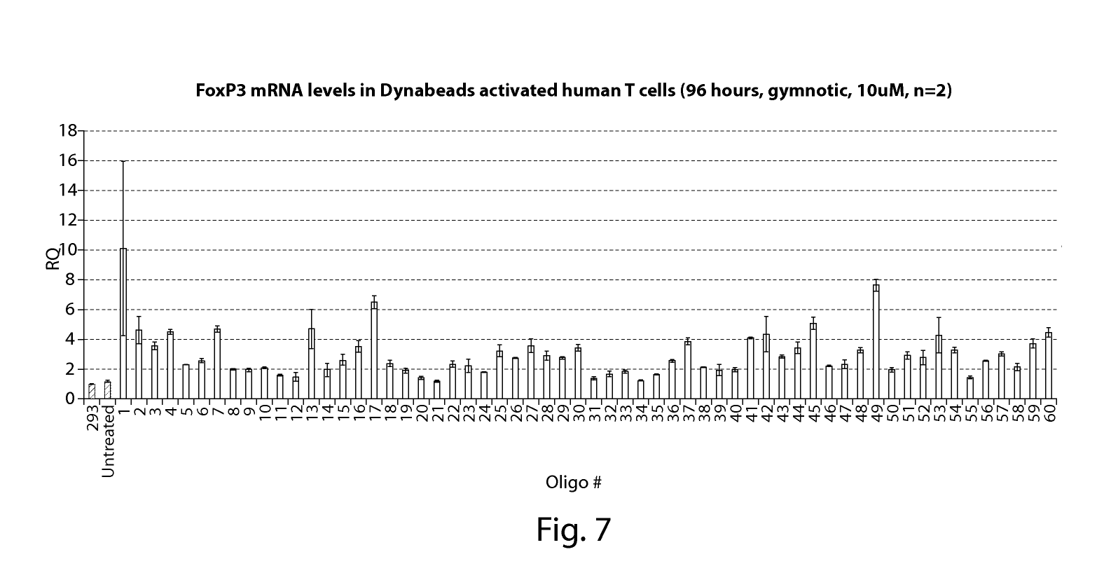

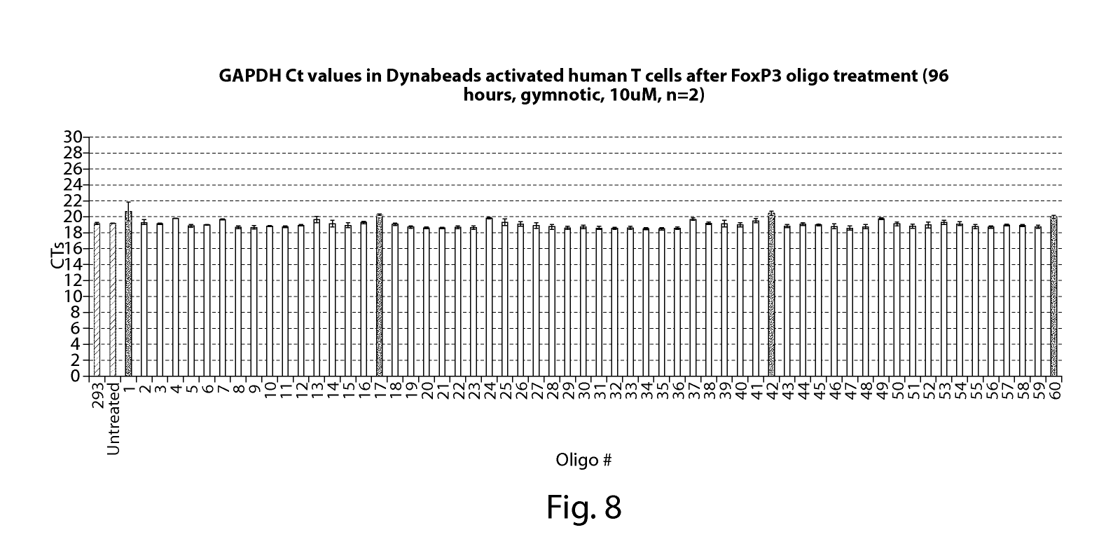

Next, human T cells were activated either using PMA/Ionomycin or dynabeads. Activation of T cells was confirmed by measuring CD62L and CD69 or CD62L and CD25 mRNA levels (FIGS. 3 and 4). FOXP3 oligos from Table 4 (FOXP3-01 to FOXP3-60) were delivered gymnotically at 10 μM to the activated T cells. The FOXP3 oligos showed 2-6 fold Foxp3 mRNA upregulation compared to a negative control oligo (293) in PMA/Ionomycin activated cells (FIG. 6). The FOXP3 oligos showed 2-8 fold Foxp3 mRNA upregulation compared to a negative control oligo (293) in Dynabead activated T cells (FIG. 7). In the dynabead-activated T cells, housekeeper gene (GAPDH) Ct levels were very consistent between treatments (FIG. 8).

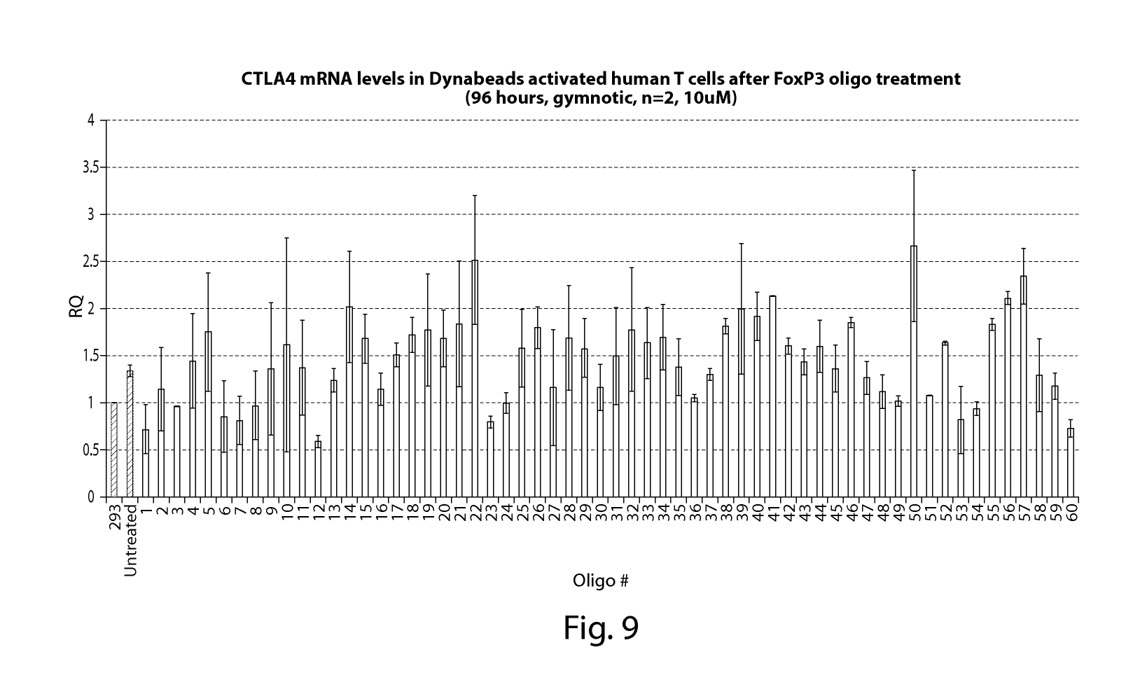

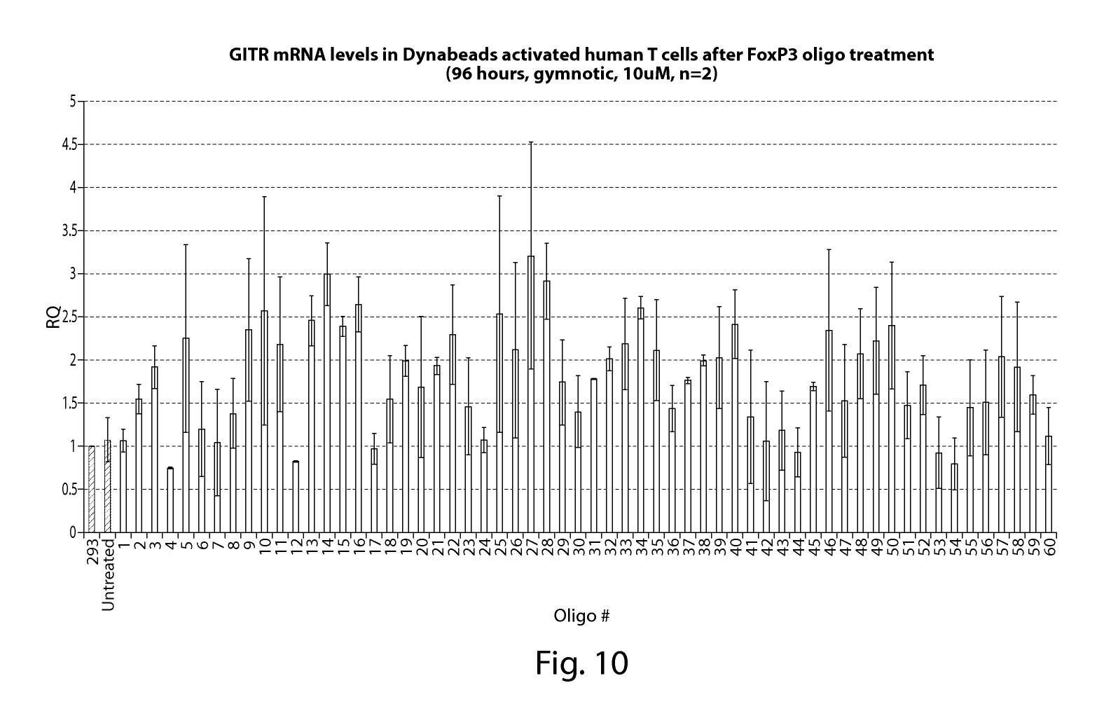

The level of CTLA4 mRNA and GITR mRNA, both Treg biomarkers, were also measured after delivery of the FOXP3 oligos to Dynabead activated T cells. The FOXP3 oligos were shown, in general, to upregulate CTLA4 mRNA levels after 96 hours of treatment (FIG. 9). The FOXP3 oligos were shown, in general, to upregulate GITR mRNA levels after 96 hours of treatment, with over 20 oligos showing upregulation (FIG. 10).

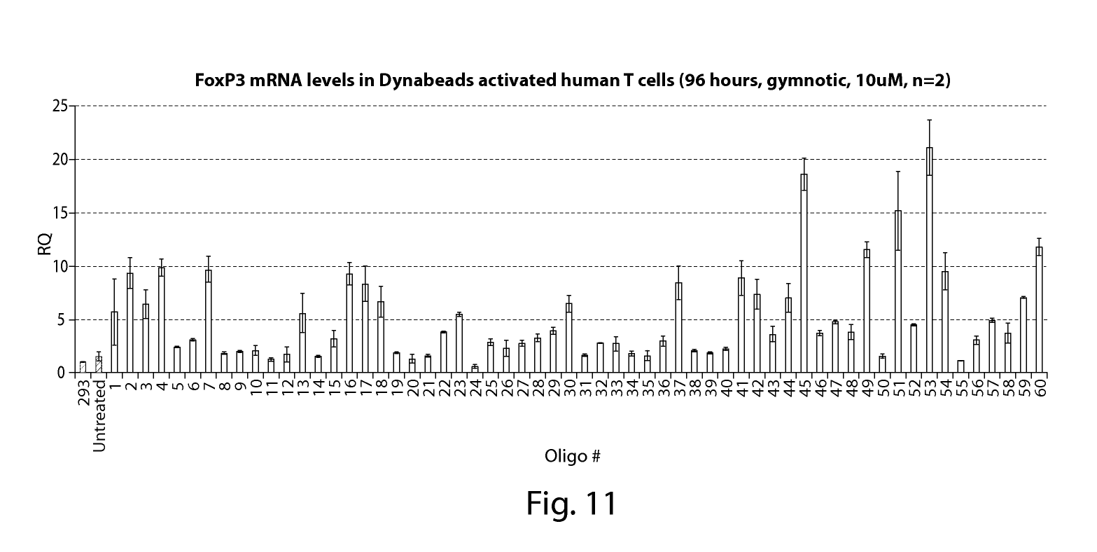

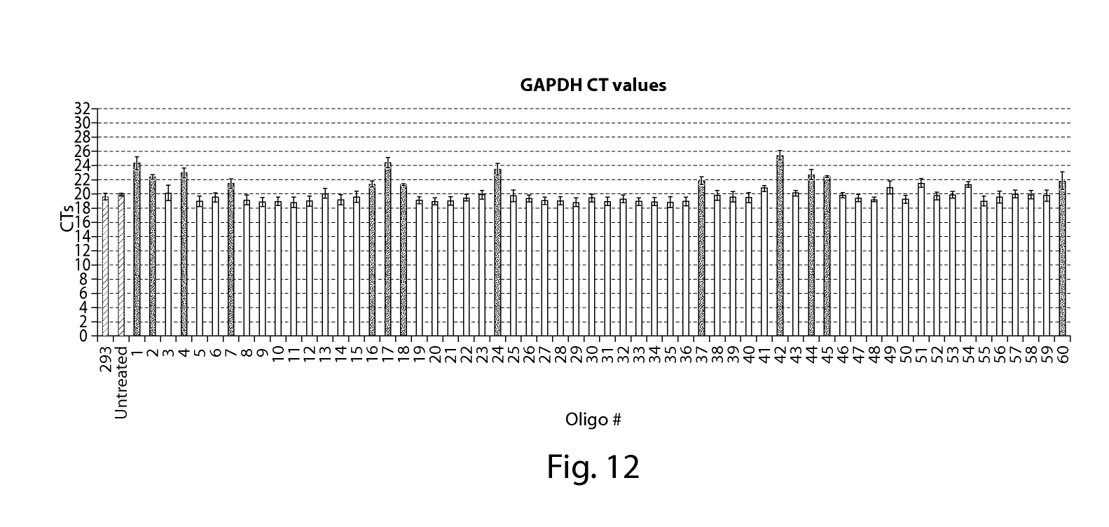

A second experiment was performed to confirm that the FOXP3 oligos upregulated FOXP3 mRNA levels Dynabead activated T cells. In general, the FOXP3 oligos upregulated FOXP3 mRNA levels by 2-10 fold in the activated T cells (FIG. 11). FOXP3 oligos that caused housekeeper gene changes of more than 1.5 Cts were not considered positives (FIG. 12).

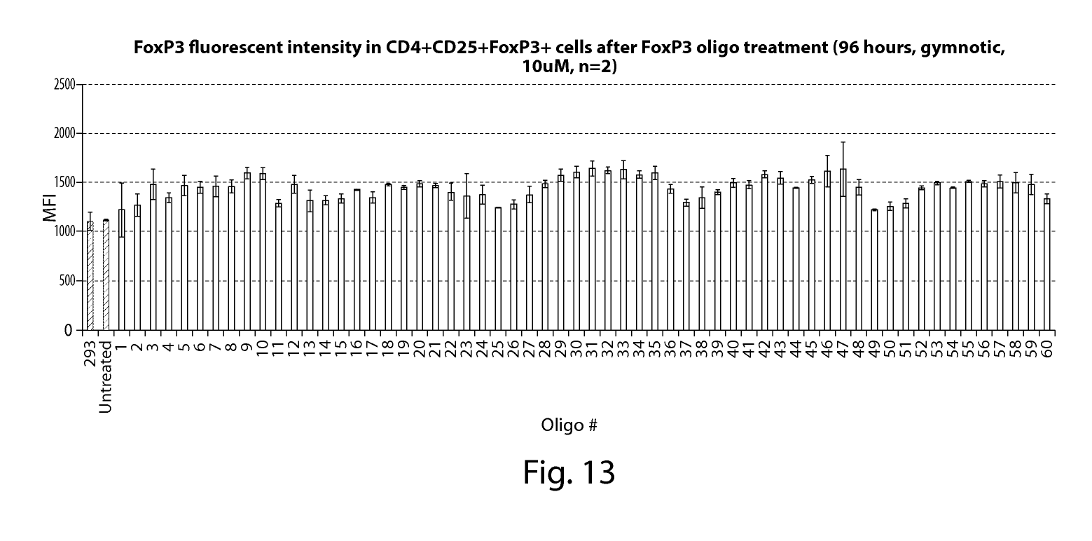

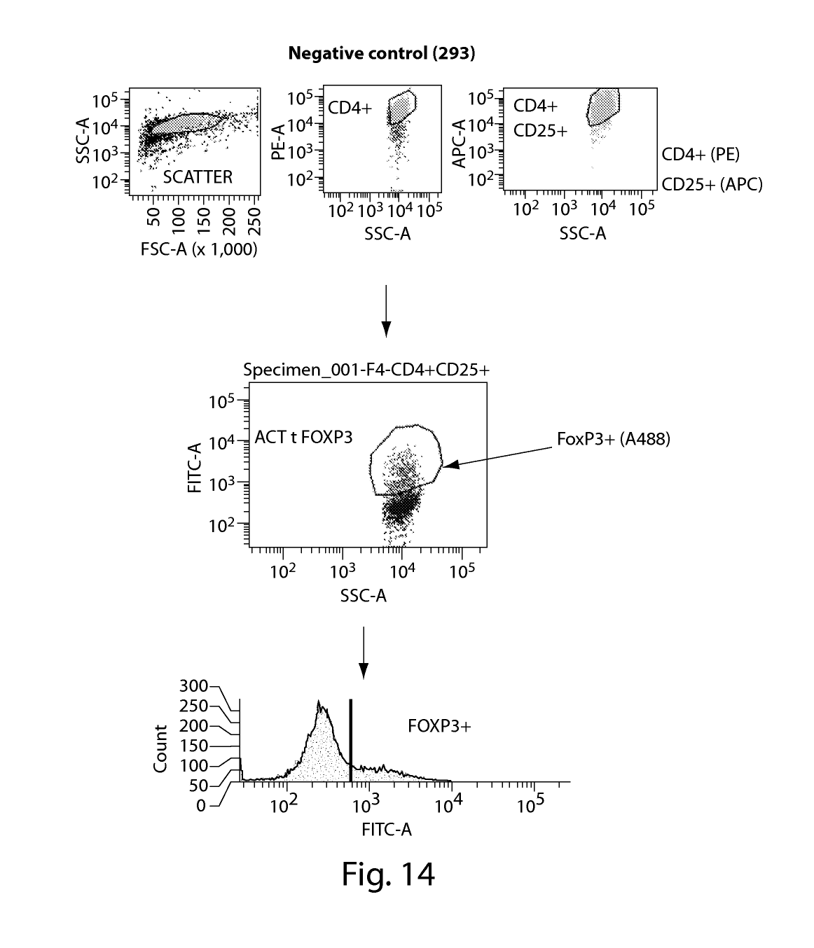

Next, the level of Foxp3 protein in oligo treated activated human T cells was detected using flow cytometry. Foxp3 protein levels were measured in cells that were CD4+, CD25+, and FoxP3+. In general, FOXP3 oligos (e.g., FOXP3-2 to FOXP3-60) were found to increase Foxp3 protein levels in the triple positive human T cells (FIG. 13). Raw flow cytometry data from an exemplary oligo, FOXP3-35, is shown in FIG. 14, demonstrating the difference in FOXP3 expression compared to cells treated with a negative control oligo (293).

The percentage of triple positive Treg cells (CD4+CD25+FoxP3+) compared to the total cell population was next investigated. It was found that several oligos (e.g., FOXP3-3, FOXP3-5 to FOXP3-44, FOXP3-46 to FOXP3-50, FOXP3-52-60) increased the Treg cell population by more than 2 fold compared to a oligo control.

In some embodiments, several FOXP3 oligos were selected as possible lead molecules according to the following criteria: FoxP3 mRNA and protein levels, percent of Tregs within the total cell population and CTLA4/GITR mRNA expression. A summary of results from oligos from two experiments is provided in Tables 5 and 6. Oligos FOXP3-28, FOXP3-29, FOXP3-30 and FOXP3-57 showed positive biomarkers expression (i.e., met the criteria) in both experiments.

TABLE 5
Experiment #1 exemplary results

| | FoxP3 mRNA | | FoxP3 MFI | CTLA4 mRNA | GITR mRNA |
|---|---|---|---|---|---|
| | fold | Tregs % fold | fold | fold | fold |
| | upregulation | upregulation | upregulation | upregulation | upregulation |
| | compared to | compared to | compared to | compared to | compared to |
| | control oligo | control oligo | control oligo | control oligo | control oligo |
| Oligo # | 293 | 293 | 293 | 293 | 293 |
| 293 | 1 | 1 | 1 | 1 | 1 |
| Untreated | 1 | 1 | 1 | 1 | 1.1 |
| 13 | 5.5 | 1.8 | 1.2 | 1.3 | 2.4 |
| 22 | 2.2 | 1.8 | 1.2 | 1.3 | 2.4 |
| 23 | 3.5 | 1.8 | 1.3 | 0.8 | 1.5 |
| 28 | 3 | 1.9 | 1.4 | 1.7 | 2.8 |
| 29 | 3 | 2.1 | 1.5 | 1.6 | 1.7 |
| 30 | 4.5 | 2.1 | 1.5 | 1.2 | 1.4 |
| 49 | 10 | 1.2 | 1.2 | 1 | 2.2 |
| 52 | 3.5 | 1.8 | 1.5 | 1.6 | 1.7 |
| 54 | 5 | 1.8 | 1.5 | 0.9 | 0.9 |
| 57 | 2.6 | 1.8 | 1.6 | 2.3 | 2 |
| 59 | 3.8 | 1.8 | 1.7 | 1.7 | 1.6 |

MFI = Mean Fluorescence Intensity

TABLE 6
Experiment #2 results

| | FoxP3 mRNA | | FoxP3 MFI | CTLA4 mRNA | GITR mRNA |
|---|---|---|---|---|---|
| | fold | Tregs % fold | fold | fold | fold |
| | upregulation | upregulation | upregulation | upregulation | upregulation |
| | compared to | compared to | compared to | compared to | compared to |
| | control oligo | control oligo | control oligo | control oligo | control oligo |
| Oligo # | 293 | 293 | 293 | 293 | 293 |
| 293 | 1 | 1 | 1 | 1 | 1 |
| Untreated | 1 | 1 | 1 | 1 | 1 |
| 3 | 3.9 | 1.7 | 1.4 | 3.8 | 2 |
| 5 | 2.1 | 1.6 | 1.4 | 2.3 | 1.9 |
| 15 | 2.2 | 1.6 | 1.5 | 2.1 | 1.6 |
| 26 | 2.2 | 1.5 | 1.4 | 3.8 | 1.5 |
| 27 | 3.8 | 1.6 | 1.4 | 3.2 | 3.8 |
| 28 | 2.2 | 1.6 | 1.4 | 3.4 | 3.8 |
| 29 | 2.2 | 1.6 | 1.5 | 4.4 | 4.2 |
| 30 | 3.8 | 1.7 | 1.4 | 3.1 | 2 |
| 38 | 2 | 1.5 | 1.3 | 1.9 | 1.9 |
| 41 | 4 | 1.7 | 1.5 | 5.1 | 2.3 |
| 53 | 4.1 | 2.7 | 1.5 | 2.1 | 2.1 |
| 57 | 3.1 | 1.1 | 1.3 | 2.9 | 2.9 |

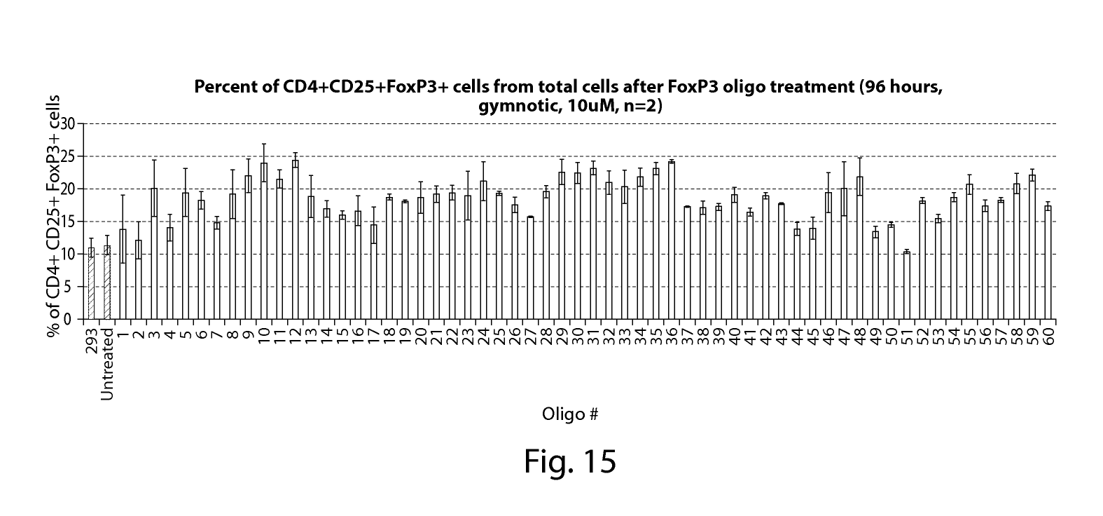

Oligos that were selected based on triple positive markers (CD4+/CD25+/FoxP3+) upregulated IL-10 protein, an immunosuppressive cytokine (FIG. 16). In summary, the results in this Example show that FOXP3 oligos are capable of upregulating FOXP3, as well as several Treg biomarkers and IL-10.

### Example 3

### Methods:

Mice (n=4) were treated with a single dose of 25 mg/kg gapmer targeting MALAT-1. Blood and liver were harvested after 1, 5 and 7 days from dosing. CD4+ cells were sorted from blood and MALAT-1 mRNA was measured by qPCR.

### Results:

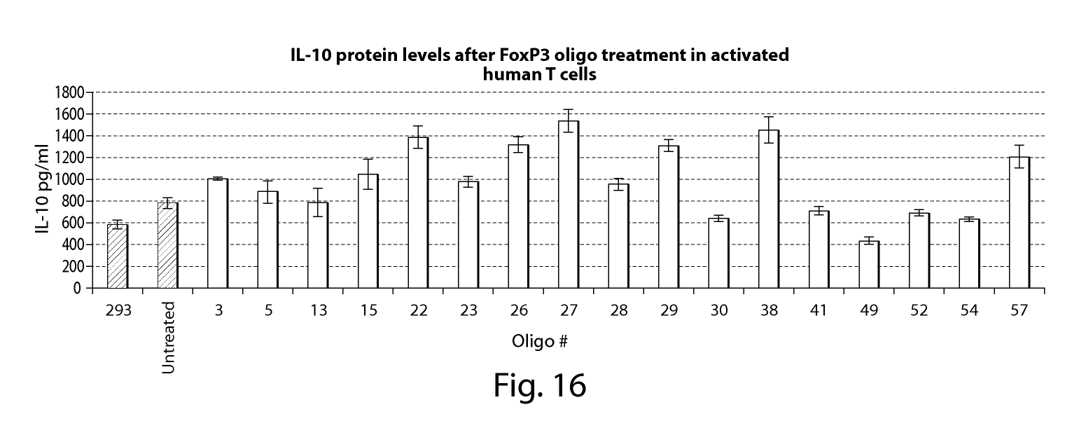

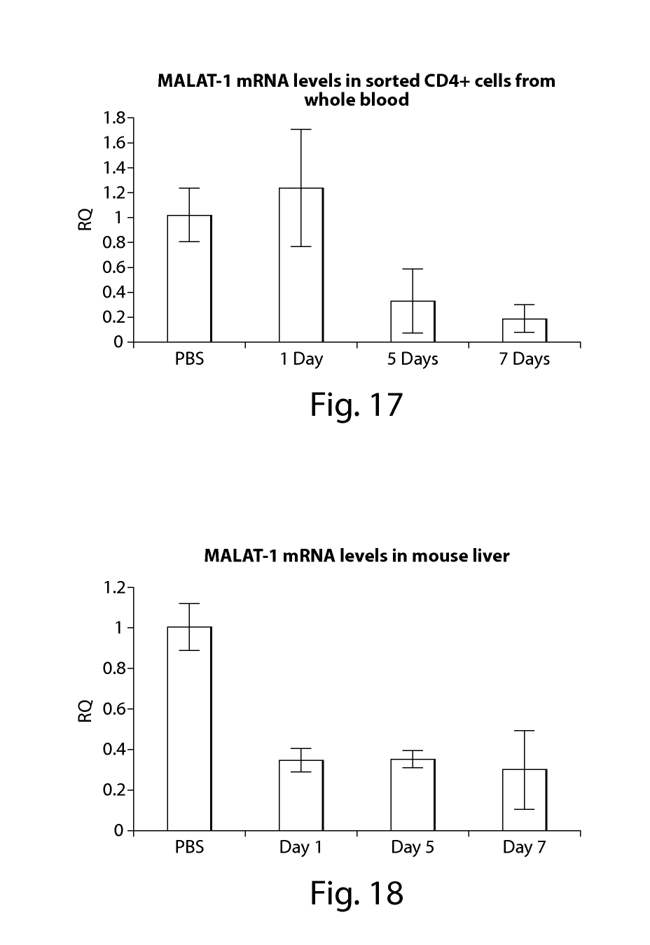

MALAT-1 gapmers were used to show that oligos can be delivered in vivo to T cells. It was shown that a single dose of MALAT-1 gapmer oligonucleotides could reduce levels of MALAT-1 mRNA in vivo in CD4+ T cells and in the liver (FIGS. 17 and 18). These results show that oligos can be successfully delivered to T cells in vivo.

### Example 4

Potential lead oligos identified in Example 2 are tested in mouse models, as these oligos were designed to have 1 or 0 mismatches with the mouse FoxP3 gene (see Table 7 below). Exemplary mouse models include GFP/RFP Treg reporter mice, EAE (multiple sclerosis) and NOD (type 1 diabetes) mouse models, mouse inflammatory disease models, and humanized mouse models. Exemplary inflammatory disease models include graft versus host disease (GvHD) models, inflammatory bowel disease (IBD) models such as models of Crohn's disease and ulcerative colitis, rheumatoid arthritis models and psoriasis models. GvHD models include several models involving introduction of donor cells or tissues into a MHC mismatched or miHA mismatched host, e.g., C57/Bl6(H2b) donor strain splenocytes or T cells into BALB/c(H2d) recipient strain or B10.Br(H2k) donor strain bone marrow cells or T cells into BALB.K(H2k) recipient strain (see, e.g., Schroeder et al. Dis Model Mech. May 2011; 4(3): 318-333). IBD models include genetic models IL-10R2^{-/-}×dominant negative TGFβRII mice, SAMP1/Yit, Mdr1a^{-/-}, IKKγ^{-/-}, and chemical agent models Dextran Sodium Sulfate, 2,4,6-trinitrobenzenesulfonic acid, and oxazolone (see, e.g., Mizoguchi. Prog Mol Biol Transl Sci. 2012; 105:263-320). Rheumatoid arthritis models include Collagen-induced arthritis, collagen-antibody induced arthritis, inflammatory arthritis primed with an antigen (e.g. methylated BSA in complete Freund's adjuvant), TNF-a transgenic mice, SKG mice, SCID mice, DR4-CD4 mice, and DNase II^{-/-}IFN-IR^{-/-} mice (see, e.g., Asquith et al. Eur. J. Immunol. 2009. 39: 1991-2058). Psoriasis models include Ttc7^{fsn}/Ttc7^{fsn} mice, K5-Stat3C mice, K14-IL-20 mice, K14-IL-6 mice, K5-latent TGFbeta1 mice, K10-BMP-6 mice, K14-IL1alpha mice, K14-VEGF mice, IL1-ra knockout mice, IRF-2 knockout mice, and IKK2 knockout mice (see, e.g., Gudjonsson et al. J Invest Dermatol. 2007 June; 127(6):1292-308). Appropriate mouse models are also available, for example, from the Jackson Laboratory (Bar Harbor, Me.) or another commercial source.

TABLE 7
FOXP3 oligo number of mismatches with mouse FoxP3 gene.

| Oligo # | # of mismatches |
|---|---|
| FOXP3-03 | 1 |
| FOXP3-05 | 1 |
| FOXP3-13 | 1 |
| FOXP3-15 | 1 |
| FOXP3-22 | 1 |
| FOXP3-23 | 1 |
| FOXP3-26 | 0 |
| FOXP3-27 | 0 |
| FOXP3-28 | 0 |
| FOXP3-29 | 1 |
| FOXP3-30 | 1 |
| FOXP3-38 | 1 |
| FOXP3-41 | 1 |
| FOXP3-49 | 1 |
| FOXP3-50 | 1 |
| FOXP3-54 | 1 |
| FOXP3-57 | 0 |

### Example 5

Gapmers were designed to target and degrade human EZH1 and EZH2 mRNA. Gapmers were used to evaluate the extent to which FOXP3 expression is regulated by EZH1 and/or EZH2.

TABLE 8
EZH1 and EZH2 gapmers

| | Base | | |
|---|---|---|---|
| OligoID | Sequence | Formatted Sequence* | SeqID |
| EZH1- | GACACGAA | lnaGs; lnaAs; lnaCs; lnaAs; | 45714 |
| 28 m08 | ATCACGCA | dCs; dGs; dAs; dAs; dAs; | |
| | T | dTs; dCs; dAs; dCs; lnaGs; | |
| | | lnaCs; lnaAs; lnaT-Sup | |
| EZH1- | CGACACGA | lnaCs; lnaGs; lnaAs; lnaCs; | 45715 |
| 29 m08 | AATCACGC | dAs; dCs; dGs; dAs; dAs; | |
| | A | dAs; dTs; dCs; dAs; lnaCs; | |
| | | lnaGs; lnaCs; lnaA-Sup | |
| EZH2- | GATTTTAC | lnaGs; lnaAs; lnaTs; lnaTs; | 45716 |
| 09 m08 | ACGCTTCC | dTs; dTs; dAs; dCs; dAs; | |
| | G | dCs; dGs; dCs; dTs; lnaTs; | |
| | | lnaCs; lnaCs; lnaG-Sup | |
| EZH2- | CTTTCGAT | lnaCs; lnaTs; lnaTs; lnaTs; | 45717 |
| 38 m08 | GCCGACAT | dCs; dGs; dAs; dTs; dGs; | |
| | A | dCs; dCs; dGs; dAs; lnaCs; | |
| | | lnaAs; lnaTs; lnaA-Sup | |

*Table 3 provides a description of the nucleotide analogs, modifications and intranucleotide linkages used for certain oligonucleotides tested and described in Table 8.

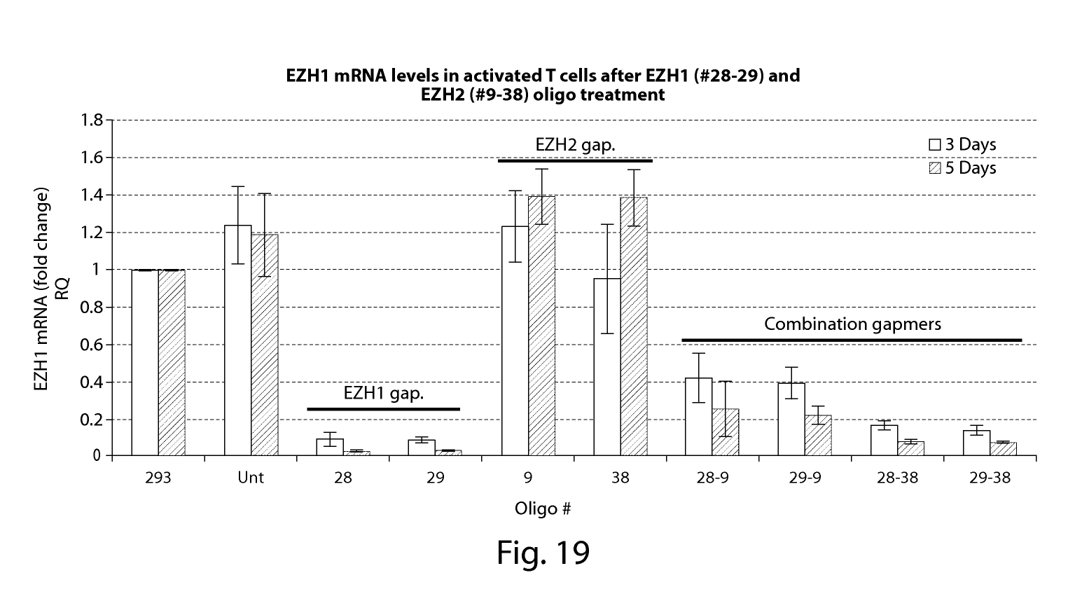

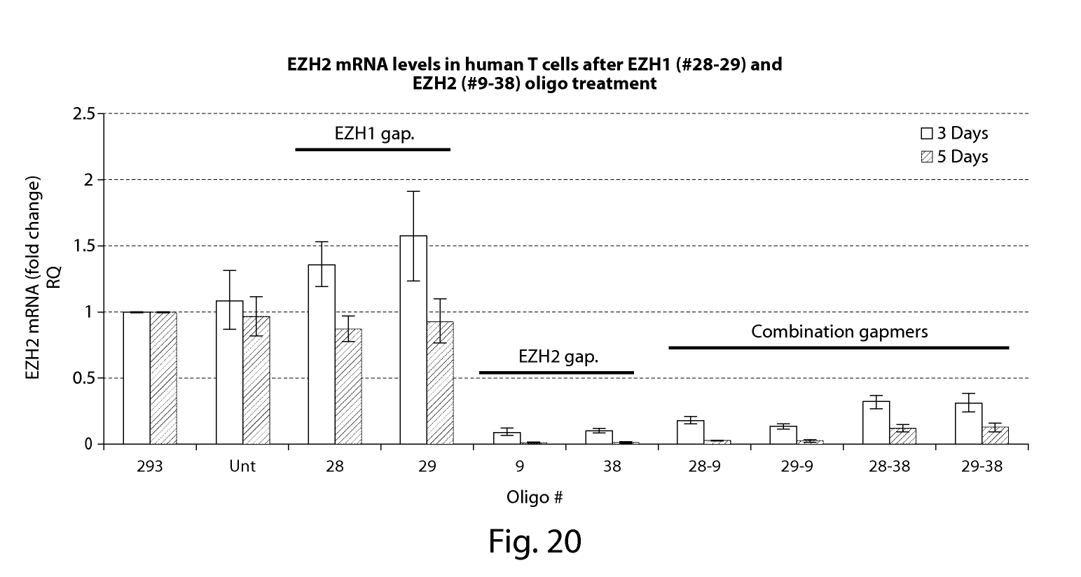

Gapmers targeting EZH1 reduced levels of EZH1 mRNA levels in human T cells by 90-95% after 5 days at 10 μM (unassisted uptake) when compared to negative control (293) (FIG. 19). At 5 μM (combination oligos) levels of EZH1 were decreased between 60 and 90% depending on gapmer and time (FIG. 19). EZH2 gapmers decreased EZH2 mRNA levels up to 99% after 5 days at 10 μM when compared to negative control (293) (FIG. 20). Gapmer combinations decreased EZH2 mRNA levels by 75% (FIG. 20).

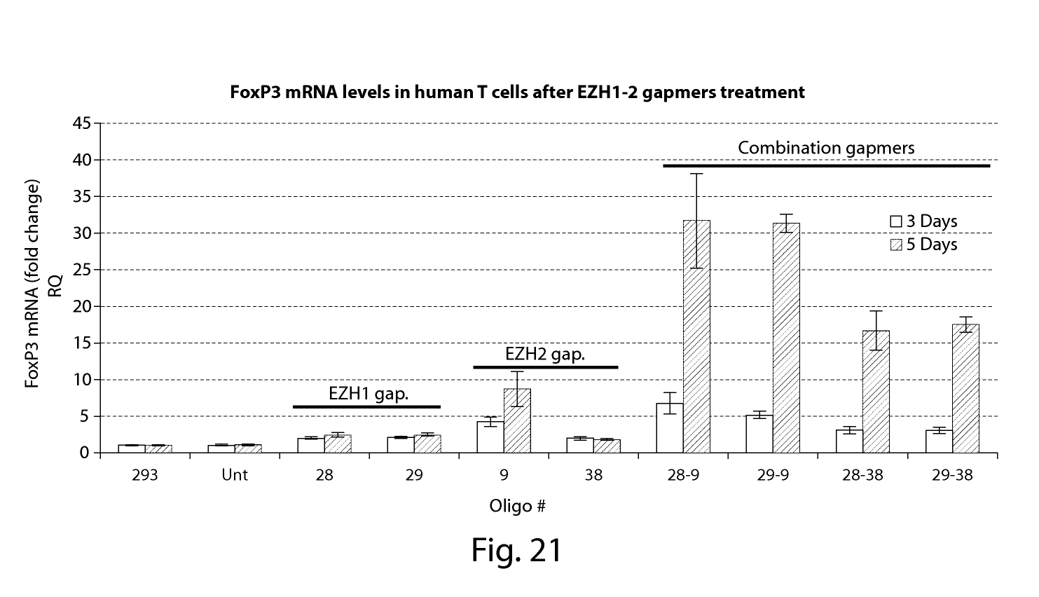

EZH1 gapmers increased FoxP3 mRNA levels about 2 fold when compared to negative control (FIG. 21). FoxP3 mRNA levels increased up to 10 fold by EZH2 gapmer #9 (FIG. 21). The effect of the EZH1 and EZH2 gapmer combinations (e.g., 28-9, 29-9, 28-38, and 29-38) on FoxP3 mRNA levels was far greater than either alone, appearing to be synergistic (FIG. 21).

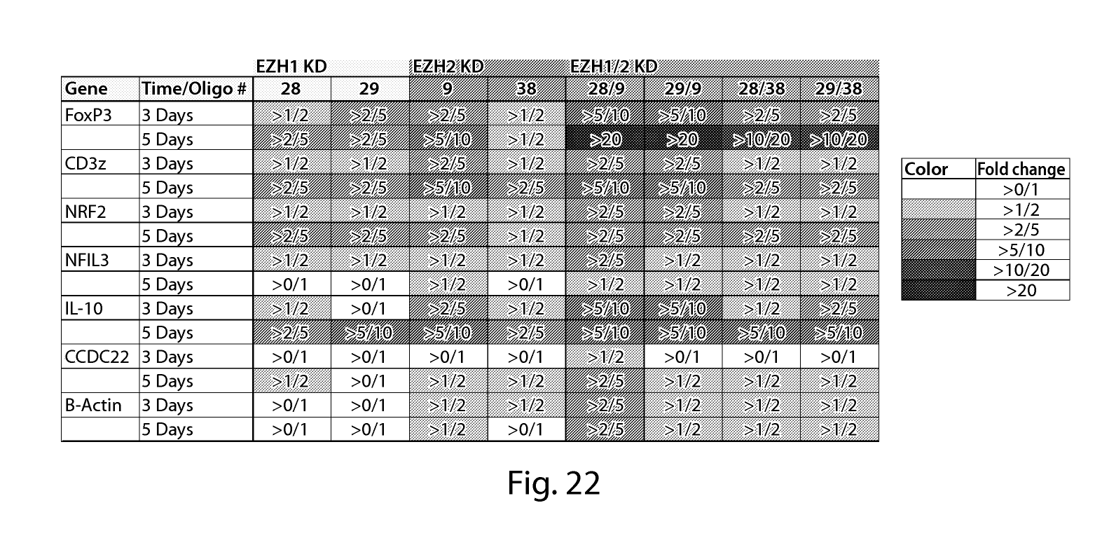

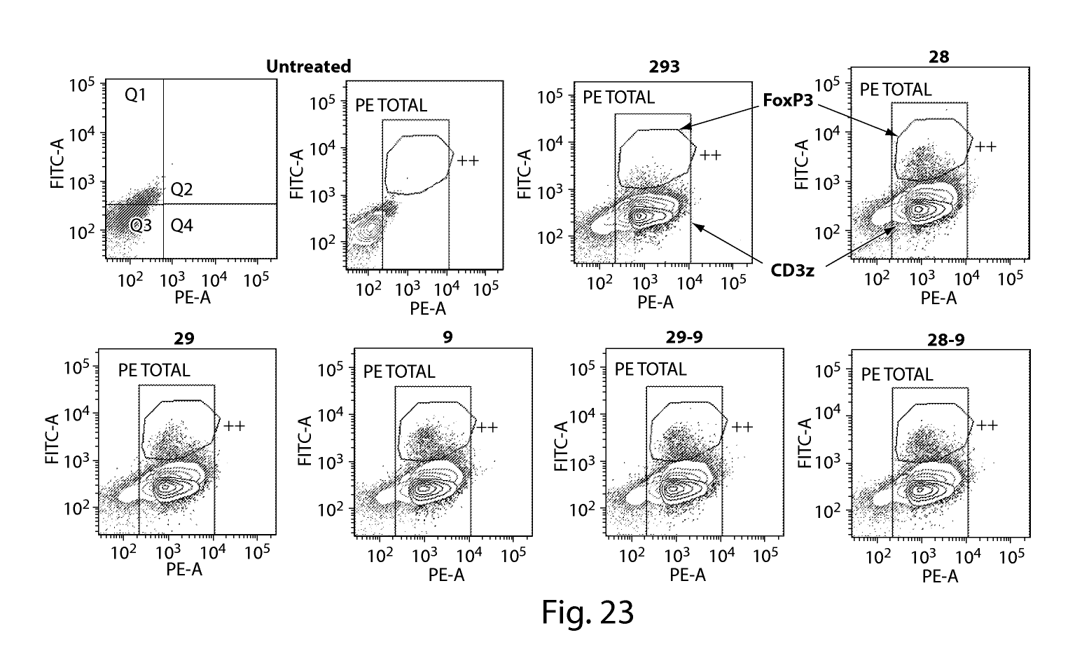

Several T cell related genes were tested for gene expression levels after EZH1/2 gapmer treatment. Foxp3 showed a higher increase in mRNA levels after EZH1/2 KD. Again, a possible synergistic effect was observed when EZH1 and EZH2 gapmers were combined (FIG. 22). Treatment with EZH1 and EZH2 gapmers also increased the percent of cells double positive (++) for Foxp3 and CD3z (FIG. 23).

### Tables

TABLE 1
Hexamers that are not seed sequences of human miRNAs

AAAAAA, AAAAAG, AAAACA, AAAAGA, AAAAGC, AAAAGG, AAAAUA, AAACAA, AAACAC, AAACAG,
AAACAU, AAACCC, AAACCU, AAACGA, AAACGC, AAACGU, AAACUA, AAACUC, AAACUU, AAAGAU,
AAAGCC, AAAGGA, AAAGGG, AAAGUC, AAAUAC, AAAUAU, AAAUCG, AAAUCU, AAAUGC, AAAUGU,
AAAUUA, AAAUUG, AACAAC, AACAAG, AACAAU, AACACA, AACACG, AACAGA, AACAGC, AACAGG,
AACAUC, AACAUG, AACCAA, AACCAC, AACCAG, AACCAU, AACCCC, AACCCG, AACCGA, AACCGC,
AACCGG, AACCUA, AACCUU, AACGAA, AACGAC, AACGAG, AACGAU, AACGCU, AACGGG, AACGGU,
AACGUA, AACGUC, AACGUG, AACGUU, AACUAU, AACUCA, AACUCC, AACUCG, AACUGA, AACUGC,
AACUGU, AACUUA, AACUUC, AACUUG, AACUUU, AAGAAA, AAGAAG, AAGAAU, AAGACG, AAGAGA,
AAGAGC, AAGAGG, AAGAGU, AAGAUU, AAGCAA, AAGCAC, AAGCAG, AAGCAU, AAGCCA, AAGCCC,
AAGCCG, AAGCCU, AAGCGA, AAGCGG, AAGCGU, AAGCUA, AAGGAA, AAGGAC, AAGGCU, AAGGGC,
AAGGGU, AAGGUU, AAGUAA, AAGUAC, AAGUAU, AAGUCC, AAGUCG, AAGUGA, AAGUGG, AAGUUA,
AAGUUU, AAUAAA, AAUAAC, AAUAAG, AAUAAU, AAUACA, AAUACC, AAUACG, AAUAGA, AAUAGC,
AAUAGG, AAUAGU, AAUAUC, AAUAUU, AAUCAA, AAUCAU, AAUCCA, AAUCCC, AAUCCG, AAUCGA,
AAUCGC, AAUCGU, AAUCUA, AAUCUG, AAUCUU, AAUGAA, AAUGAC, AAUGAG, AAUGAU, AAUGCG,
AAUGCU, AAUGGA, AAUGGU, AAUGUA, AAUGUC, AAUGUG, AAUUAA, AAUUAC, AAUUAG, AAUUCC,
AAUUCG, AAUUGA, AAUUGG, AAUUGU, AAUUUC, AAUUUG, ACAAAA, ACAAAC, ACAAAG, ACAAAU,
ACAACC, ACAACG, ACAACU, ACAAGA, ACAAGC, ACAAGU, ACAAUC, ACAAUG, ACAAUU, ACACAG,
ACACCA, ACACCC, ACACCG, ACACCU, ACACGA, ACACGC, ACACGU, ACACUC, ACACUG, ACACUU,
ACAGAA, ACAGAC, ACAGCC, ACAGCG, ACAGCU, ACAGGG, ACAGUC, ACAGUG, ACAGUU, ACAUAA,
ACAUAC, ACAUCC, ACAUCG, ACAUCU, ACAUGA, ACAUGC, ACAUGU, ACAUUG, ACAUUU, ACCAAA,
ACCAAC, ACCAAG, ACCAAU, ACCACC, ACCACG, ACCAGA, ACCAGU, ACCAUA, ACCAUG, ACCAUU,
ACCCAA, ACCCAC, ACCCCA, ACCCCG, ACCCGA, ACCCGC, ACCCUA, ACCCUC, ACCCUU, ACCGAA,
ACCGAC, ACCGAU, ACCGCA, ACCGCC, ACCGCG, ACCGCU, ACCGGA, ACCGGC, ACCGGU, ACCGUA,
ACCGUC, ACCGUG, ACCGUU, ACCUAA, ACCUAC, ACCUAG, ACCUAU, ACCUCA, ACCUCC, ACCUCG,
ACCUCU, ACCUGA, ACCUGC, ACCUGU, ACCUUA, ACCUUC, ACCUUU, ACGAAA, ACGAAC, ACGAAG,
ACGAAU, ACGACA, ACGACC, ACGACG, ACGACU, ACGAGA, ACGAGC, ACGAGG, ACGAGU, ACGAUA,
ACGAUC, ACGAUG, ACGAUU, ACGCAA, ACGCAG, ACGCAU, ACGCCC, ACGCCG, ACGCCU, ACGCGA,
ACGCGG, ACGCGU, ACGCUA, ACGCUG, ACGCUU, ACGGAA, ACGGAC, ACGGAG, ACGGAU, ACGGCC,
ACGGCG, ACGGCU, ACGGGC, ACGGGG, ACGGGU, ACGGUA, ACGGUC, ACGGUG, ACGGUU, ACGUAA,
ACGUAC, ACGUAU, ACGUCC, ACGUCG, ACGUCU, ACGUGA, ACGUGC, ACGUGG, ACGUGU, ACGUUA,
ACGUUC, ACGUUG, ACGUUU, ACUAAA, ACUAAG, ACUAAU, ACUACA, ACUACC, ACUACG, ACUACU,
ACUAGG, ACUAUC, ACUAUG, ACUAUU, ACUCAU, ACUCCC, ACUCCG, ACUCCU, ACUCGA, ACUCGC,
ACUCGG, ACUCUC, ACUCUU, ACUGAG, ACUGAU, ACUGCC, ACUGCG, ACUGCU, ACUGGG, ACUGGU,
ACUGUC, ACUUAA, ACUUAC, ACUUAU, ACUUCA, ACUUCC, ACUUCG, ACUUCU, ACUUGA, ACUUGC,
ACUUGU, ACUUUA, ACUUUC, ACUUUG, AGAAAA, AGAAAC, AGAAAG, AGAACC, AGAACG, AGAACU,
AGAAGC, AGAAGU, AGAAUA, AGAAUC, AGAAUG, AGAAUU, AGACAA, AGACAC, AGACAU, AGACCA,
AGACCC, AGACCG, AGACCU, AGACGA, AGACGC, AGACGU, AGACUA, AGACUC, AGACUU, AGAGAC,
AGAGAG, AGAGAU, AGAGCC, AGAGCG, AGAGCU, AGAGGC, AGAGGG, AGAGGU, AGAGUA, AGAGUU,
AGAUAC, AGAUAG, AGAUAU, AGAUCC, AGAUCG, AGAUCU, AGAUGA, AGAUGC, AGAUGG, AGAUUA,
AGAUUC, AGAUUG, AGAUUU, AGCAAC, AGCACA, AGCACG, AGCACU, AGCAGA, AGCAUA, AGCAUC,
AGCAUG, AGCCAA, AGCCAU, AGCCCA, AGCCGA, AGCCGC, AGCCGG, AGCCGU, AGCCUA, AGCCUC,
AGCGAA, AGCGAG, AGCGAU, AGCGCA, AGCGCC, AGCGCG, AGCGCU, AGCGGA, AGCGGC, AGCGGU,
AGCGUA, AGCGUC, AGCGUG, AGCGUU, AGCUAA, AGCUAC, AGCUAG, AGCUAU, AGCUCA, AGCUCC,
AGCUCG, AGCUCU, AGCUGA, AGCUGG, AGCUGU, AGCUUC, AGCUUU, AGGAAU, AGGACC, AGGACG,
AGGAGA, AGGAGU, AGGAUA, AGGCAA, AGGCAU, AGGCCG, AGGCGA, AGGCGC, AGGCGG, AGGCUA,
AGGCUC, AGGCUU, AGGGAC, AGGGAU, AGGGGA, AGGGGU, AGGGUA, AGGGUG, AGGUAA,
AGGUAC, AGGUCA, AGGUCC, AGGUCU, AGGUGA, AGGUGC, AGGUGG, AGGUGU, AGGUUC,
AGGUUG, AGUAAA, AGUAAG, AGUAAU, AGUACA, AGUACG, AGUAGC, AGUAGG, AGUAUA, AGUAUC,
AGUAUG, AGUAUU, AGUCAA, AGUCAC, AGUCAG, AGUCAU, AGUCCA, AGUCCG, AGUCCU, AGUCGA,
AGUCGC, AGUCGG, AGUCGU, AGUCUA, AGUCUC, AGUCUG, AGUCUU, AGUGAA, AGUGAC, AGUGCG,
AGUGGG, AGUGUC, AGUUAA, AGUUAC, AGUUAG, AGUUCC, AGUUCG, AGUUGA, AGUUGC,
AGUUGU, AGUUUA, AGUUUC, AGUUUG, AGUUUU, AUAAAC, AUAAAU, AUAACA, AUAACC, AUAACG,
AUAACU, AUAAGA, AUAAGC, AUAAGG, AUAAGU, AUAAUC, AUAAUG, AUAAUU, AUACAC, AUACAG,
AUACAU, AUACCA, AUACCC, AUACCG, AUACGA, AUACGC, AUACGG, AUACGU, AUACUA, AUACUC,
AUACUG, AUACUU, AUAGAA, AUAGAC, AUAGAU, AUAGCA, AUAGCG, AUAGCU, AUAGGA, AUAGGU,
AUAGUA, AUAGUC, AUAGUG, AUAGUU, AUAUAC, AUAUAG, AUAUCC, AUAUCG, AUAUCU, AUAUGA,
AUAUGC, AUAUGG, AUAUGU, AUAUUC, AUAUUG, AUAUUU, AUCAAA, AUCAAC, AUCAAG, AUCAAU,
AUCACA, AUCACC, AUCACG, AUCAGC, AUCAGG, AUCCAA, AUCCAU, AUCCCC, AUCCCG, AUCCGA,
AUCCGC, AUCCGG, AUCCUA, AUCCUC, AUCCUG, AUCGAA, AUCGAC, AUCGAG, AUCGAU, AUCGCA,
AUCGCC, AUCGCG, AUCGCU, AUCGGC, AUCGGG, AUCGGU, AUCGUC, AUCGUG, AUCGUU, AUCUAA,
AUCUAC, AUCUAG, AUCUAU, AUCUCC, AUCUCG, AUCUGU, AUCUUG, AUCUUU, AUGAAA, AUGAAC,
AUGAAG, AUGAAU, AUGACC, AUGACU, AUGAGG, AUGAGU, AUGAUA, AUGAUC, AUGAUU, AUGCAA,
AUGCAG, AUGCCA, AUGCCC, AUGCCG, AUGCGA, AUGCGG, AUGCGU, AUGCUC, AUGCUU, AUGGAC,
AUGGCC, AUGGGA, AUGGGC, AUGGGU, AUGGUC, AUGGUG, AUGUAC, AUGUAU, AUGUCA,
AUGUCC, AUGUCG, AUGUGU, AUGUUA, AUGUUC, AUUAAA, AUUAAC, AUUAAG, AUUAAU, AUUACA,
AUUACC, AUUACG, AUUACU, AUUAGA, AUUAGC, AUUAGG, AUUAGU, AUUAUA, AUUAUC, AUUAUG,
AUUCAC, AUUCCA, AUUCCG, AUUCCU, AUUCGA, AUUCGC, AUUCGG, AUUCGU, AUUCUA, AUUCUC,
AUUCUU, AUUGAA, AUUGAC, AUUGAU, AUUGCC, AUUGCG, AUUGCU, AUUGGA, AUUGGC,
AUUGGG, AUUGGU, AUUGUA, AUUGUC, AUUGUG, AUUGUU, AUUUAA, AUUUAG, AUUUAU,
AUUUCC, AUUUCG, AUUUCU, AUUUGA, AUUUGC, AUUUGU, AUUUUA, AUUUUC, AUUUUG,
AUUUUU, CAAAAG, CAAACA, CAAACC, CAAACG, CAAACU, CAAAGA, CAAAGG, CAAAUA, CAAAUU,
CAACAC, CAACAU, CAACCA, CAACCC, CAACCG, CAACGA, CAACGC, CAACGG, CAACGU, CAACUA,
CAACUC, CAACUG, CAACUU, CAAGAA, CAAGAC, CAAGAU, CAAGCA, CAAGCC, CAAGCG, CAAGCU,
CAAGGA, CAAGGG, CAAGUC, CAAGUG, CAAGUU, CAAUAA, CAAUAC, CAAUAG, CAAUCC, CAAUCG,
CAAUCU, CAAUGA, CAAUGC, CAAUGG, CAAUGU, CAAUUC, CAAUUG, CAAUUU, CACAAU, CACACA,
CACACG, CACACU, CACAGA, CACAGC, CACAGG, CACAUA, CACAUC, CACAUU, CACCAA, CACCAC,
CACCAU, CACCCA, CACCCC, CACCCG, CACCGA, CACCGC, CACCGG, CACCGU, CACCUA, CACCUU,
CACGAA, CACGAC, CACGAG, CACGAU, CACGCA, CACGCC, CACGCU, CACGGA, CACGGC, CACGGG,
CACGGU, CACGUA, CACGUC, CACGUG, CACGUU, CACUAA, CACUAG, CACUAU, CACUCA, CACUCG,
CACUGA, CACUGC, CACUGG, CACUUA, CACUUC, CACUUU, CAGAAA, CAGAAG, CAGAAU, CAGACC,
CAGACG, CAGAGC, CAGAUA, CAGAUC, CAGCCG, CAGCCU, CAGCGA, CAGCGC, CAGCGG, CAGCGU,
CAGCUC, CAGCUU, CAGGAU, CAGGGG, CAGGGU, CAGGUA, CAGGUC, CAGGUU, CAGUAC, CAGUCG,
CAGUUG, CAUAAA, CAUAAC, CAUAAG, CAUAAU, CAUACA, CAUACC, CAUACG, CAUACU, CAUAGA,
CAUAGG, CAUAGU, CAUAUA, CAUAUC, CAUAUG, CAUCAA, CAUCAC, CAUCAG, CAUCAU, CAUCCA,
CAUCCC, CAUCCG, CAUCGA, CAUCGC, CAUCGG, CAUCGU, CAUCUA, CAUCUC, CAUCUG, CAUCUU,
CAUGAA, CAUGAC, CAUGAG, CAUGAU, CAUGCA, CAUGCC, CAUGCG, CAUGCU, CAUGGC, CAUGGG,
CAUGGU, CAUGUA, CAUGUC, CAUGUU, CAUUAA, CAUUAC, CAUUAG, CAUUCA, CAUUCC, CAUUCG,
CAUUCU, CAUUGA, CAUUGG, CAUUUC, CAUUUG, CAUUUU, CCAAAA, CCAAAC, CCAAAG, CCAAAU,
CCAACA, CCAACC, CCAACG, CCAACU, CCAAGA, CCAAGC, CCAAGG, CCAAUC, CCAAUG, CCAAUU,
CCACAA, CCACAC, CCACAG, CCACAU, CCACCA, CCACCC, CCACCG, CCACCU, CCACGA, CCACGC,
CCACGG, CCACGU, CCACUA, CCACUC, CCACUU, CCAGAA, CCAGAC, CCAGAG, CCAGCC, CCAGGU,
CCAGUC, CCAGUU, CCAUAA, CCAUAC, CCAUAG, CCAUAU, CCAUCA, CCAUCC, CCAUCU, CCAUGA,
CCAUGC, CCAUGG, CCAUUC, CCAUUG, CCAUUU, CCCAAC, CCCAAG, CCCAAU, CCCACA, CCCAGA,
CCCAGC, CCCAGU, CCCAUA, CCCAUC, CCCAUG, CCCAUU, CCCCAA, CCCCAG, CCCCAU, CCCCCC,
CCCCCG, CCCCCU, CCCCGA, CCCCGC, CCCCGU, CCCCUA, CCCCUC, CCCGAA, CCCGAC, CCCGAU,
CCCGCA, CCCGCU, CCCGGA, CCCGGC, CCCGUA, CCCGUG, CCCGUU, CCCUAA, CCCUAG, CCCUCA,
CCCUCU, CCCUGC, CCCUUA, CCCUUC, CCCUUU, CCGAAA, CCGAAC, CCGAAU, CCGACA, CCGACC,
CCGACG, CCGACU, CCGAGA, CCGAGG, CCGAGU, CCGAUA, CCGAUC, CCGAUG, CCGAUU, CCGCAA,
CCGCAC, CCGCAG, CCGCAU, CCGCCA, CCGCCC, CCGCCG, CCGCCU, CCGCGA, CCGCGC, CCGCGG,
CCGCGU, CCGCUA, CCGCUC, CCGCUG, CCGCUU, CCGGAA, CCGGAU, CCGGCA, CCGGCC, CCGGCG,
CCGGCU, CCGGGA, CCGGGC, CCGGGG, CCGGGU, CCGGUA, CCGGUC, CCGGUG, CCGUAA, CCGUAG,
CCGUAU, CCGUCA, CCGUCC, CCGUCG, CCGUGA, CCGUGU, CCGUUA, CCGUUC, CCGUUG, CCGUUU,
CCUAAC, CCUAAG, CCUAAU, CCUACA, CCUACC, CCUACG, CCUACU, CCUAGA, CCUAGC, CCUAGG,
CCUAGU, CCUAUA, CCUAUC, CCUAUG, CCUAUU, CCUCAA, CCUCAC, CCUCAG, CCUCAU, CCUCCA,
CCUCCC, CCUCCG, CCUCGA, CCUCGC, CCUCGG, CCUCGU, CCUCUA, CCUCUG, CCUGAC, CCUGAU,
CCUGCA, CCUGGG, CCUGGU, CCUGUU, CCUUAA, CCUUAC, CCUUAG, CCUUAU, CCUUCG, CCUUGA,
CCUUGU, CCUUUA, CCUUUC, CCUUUU, CGAAAA, CGAAAC, CGAAAG, CGAAAU, CGAACA, CGAACC,
CGAACG, CGAACU, CGAAGA, CGAAGC, CGAAGG, CGAAGU, CGAAUA, CGAAUC, CGAAUG, CGAAUU,
CGACAA, CGACAC, CGACAU, CGACCA, CGACCU, CGACGA, CGACGC, CGACGG, CGACGU, CGACUA,
CGACUG, CGACUU, CGAGAA, CGAGAC, CGAGAG, CGAGAU, CGAGCA, CGAGCC, CGAGCG, CGAGCU,
CGAGGC, CGAGGG, CGAGGU, CGAGUA, CGAGUC, CGAGUG, CGAGUU, CGAUAA, CGAUAC, CGAUAG,
CGAUAU, CGAUCA, CGAUCC, CGAUCG, CGAUCU, CGAUGA, CGAUGC, CGAUGG, CGAUGU, CGAUUA,
CGAUUC, CGAUUG, CGAUUU, CGCAAA, CGCAAC, CGCAAG, CGCAAU, CGCACA, CGCACC, CGCACG,
CGCAGA, CGCAGC, CGCAGG, CGCAGU, CGCAUA, CGCAUC, CGCAUG, CGCAUU, CGCCAA, CGCCAC,
CGCCAG, CGCCAU, CGCCCA, CGCCCC, CGCCCG, CGCCGA, CGCCGC, CGCCGG, CGCCGU, CGCCUA,
CGCCUG, CGCCUU, CGCGAA, CGCGAC, CGCGAG, CGCGAU, CGCGCA, CGCGCC, CGCGCG, CGCGCU,
CGCGGA, CGCGGC, CGCGGG, CGCGGU, CGCGUA, CGCGUC, CGCGUG, CGCGUU, CGCUAA, CGCUAC,
CGCUAG, CGCUAU, CGCUCA, CGCUCC, CGCUCG, CGCUCU, CGCUGA, CGCUGC, CGCUGG, CGCUGU,
CGCUUA, CGCUUC, CGCUUG, CGGAAA, CGGAAC, CGGAAG, CGGACA, CGGACC, CGGACG, CGGACU,
CGGAGC, CGGAGG, CGGAGU, CGGAUA, CGGAUU, CGGCAA, CGGCAC, CGGCAG, CGGCCA, CGGCCC,
CGGCCG, CGGCGC, CGGCGG, CGGCGU, CGGCUA, CGGCUC, CGGCUG, CGGCUU, CGGGAA, CGGGAC,
CGGGAG, CGGGAU, CGGGCA, CGGGCC, CGGGCG, CGGGCU, CGGGGU, CGGGUA, CGGGUC, CGGGUG,
CGGUAA, CGGUAC, CGGUAG, CGGUAU, CGGUCA, CGGUCG, CGGUCU, CGGUGA, CGGUGG, CGGUGU,
CGGUUA, CGGUUC, CGGUUG, CGGUUU, CGUAAA, CGUAAC, CGUAAG, CGUAAU, CGUACA, CGUACG,
CGUACU, CGUAGA, CGUAGC, CGUAGG, CGUAGU, CGUAUA, CGUAUC, CGUAUG, CGUAUU, CGUCAA,
CGUCAC, CGUCAG, CGUCAU, CGUCCA, CGUCCC, CGUCCG, CGUCCU, CGUCGA, CGUCGG, CGUCGU,
CGUCUA, CGUCUC, CGUCUG, CGUCUU, CGUGAA, CGUGAC, CGUGAG, CGUGAU, CGUGCC, CGUGCG,
CGUGCU, CGUGGA, CGUGGG, CGUGGU, CGUGUA, CGUGUG, CGUUAA, CGUUAC, CGUUAG,
CGUUAU, CGUUCA, CGUUCC, CGUUCG, CGUUCU, CGUUGA, CGUUGC, CGUUGU, CGUUUA, CGUUUC,
CGUUUU, CUAAAA, CUAAAC, CUAAAU, CUAACA, CUAACC, CUAACG, CUAACU, CUAAGA, CUAAGC,
CUAAGU, CUAAUA, CUAAUC, CUAAUG, CUACAC, CUACAU, CUACCA, CUACCC, CUACCG, CUACCU,
CUACGA, CUACGC, CUACGG, CUACGU, CUACUA, CUACUC, CUACUG, CUAGAA, CUAGAG, CUAGAU,
CUAGCA, CUAGCC, CUAGCG, CUAGCU, CUAGGA, CUAGGG, CUAGGU, CUAGUG, CUAGUU, CUAUAA,
CUAUAG, CUAUAU, CUAUCA, CUAUCC, CUAUCG, CUAUCU, CUAUGA, CUAUGC, CUAUGG, CUAUGU,
CUAUUA, CUAUUG, CUCAAC, CUCAAG, CUCAAU, CUCACC, CUCACG, CUCAGC, CUCAUA, CUCAUC,
CUCAUG, CUCAUU, CUCCAC, CUCCCC, CUCCCG, CUCCGA, CUCCGC, CUCCGG, CUCCUA, CUCCUC,
CUCCUU, CUCGAA, CUCGAC, CUCGAG, CUCGAU, CUCGCA, CUCGCC, CUCGCG, CUCGGG, CUCGGU,
CUCGUA, CUCGUC, CUCGUG, CUCGUU, CUCUAA, CUCUAC, CUCUAU, CUCUCA, CUCUCC, CUCUCU,
CUCUGC, CUCUGU, CUCUUA, CUCUUG, CUGAAG, CUGACC, CUGACG, CUGAGC, CUGAUA, CUGAUC,
CUGCCG, CUGCCU, CUGCGA, CUGCUA, CUGCUU, CUGGAG, CUGGAU, CUGGCG, CUGGGU, CUGUAC,
CUGUCA, CUGUCC, CUGUCG, CUGUGG, CUGUGU, CUGUUA, CUGUUU, CUUAAC, CUUAAG, CUUAAU,
CUUACC, CUUACG, CUUAGA, CUUAGC, CUUAGG, CUUAGU, CUUAUA, CUUAUC, CUUAUG, CUUAUU,
CUUCAG, CUUCAU, CUUCCA, CUUCCC, CUUCCG, CUUCCU, CUUCGA, CUUCGC, CUUCGG, CUUCGU,
CUUCUA, CUUGAC, CUUGAG, CUUGAU, CUUGCA, CUUGCC, CUUGCG, CUUGCU, CUUGGC, CUUGGU,
CUUGUU, CUUUAC, CUUUAG, CUUUAU, CUUUCA, CUUUCG, CUUUCU, CUUUGA, CUUUGC, CUUUGU,
CUUUUA, CUUUUC, CUUUUG, CUUUUU, GAAAAA, GAAAAG, GAAAAU, GAAACC, GAAACG, GAAAGA,
GAAAGC, GAAAGU, GAAAUA, GAAAUC, GAAAUG, GAAAUU, GAACAA, GAACAC, GAACAG, GAACAU,
GAACCA, GAACCC, GAACCG, GAACCU, GAACGA, GAACGC, GAACGG, GAACGU, GAACUA, GAACUG,
GAACUU, GAAGAC, GAAGAG, GAAGCA, GAAGCG, GAAGCU, GAAGUC, GAAUAA, GAAUAC, GAAUAG,
GAAUAU, GAAUCC, GAAUCG, GAAUCU, GAAUGA, GAAUGC, GAAUGU, GAAUUA, GAAUUC, GAAUUU,
GACAAA, GACAAG, GACAAU, GACACC, GACAGA, GACAGG, GACAUA, GACAUG, GACAUU, GACCAA,
GACCAC, GACCAG, GACCCA, GACCCC, GACCCG, GACCGC, GACCGG, GACCGU, GACCUA, GACCUC,
GACCUU, GACGAA, GACGAC, GACGAG, GACGAU, GACGCA, GACGCC, GACGCG, GACGCU, GACGGA,
GACGGC, GACGGG, GACGGU, GACGUA, GACGUC, GACGUG, GACGUU, GACUAA, GACUAC, GACUAG,
GACUAU, GACUCA, GACUCC, GACUCG, GACUGG, GACUGU, GACUUA, GACUUG, GACUUU, GAGAAU,
GAGAGA, GAGAGC, GAGAGG, GAGAUA, GAGAUC, GAGCAA, GAGCAU, GAGCCA, GAGCGA, GAGCGG,
GAGCGU, GAGGGU, GAGGUC, GAGGUG, GAGUAA, GAGUAG, GAGUCC, GAGUUC, GAGUUU,
GAUAAA, GAUAAC, GAUAAG, GAUAAU, GAUACA, GAUACC, GAUACG, GAUACU, GAUAGA, GAUAGC,
GAUAGG, GAUAGU, GAUAUA, GAUCAA, GAUCAC, GAUCAU, GAUCCA, GAUCCC, GAUCCU, GAUCGC,
GAUCGG, GAUCGU, GAUCUA, GAUCUG, GAUCUU, GAUGAA, GAUGAC, GAUGAG, GAUGCA, GAUGCC,
GAUGCG, GAUGCU, GAUGGC, GAUGGG, GAUGGU, GAUGUG, GAUGUU, GAUUAA, GAUUAC,
GAUUAG, GAUUAU, GAUUCA, GAUUCG, GAUUCU, GAUUGA, GAUUGC, GAUUUA, GAUUUC,
GAUUUG, GAUUUU, GCAAAC, GCAAAG, GCAAAU, GCAACA, GCAACC, GCAAGC, GCAAGU, GCAAUA,
GCAAUC, GCAAUG, GCAAUU, GCACAA, GCACAC, GCACAG, GCACCC, GCACCG, GCACCU, GCACGA,
GCACGC, GCACGU, GCACUA, GCACUC, GCACUG, GCACUU, GCAGAU, GCAGCC, GCAGCG, GCAGGC,
GCAGUA, GCAGUC, GCAGUG, GCAGUU, GCAUAA, GCAUAG, GCAUAU, GCAUCG, GCAUCU, GCAUGA,
GCAUGC, GCAUGG, GCAUGU, GCAUUA, GCAUUC, GCAUUG, GCAUUU, GCCAAA, GCCAAC, GCCAAU,
GCCACA, GCCACC, GCCACG, GCCAGA, GCCAGU, GCCAUA, GCCAUC, GCCAUG, GCCAUU, GCCCAA,
GCCCAC, GCCCAG, GCCCCG, GCCCGA, GCCCGG, GCCCGU, GCCGAA, GCCGAC, GCCGAG, GCCGAU,
GCCGCA, GCCGCU, GCCGGA, GCCGGC, GCCGGG, GCCGGU, GCCGUA, GCCGUC, GCCGUG, GCCGUU,
GCCUAA, GCCUAU, GCCUCA, GCCUCC, GCCUCG, GCCUGA, GCCUUA, GCCUUU, GCGAAA, GCGAAC,
GCGAAG, GCGAAU, GCGACC, GCGACG, GCGACU, GCGAGA, GCGAGC, GCGAGG, GCGAGU, GCGAUA,
GCGAUC, GCGAUG, GCGAUU, GCGCAA, GCGCAC, GCGCAG, GCGCAU, GCGCCA, GCGCCC, GCGCCU,
GCGCGA, GCGCGU, GCGCUA, GCGCUC, GCGCUG, GCGCUU, GCGGAA, GCGGAC, GCGGAU, GCGGCA,
GCGGCC, GCGGCU, GCGGGA, GCGGUA, GCGGUC, GCGGUU, GCGUAA, GCGUAC, GCGUAG, GCGUAU,
GCGUCA, GCGUCC, GCGUCG, GCGUCU, GCGUGA, GCGUGC, GCGUGG, GCGUGU, GCGUUA, GCGUUC,
GCGUUG, GCGUUU, GCUAAA, GCUAAC, GCUAAG, GCUAAU, GCUACC, GCUACG, GCUACU, GCUAGA,
GCUAGG, GCUAGU, GCUAUA, GCUAUC, GCUAUU, GCUCAA, GCUCAC, GCUCAG, GCUCAU, GCUCCA,
GCUCCC, GCUCCG, GCUCGA, GCUCGC, GCUCGU, GCUCUA, GCUCUC, GCUCUU, GCUGAA, GCUGAC,
GCUGAU, GCUGCA, GCUGCC, GCUGCG, GCUGCU, GCUGUG, GCUGUU, GCUUAC, GCUUAG, GCUUAU,
GCUUCA, GCUUCG, GCUUGA, GCUUGG, GCUUGU, GCUUUA, GCUUUG, GGAAAG, GGAACA, GGAACC,
GGAACG, GGAACU, GGAAGU, GGAAUA, GGAAUC, GGAAUU, GGACAA, GGACAC, GGACAG, GGACAU,
GGACCG, GGACGA, GGACGC, GGACGU, GGACUA, GGACUC, GGACUU, GGAGAC, GGAGCA, GGAGCG,
GGAGGG, GGAGUA, GGAUAA, GGAUAC, GGAUCA, GGAUCC, GGAUCG, GGAUCU, GGAUGC, GGAUUA,
GGAUUG, GGCAAU, GGCACA, GGCACU, GGCAGA, GGCAUA, GGCAUC, GGCCAC, GGCCAG, GGCCCC,
GGCCGA, GGCCGC, GGCCGU, GGCCUA, GGCCUG, GGCCUU, GGCGAA, GGCGAG, GGCGAU, GGCGCA,
GGCGCU, GGCGGU, GGCGUA, GGCGUC, GGCGUG, GGCGUU, GGCUAA, GGCUAC, GGCUAG, GGCUAU,
GGCUCC, GGCUCG, GGCUGA, GGCUUA, GGCUUC, GGCUUG, GGGAAU, GGGACA, GGGAGA, GGGAGU,
GGGAUA, GGGAUU, GGGCAA, GGGCAC, GGGCAG, GGGCCG, GGGCGG, GGGGCC, GGGGGG,
GGGGGU, GGGGUA, GGGUAC, GGGUAU, GGGUCA, GGGUCC, GGGUCG, GGGUGA, GGGUGC,
GGGUUA, GGGUUG, GGUAAA, GGUAAC, GGUAAG, GGUAAU, GGUACA, GGUACC, GGUACG,
GGUACU, GGUAGC, GGUAGG, GGUAGU, GGUAUA, GGUAUC, GGUAUG, GGUCAA, GGUCAC,
GGUCAG, GGUCAU, GGUCCA, GGUCCG, GGUCCU, GGUCGA, GGUCGC, GGUCGG, GGUCGU, GGUCUC,
GGUCUU, GGUGAA, GGUGAC, GGUGAU, GGUGCA, GGUGCC, GGUGGC, GGUGUA, GGUGUC,
GGUUAA, GGUUAG, GGUUAU, GGUUCA, GGUUCC, GGUUCG, GGUUGC, GGUUUC, GGUUUU,
GUAAAA, GUAAAG, GUAAAU, GUAACC, GUAACG, GUAACU, GUAAGA, GUAAGC, GUAAGG, GUAAGU,
GUAAUA, GUAAUC, GUAAUG, GUAAUU, GUACAA, GUACAC, GUACAG, GUACAU, GUACCA, GUACCC,
GUACCG, GUACCU, GUACGA, GUACGC, GUACGG, GUACGU, GUACUA, GUACUC, GUACUG, GUACUU,
GUAGAA, GUAGAC, GUAGCA, GUAGCC, GUAGCG, GUAGCU, GUAGGA, GUAGGC, GUAGGG,
GUAGGU, GUAGUA, GUAGUC, GUAUAA, GUAUAC, GUAUAG, GUAUAU, GUAUCA, GUAUCG,
GUAUCU, GUAUGA, GUAUGC, GUAUGG, GUAUUA, GUAUUG, GUAUUU, GUCAAA, GUCAAG,
GUCAAU, GUCACA, GUCACC, GUCACG, GUCAGA, GUCAGC, GUCAGG, GUCAUA, GUCAUC, GUCAUG,
GUCCAA, GUCCAC, GUCCAU, GUCCCC, GUCCCU, GUCCGA, GUCCGC, GUCCGG, GUCCGU, GUCCUA,
GUCCUG, GUCCUU, GUCGAA, GUCGAC, GUCGAG, GUCGAU, GUCGCA, GUCGCC, GUCGCG, GUCGCU,
GUCGGA, GUCGGC, GUCGGG, GUCGGU, GUCGUA, GUCGUC, GUCGUU, GUCUAA, GUCUAG, GUCUCA,
GUCUCC, GUCUCG, GUCUGA, GUCUGG, GUCUGU, GUCUUC, GUCUUU, GUGAAA, GUGAAC, GUGAAG,
GUGACC, GUGACG, GUGAGA, GUGAGC, GUGAGU, GUGAUC, GUGAUG, GUGAUU, GUGCAC,
GUGCAU, GUGCCC, GUGCCG, GUGCGA, GUGCGG, GUGCGU, GUGCUA, GUGCUC, GUGCUG,
GUGGAG, GUGGCG, GUGGCU, GUGGGU, GUGGUC, GUGGUG, GUGUAA, GUGUAG, GUGUCG,
GUGUGA, GUGUGC, GUGUGU, GUGUUG, GUGUUU, GUUAAA, GUUAAC, GUUAAG, GUUACA,
GUUACC, GUUACG, GUUACU, GUUAGA, GUUAGC, GUUAGU, GUUAUA, GUUAUC, GUUAUG,
GUUAUU, GUUCAA, GUUCAC, GUUCAG, GUUCCA, GUUCCG, GUUCGA, GUUCGC, GUUCGG, GUUCGU,
GUUCUA, GUUCUG, GUUGAA, GUUGAC, GUUGAG, GUUGAU, GUUGCG, GUUGCU, GUUGGA,
GUUGGC, GUUGGU, GUUGUC, GUUGUG, GUUGUU, GUUUAA, GUUUAC, GUUUAG, GUUUAU,
GUUUCA, GUUUCC, GUUUCU, GUUUGA, GUUUGC, GUUUGG, GUUUGU, GUUUUA, GUUUUC,
GUUUUU, UAAAAA, UAAAAC, UAAAAG, UAAAAU, UAAACA, UAAACC, UAAACG, UAAACU, UAAAGA,
UAAAGG, UAAAGU, UAAAUA, UAAAUC, UAAAUG, UAAAUU, UAACAA, UAACAC, UAACAG, UAACCA,
UAACCC, UAACCG, UAACCU, UAACGA, UAACGC, UAACGG, UAACGU, UAACUA, UAACUG, UAACUU,
UAAGAG, UAAGAU, UAAGCA, UAAGCC, UAAGCG, UAAGCU, UAAGGA, UAAGGC, UAAGGG, UAAGGU,
UAAGUA, UAAGUC, UAAGUG, UAAGUU, UAAUAA, UAAUCA, UAAUCC, UAAUCG, UAAUCU, UAAUGA,
UAAUGG, UAAUGU, UAAUUA, UAAUUC, UAAUUG, UACAAC, UACAAG, UACAAU, UACACC, UACACG,
UACACU, UACAGA, UACAGC, UACAUA, UACAUC, UACAUU, UACCAA, UACCAC, UACCAG, UACCAU,
UACCCC, UACCCG, UACCCU, UACCGA, UACCGC, UACCGG, UACCGU, UACCUA, UACCUG, UACGAA,
UACGAC, UACGAG, UACGAU, UACGCA, UACGCC, UACGCG, UACGCU, UACGGC, UACGGG, UACGGU,
UACGUA, UACGUC, UACGUG, UACGUU, UACUAA, UACUAC, UACUAG, UACUAU, UACUCA, UACUCC,
UACUCG, UACUCU, UACUGA, UACUGC, UACUGG, UACUUA, UACUUG, UACUUU, UAGAAA, UAGAAG,
UAGAAU, UAGACA, UAGACG, UAGAGA, UAGAGC, UAGAGU, UAGAUA, UAGAUC, UAGAUG, UAGCAU,
UAGCCC, UAGCCG, UAGCCU, UAGCGA, UAGCGC, UAGCGU, UAGCUA, UAGCUC, UAGCUG, UAGGAA,
UAGGAU, UAGGCG, UAGGCU, UAGGGU, UAGGUC, UAGGUG, UAGGUU, UAGUAA, UAGUAC,
UAGUAG, UAGUAU, UAGUCA, UAGUCG, UAGUGU, UAGUUA, UAGUUC, UAGUUG, UAGUUU,
UAUAAC, UAUAAG, UAUACU, UAUAGA, UAUAGC, UAUAGG, UAUAGU, UAUAUA, UAUAUC, UAUAUG,
UAUAUU, UAUCAA, UAUCAC, UAUCAU, UAUCCA, UAUCCC, UAUCCG, UAUCCU, UAUCGA, UAUCGC,
UAUCGG, UAUCGU, UAUCUA, UAUCUC, UAUCUG, UAUCUU, UAUGAA, UAUGAC, UAUGAG,
UAUGAU, UAUGCA, UAUGCG, UAUGCU, UAUGGA, UAUGGC, UAUGUC, UAUGUG, UAUGUU,
UAUUAG, UAUUCA, UAUUCC, UAUUCG, UAUUCU, UAUUGA, UAUUGG, UAUUUA, UAUUUC,
UAUUUG, UAUUUU, UCAAAA, UCAAAC, UCAAAG, UCAACC, UCAACU, UCAAGA, UCAAGC, UCAAUA,
UCAAUC, UCAAUG, UCAAUU, UCACCC, UCACCG, UCACCU, UCACGA, UCACGC, UCACGG, UCACGU,
UCACUA, UCACUC, UCACUU, UCAGAA, UCAGAC, UCAGAG, UCAGCG, UCAGCU, UCAGGA, UCAGGC,
UCAGGU, UCAGUC, UCAGUU, UCAUAA, UCAUCA, UCAUCC, UCAUCG, UCAUGC, UCAUGG, UCAUGU,
UCAUUA, UCAUUG, UCCAAA, UCCAAC, UCCAAG, UCCAAU, UCCACA, UCCACC, UCCACG, UCCAGC,
UCCAGG, UCCAUA, UCCAUC, UCCAUU, UCCCAA, UCCCAG, UCCCAU, UCCCCC, UCCCCG, UCCCCU,
UCCCGA, UCCCGC, UCCCGG, UCCCGU, UCCCUA, UCCCUC, UCCGAA, UCCGAC, UCCGAG, UCCGAU,
UCCGCA, UCCGCC, UCCGGA, UCCGGC, UCCGGU, UCCGUA, UCCGUC, UCCGUG, UCCUAA, UCCUCA,
UCCUCG, UCCUCU, UCCUGC, UCCUGU, UCCUUA, UCCUUC, UCCUUU, UCGAAA, UCGAAC, UCGAAG,
UCGAAU, UCGACA, UCGACC, UCGACG, UCGACU, UCGAGA, UCGAGC, UCGAGG, UCGAUA, UCGAUC,
UCGAUG, UCGAUU, UCGCAA, UCGCAC, UCGCAG, UCGCAU, UCGCCA, UCGCCC, UCGCCG, UCGCCU,
UCGCGA, UCGCGC, UCGCGU, UCGCUA, UCGCUC, UCGGAA, UCGGAC, UCGGAG, UCGGAU, UCGGCA,
UCGGCU, UCGGGG, UCGGGU, UCGGUC, UCGGUG, UCGGUU, UCGUAA, UCGUAC, UCGUAG,
UCGUAU, UCGUCA, UCGUCC, UCGUCG, UCGUCU, UCGUGA, UCGUGU, UCGUUA, UCGUUC, UCGUUG,
UCGUUU, UCUAAC, UCUAAG, UCUAAU, UCUACA, UCUACC, UCUACG, UCUACU, UCUAGC, UCUAGG,
UCUAGU, UCUAUA, UCUAUC, UCUAUG, UCUAUU, UCUCAG, UCUCAU, UCUCCG, UCUCGC, UCUCGG,
UCUCGU, UCUCUC, UCUGAA, UCUGAU, UCUGCA, UCUGCG, UCUGCU, UCUGGC, UCUGGU, UCUGUC,
UCUGUG, UCUGUU, UCUUAA, UCUUAC, UCUUAG, UCUUAU, UCUUCA, UCUUCC, UCUUCG, UCUUCU,
UCUUGC, UCUUGG, UCUUGU, UCUUUA, UCUUUC, UCUUUG, UCUUUU, UGAAAA, UGAAAC,
UGAACA, UGAACC, UGAAGG, UGAAUC, UGAAUG, UGACAA, UGACAC, UGACAG, UGACCA, UGACCC,
UGACCG, UGACGA, UGACGC, UGACGG, UGACGU, UGACUA, UGACUC, UGACUU, UGAGAG, UGAGAU,
UGAGCA, UGAGCC, UGAGCU, UGAGGC, UGAGGU, UGAGUA, UGAGUU, UGAUAC, UGAUAG,
UGAUAU, UGAUCA, UGAUCG, UGAUCU, UGAUGA, UGAUGC, UGAUGG, UGAUGU, UGAUUA,
UGAUUC, UGAUUG, UGAUUU, UGCAAC, UGCAAG, UGCACA, UGCACG, UGCAGG, UGCAGU, UGCAUC,
UGCCCA, UGCCCC, UGCCCG, UGCCGA, UGCCGC, UGCCGG, UGCCGU, UGCCUA, UGCCUC, UGCCUG,
UGCCUU, UGCGAA, UGCGAC, UGCGAU, UGCGCC, UGCGCG, UGCGCU, UGCGGC, UGCGGG, UGCGGU,
UGCGUA, UGCGUC, UGCGUG, UGCGUU, UGCUAC, UGCUAU, UGCUCC, UGCUCG, UGCUGC, UGCUGG,
UGCUGU, UGCUUA, UGCUUU, UGGAAC, UGGAAG, UGGAGC, UGGAUC, UGGAUU, UGGCAA,
UGGCAC, UGGCAG, UGGCCG, UGGCCU, UGGCGA, UGGCGC, UGGCGU, UGGCUA, UGGCUC, UGGCUU,
UGGGAA, UGGGCA, UGGGCC, UGGGGC, UGGGUC, UGGUAA, UGGUAG, UGGUAU, UGGUCC,
UGGUCG, UGGUCU, UGGUGA, UGGUGC, UGGUGG, UGGUGU, UGGUUA, UGGUUG, UGUAAA,
UGUAAC, UGUAAG, UGUACC, UGUACG, UGUACU, UGUAGA, UGUAGC, UGUAGU, UGUAUC,
UGUAUU, UGUCAA, UGUCAC, UGUCAG, UGUCAU, UGUCCA, UGUCCC, UGUCCG, UGUCGA, UGUCGC,
UGUCGG, UGUCGU, UGUCUA, UGUCUC, UGUGAC, UGUGAG, UGUGAU, UGUGCA, UGUGGU,
UGUGUA, UGUGUU, UGUUAC, UGUUAG, UGUUAU, UGUUCA, UGUUCC, UGUUCG, UGUUGG,
UGUUGU, UGUUUA, UGUUUC, UGUUUG, UGUUUU, UUAAAA, UUAAAC, UUAAAG, UUAAAU,
UUAACC, UUAACG, UUAACU, UUAAGU, UUAAUA, UUAAUC, UUAAUG, UUAAUU, UUACAA, UUACAC,
UUACAG, UUACAU, UUACCA, UUACCC, UUACCG, UUACCU, UUACGA, UUACGC, UUACGG, UUACGU,
UUACUA, UUACUC, UUACUG, UUACUU, UUAGAA, UUAGAC, UUAGCC, UUAGCG, UUAGCU, UUAGGC,
UUAGGU, UUAGUA, UUAGUC, UUAGUU, UUAUAA, UUAUAC, UUAUAG, UUAUAU, UUAUCC,
UUAUCG, UUAUCU, UUAUGA, UUAUGG, UUAUGU, UUAUUA, UUAUUC, UUAUUG, UUAUUU,
UUCAAC, UUCAAU, UUCACA, UUCACC, UUCACG, UUCACU, UUCAGC, UUCAGG, UUCAGU, UUCAUA,
UUCAUC, UUCAUG, UUCAUU, UUCCAA, UUCCCA, UUCCCG, UUCCGA, UUCCGU, UUCCUU, UUCGAA,
UUCGAC, UUCGAG, UUCGAU, UUCGCA, UUCGCC, UUCGCG, UUCGCU, UUCGGA, UUCGGC, UUCGGG,
UUCGGU, UUCGUA, UUCGUC, UUCGUG, UUCGUU, UUCUAC, UUCUAG, UUCUCA, UUCUCG,
UUCUGG, UUCUUA, UUCUUU, UUGAAA, UUGAAC, UUGAAG, UUGAAU, UUGACC, UUGACG,
UUGACU, UUGAGA, UUGAGC, UUGAGU, UUGAUA, UUGAUC, UUGAUG, UUGAUU, UUGCAA,
UUGCAC, UUGCAG, UUGCAU, UUGCCC, UUGCCG, UUGCGA, UUGCGC, UUGCGG, UUGCGU, UUGCUA,
UUGCUC, UUGCUG, UUGCUU, UUGGAA, UUGGAG, UUGGCC, UUGGCG, UUGGCU, UUGGGC,
UUGGGU, UUGGUA, UUGGUG, UUGUAA, UUGUAC, UUGUCA, UUGUCG, UUGUCU, UUGUGC,
UUGUGG, UUGUUA, UUGUUG, UUGUUU, UUUAAA, UUUAAC, UUUAAG, UUUAAU, UUUACA,
UUUACC, UUUACG, UUUACU, UUUAGA, UUUAGC, UUUAGG, UUUAGU, UUUAUA, UUUAUC,
UUUAUG, UUUAUU, UUUCAU, UUUCCA, UUUCCG, UUUCCU, UUUCGA, UUUCGC, UUUCGG,
UUUCGU, UUUCUA, UUUCUC, UUUCUG, UUUCUU, UUUGAA, UUUGAC, UUUGAG, UUUGAU,
UUUGCC, UUUGCU, UUUGGA, UUUGGC, UUUGGG, UUUGGU, UUUGUA, UUUGUC, UUUGUU,
UUUUAA, UUUUAG, UUUUAU, UUUUCC, UUUUCG, UUUUCU, UUUUGA, UUUUGC, UUUUGG,
UUUUGU, UUUUUA, UUUUUC, UUUUUU

TABLE 2
Experimental evaluation of single stranded oligonucleotides.

| | Oligo | | Avg RQ | | | | | Time | Assay |
|---|---|---|---|---|---|---|---|---|---|
| Seq ID | Name | Avg RQ | SD | Target | Probe | [oligo] | cell line | (hr) | Type |
| 45714 | unc- | 1 | 0 | FOXP3 | FOXP3 | 10000 | huTcell+ | 48 | qRTPCR |
| | 293 | | | | | | | | |
| | m01 | | | | | | | | |
| | Ctrl Un | 0.86831487 | 0.28399334 | FOXP3 | FOXP3 | 10000 | huTcell+ | 48 | qRTPCR |
| 42428 | FOXP3- | 3.04123508 | 1.41567948 | FOXP3 | FOXP3 | 10000 | huTcell+ | 48 | qRTPCR |
| | 01 m01 | | | | | | | | |
| 42429 | FOXP3- | 0.8050441 | 0.10470739 | FOXP3 | FOXP3 | 10000 | huTcell+ | 48 | qRTPCR |
| | 02 m01 | | | | | | | | |
| 42431 | FOXP3- | 0.60289195 | 0.00365975 | FOXP3 | FOXP3 | 10000 | huTcell+ | 48 | qRTPCR |
| | 03 m01 | | | | | | | | |
| 42427 | FOXP3- | 2.00872837 | 0.5894724 | FOXP3 | FOXP3 | 10000 | huTcell+ | 48 | qRTPCR |
| | 04 m01 | | | | | | | | |
| 42426 | FOXP3- | 3.63267026 | 0.40296967 | FOXP3 | FOXP3 | 10000 | huTcell+ | 48 | qRTPCR |
| | 05 m01 | | | | | | | | |
| 42420 | FOXP3- | 3.7983637 | 0.18781968 | FOXP3 | FOXP3 | 10000 | huTcell+ | 48 | qRTPCR |
| | 06 m01 | | | | | | | | |
| 42381 | FOXP3- | 1.93225341 | 0.78123517 | FOXP3 | FOXP3 | 10000 | huTcell+ | 48 | qRTPCR |
| | 07 m01 | | | | | | | | |
| 42862 | FOXP3- | 3.1661807 | 0.03834323 | FOXP3 | FOXP3 | 10000 | huTcell+ | 48 | qRTPCR |
| | 08 m01 | | | | | | | | |
| 42861 | FOXP3- | 1.90576687 | 0.66711136 | FOXP3 | FOXP3 | 10000 | huTcell+ | 48 | qRTPCR |
| | 09 m01 | | | | | | | | |
| 42859 | FOXP3- | 1.86380551 | 0.08363845 | FOXP3 | FOXP3 | 10000 | huTcell+ | 48 | qRTPCR |
| | 10 m01 | | | | | | | | |
| 42858 | FOXP3- | 3.13763788 | 1.08606849 | FOXP3 | FOXP3 | 10000 | huTcell+ | 48 | qRTPCR |
| | 11 m01 | | | | | | | | |
| 42854 | FOXP3- | 1.91683566 | 0.19100954 | FOXP3 | FOXP3 | 10000 | huTcell+ | 48 | qRTPCR |
| | 12 m01 | | | | | | | | |
| 41897 | FOXP3- | 3.82088674 | 0.32436208 | FOXP3 | FOXP3 | 10000 | huTcell+ | 48 | qRTPCR |
| | 13 m01 | | | | | | | | |
| 41052 | FOXP3- | 1.71924737 | 0.28439737 | FOXP3 | FOXP3 | 10000 | huTcell+ | 48 | qRTPCR |
| | 14 m01 | | | | | | | | |
| 41877 | FOXP3- | 1.68978766 | 0.05339158 | FOXP3 | FOXP3 | 10000 | huTcell+ | 48 | qRTPCR |
| | 15 m01 | | | | | | | | |
| 43243 | FOXP3- | 1.32422504 | 0.11241901 | FOXP3 | FOXP3 | 10000 | huTcell+ | 48 | qRTPCR |
| | 16 m01 | | | | | | | | |
| 43375 | FOXP3- | 2.96627721 | 0.80602523 | FOXP3 | FOXP3 | 10000 | huTcell+ | 48 | qRTPCR |
| | 17 m01 | | | | | | | | |
| 43376 | FOXP3- | 2.48879558 | 0.31376938 | FOXP3 | FOXP3 | 10000 | huTcell+ | 48 | qRTPCR |
| | 18 m01 | | | | | | | | |
| 43379 | FOXP3- | 1.62176367 | 0.07036153 | FOXP3 | FOXP3 | 10000 | huTcell+ | 48 | qRTPCR |
| | 19 m01 | | | | | | | | |
| 43380 | FOXP3- | 0.98364872 | 0.35941408 | FOXP3 | FOXP3 | 10000 | huTcell+ | 48 | qRTPCR |
| | 20 m01 | | | | | | | | |
| 43381 | FOXP3- | 2.42135072 | 0.86070806 | FOXP3 | FOXP3 | 10000 | huTcell+ | 48 | qRTPCR |
| | 21 m01 | | | | | | | | |
| 43216 | FOXP3- | 4.40567095 | 0.52743242 | FOXP3 | FOXP3 | 10000 | huTcell+ | 48 | qRTPCR |
| | 22 m01 | | | | | | | | |
| 43215 | FOXP3- | 5.25941152 | 1.28342081 | FOXP3 | FOXP3 | 10000 | huTcell+ | 48 | qRTPCR |
| | 23 m01 | | | | | | | | |
| 43209 | FOXP3- | 0.84410485 | 0.11625078 | FOXP3 | FOXP3 | 10000 | huTcell+ | 48 | qRTPCR |
| | 24 m01 | | | | | | | | |
| 43207 | FOXP3- | 1.97006843 | 0.70813234 | FOXP3 | FOXP3 | 10000 | huTcell+ | 48 | qRTPCR |
| | 25 m01 | | | | | | | | |
| 43205 | FOXP3- | 1.41020745 | 0.5560462 | FOXP3 | FOXP3 | 10000 | huTcell+ | 48 | qRTPCR |
| | 26 m01 | | | | | | | | |
| 43204 | FOXP3- | 2.31376112 | 0.88936632 | FOXP3 | FOXP3 | 10000 | huTcell+ | 48 | qRTPCR |
| | 27 m01 | | | | | | | | |
| 43203 | FOXP3- | 2.31114582 | 0.95718326 | FOXP3 | FOXP3 | 10000 | huTcell+ | 48 | qRTPCR |
| | 28 m01 | | | | | | | | |
| 42533 | FOXP3- | 2.42133228 | 0.8569018 | FOXP3 | FOXP3 | 10000 | huTcell+ | 48 | qRTPCR |
| | 29 m01 | | | | | | | | |
| 42535 | FOXP3- | 2.84205507 | 0.73556048 | FOXP3 | FOXP3 | 10000 | huTcell+ | 48 | qRTPCR |
| | 30 m01 | | | | | | | | |
| 42580 | FOXP3- | 1.68295946 | 0.31974715 | FOXP3 | FOXP3 | 10000 | huTcell+ | 48 | qRTPCR |
| | 31 m01 | | | | | | | | |
| 42579 | FOXP3- | 1.8023751 | 0.3645291 | FOXP3 | FOXP3 | 10000 | huTcell+ | 48 | qRTPCR |
| | 32 m01 | | | | | | | | |
| 42578 | FOXP3- | 1.50499095 | 0.35245102 | FOXP3 | FOXP3 | 10000 | huTcell+ | 48 | qRTPCR |
| | 33 m01 | | | | | | | | |
| 42576 | FOXP3- | 1.55410054 | 0.47399665 | FOXP3 | FOXP3 | 10000 | huTcell+ | 48 | qRTPCR |
| | 34 m01 | | | | | | | | |
| 42575 | FOXP3- | 1.94173584 | 0.79237942 | FOXP3 | FOXP3 | 10000 | huTcell+ | 48 | qRTPCR |
| | 35 m01 | | | | | | | | |
| 42574 | FOXP3- | 1.78502753 | 0.56168053 | FOXP3 | FOXP3 | 10000 | huTcell+ | 48 | qRTPCR |
| | 36 m01 | | | | | | | | |
| 42572 | FOXP3- | 1.18497696 | 0.63819605 | FOXP3 | FOXP3 | 10000 | huTcell+ | 48 | qRTPCR |
| | 37 m01 | | | | | | | | |
| 42571 | FOXP3- | 2.8387527 | 0.16520034 | FOXP3 | FOXP3 | 10000 | huTcell+ | 48 | qRTPCR |
| | 38 m01 | | | | | | | | |
| 42570 | FOXP3- | 2.10550749 | 0.99999309 | FOXP3 | FOXP3 | 10000 | huTcell+ | 48 | qRTPCR |
| | 39 m01 | | | | | | | | |
| 42569 | FOXP3- | 2.16167457 | 0.41220644 | FOXP3 | FOXP3 | 10000 | huTcell+ | 48 | qRTPCR |
| | 40 m01 | | | | | | | | |
| 42568 | FOXP3- | 2.15960714 | 0.14639811 | FOXP3 | FOXP3 | 10000 | huTcell+ | 48 | qRTPCR |
| | 41 m01 | | | | | | | | |
| 42567 | FOXP3- | 2.01961568 | 0.24503396 | FOXP3 | FOXP3 | 10000 | huTcell+ | 48 | qRTPCR |
| | 42 m01 | | | | | | | | |
| 43447 | FOXP3- | 1.40341903 | 0.43004175 | FOXP3 | FOXP3 | 10000 | huTcell+ | 48 | qRTPCR |
| | 43 m01 | | | | | | | | |
| 43450 | FOXP3- | 2.68263331 | 0.86376389 | FOXP3 | FOXP3 | 10000 | huTcell+ | 48 | qRTPCR |
| | 44 m01 | | | | | | | | |
| 43452 | FOXP3- | 1.29728052 | 0.07607689 | FOXP3 | FOXP3 | 10000 | huTcell+ | 48 | qRTPCR |
| | 45 m01 | | | | | | | | |
| 43455 | FOXP3- | 1.69333468 | 0.28727806 | FOXP3 | FOXP3 | 10000 | huTcell+ | 48 | qRTPCR |
| | 46 m01 | | | | | | | | |
| 43456 | FOXP3- | 3.98814452 | 1.11553672 | FOXP3 | FOXP3 | 10000 | huTcell+ | 48 | qRTPCR |
| | 47 m01 | | | | | | | | |
| 43458 | FOXP3- | 1.6118209 | 0.51299822 | FOXP3 | FOXP3 | 10000 | huTcell+ | 48 | qRTPCR |
| | 48 m01 | | | | | | | | |
| 43459 | FOXP3- | 2.71141213 | 0.83492691 | FOXP3 | FOXP3 | 10000 | huTcell+ | 48 | qRTPCR |
| | 49 m01 | | | | | | | | |
| 36466 | FOXP3- | 5.67570129 | 0.17800894 | FOXP3 | FOXP3 | 10000 | huTcell+ | 48 | qRTPCR |
| | 50 m01 | | | | | | | | |
| 36439 | FOXP3- | 0.77547248 | 0.11344935 | FOXP3 | FOXP3 | 10000 | huTcell+ | 48 | qRTPCR |
| | 51 m01 | | | | | | | | |
| 36437 | FOXP3- | 1.66635528 | 0.18881864 | FOXP3 | FOXP3 | 10000 | huTcell+ | 48 | qRTPCR |
| | 52 m01 | | | | | | | | |
| 29244 | FOXP3- | 4.78589887 | 0.05731016 | FOXP3 | FOXP3 | 10000 | huTcell+ | 48 | qRTPCR |
| | 53 m01 | | | | | | | | |
| 29253 | FOXP3- | 1.49697594 | 0.51286873 | FOXP3 | FOXP3 | 10000 | huTcell+ | 48 | qRTPCR |
| | 54 m01 | | | | | | | | |
| 41675 | FOXP3- | 1.34388255 | 0.16698218 | FOXP3 | FOXP3 | 10000 | huTcell+ | 48 | qRTPCR |
| | 55 m01 | | | | | | | | |
| 43610 | FOXP3- | 3.19719516 | 0.03774779 | FOXP3 | FOXP3 | 10000 | huTcell+ | 48 | qRTPCR |
| | 56 m01 | | | | | | | | |
| 43612 | FOXP3- | 1.16023805 | 0.63606665 | FOXP3 | FOXP3 | 10000 | huTcell+ | 48 | qRTPCR |
| | 57 m01 | | | | | | | | |
| 43613 | FOXP3- | 1.32913775 | 0.75851478 | FOXP3 | FOXP3 | 10000 | huTcell+ | 48 | qRTPCR |
| | 58 m01 | | | | | | | | |
| 43616 | FOXP3- | 0.69574924 | 0.28991794 | FOXP3 | FOXP3 | 10000 | huTcell+ | 48 | qRTPCR |
| | 59 m01 | | | | | | | | |
| 40694 | FOXP3- | 1.43965065 | 0.31103627 | FOXP3 | FOXP3 | 10000 | huTcell+ | 48 | qRTPCR |
| | 60 m01 | | | | | | | | |
| 45714 | unc- | 1 | 0 | FOXP3 | FOXP3 | 10000 | huTcell+ | 48 | qRTPCR |
| | 293 | | | | | | | | |
| | m01 | | | | | | | | |
| | Ctrl Un | 1.01249113 | 0.14941646 | FOXP3 | FOXP3 | 10000 | huTcell+ | 48 | qRTPCR |
| 42428 | FOXP3- | 1.01421223 | 0.20838028 | FOXP3 | FOXP3 | 10000 | huTcell+ | 48 | qRTPCR |
| | 01 m01 | | | | | | | | |
| 42429 | FOXP3- | 0.80548151 | 0.16301023 | FOXP3 | FOXP3 | 10000 | huTcell+ | 48 | qRTPCR |
| | 02 m01 | | | | | | | | |
| 42431 | FOXP3- | 1.85375015 | 0.49859871 | FOXP3 | FOXP3 | 10000 | huTcell+ | 48 | qRTPCR |
| | 03 m01 | | | | | | | | |
| 42427 | FOXP3- | 1.03803618 | 0.30098857 | FOXP3 | FOXP3 | 10000 | huTcell+ | 48 | qRTPCR |
| | 04 m01 | | | | | | | | |
| 42426 | FOXP3- | 1.18949951 | 0.33746604 | FOXP3 | FOXP3 | 10000 | huTcell+ | 48 | qRTPCR |
| | 05 m01 | | | | | | | | |
| 42420 | FOXP3- | 1.49213089 | 0.3555691 | FOXP3 | FOXP3 | 10000 | huTcell+ | 48 | qRTPCR |
| | 06 m01 | | | | | | | | |
| 42381 | FOXP3- | 1.04465779 | 0.20591828 | FOXP3 | FOXP3 | 10000 | huTcell+ | 48 | qRTPCR |
| | 07 m01 | | | | | | | | |
| 42862 | FOXP3- | 1.20846974 | 0.30870018 | FOXP3 | FOXP3 | 10000 | huTcell+ | 48 | qRTPCR |
| | 08 m01 | | | | | | | | |
| 42861 | FOXP3- | 1.06823309 | 0.01223336 | FOXP3 | FOXP3 | 10000 | huTcell+ | 48 | qRTPCR |
| | 09 m01 | | | | | | | | |
| 42859 | FOXP3- | 1.18996991 | 1.00158697 | FOXP3 | FOXP3 | 10000 | huTcell+ | 48 | qRTPCR |
| | 10 m01 | | | | | | | | |
| 42858 | FOXP3- | 1.01995327 | 0.24692075 | FOXP3 | FOXP3 | 10000 | huTcell+ | 48 | qRTPCR |
| | 11 m01 | | | | | | | | |
| 42854 | FOXP3- | 0.9671974 | 0.33340657 | FOXP3 | FOXP3 | 10000 | huTcell+ | 48 | qRTPCR |
| | 12 m01 | | | | | | | | |
| 41897 | FOXP3- | 2.08618587 | 0.97479621 | FOXP3 | FOXP3 | 10000 | huTcell+ | 48 | qRTPCR |
| | 13 m01 | | | | | | | | |
| 41052 | FOXP3- | 1.56270566 | 0.50779572 | FOXP3 | FOXP3 | 10000 | huTcell+ | 48 | qRTPCR |
| | 14 m01 | | | | | | | | |
| 41877 | FOXP3- | 1.24448641 | 0.38126836 | FOXP3 | FOXP3 | 10000 | huTcell+ | 48 | qRTPCR |
| | 15 m01 | | | | | | | | |
| 43243 | FOXP3- | 1.09482815 | 0.12860521 | FOXP3 | FOXP3 | 10000 | huTcell+ | 48 | qRTPCR |
| | 16 m01 | | | | | | | | |
| 43375 | FOXP3- | 0.75611626 | 0.06899775 | FOXP3 | FOXP3 | 10000 | huTcell+ | 48 | qRTPCR |
| | 17 m01 | | | | | | | | |
| 43376 | FOXP3- | 1.38423343 | 0.23242212 | FOXP3 | FOXP3 | 10000 | huTcell+ | 48 | qRTPCR |
| | 18 m01 | | | | | | | | |
| 43379 | FOXP3- | 2.23219084 | 0.48397258 | FOXP3 | FOXP3 | 10000 | huTcell+ | 48 | qRTPCR |
| | 19 m01 | | | | | | | | |
| 43380 | FOXP3- | 0.77319797 | 0.12468607 | FOXP3 | FOXP3 | 10000 | huTcell+ | 48 | qRTPCR |
| | 20 m01 | | | | | | | | |
| 43381 | FOXP3- | 0.92951965 | 0.36167181 | FOXP3 | FOXP3 | 10000 | huTcell+ | 48 | qRTPCR |
| | 21 m01 | | | | | | | | |
| 43216 | FOXP3- | 2.19209374 | 0.33354356 | FOXP3 | FOXP3 | 10000 | huTcell+ | 48 | qRTPCR |
| | 22 m01 | | | | | | | | |
| 43215 | FOXP3- | 4.60769744 | 1.32033965 | FOXP3 | FOXP3 | 10000 | huTcell+ | 48 | qRTPCR |
| | 23 m01 | | | | | | | | |
| 43209 | FOXP3- | 0.18862818 | 0.07780705 | FOXP3 | FOXP3 | 10000 | huTcell+ | 48 | qRTPCR |
| | 24 m01 | | | | | | | | |
| 43207 | FOXP3- | 0.53198871 | 0.0250394 | FOXP3 | FOXP3 | 10000 | huTcell+ | 48 | qRTPCR |
| | 25 m01 | | | | | | | | |
| 43205 | FOXP3- | 0.78569731 | 0.05157862 | FOXP3 | FOXP3 | 10000 | huTcell+ | 48 | qRTPCR |
| | 26 m01 | | | | | | | | |
| 43204 | FOXP3- | 1.4702824 | 0.16242886 | FOXP3 | FOXP3 | 10000 | huTcell+ | 48 | qRTPCR |
| | 27 m01 | | | | | | | | |
| 43203 | FOXP3- | 1.83244549 | 0.15170473 | FOXP3 | FOXP3 | 10000 | huTcell+ | 48 | qRTPCR |
| | 28 m01 | | | | | | | | |
| 42533 | FOXP3- | 2.14671471 | 0.65415214 | FOXP3 | FOXP3 | 10000 | huTcell+ | 48 | qRTPCR |
| | 29 m01 | | | | | | | | |
| 42535 | FOXP3- | 1.98183515 | 0.70000334 | FOXP3 | FOXP3 | 10000 | huTcell+ | 48 | qRTPCR |
| | 30 m01 | | | | | | | | |
| 42580 | FOXP3- | 0.80553315 | 0.36415893 | FOXP3 | FOXP3 | 10000 | huTcell+ | 48 | qRTPCR |
| | 31 m01 | | | | | | | | |
| 42579 | FOXP3- | 1.11788013 | 0.41330598 | FOXP3 | FOXP3 | 10000 | huTcell+ | 48 | qRTPCR |
| | 32 m01 | | | | | | | | |
| 42578 | FOXP3- | 1.52166183 | 0.81182555 | FOXP3 | FOXP3 | 10000 | huTcell+ | 48 | qRTPCR |
| | 33 m01 | | | | | | | | |
| 42576 | FOXP3- | 0.90079369 | 0.36068588 | FOXP3 | FOXP3 | 10000 | huTcell+ | 48 | qRTPCR |
| | 34 m01 | | | | | | | | |
| 42575 | FOXP3- | 0.9535541 | 0.34234744 | FOXP3 | FOXP3 | 10000 | huTcell+ | 48 | qRTPCR |
| | 35 m01 | | | | | | | | |
| 42574 | FOXP3- | 0.76441529 | 0.28279382 | FOXP3 | FOXP3 | 10000 | huTcell+ | 48 | qRTPCR |
| | 36 m01 | | | | | | | | |
| 42572 | FOXP3- | 1.49147643 | 0.15811177 | FOXP3 | FOXP3 | 10000 | huTcell+ | 48 | qRTPCR |
| | 37 m01 | | | | | | | | |
| 42571 | FOXP3- | 1.66272868 | 0.71072917 | FOXP3 | FOXP3 | 10000 | huTcell+ | 48 | qRTPCR |
| | 38 m01 | | | | | | | | |
| 42570 | FOXP3- | 2.12350232 | 0.20077097 | FOXP3 | FOXP3 | 10000 | huTcell+ | 48 | qRTPCR |
| | 39 m01 | | | | | | | | |
| 42569 | FOXP3- | 1.24001965 | 0.27790611 | FOXP3 | FOXP3 | 10000 | huTcell+ | 48 | qRTPCR |
| | 40 m01 | | | | | | | | |
| 42568 | FOXP3- | 1.99692286 | 1.69550223 | FOXP3 | FOXP3 | 10000 | huTcell+ | 48 | qRTPCR |
| | 41 m01 | | | | | | | | |
| 42567 | FOXP3- | 0.78743477 | 0.19946904 | FOXP3 | FOXP3 | 10000 | huTcell+ | 48 | qRTPCR |
| | 42 m01 | | | | | | | | |
| 43447 | FOXP3- | 0.7247555 | 0.18846039 | FOXP3 | FOXP3 | 10000 | huTcell+ | 48 | qRTPCR |
| | 43 m01 | | | | | | | | |
| 43450 | FOXP3- | 1.08905435 | 0.03216293 | FOXP3 | FOXP3 | 10000 | huTcell+ | 48 | qRTPCR |
| | 44 m01 | | | | | | | | |
| 43452 | FOXP3- | 1.13227275 | 0.48605068 | FOXP3 | FOXP3 | 10000 | huTcell+ | 48 | qRTPCR |
| | 45 m01 | | | | | | | | |
| 43455 | FOXP3- | 0.66689557 | 0.13950169 | FOXP3 | FOXP3 | 10000 | huTcell+ | 48 | qRTPCR |
| | 46 m01 | | | | | | | | |
| 43456 | FOXP3- | 1.62059627 | 0.2706113 | FOXP3 | FOXP3 | 10000 | huTcell+ | 48 | qRTPCR |
| | 47 m01 | | | | | | | | |
| 43458 | FOXP3- | 1.42062488 | 0.47050077 | FOXP3 | FOXP3 | 10000 | huTcell+ | 48 | qRTPCR |
| | 48 m01 | | | | | | | | |
| 43459 | FOXP3- | 5.04482435 | 0.75486048 | FOXP3 | FOXP3 | 10000 | huTcell+ | 48 | qRTPCR |
| | 49 m01 | | | | | | | | |
| 36466 | FOXP3- | 1.75784419 | 0.20278994 | FOXP3 | FOXP3 | 10000 | huTcell+ | 48 | qRTPCR |
| | 50 m01 | | | | | | | | |
| 36439 | FOXP3- | 0.5987679 | 0.19284557 | FOXP3 | FOXP3 | 10000 | huTcell+ | 48 | qRTPCR |
| | 51 m01 | | | | | | | | |
| 36437 | FOXP3- | 1.51000454 | 0.49882253 | FOXP3 | FOXP3 | 10000 | huTcell+ | 48 | qRTPCR |
| | 52 m01 | | | | | | | | |
| 29244 | FOXP3- | 1.56079422 | 0.22121849 | FOXP3 | FOXP3 | 10000 | huTcell+ | 48 | qRTPCR |
| | 53 m01 | | | | | | | | |
| 29253 | FOXP3- | 1.77819386 | 0.12532551 | FOXP3 | FOXP3 | 10000 | huTcell+ | 48 | qRTPCR |
| | 54 m01 | | | | | | | | |
| 41675 | FOXP3- | 0.74694704 | 0.04752939 | FOXP3 | FOXP3 | 10000 | huTcell+ | 48 | qRTPCR |
| | 55 m01 | | | | | | | | |
| 43610 | FOXP3- | 1.45116927 | 0.30495519 | FOXP3 | FOXP3 | 10000 | huTcell+ | 48 | qRTPCR |
| | 56 m01 | | | | | | | | |
| 43612 | FOXP3- | 0.67204413 | 0.26697083 | FOXP3 | FOXP3 | 10000 | huTcell+ | 48 | qRTPCR |
| | 57 m01 | | | | | | | | |
| 43613 | FOXP3- | 1.25708457 | 0.37514513 | FOXP3 | FOXP3 | 10000 | huTcell+ | 48 | qRTPCR |
| | 58 m01 | | | | | | | | |
| 43616 | FOXP3- | 2.24905264 | 0.0058918 | FOXP3 | FOXP3 | 10000 | huTcell+ | 48 | qRTPCR |
| | 59 m01 | | | | | | | | |
| 40694 | FOXP3- | 0.1994952 | 0.08969861 | FOXP3 | FOXP3 | 10000 | huTcell+ | 48 | qRTPCR |
| | 60 m01 | | | | | | | | |
| 41897 | FOXP3- | 1 | 0 | FOXP3 | FOXP3 | 0 | huTcell+ | 48 | qRTPCR |
| | 13 m01 | | | | | | | | |
| 41897 | FOXP3- | 0.87329521 | 0.15304712 | FOXP3 | FOXP3 | 32 | huTcell+ | 48 | qRTPCR |
| | 13 m01 | | | | | | | | |
| 41897 | FOXP3- | 1.41798849 | 0.0846702 | FOXP3 | FOXP3 | 160 | huTcell+ | 48 | qRTPCR |
| | 13 m01 | | | | | | | | |
| 41897 | FOXP3- | 0.83850744 | 0.33181934 | FOXP3 | FOXP3 | 800 | huTcell+ | 48 | qRTPCR |
| | 13 m01 | | | | | | | | |
| 41897 | FOXP3- | 2.23789843 | 0.93765398 | FOXP3 | FOXP3 | 4000 | huTcell+ | 48 | qRTPCR |
| | 13 m01 | | | | | | | | |
| 41897 | FOXP3- | 2.65002339 | 0.27289354 | FOXP3 | FOXP3 | 20000 | huTcell+ | 48 | qRTPCR |
| | 13 m01 | | | | | | | | |
| 43216 | FOXP3- | 1 | 0 | FOXP3 | FOXP3 | 0 | huTcell+ | 48 | qRTPCR |
| | 22 m01 | | | | | | | | |
| 43216 | FOXP3- | 0.79454623 | 0.10611272 | FOXP3 | FOXP3 | 32 | huTcell+ | 48 | qRTPCR |
| | 22 m01 | | | | | | | | |
| 43216 | FOXP3- | 1.08080897 | 0.23718635 | FOXP3 | FOXP3 | 160 | huTcell+ | 48 | qRTPCR |
| | 22 m01 | | | | | | | | |
| 43216 | FOXP3- | 0.76560804 | 0.00085315 | FOXP3 | FOXP3 | 800 | huTcell+ | 48 | qRTPCR |
| | 22 m01 | | | | | | | | |
| 43216 | FOXP3- | 9.52683765 | 0.25045621 | FOXP3 | FOXP3 | 4000 | huTcell+ | 48 | qRTPCR |
| | 22 m01 | | | | | | | | |
| 43216 | FOXP3- | 34.6761694 | 10.0353302 | FOXP3 | FOXP3 | 20000 | huTcell+ | 48 | qRTPCR |
| | 22 m01 | | | | | | | | |
| 43215 | FOXP3- | 1 | 0 | FOXP3 | FOXP3 | 0 | huTcell+ | 48 | qRTPCR |
| | 23 m01 | | | | | | | | |
| 43215 | FOXP3- | 2.25679314 | 1.7897399 | FOXP3 | FOXP3 | 32 | huTcell+ | 48 | qRTPCR |
| | 23 m01 | | | | | | | | |
| 43215 | FOXP3- | 1.41376849 | 0.84244148 | FOXP3 | FOXP3 | 160 | huTcell+ | 48 | qRTPCR |
| | 23 m01 | | | | | | | | |
| 43215 | FOXP3- | 3.44934969 | 0.18645628 | FOXP3 | FOXP3 | 800 | huTcell+ | 48 | qRTPCR |
| | 23 m01 | | | | | | | | |
| 43215 | FOXP3- | 2.60472728 | 0.24143156 | FOXP3 | FOXP3 | 4000 | huTcell+ | 48 | qRTPCR |
| | 23 m01 | | | | | | | | |
| 43215 | FOXP3- | 21.9984023 | 7.3981651 | FOXP3 | FOXP3 | 20000 | huTcell+ | 48 | qRTPCR |
| | 23 m01 | | | | | | | | |
| 43203 | FOXP3- | 1 | 0 | FOXP3 | FOXP3 | 0 | huTcell+ | 48 | qRTPCR |
| | 28 m01 | | | | | | | | |
| 43203 | FOXP3- | 1.03758152 | 0.13252612 | FOXP3 | FOXP3 | 32 | huTcell+ | 48 | qRTPCR |
| | 28 m01 | | | | | | | | |
| 43203 | FOXP3- | 0.88229811 | 0.34622482 | FOXP3 | FOXP3 | 160 | huTcell+ | 48 | qRTPCR |
| | 28 m01 | | | | | | | | |
| 43203 | FOXP3- | 3.65532124 | 3.11662006 | FOXP3 | FOXP3 | 800 | huTcell+ | 48 | qRTPCR |
| | 28 m01 | | | | | | | | |
| 43203 | FOXP3- | 1.96077256 | 0.8421269 | FOXP3 | FOXP3 | 4000 | huTcell+ | 48 | qRTPCR |
| | 28 m01 | | | | | | | | |
| 43203 | FOXP3- | 2.39760299 | 0.64192758 | FOXP3 | FOXP3 | 20000 | huTcell+ | 48 | qRTPCR |
| | 28 m01 | | | | | | | | |
| 42533 | FOXP3- | 1 | | FOXP3 | FOXP3 | 0 | huTcell+ | 48 | qRTPCR |
| | 29 m01 | | | | | | | | |
| 42533 | FOXP3- | 1.16880692 | | FOXP3 | FOXP3 | 32 | huTcell+ | 48 | qRTPCR |
| | 29 m01 | | | | | | | | |
| 42533 | FOXP3- | 1.2862768 | | FOXP3 | FOXP3 | 160 | huTcell+ | 48 | qRTPCR |
| | 29 m01 | | | | | | | | |
| 42533 | FOXP3- | 1.19650249 | | FOXP3 | FOXP3 | 800 | huTcell+ | 48 | qRTPCR |
| | 29 m01 | | | | | | | | |
| 42533 | FOXP3- | 1.09113218 | | FOXP3 | FOXP3 | 4000 | huTcell+ | 48 | qRTPCR |
| | 29 m01 | | | | | | | | |
| 42533 | FOXP3- | 5.31900948 | | FOXP3 | FOXP3 | 20000 | huTcell+ | 48 | qRTPCR |
| | 29 m01 | | | | | | | | |
| 42535 | FOXP3- | 1 | 0 | FOXP3 | FOXP3 | 0 | huTcell+ | 48 | qRTPCR |
| | 30 m01 | | | | | | | | |
| 42535 | FOXP3- | 0.57034656 | 0.32569034 | FOXP3 | FOXP3 | 32 | huTcell+ | 48 | qRTPCR |
| | 30 m01 | | | | | | | | |
| 42535 | FOXP3- | 0.99605844 | 0.77613072 | FOXP3 | FOXP3 | 160 | huTcell+ | 48 | qRTPCR |
| | 30 m01 | | | | | | | | |
| 42535 | FOXP3- | 0.68412094 | 0.42571495 | FOXP3 | FOXP3 | 800 | huTcell+ | 48 | qRTPCR |
| | 30 m01 | | | | | | | | |
| 42535 | FOXP3- | 1.39510325 | 0.05068993 | FOXP3 | FOXP3 | 4000 | huTcell+ | 48 | qRTPCR |
| | 30 m01 | | | | | | | | |
| 42535 | FOXP3- | 4.50897649 | 2.92595793 | FOXP3 | FOXP3 | 20000 | huTcell+ | 48 | qRTPCR |
| | 30 m01 | | | | | | | | |
| 43459 | FOXP3- | 1 | 0 | FOXP3 | FOXP3 | 0 | huTcell+ | 48 | qRTPCR |
| | 49 m01 | | | | | | | | |
| 43459 | FOXP3- | 1.18302438 | 0.48418155 | FOXP3 | FOXP3 | 32 | huTcell+ | 48 | qRTPCR |
| | 49 m01 | | | | | | | | |
| 43459 | FOXP3- | 1.63764765 | 0.11633382 | FOXP3 | FOXP3 | 160 | huTcell+ | 48 | qRTPCR |
| | 49 m01 | | | | | | | | |
| 43459 | FOXP3- | 2.39939746 | 1.31928791 | FOXP3 | FOXP3 | 800 | huTcell+ | 48 | qRTPCR |
| | 49 m01 | | | | | | | | |
| 43459 | FOXP3- | 3.73578628 | 0.74676582 | FOXP3 | FOXP3 | 4000 | huTcell+ | 48 | qRTPCR |
| | 49 m01 | | | | | | | | |
| 43459 | FOXP3- | 2.79836436 | 0.09852596 | FOXP3 | FOXP3 | 20000 | huTcell+ | 48 | qRTPCR |
| | 49 m01 | | | | | | | | |
| 36466 | FOXP3- | 1 | 0 | FOXP3 | FOXP3 | 0 | huTcell+ | 48 | qRTPCR |
| | 50 m01 | | | | | | | | |
| 36466 | FOXP3- | 1.13875449 | 0.0813526 | FOXP3 | FOXP3 | 32 | huTcell+ | 48 | qRTPCR |
| | 50 m01 | | | | | | | | |
| 36466 | FOXP3- | 1.03665859 | 0.67548254 | FOXP3 | FOXP3 | 160 | huTcell+ | 48 | qRTPCR |
| | 50 m01 | | | | | | | | |
| 36466 | FOXP3- | 1.26553217 | 0.04819054 | FOXP3 | FOXP3 | 800 | huTcell+ | 48 | qRTPCR |
| | 50 m01 | | | | | | | | |
| 36466 | FOXP3- | 2.11758319 | 0.01763026 | FOXP3 | FOXP3 | 4000 | huTcell+ | 48 | qRTPCR |
| | 50 m01 | | | | | | | | |
| 36466 | FOXP3- | 1.81747187 | 0.80710599 | FOXP3 | FOXP3 | 20000 | huTcell+ | 48 | qRTPCR |
| | 50 m01 | | | | | | | | |
| 41897 | FOXP3- | 1 | 0 | FOXP3 | IL2RA | 0 | huTcell+ | 48 | qRTPCR |
| | 13 m01 | | | | | | | | |
| 41897 | FOXP3- | 1 | 0 | FOXP3 | GITR | 0 | huTcell+ | 48 | qRTPCR |
| | 13 m01 | | | | | | | | |
| 41897 | FOXP3- | 0.52501902 | 0.04814779 | FOXP3 | IL2RA | 4000 | huTcell+ | 48 | qRTPCR |
| | 13 m01 | | | | | | | | |
| 41897 | FOXP3- | 2.18467964 | 0.04677421 | FOXP3 | GITR | 4000 | huTcell+ | 48 | qRTPCR |
| | 13 m01 | | | | | | | | |
| 43216 | FOXP3- | 1 | 0 | FOXP3 | IL2RA | 0 | huTcell+ | 48 | qRTPCR |
| | 22 m01 | | | | | | | | |
| 43216 | FOXP3- | 1 | 0 | FOXP3 | GITR | 0 | huTcell+ | 48 | qRTPCR |
| | 22 m01 | | | | | | | | |
| 43216 | FOXP3- | 0.53622332 | 0.00683636 | FOXP3 | IL2RA | 4000 | huTcell+ | 48 | qRTPCR |
| | 22 m01 | | | | | | | | |
| 43216 | FOXP3- | 1.24288249 | 0.5601923 | FOXP3 | GITR | 4000 | huTcell+ | 48 | qRTPCR |
| | 22 m01 | | | | | | | | |
| 43215 | FOXP3- | 1 | | FOXP3 | IL2RA | 0 | huTcell+ | 48 | qRTPCR |
| | 23 m01 | | | | | | | | |
| 43215 | FOXP3- | 1 | 0 | FOXP3 | GITR | 0 | huTcell+ | 48 | qRTPCR |
| | 23 m01 | | | | | | | | |
| 43215 | FOXP3- | 0.49775779 | 0.022437 | FOXP3 | IL2RA | 4000 | huTcell+ | 48 | qRTPCR |
| | 23 m01 | | | | | | | | |
| 43215 | FOXP3- | 1.81662608 | 0.25980483 | FOXP3 | GITR | 4000 | huTcell+ | 48 | qRTPCR |
| | 23 m01 | | | | | | | | |
| 43203 | FOXP3- | 1 | 0 | FOXP3 | IL2RA | 0 | huTcell+ | 48 | qRTPCR |
| | 28 m01 | | | | | | | | |
| 43203 | FOXP3- | 1 | 0 | FOXP3 | GITR | 0 | huTcell+ | 48 | qRTPCR |
| | 28 m01 | | | | | | | | |
| 43203 | FOXP3- | 0.57528551 | 0.01462293 | FOXP3 | IL2RA | 4000 | huTcell+ | 48 | qRTPCR |
| | 28 m01 | | | | | | | | |
| 43203 | FOXP3- | 1.80449429 | 0.42917742 | FOXP3 | GITR | 4000 | huTcell+ | 48 | qRTPCR |
| | 28 m01 | | | | | | | | |
| 42533 | FOXP3- | 1 | 0 | FOXP3 | IL2RA | 0 | huTcell+ | 48 | qRTPCR |
| | 29 m01 | | | | | | | | |
| 42533 | FOXP3- | 1 | 0 | FOXP3 | GITR | 0 | huTcell+ | 48 | qRTPCR |
| | 29 m01 | | | | | | | | |
| 42533 | FOXP3- | 0.94465714 | 0.14681686 | FOXP3 | IL2RA | 4000 | huTcell+ | 48 | qRTPCR |
| | 29 m01 | | | | | | | | |
| 42533 | FOXP3- | 1.10066946 | 0.14692655 | FOXP3 | GITR | 4000 | huTcell+ | 48 | qRTPCR |
| | 29 m01 | | | | | | | | |
| 42535 | FOXP3- | 1 | 0 | FOXP3 | IL2RA | 0 | huTcell+ | 48 | qRTPCR |
| | 30 m01 | | | | | | | | |
| 42535 | FOXP3- | 1 | 0 | FOXP3 | GITR | 0 | huTcell+ | 48 | qRTPCR |
| | 30 m01 | | | | | | | | |
| 42535 | FOXP3- | 0.60238158 | 0.10681676 | FOXP3 | IL2RA | 4000 | huTcell+ | 48 | qRTPCR |
| | 30 m01 | | | | | | | | |
| 42535 | FOXP3- | 1.16496654 | 0.0896547 | FOXP3 | GITR | 4000 | huTcell+ | 48 | qRTPCR |
| | 30 m01 | | | | | | | | |
| 43459 | FOXP3- | 1 | 0 | FOXP3 | IL2RA | 0 | huTcell+ | 48 | qRTPCR |
| | 49 m01 | | | | | | | | |
| 43459 | FOXP3- | 1 | 0 | FOXP3 | GITR | 0 | huTcell+ | 48 | qRTPCR |
| | 49 m01 | | | | | | | | |
| 43459 | FOXP3- | 0.98563279 | 0 | FOXP3 | IL2RA | 4000 | huTcell+ | 48 | qRTPCR |
| | 49 m01 | | | | | | | | |
| 43459 | FOXP3- | 0.42633761 | 0.14166645 | FOXP3 | GITR | 4000 | huTcell+ | 48 | qRTPCR |
| | 49 m01 | | | | | | | | |
| 36466 | FOXP3- | 1 | 0 | FOXP3 | IL2RA | 0 | huTcell+ | 48 | qRTPCR |
| | 50 m01 | | | | | | | | |
| 36466 | FOXP3- | 1 | 0 | FOXP3 | GITR | 0 | huTcell+ | 48 | qRTPCR |
| | 50 m01 | | | | | | | | |
| 36466 | FOXP3- | 0.72010675 | 0.14187437 | FOXP3 | IL2RA | 4000 | huTcell+ | 48 | qRTPCR |
| | 50 m01 | | | | | | | | |
| 36466 | FOXP3- | 0.42291963 | 0.44260683 | FOXP3 | GITR | 4000 | huTcell+ | 48 | qRTPCR |
| | 50 m01 | | | | | | | | |
| 41897 | FOXP3- | 1 | 0 | FOXP3 | IL10 | 0 | huTcell+ | 48 | qRTPCR |
| | 13 m01 | | | | | | | | |
| 41897 | FOXP3- | 0.2719813 | 0.14740902 | FOXP3 | IL10 | 4000 | huTcell+ | 48 | qRTPCR |
| | 13 m01 | | | | | | | | |
| 43216 | FOXP3- | 1 | 0 | FOXP3 | IL10 | 0 | huTcell+ | 48 | qRTPCR |
| | 22 m01 | | | | | | | | |
| 43216 | FOXP3- | 0.45881437 | 0.11316447 | FOXP3 | IL10 | 4000 | huTcell+ | 48 | qRTPCR |
| | 22 m01 | | | | | | | | |
| 43215 | FOXP3- | 1 | 0 | FOXP3 | IL10 | 0 | huTcell+ | 48 | qRTPCR |
| | 23 m01 | | | | | | | | |
| 43215 | FOXP3- | 0.44853129 | 0.2508683 | FOXP3 | IL10 | 4000 | huTcell+ | 48 | qRTPCR |
| | 23 m01 | | | | | | | | |
| 43203 | FOXP3- | 1 | 0 | FOXP3 | IL10 | 0 | huTcell+ | 48 | qRTPCR |
| | 28 m01 | | | | | | | | |
| 43203 | FOXP3- | 0.64629725 | 0.69484085 | FOXP3 | IL10 | 4000 | huTcell+ | 48 | qRTPCR |
| | 28 m01 | | | | | | | | |
| 42533 | FOXP3- | 1 | 0 | FOXP3 | IL10 | 0 | huTcell+ | 48 | qRTPCR |
| | 29 m01 | | | | | | | | |
| 42533 | FOXP3- | 0.78210159 | 0.09875678 | FOXP3 | IL10 | 4000 | huTcell+ | 48 | qRTPCR |
| | 29 m01 | | | | | | | | |
| 42535 | FOXP3- | 1 | 0 | FOXP3 | IL10 | 0 | huTcell+ | 48 | qRTPCR |
| | 30 m01 | | | | | | | | |
| 42535 | FOXP3- | 0.22048642 | 0.18358706 | FOXP3 | IL10 | 4000 | huTcell+ | 48 | qRTPCR |
| | 30 m01 | | | | | | | | |
| 43459 | FOXP3- | 1 | 0 | FOXP3 | IL10 | 0 | huTcell+ | 48 | qRTPCR |
| | 49 m01 | | | | | | | | |
| 43459 | FOXP3- | 0.51181181 | 0.14571517 | FOXP3 | IL10 | 4000 | huTcell+ | 48 | qRTPCR |
| | 49 m01 | | | | | | | | |
| 36466 | FOXP3- | 1 | 0 | FOXP3 | IL10 | 0 | huTcell+ | 48 | qRTPCR |
| | 50 m01 | | | | | | | | |
| 36466 | FOXP3- | 0.22783415 | 0.04265941 | FOXP3 | IL10 | 4000 | huTcell+ | 48 | qRTPCR |
| | 50 m01 | | | | | | | | |
| 41897 | FOXP3- | 1 | 0 | FOXP3 | CCDC22 | 0 | huTcell+ | 48 | qRTPCR |
| | 13 m01 | | | | | | | | |
| 41897 | FOXP3- | 0.99497325 | 0.11794901 | FOXP3 | CCDC22 | 4000 | huTcell+ | 48 | qRTPCR |
| | 13 m01 | | | | | | | | |
| 43216 | FOXP3- | 1 | 0 | FOXP3 | CCDC22 | 0 | huTcell+ | 48 | qRTPCR |
| | 22 m01 | | | | | | | | |
| 43216 | FOXP3- | 1.91996125 | 0.51849461 | FOXP3 | CCDC22 | 4000 | huTcell+ | 48 | qRTPCR |
| | 22 m01 | | | | | | | | |
| 43215 | FOXP3- | 1 | 0 | FOXP3 | CCDC22 | 0 | huTcell+ | 48 | qRTPCR |
| | 23 m01 | | | | | | | | |
| 43215 | FOXP3- | 2.02080353 | 0.04661423 | FOXP3 | CCDC22 | 4000 | huTcell+ | 48 | qRTPCR |
| | 23 m01 | | | | | | | | |
| 43203 | FOXP3- | 1 | 0 | FOXP3 | CCDC22 | 0 | huTcell+ | 48 | qRTPCR |
| | 28 m01 | | | | | | | | |
| 43203 | FOXP3- | 1.08719018 | 0.38056095 | FOXP3 | CCDC22 | 4000 | huTcell+ | 48 | qRTPCR |
| | 28 m01 | | | | | | | | |
| 42533 | FOXP3- | 1 | 0 | FOXP3 | CCDC22 | 0 | huTcell+ | 48 | qRTPCR |
| | 29 m01 | | | | | | | | |
| 42533 | FOXP3- | 0.95815978 | 0.06806494 | FOXP3 | CCDC22 | 4000 | huTcell+ | 48 | qRTPCR |
| | 29 m01 | | | | | | | | |
| 42535 | FOXP3- | 1 | 0 | FOXP3 | CCDC22 | 0 | huTcell+ | 48 | qRTPCR |
| | 30 m01 | | | | | | | | |
| 42535 | FOXP3- | 0.7900138 | 0.03245348 | FOXP3 | CCDC22 | 4000 | huTcell+ | 48 | qRTPCR |
| | 30 m01 | | | | | | | | |
| 43459 | FOXP3- | 1 | 0 | FOXP3 | CCDC22 | 0 | huTcell+ | 48 | qRTPCR |
| | 49 m01 | | | | | | | | |
| 43459 | FOXP3- | 0.83390492 | 0.43203537 | FOXP3 | CCDC22 | 4000 | huTcell+ | 48 | qRTPCR |
| | 49 m01 | | | | | | | | |
| 36466 | FOXP3- | 1 | 0 | FOXP3 | CCDC22 | 0 | huTcell+ | 48 | qRTPCR |
| | 50 m01 | | | | | | | | |
| 36466 | FOXP3- | 0.36224221 | 0.06333645 | FOXP3 | CCDC22 | 4000 | huTcell+ | 48 | qRTPCR |
| | 50 m01 | | | | | | | | |

TABLE 3
A listing of oligonucleotide modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA w/3′ phosphate |
| dT | DNA w/3′ phosphate |
| dU | deoxyuridine w/3′ phosphate |
| d5mCs | deoxy-5-methylcytidine w/3′ thiophosphate |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| InaAs | LNA w/3′ thiophosphate |
| InaCs | LNA w/3′ thiophosphate |
| InaGs | LNA w/3′ thiophosphate |
| InaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| InaAs-Sup | LNA w/3′ thiophosphate at 3′ terminus |
| InaCs-Sup | LNA w/3′ thiophosphate at 3′ terminus |
| InaGs-Sup | LNA w/3′ thiophosphate at 3′ terminus |
| InaTs-Sup | LNA w/3′ thiophosphate at 3′ terminus |
| InaA-Sup | LNA w/3′ OH at 3′ terminus |
| InaC-Sup | LNA w/3′ OH at 3′ terminus |
| InaG-Sup | LNA w/3′ OH at 3′ terminus |
| InaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |
| dU | deoxyuridine w/3′ OH at 3′ terminus |
| rA | RNA w/3′ phosphate |
| rC | RNA w/3′ phosphate |
| rG | RNA w/3′ phosphate |
| rU | RNA w/3′ phosphate |

TABLE 4
Formatted oligonucleotide sequences showing nucleotide modifications.

| OligoID | Base Sequence | Formatted Sequence | SeqID |
|---|---|---|---|
| FOXP3-01 | CCTCGATGGTC | lnaCs; omeCs; lnaTs; omeCs; lnaGs; omeAs; lnaTs; omeGs; lnaGs; | 42428 |
| m01 | TGGA | omeUs; lnaCs; omeUs; lnaGs; omeGs; lnaA-Sup | |
| FOXP3-02 | CTCGATGGTCT | lnaCs; omeUs; lnaCs; omeGs; lnaAs; omeUs; lnaGs; omeGs; lnaTs; | 42429 |
| m01 | GGAT | omeCs; lnaTs; omeGs; lnaGs; omeAs; lnaT-Sup | |
| FOXP3-03 | CGATGGTCTGG | lnaCs; omeGs; lnaAs; omeUs; lnaGs; omeGs; lnaTs; omeCs; lnaTs; | 42431 |
| m01 | ATGA | omeGs; lnaGs; omeAs; lnaTs; omeGs; lnaA-Sup | |
| FOXP3-04 | TCCTCGATGGT | lnaTs; omeCs; lnaCs; omeUs; lnaCs; omeGs; lnaAs; omeUs; lnaGs; | 42427 |
| m01 | CTGG | omeGs; lnaTs; omeCs; lnaTs; omeGs; lnaG-Sup | |
| FOXP3-05 | GTCCTCGATGG | lnaGs; omeUs; lnaCs; omeCs; lnaTs; omeCs; lnaGs; omeAs; lnaTs; | 42426 |
| m01 | TCTG | omeGs; lnaGs; omeUs; lnaCs; omeUs; lnaG-Sup | |
| FOXP3-06 | GCCTGTGTCCT | lnaGs; omeCs; lnaCs; omeUs; lnaGs; omeUs; lnaGs; omeUs; lnaCs; | 42420 |
| m01 | CGAT | omeCs; lnaTs; omeCs; lnaGs; omeAs; lnaT-Sup | |
| FOXP3-07 | CACCTGCTCCT | lnaCs; omeAs; lnaCs; omeCs; lnaTs; omeGs; lnaCs; omeUs; lnaCs; | 42381 |
| m01 | CGAG | omeCs; lnaTs; omeCs; lnaGs; omeAs; lnaG-Sup | |
| FOXP3-08 | ATCAGTCACCG | lnaAs; omeUs; lnaCs; omeAs; lnaGs; omeUs; lnaCs; omeAs; lnaCs; | 42862 |
| m01 | CAAA | omeCs; lnaGs; omeCs; lnaAs; omeAs; lnaA-Sup | |
| FOXP3-09 | CATCAGTCACC | lnaCs; omeAs; lnaTs; omeCs; lnaAs; omeGs; lnaTs; omeCs; lnaAs; | 42861 |
| m01 | GCAA | omeCs; lnaCs; omeGs; lnaCs; omeAs; lnaA-Sup | |
| FOXP3-10 | CTCATCAGTCA | lnaCs; omeUs; lnaCs; omeAs; lnaTs; omeCs; lnaAs; omeGs; lnaTs; | 42859 |
| m01 | CCGC | omeCs; lnaAs; omeCs; lnaCs; omeGs; lnaC-Sup | |
| FOXP3-11 | GCTCATCAGTC | lnaGs; omeCs; lnaTs; omeCs; lnaAs; omeUs; lnaCs; omeAs; lnaGs; | 42858 |
| m01 | ACCG | omeUs; lnaCs; omeAs; lnaCs; omeCs; lnaG-Sup | |
| FOXP3-12 | ACAAGCTCATC | lnaAs; omeCs; lnaAs; omeAs; lnaGs; omeCs; lnaTs; omeCs; lnaAs; | 42854 |
| m01 | AGTC | omeUs; lnaCs; omeAs; lnaGs; omeUs; lnaC-Sup | |
| FOXP3-13 | GCTCGGTAGTC | lnaGs; omeCs; lnaTs; omeCs; lnaGs; omeGs; lnaTs; omeAs; lnaGs; | 41897 |
| m01 | CTCC | omeUs; lnaCs; omeCs; lnaTs; omeCs; lnaC-Sup | |
| FOXP3-14 | AGAGCCTTCAC | lnaAs; omeGs; lnaAs; omeGs; lnaCs; omeCs; lnaTs; omeUs; lnaCs: | 41052 |
| m01 | AACC | omeAs; lnaCs; omeAs; lnaAs; omeCs; lnaC-Sup | |
| FOXP3-15 | CAGCGTTCTCC | lnaCs; omeAs; lnaGs; omeCs; lnaGs; omeUs; lnaTs; omeCs; lnaTs: | 41877 |
| m01 | TGGC | omeCs; lnaCs; omeUs; lnaGs; omeGs; lnaC-Sup | |
| FOXP3-16 | GTGTAGGCCAG | lnaGs; omeUs; lnaGs; omeUs; lnaAs; omeGs; lnaGs; omeCs; lnaCs; | 43243 |
| m01 | CCGG | omeAs; lnaGs; omeCs; lnaCs; omeGs; lnaG-Sup | |
| FOXP3-17 | CAGCTGCTTAT | lnaCs; omeAs; lnaGs; omeCs; lnaTs; omeGs; lnaCs; omeUs; lnaTs; | 43375 |
| m01 | AGAC | omeAs; lnaTs; omeAs; lnaGs; omeAs; lnaC-Sup | |
| FOXP3-18 | AGCTGCTTATA | lnaAs; omeGs; lnaCs; omeUs; lnaGs; omeCs; lnaTs; omeUs; lnaAs; | 43376 |
| m01 | GACC | omeUs; lnaAs; omeGs; lnaAs; omeCs; lnaC-Sup | |
| FOXP3-19 | TGCTTATAGAC | lnaTs; omeGs; lnaCs; omeUs; lnaTs; omeAs; lnaTs; omeAs; lnaGs; | 43379 |
| m01 | CTCC | omeAs; lnaCs; omeCs; lnaTs; omeCs; lnaC-Sup | |
| FOXP3-20 | GCTTATAGACC | lnaGs; omeCs; lnaTs; omeUs; lnaAs; omeUs; lnaAs; omeGs; lnaAs; | 43380 |
| m01 | TCCT | omeCs; lnaCs; omeUs; lnaCs; omeCs; lnaT-Sup | |
| FOXP3-21 | CTTATAGACCT | lnaCs; omeUs; lnaTs; omeAs; lnaTs; omeAs; lnaGs; omeAs; lnaCs; | 43381 |
| m01 | CCTC | omeCs; lnaTs; omeCs; lnaCs; omeUs; lnaC-Sup | |
| FOXP3-22 | TTGCCCACGAT | lnaTs; omeUs; lnaGs; omeCs; lnaCs; omeCs; lnaAs; omeCs; lnaGs; | 43216 |
| m01 | CTCC | omeAs; lnaTs; omeCs; lnaTs; omeCs; lnaC-Sup | |
| FOXP3-23 | GTTGCCCACGA | lnaGs; omeUs; lnaTs; omeGs; lnaCs; omeCs; lnaCs; omeAs; lnaCs; | 43215 |
| m01 | TCTC | omeGs; lnaAs; omeUs; lnaCs; omeUs; lnaC-Sup | |
| FOXP3-24 | CCGGATGTTGC | lnaCs; omeCs; lnaGs; omeGs; lnaAs; omeUs; lnaGs; omeUs; lnaTs; | 43209 |
| m01 | CCAC | omeGs; lnaCs; omeCs; lnaCs; omeAs; lnaC-Sup | |
| FOXP3-25 | TTCCGGATGTT | lnaTs; omeUs; lnaCs; omeCs; lnaGs; omeGs; lnaAs; omeUs; lnaGs; | 43207 |
| m01 | GCCC | omeUs; lnaTs; omeGs; lnaCs; omeCs; lnaC-Sup | |
| FOXP3-26 | GCTTCCGGATG | lnaGs; omeCs; lnaTs; omeUs; lnaCs; omeCs; lnaGs; omeGs; lnaAs; | 43205 |
| m01 | TTGC | omeUs; lnaGs; omeUs; lnaTs; omeGs; lnaC-Sup | |
| FOXP3-27 | TGCTTCCGGAT | lnaTs; omeGs; lnaCs; omeUs; lnaTs; omeCs; lnaCs; omeGs; lnaGs; | 43204 |
| m01 | GTTG | omeAs; lnaTs; omeGs; lnaTs; omeUs; lnaG-Sup | |
| FOXP3-28 | CTGCTTCCGGA | lnaCs; omeUs; lnaGs; omeCs; lnaTs; omeUs; lnaCs; omeCs; lnaGs; | 43203 |
| m01 | TGTT | omeGs; lnaAs; omeUs; lnaGs; omeUs; lnaT-Sup | |
| FOXP3-29 | CCCCTCACCTC | lnaCs; omeCs; lnaCs; omeCs; lnaTs; omeCs; lnaAs; omeCs; lnaCs; | 42533 |
| m01 | GTGC | omeUs; lnaCs; omeGs; lnaTs; omeGs; lnaC-Sup | |
| FOXP3-30 | CCTCACCTCGT | lnaCs; omeCs; lnaTs; omeCs; lnaAs; omeCs; lnaCs; omeUs; lnaCs; | 42535 |
| m01 | GCAG | omeGs; lnaTs; omeGs; lnaCs; omeAs; lnaG-Sup | |
| FOXP3-31 | AGCATCGTCCT | lnaAs; omeGs; lnaCs; omeAs; lnaTs; omeCs; lnaGs; omeUs; lnaCs; | 42580 |
| m01 | TCTT | omeCs; lnaTs; omeUs; lnaCs; omeUs; lnaT-Sup | |
| FOXP3-32 | CAGCATCGTCC | lnaCs; omeAs; lnaGs; omeCs; lnaAs; omeUs; lnaCs; omeGs; lnaTs; | 42579 |
| m01 | TTCT | omeCs; lnaCs; omeUs; lnaTs; omeCs; lnaT-Sup | |
| FOXP3-33 | ACAGCATCGTC | lnaAs; omeCs; lnaAs; omeGs; lnaCs; omeAs; lnaTs; omeCs; lnaGs; | 42578 |
| m01 | CTTC | omeUs; lnaCs; omeCs; lnaTs; omeUs; lnaC-Sup | |
| FOXP3-34 | GAACAGCATCG | lnaGs; omeAs; lnaAs; omeCs; lnaAs; omeGs; lnaCs; omeAs; lnaTs; | 42576 |
| m01 | TCCT | omeCs; lnaGs; omeUs; lnaCs; omeCs; lnaT-Sup | |
| FOXP3-35 | CGAACAGCATC | lnaCs; omeGs; lnaAs; omeAs; lnaCs; omeAs; lnaGs; omeCs; lnaAs; | 42575 |
| m01 | GTCC | omeUs; lnaCs; omeGs; lnaTs; omeCs; lnaC-Sup | |
| FOXP3-36 | CCGAACAGCAT | lnaCs; omeCs; lnaGs; omeAs; lnaAs; omeCs; lnaAs; omeGs; lnaCs; | 42574 |
| m01 | CGTC | omeAs; lnaTs; omeCs; lnaGs; omeUs; lnaC-Sup | |
| FOXP3-37 | TTCCGAACAGC | lnaTs; omeUs; lnaCs; omeCs; lnaGs; omeAs; lnaAs; omeCs; lnaAs; | 42572 |
| m01 | ATCG | omeGs; lnaCs; omeAs; lnaTs; omeCs; lnaG-Sup | |
| FOXP3-38 | CTTCCGAACAG | lnaCs; omeUs; lnaTs; omeCs; lnaCs; omeGs; lnaAs; omeAs; lnaCs; | 42571 |
| m01 | CATC | omeAs; lnaGs; omeCs; lnaAs; omeUs; lnaC-Sup | |
| FOXP3-39 | CCTTCCGAACA | lnaCs; omeCs; lnaTs; omeUs; lnaCs; omeCs; lnaGs; omeAs; lnaAs; | 42570 |
| m01 | GCAT | omeCs; lnaAs; omeGs; lnaCs; omeAs; lnaT-Sup | |
| FOXP3-40 | GCCTTCCGAAC | lnaGs; omeCs; lnaCs; omeUs; lnaTs; omeCs; lnaCs; omeGs; lnaAs; | 42569 |
| m01 | AGCA | omeAs; lnaCs; omeAs; lnaGs; omeCs; lnaA-Sup | |
| FOXP3-41 | GGCCTTCCGAA | lnaGs; omeGs; lnaCs; omeCs; lnaTs; omeUs; lnaCs; omeCs; lnaGs; | 42568 |
| m01 | CAGC | omeAs; lnaAs; omeCs; lnaAs; omeGs; lnaC-Sup | |
| FOXP3-42 | AGGCCTTCCGA | lnaAs; omeGs; lnaGs; omeCs; lnaCs; omeUs; lnaTs; omeCs; lnaCs; | 42567 |
| m01 | ACAG | omeGs; lnaAs; omeAs; lnaCs; omeAs; lnaG-Sup | |
| FOXP3-43 | GATCTCTGCCA | lnaGs; omeAs; lnaTs; omeCs; lnaTs; omeCs; lnaTs; omeGs; lnaCs; | 43447 |
| m01 | GCCG | omeCs; lnaAs; omeGs; lnaCs; omeCs; lnaG-Sup | |
| FOXP3-44 | CTCTGCCAGCC | lnaCs; omeUs; lnaCs; omeUs; lnaGs; omeCs; lnaCs; omeAs; lnaGs; | 43450 |
| m01 | GTCG | omeCs; lnaCs; omeGs; lnaTs; omeCs; lnaG-Sup | |
| FOXP3-45 | CTGCCAGCCGT | lnaCs; omeUs; lnaGs; omeCs; lnaCs; omeAs; lnaGs; omeCs; lnaCs; | 43452 |
| m01 | CGAG | omeGs; lnaTs; omeCs; lnaGs; omeAs; lnaG-Sup | |
| FOXP3-46 | CCAGCCGTCGA | lnaCs; omeCs; lnaAs; omeGs; lnaCs; omeCs; lnaGs; omeUs; lnaCs; | 43455 |
| m01 | GAAG | omeGs; lnaAs; omeGs; lnaAs; omeAs; lnaG-Sup | |
| FOXP3-47 | CAGCCGTCGAG | lnaCs; omeAs; lnaGs; omeCs; lnaCs; omeGs; lnaTs; omeCs; lnaGs; | 43456 |
| m01 | AAGA | omeAs; lnaGs; omeAs; lnaAs; omeGs; lnaA-Sup | |
| FOXP3-48 | GCCGTCGAGAA | lnaGs; omeCs; lnaCs; omeGs; lnaTs; omeCs; lnaGs; omeAs; lnaGs; | 43458 |
| m01 | GATT | omeAs; lnaAs; omeGs; lnaAs; omeUs; lnaT-Sup | |
| FOXP3-49 | CCGTCGAGAAG | lnaCs; omeCs; lnaGs; omeUs; lnaCs; omeGs; lnaAs; omeGs; lnaAs; | 43459 |
| m01 | ATTC | omeAs; lnaGs; omeAs; lnaTs; omeUs; lnaC-Sup | |
| FOXP3-50 | CTTCGAAGAGC | lnaCs; omeUs; lnaTs; omeCs; lnaGs; omeAs; lnaAs; omeGs; lnaAs; | 36466 |
| m01 | CAGA | omeGs; lnaCs; omeCs; lnaAs; omeGs; lnaA-Sup | |
| FOXP3-51 | CTGCAAGTGGC | lnaCs; omeUs; lnaGs; omeCs; lnaAs; omeAs; lnaGs; omeUs; lnaGs; | 36439 |
| m01 | CCGG | omeGs; lnaCs; omeCs; lnaCs; omeGs; lnaG-Sup | |
| FOXP3-52 | GTCTGCAAGTG | lnaGs; omeUs; lnaCs; omeUs; lnaGs; omeCs; lnaAs; omeAs; lnaGs; | 36437 |
| m01 | GCCC | omeUs; lnaGs; omeGs; lnaCs; omeCs; lnaC-Sup | |
| FOXP3-53 | TGTACACAGCT | lnaTs; omeGs; lnaTs; omeAs; lnaCs; omeAs; lnaCs; omeAs; lnaGs; | 29244 |
| m01 | GGCG | omeCs; lnaTs; omeGs; lnaGs; omeCs; lnaG-Sup | |
| FOXP3-54 | CTGGCGTTTAA | lnaCs; omeUs; lnaGs; omeGs; lnaCs; omeGs; lnaTs; omeUs; lnaTs; | 29253 |
| m01 | TAAT | omeAs; lnaAs; omeUs; lnaAs; omeAs; lnaT-Sup | |
| FOXP3-55 | GCTGGACTATC | lnaGs; omeCs; lnaTs; omeGs; lnaGs; omeAs; lnaCs; omeUs; lnaAs; | 41675 |
| m01 | ACCC | omeUs; lnaCs; omeAs; lnaCs; omeCs; lnaC-Sup | |
| FOXP3-56 | GAGGTGGCGG | lnaGs; omeAs; lnaGs; omeGs; lnaTs; omeGs; lnaGs; omeCs; lnaGs; | 43610 |
| m01 | TACTC | omeGs; lnaTs; omeAs; lnaCs; omeUs; lnaC-Sup | |
| FOXP3-57 | GGTGGCGGTA | lnaGs; omeGs; lnaTs; omeGs; lnaGs; omeCs; lnaGs; omeGs; lnaTs; | 43612 |
| m01 | CTCAG | omeAs; lnaCs; omeUs; lnaCs; omeAs; lnaG-Sup | |
| FOXP3-58 | GTGGCGGTACT | lnaGs; omeUs; lnaGs; omeGs; lnaCs; omeGs; lnaGs; omeUs; lnaAs; | 43613 |
| m01 | CAGC | omeCs; lnaTs; omeCs; lnaAs; omeGs; lnaC-Sup | |
| FOXP3-59 | GCGGTACTCAG | lnaGs; omeCs; lnaGs; omeGs; lnaTs; omeAs; lnaCs; omeUs; lnaCs; | 43616 |
| m01 | CGAG | omeAs; lnaGs; omeCs; lnaGs; omeAs; lnaG-Sup | |
| FOXP3-60 | GTGGACCGTG | lnaGs; omeUs; lnaGs; omeGs; lnaAs; omeCs; lnaCs; omeGs; lnaTs; | 40694 |
| m01 | GATGA | omeGs; lnaGs; omeAs; lnaTs; omeGs; lnaA-Sup | |

BRIEF DESCRIPTION OF THE SEQUENCE LISTING

| SeqID | Chrom | gene | Chr.Start | Chr.End | strand | Organism |
|---|---|---|---|---|---|---|
| 1 | chrX | FOXP3 | 49094896 | 49133288 | − | Homo sapiens |
| 2 | chrX | FOXP3 | 49094896 | 49133288 | + | Homo sapiens |
| 3 | chrX | Foxp3 | 7567675 | 7607243 | + | Mus musculus |
| 4 | chrX | Foxp3 | 7567675 | 7607243 | − | Mus musculus |
| 5 | chrX | FOXP3 | 49091852 | 49146158 | + | Homo sapiens |
| 6 | chrX | FOXP3 | 49105387 | 49126985 | + | Homo sapiens |
| 7 | chrX | FOXP3 | 49105442 | 49121156 | + | Homo sapiens |
| 8 | chrX | FOXP3 | 49131266 | 49131313 | + | Homo sapiens |
| 9 | chrX | FOXP3 | 49131123 | 49131172 | + | Homo sapiens |
| 10 | chrX | FOXP3 | 49127994 | 49128033 | + | Homo sapiens |
| 11 | chrX | FOXP3 | 49127843 | 49127890 | + | Homo sapiens |
| 12 | chrX | FOXP3 | 49127628 | 49127670 | + | Homo sapiens |
| 13 | chrX | FOXP3 | 49124798 | 49124897 | + | Homo sapiens |
| 14 | chrX | FOXP3 | 49123918 | 49123965 | + | Homo sapiens |
| 15 | chrX | FOXP3 | 49120701 | 49120753 | + | Homo sapiens |
| 16 | chrX | FOXP3 | 49118531 | 49118555 | + | Homo sapiens |
| 17 | chrX | FOXP3 | 49115652 | 49115685 | + | Homo sapiens |
| 18 | chrX | FOXP3 | 49112995 | 49113044 | + | Homo sapiens |
| 19 | chrX | FOXP3 | 49112863 | 49112906 | + | Homo sapiens |
| 20 | chrX | FOXP3 | 49112637 | 49112717 | + | Homo sapiens |
| 21 | chrX | FOXP3 | 49107522 | 49107575 | + | Homo sapiens |
| 22 | chrX | FOXP3 | 49106607 | 49106653 | + | Homo sapiens |
| 23 | chrX | FOXP3 | 49106128 | 49106175 | + | Homo sapiens |
| 24 | chrX | FOXP3 | 49105839 | 49105886 | + | Homo sapiens |
| 25 | chrX | FOXP3 | 49105669 | 49105701 | + | Homo sapiens |
| 26 | chrX | FOXP3 | 49105241 | 49105285 | + | Homo sapiens |
| 27 | chrX | FOXP3 | 49129266 | 49133313 | + | Homo sapiens |
| 28 | chrX | FOXP3 | 49129123 | 49133172 | + | Homo sapiens |
| 29 | chrX | FOXP3 | 49125994 | 49130033 | + | Homo sapiens |
| 30 | chrX | FOXP3 | 49125843 | 49129890 | + | Homo sapiens |
| 31 | chrX | FOXP3 | 49125628 | 49129670 | + | Homo sapiens |
| 32 | chrX | FOXP3 | 49122798 | 49126897 | + | Homo sapiens |
| 33 | chrX | FOXP3 | 49121918 | 49125965 | + | Homo sapiens |
| 34 | chrX | FOXP3 | 49118701 | 49122753 | + | Homo sapiens |
| 35 | chrX | FOXP3 | 49116531 | 49120555 | + | Homo sapiens |
| 36 | chrX | FOXP3 | 49113652 | 49117685 | + | Homo sapiens |
| 37 | chrX | FOXP3 | 49110995 | 49115044 | + | Homo sapiens |
| 38 | chrX | FOXP3 | 49110863 | 49114906 | + | Homo sapiens |
| 39 | chrX | FOXP3 | 49110637 | 49114717 | + | Homo sapiens |
| 40 | chrX | FOXP3 | 49105522 | 49109575 | + | Homo sapiens |
| 41 | chrX | FOXP3 | 49104607 | 49108653 | + | Homo sapiens |
| 42 | chrX | FOXP3 | 49104128 | 49108175 | + | Homo sapiens |
| 43 | chrX | FOXP3 | 49103839 | 49107886 | + | Homo sapiens |
| 44 | chrX | FOXP3 | 49103669 | 49107701 | + | Homo sapiens |
| 45 | chrX | FOXP3 | 49103241 | 49107285 | + | Homo sapiens |
| 46 | chrX | FOXP3 | 49091852 | 49146158 | − | Homo sapiens |
| 47 | chrX | FOXP3 | 49105387 | 49126985 | − | Homo sapiens |
| 48 | chrX | FOXP3 | 49127432 | 49127481 | − | Homo sapiens |
| 49 | chrX | FOXP3 | 49127343 | 49127398 | − | Homo sapiens |
| 50 | chrX | FOXP3 | 49117756 | 49117794 | − | Homo sapiens |
| 51 | chrX | FOXP3 | 49100610 | 49100635 | − | Homo sapiens |
| 52 | chrX | FOXP3 | 49100129 | 49100194 | − | Homo sapiens |
| 53 | chrX | FOXP3 | 49099553 | 49099595 | − | Homo sapiens |
| 54 | chrX | FOXP3 | 49125432 | 49129481 | − | Homo sapiens |
| 55 | chrX | FOXP3 | 49125343 | 49129398 | − | Homo sapiens |
| 56 | chrX | FOXP3 | 49115756 | 49119794 | − | Homo sapiens |
| 57 | chrX | FOXP3 | 49098610 | 49102635 | − | Homo sapiens |
| 58 | chrX | FOXP3 | 49098129 | 49102194 | − | Homo sapiens |
| 59 | chrX | FOXP3 | 49097553 | 49101595 | − | Homo sapiens |

Single Strand Oligonucleotides (Antisense Strand of Target Gene)
SeqID range: 60-16461
SeqIDs w/o G Runs:
60-81, 96-201, 215-232, 255-378, 392-600, 616-654, 678-685, 699-745,
759-781, 795-802, 816-876, 902-915, 930-950, 970-1006, 1021-1044,
1058-1196, 1216-1248, 1264-1307, 1321-1322, 1342-1448, 1463-1938,
1952-2189, 2204-2289, 2303-2315, 2330-2416, 2430-2439, 2453-2646,
2671-2717, 2731-2846, 2868-2889, 2903-2912, 2926-2995, 3009-3055,
3075-3127, 3141-3142, 3156-3161, 3175-3182, 3196-3311, 3325-3345,
3360-3399, 3413-3455, 3469-3520, 3534, 3554-3603, 3618-3756,
3770-3803, 3817-3895, 3909-3973, 3987-4087, 4101-4147, 4163-4223,
4237, 4251-4304, 4318-4374, 4409-4488, 4502-4540, 4556-4567,
4587-4633, 4647-4692, 4707-4864, 4894-4957, 4972-4997, 5011-5075,
5089-5100, 5124-5167, 5185-5201, 5238-5245, 5259-5291, 5305-5382,
5397-5449, 5470-5512, 5537-5616, 5630-5669, 5683-5743, 5768-5794,
5822-5889, 5915-5937, 5961-5998, 6012-6016, 6030-6065, 6079-6087,
6127-6167, 6182-6186, 6200-6227, 6244-6289, 6313-6328, 6343-6351,
6365-6367, 6395-6418, 6433-6444, 6458-6488, 6502-6771, 6785-6967,
6981-6984, 6998-7543, 7554-7561, 7575-7578, 7592-7611, 7625-7760,
7774-7840, 7855-8099, 8130-8275, 8289-8420, 8434-8548, 8563-8617,
8631-8632, 8659-8844, 8858-8862, 8877-8895, 8909-8937, 8957-9073,
9087-9091, 9106-9114, 9138-9158, 9174-9185, 9208-9230, 9244-9261,
9276-9342, 9357-9361, 9376-9384, 9399-9443, 9469-9491, 9507-9565,
9579-9609, 9623-9707, 9722-9801, 9825-9872, 9886-9950, 9974-10033,
10047-10065, 10079-10136, 10163-10174, 10201-10207, 10221-10226,
10240-10254, 10278-10280, 10296-10304, 10318-10346, 10360-10632,
10648-10691, 10706-10743, 10763-10772, 10786-10808, 10824-10854,
10868-10881, 10897-10922, 10959-10971, 10985-11116, 11131-11148,
11162-11208, 11222-11246, 11260-11428, 11443, 11458-11517, 11531,
11547, 11563-11602, 11617-11685, 11699-11800, 11814-12163,
12175-12237, 12251-12853, 12856-12906, 12920-12952, 12966-12992,
13006-13017, 13031-13521, 13533-13588, 13597-13635, 13648-13660,
13674-13816, 13830-13841, 13856-13893, 13907-14033, 14047-14096,
14139-14174, 14188-14237, 14251-14272, 14286-14300, 14318-14320,
14340-14410, 14424-14483, 14506-14578, 14592-14599, 14634-14703,
14717-14730, 14746-14864, 14883-15085, 15108-15152, 15172-15196,
15233-15273, 15293-15314, 15387-15506, 15520-15560, 15574-15593,
15607-15615, 15651-15652, 15666-15860, 15874-16122, 16141-16163,
16177-16246, 16272-16285, 16299-16370, 16384-16420, 16426-16443,
16451-16453, 16455-16461
SeqIDs w/o miR Seeds:
60-65, 68-69, 74, 77, 79, 88-91, 93-94, 97, 99-100, 103, 105-106,
108, 110-112, 115-127, 130, 134-141, 144-145, 148-149, 152, 154-157,
159, 161-165, 167-169, 172-173, 176, 180-183, 185, 187-190, 193-195,
202-205, 207-210, 212-216, 218, 220, 222-225, 228, 230-234, 236,
238-239, 241, 245, 248-259, 262-264, 266-270, 273-274, 277-280,
282-286, 290-296, 298-301, 303-309, 312-314, 316-320, 322, 324,
326-329, 334, 336, 338-339, 341, 343-349, 351, 354-360, 362-368,
370-373, 376, 378-379, 382-386, 389-392, 394, 396, 399-400, 402-403,
405-411, 413-421, 423, 426, 429, 431-432, 434-435, 437, 441, 443,
446-447, 449-457, 459-462, 464-468, 470, 472-478, 480-484, 487-489,
492-494, 497, 499-504, 506-509, 511-512, 514-517, 520-525, 527, 530,
534, 538, 540-541, 545-550, 552, 557-570, 575, 577-580, 582-588,
592-594, 596, 599-600, 602, 604, 607, 609-610, 612, 614, 618-624,
627-628, 630-634, 636, 638, 641-645, 648-651, 653-654, 656-657
659-661, 663-664, 666, 674-676, 678-680, 683-687, 689, 696-699,
702-712, 716-718, 722, 728-733, 736, 741-742, 745-746, 751, 753,
756-757, 760, 762-764, 766-767, 769-775, 778, 781-782, 785-786,
788-789, 791-792, 794-796, 799-800, 802, 807-811, 813-814, 817-822,
824, 827-830, 833-835, 837-841, 843, 846-853, 860, 862, 866-873,
875-877, 881, 883-884, 886-891, 893-894, 896-898, 902, 905-913, 915,
919-920, 923-927, 929, 931, 934, 938-942, 944, 947-951, 956, 959,
963, 965-973, 975, 979, 981-983, 986, 990, 999-1001, 1003-1004,
1006-1007, 1012-1013, 1016-1018, 1021, 1023, 1025, 1028-1029,
1031-1037, 1040, 1042-1044, 1048, 1051-1053, 1056, 1058, 1060-1065,
1070-1074, 1076, 1079-1081, 1084-1095, 1097-1108, 1110, 1112-1113,
1115-1118, 1121-1128, 1130, 1132, 1138, 1140-1143, 1151, 1153-1154,
1159, 1162, 1165-1166, 1168-1169, 1171, 1177, 1179-1181, 1183-1185,
1187-1188, 1192-1194, 1196-1199, 1201-1202, 1204-1205, 1209-1211,
1213-1214, 1216-1218, 1221, 1225-1231, 1234, 1236-1237, 1239-1241,
1246-1250, 1252, 1256-1265, 1267, 1269, 1271, 1273-1275, 1277-1287,
1291, 1295-1301, 1303-1307, 1312-1321, 1325, 1329, 1332-1333, 1335,
1338, 1344, 1346-1347, 1349, 1351, 1354-1363, 1367, 1370, 1373,
1375-1378, 1380-1387, 1389, 1393-1395, 1397-1398, 1400-1403, 1405,
1408, 1411-1413, 1415-1420, 1422, 1426, 1428-1429, 1433, 1435,
1437-1441, 1443-1444, 1453-1456, 1458, 1460-1461, 1464-1466,
1469-1470, 1472-1476, 1478-1479, 1481, 1484-1486, 1488, 1491-1493,
1495, 1497, 1499-1502, 1504, 1506-1513, 1515-1522, 1524-1527,
1530-1531, 1533, 1535, 1542-1544, 1550, 1555, 1558-1559, 1562-1565,
1567, 1569-1572, 1576, 1579, 1582-1584, 1586, 1590-1592, 1594, 1596,
1601-1603, 1605-1610, 1612-1615, 1617-1618, 1620, 1622-1623, 1625,
1627-1630, 1632, 1635, 1638, 1641, 1643, 1645-1656, 1660, 1665,
1668-1669, 1672, 1677-1679, 1681-1682, 1684, 1687-1690, 1694-1700,
1702-1703, 1705, 1707-1709, 1711, 1714, 1719, 1721-1726, 1732, 1734,
1739-1740, 1743-1744, 1746, 1749-1750, 1752, 1755, 1757-1759, 1761,
1763-1767, 1769, 1771, 1776, 1779-1780, 1784, 1786, 1790, 1793-1794,
1797-1800, 1803-1805, 1807-1809, 1811-1814, 1816, 1820, 1823, 1825,
1837-1838, 1840-1845, 1847-1852, 1854-1856, 1858-1859, 1862,
1868-1869, 1873-1876, 1878, 1881, 1887-1888, 1890-1898, 1900,
1902-1903, 1905, 1912-1915, 1921-1924, 1926, 1928, 1931-1933,
1935-1937, 1940-1942, 1947, 1951, 1955, 1960-1962, 1964, 1967,
1971-1973, 1977, 1979, 1981, 1983, 1986-1988, 1990-1991, 1994-1997,
1999-2000, 2004-2006, 2008, 2011-2013, 2015-2020, 2022, 2024, 2026,
2028-2029, 2032, 2036, 2038-2044, 2046, 2048-2049, 2051-2052, 2054,
2056, 2059-2064, 2066-2071, 2073, 2075-2076, 2078, 2080, 2083, 2085,
2087, 2090-2091, 2093-2097, 2099, 2101, 2103, 2105-2109, 2112, 2114,
2119-2120, 2122, 2124, 2126-2127, 2130-2131, 2133-2135, 2138-2142,
2144-2145, 2148, 2150-2152, 2154-2160, 2162-2166, 2169, 2172-2173,
2175-2176, 2178, 2181-2184, 2186-2191, 2193, 2195-2201, 2203-2215,
2217-2218, 2223-2224, 2226-2229, 2231-2236, 2238, 2240-2246,
2248-2250, 2252-2257, 2260-2262, 2266-2272, 2274, 2277-2278,
2280-2281, 2284, 2290, 2292, 2294-2296, 2300, 2303-2311, 2319-2320,
2323, 2326-2327, 2329, 2333-2336, 2338, 2341, 2343-2345, 2347,
2349-2350, 2352, 2358, 2360, 2362-2363, 2365-2368, 2373-2374,
2377-2383, 2385, 2387-2388, 2395, 2398-2405, 2408, 2411-2414,
2416-2417, 2424-2425, 2429-2433, 2435-2439, 2443-2447, 2450-2455,
2457-2467, 2469-2471, 2473, 2476, 2478-2479, 2482-2488, 2492, 2494,
2496, 2498-2499, 2501, 2503-2504, 2506-2507, 2509, 2512, 2515-2517,
2519, 2521, 2523-2527, 2531-2534, 2536-2539, 2541, 2543, 2545,
2547-2549, 2552-2560, 2563-2565, 2567-2570, 2572-2574, 2578-2581,
2584-2585, 2587-2588, 2590, 2592-2593, 2595, 2597, 2600, 2603,
2605-2606, 2609-2613, 2617, 2619-2620, 2622-2624, 2626, 2629-2631,
2633-2636, 2643-2646, 2655, 2662-2663, 2665-2667, 2670-2674,
2677-2679, 2681-2683, 2685, 2687-2688, 2690-2691, 2693-2694, 2696,
2703, 2705, 2709-2711, 2713-2718, 2721-2722, 2729, 2737, 2740,
2743-2752, 2756-2757, 2759, 2763-2768, 2770-2774, 2776-2777,
2780, 2782-2783, 2785, 2787-2790, 2795-2799, 2801, 2805, 2810-2811,
2813, 2819, 2823-2824, 2828-2832, 2839-2841, 2843, 2852, 2854-2855,
2859-2860, 2862-2864, 2868-2875, 2882-2884, 2886-2887, 2889-2890,
2900-2905, 2907, 2910, 2912-2915, 2918-2919, 2921, 2923, 2926-2927,
2929-2931, 2934-2939, 2941, 2943-2952, 2954-2956, 2958, 2960-2961,
2963-2964, 2971, 2975-2979, 2981-2982, 2984, 2988, 2993-2997, 2999,
3007-3009, 3016, 3018, 3020-3024, 3026, 3028-3030, 3032, 3034-3041,
3043-3045, 3047, 3049-3054, 3057, 3059-3061, 3069, 3071-3073,
3075-3077, 3080-3083, 3085-3089, 3091-3092, 3094-3097, 3102,
3108-3110, 3113-3114, 3120, 3123-3124, 3126, 3128, 3132, 3134, 3137,
3148, 3150-3155, 3158-3162, 3164-3165, 3168, 3171, 3175, 3182, 3185,
3188-3189, 3191, 3194-3198, 3200-3201, 3204, 3206-3207, 3209-3210,
3213, 3215-3216, 3218, 3220-3221, 3226-3227, 3231-3235, 3237, 3240,
3243-3245, 3247, 3249, 3251, 3253-3254, 3256-3262, 3264, 3271, 3273,
3276-3280, 3282-3283, 3285-3288, 3290-3291, 3294-3300, 3302-3303,
3306-3312, 3315-3323, 3328, 3330-3331, 3334-3336, 3338, 3340-3342,
3344, 3349, 3351-3353, 3355-3364, 3367-3379, 3382, 3384-3389, 3391,
3394, 3396-3397, 3399-3400, 3402, 3404-3412, 3415, 3417, 3420-3424,
3427-3429, 3431, 3434-3439, 3441-3442, 3445, 3447, 3450, 3454-3455,
3457, 3461, 3463-3466, 3472-3474, 3476-3478, 3482-3483, 3485-3486,
3488, 3491-3504, 3506, 3508-3517, 3519-3523, 3525-3526, 3529-3531,
3533, 3541, 3545-3547, 3549, 3552, 3556-3557, 3559-3562, 3565-3567,
3569, 3571, 3573-3574, 3577, 3581-3582, 3584-3585, 3587-3589, 3591,
3594-3597, 3599, 3601, 3605, 3607, 3609, 3613-3616, 3618-3620,
3622-3630, 3632-3633, 3635-3637, 3639-3640, 3642-3648, 3650-3651,
3653-3654, 3656-3657, 3661, 3664, 3666-3668, 3670-3672, 3674-3681,
3684, 3686-3691, 3693-3694, 3697, 3699-3702, 3704-3707, 3710, 3715,
3720-3722, 3725-3727, 3732-3736, 3739-3745, 3747, 3749-3753,
3758-3761, 3763-3766, 3771, 3773-3779, 3781, 3783, 3785-3786, 3788,
3790-3793, 3795, 3797, 3799, 3801, 3803, 3808, 3810, 3812, 3814,
3819-3824, 3827-3829, 3831, 3834-3835, 3840-3842, 3844, 3846-3848,
3850-3851, 3853-3860, 3862-3863, 3865-3866, 3868, 3870, 3872, 3875,
3880-3882, 3884, 3887-3890, 3892-3893, 3895, 3901-3905, 3909,
3911-3912, 3914-3919, 3922, 3926-3927, 3930-3934, 3939, 3941, 3943,
3945-3947, 3949-3950, 3952-3954, 3956, 3960, 3962, 3965-3976,
3978-3983, 3985-3994, 3996-4005, 4008, 4010, 4012-4014, 4017, 4019,
4021-4023, 4025-4032, 4034-4037, 4039-4041, 4043-4045, 4048-4049,
4052, 4054-4055, 4058, 4060-4061, 4064-4065, 4067-4069, 4073-4078,
4081, 4087-4088, 4091, 4096-4098, 4100-4103, 4106-4107, 4109, 4111,
4118-4119, 4121-4123, 4127-4128, 4130-4136, 4139-4141, 4147,
4154-4158, 4161-4163, 4165, 4171-4178, 4184-4186, 4189, 4191,
4193-4195, 4198-4199, 4201, 4203-4206, 4211, 4215, 4218-4224, 4227,
4234, 4237-4238, 4240, 4243, 4245-4246, 4248-4257, 4259, 4263-4265,
4267-4268, 4274-4275, 4277, 4280-4285, 4288-4291, 4293-4295, 4300,
4304, 4308, 4310, 4312-4316, 4319-4320, 4322-4323, 4325, 4334
4336-4340, 4343-4348, 4350, 4352, 4358, 4360, 4364-4365, 4367,
4369-4372, 4374, 4377-4378, 4381, 4385, 4389, 4391-4392, 4404, 4406,
4411-4415, 4417, 4419-4421, 4423-4424, 4426-4427, 4431-4433, 4435,
4437-4438, 4440-4441, 4443, 4446, 4450-4452, 4454-4465, 4467,
4469-4474, 4476, 4478-4479, 4482-4483, 4492-4493, 4495, 4497-4498,
4501-4505, 4507-4517, 4519-4521, 4523-4527, 4530-4533, 4536-4539,
4541, 4543, 4546, 4549-4552, 4554-4555, 4558, 4561-4563, 4565, 4569,
4572-4576, 4579-4580, 4584-4587, 4590, 4592-4595, 4597-4598,
4601-4602, 4604-4610, 4613-4614, 4616-4617, 4619-4621, 4624,
4626-4628, 4632-4635, 4637-4639, 4642-4645, 4647-4648, 4651-4653,
4656, 4658-4659, 4663-4664, 4669, 4671-4675, 4680-4681, 4683-4686,
4688, 4690-4691, 4693, 4698, 4700-4701, 4705, 4707, 4709-4712,
4714-4717, 4719, 4721-4725, 4729-4734, 4736, 4739-4741, 4744,
4746-4747, 4752, 4755, 4757, 4759, 4762-4763, 4766-4768, 4770-4773,
4776, 4779-4780, 4782, 4784, 4789, 4791, 4793-4795, 4802, 4804,
4806, 4808, 4812, 4816, 4818, 4820, 4822, 4825, 4829, 4832, 4834,
4844-4845, 4849-4851, 4855-4857, 4859-4860, 4863, 4867-4868, 4872,
4875, 4878-4880, 4884-4888, 4895-4897, 4899, 4902-4904, 4908-4910,
4913, 4916, 4921, 4923-4924, 4926, 4935-4939, 4942, 4944-4947, 4950,
4952, 4954, 4965-4967, 4970, 4976, 4980-4981, 4992-4995, 5000-5002,
5004-5007, 5009-5011, 5013-5014, 5016-5017, 5019-5021, 5024,
5026-5029, 5031-5033, 5041-5046, 5048-5052, 5055-5058, 5068-5070,
5072-5073, 5080-5083, 5085-5088, 5091-5092, 5095-5099, 5102, 5108,
5116-5123, 5125-5126, 5128, 5131-5133, 5135, 5137, 5139, 5141, 5143,
5145-5150, 5153, 5155, 5157, 5164-5165, 5167-5168, 5172, 5176-5179,
5181-5183, 5185-5188, 5190-5192, 5195, 5197, 5199-5201, 5206, 5210,
5212, 5214-5215, 5217, 5219, 5221-5226, 5231-5232, 5235, 5237-5241,
5244-5248, 5251, 5253-5267, 5269-5271, 5273-5278, 5280-5286, 5288,
5293, 5295, 5297-5298, 5300, 5302-5303, 5307-5308, 5310, 5312, 5316,
5318-5324, 5326-5332, 5334, 5336-5337, 5340-5344, 5349-5350,
5352-5353, 5360-5364, 5366-5369, 5371-5372, 5374, 5377-5383, 5388,
5390-5396, 5399-5400, 5404, 5406-5413, 5415-5418, 5423-5424, 5428,
5430-5433, 5435, 5437, 5439, 5441, 5444, 5446-5447, 5449-5454,
5461-5463, 5467, 5470-5471, 5476, 5479, 5481-5483, 5485-5487, 5492,
5495-5496, 5498-5500, 5502-5506, 5518, 5522-5523, 5529-5530,
5532-5536, 5538-5540, 5542-5547, 5553-5561, 5563-5564, 5566, 5568,
5571-5575, 5578-5579, 5583, 5586-5588, 5590-5592, 5600, 5603, 5606,
5611-5612, 5614-5615, 5618, 5620, 5627-5628, 5630, 5633-5636, 5638,
5640-5641, 5643, 5646-5651, 5664-5665, 5667, 5669, 5673, 5675,
5677-5681, 5683-5684, 5687-5689, 5691-5692, 5695-5706, 5708-5712,
5715-5717, 5719, 5721, 5723-5724, 5726, 5728-5740, 5742-5743,
5749-5756, 5765-5766, 5770-5771, 5773-5775, 5778-5780, 5782-5784,
5786-5790, 5800-5802, 5807, 5817-5819, 5821, 5825, 5831, 5834,
5836-5838, 5840-5842, 5844, 5846-5853, 5855-5870, 5872, 5874,
5876-5877, 5879-5880, 5884-5888, 5891-5893, 5896, 5901, 5905, 5907,
5909-5910, 5914, 5916-5918, 5920-5922, 5924, 5933-5935, 5940-5941,
5943-5944, 5950, 5952, 5954-5956, 5961-5965, 5967, 5969-5976,
5978-5979, 5981, 5983-5990, 5992-5997, 5999, 6005-6012, 6015-6016,
6019, 6021-6029, 6032-6033, 6038-6044, 6046-6051, 6053, 6055-6062,
6064-6065, 6067, 6070, 6072, 6074-6076, 6079-6082, 6085-6086,
6088-6090, 6098-6099, 6101, 6107-6113, 6115, 6117, 6120-6121,
6124-6128, 6130-6134, 6136, 6138-6141, 6143-6147, 6151, 6153,
6156-6157, 6159-6167, 6174-6177, 6179-6181, 6183-6184, 6186-6192,
6195, 6198-6199, 6201-6204, 6206-6214, 6217, 6219, 6221, 6223-6225,
6227-6228, 6231, 6237, 6239-6246, 6248-6251, 6254-6259, 6261-6262,
6266, 6269-6273, 6275-6276, 6282-6283, 6293, 6297-6298, 6304,
6310-6311, 6313-6321, 6324-6331, 6335, 6338, 6341, 6343, 6347-6348,
6350, 6353-6356, 6358-6359, 6361, 6364, 6368, 6371, 6374, 6376,
6379, 6382, 6386, 6388-6399, 6401-6408, 6410-6411, 6413-6418, 6427,
6430-6440, 6442-6444, 6446-6450, 6452-6453, 6455-6457, 6460-6461,
6463-6464, 6467, 6469-6477, 6479-6484, 6487-6488, 6490-6491,
6493-6494, 6496-6503, 6505-6507, 6509-6510, 6512, 6515-6517,
6520-6521, 6523-6528, 6533-6534, 6536, 6541, 6543-6549, 6551,
6553-6554, 6556-6560, 6562-6563, 6565-6566, 6569-6571, 6573-6580,
6582-6585, 6587-6588, 6590, 6594-6597, 6599-6600, 6602, 6605,
6610-6612, 6614, 6617-6618, 6621, 6623, 6626-6633, 6636-6637,
6640-6643, 6645-6646, 6648, 6650-6651, 6654-6655, 6657-6663,
6665-6666, 6668-6675, 6679, 6681-6684, 6686, 6688-6690,
6692-6693, 6696-6699, 6703-6707, 6709, 6711, 6715-6716, 6718, 6721,
6723-6726, 6728-6731, 6733, 6735-6740, 6742-6743, 6745-6749,
6755-6757, 6759, 6762-6767, 6769-6770, 6772, 6774-6775, 6777-6779,
6781, 6783-6791, 6793-6794, 6796-6797, 6799-6802, 6804-6809, 6811,
6813-6818, 6821, 6826-6827, 6829-6833, 6836-6839, 6841-6846, 6848,
6851, 6853-6857, 6859-6864, 6869, 6875-6883, 6885-6886, 6888, 6890,
6893-6896, 6898-6899, 6902, 6904-6905, 6907-6914, 6917, 6919-6920,
6922-6923, 6926-6930, 6932-6933, 6938-6942, 6944, 6948, 6950-6954,
6956-6962, 6964-6966, 6971-6973, 6975, 6977-6978, 6983, 6985,
6987-6990, 6992-7000, 7002-7007, 7009-7019, 7022-7027, 7032,
7035-7039, 7042-7043, 7045-7046, 7048-7050, 7052, 7056-7064,
7066-7068, 7070, 7073-7074, 7076-7085, 7087-7093, 7097-7098,
7100-7103, 7107-7111, 7113-7118, 7121-7124, 7127, 7129, 7132-7139,
7142-7151, 7154-7161, 7163-7165, 7167-7169, 7173, 7175-7177,
7181-7182, 7184, 7186-7189, 7192-7198, 7200-7201, 7203-7209,
7211-7212, 7215-7216, 7218-7225, 7227-7230, 7232-7234, 7237-7238,
7240-7250, 7252-7253, 7255-7257, 7263-7264, 7268-7273, 7276-7279,
7281-7282, 7284-7285, 7287-7288, 7290, 7292, 7295-7300, 7302-7305,
7307-7311, 7314, 7316-7327, 7330-7334, 7336-7338, 7340, 7342, 7344,
7347-7349, 7351, 7353-7355, 7357-7363, 7367-7370, 7372-7373, 7375,
7378-7382, 7384-7385, 7387, 7389-7400, 7403, 7406, 7409-7411,
7413-7429, 7431-7434, 7438, 7440-7441, 7445-7451, 7453-7454, 7456,
7458-7459, 7462, 7464, 7466-7467, 7470-7471, 7473-7484, 7486, 7490,
495-7496, 7498-7501, 7506-7507, 7512-7515, 7517-7523, 7525-7528,
7536-7544, 7546-7549, 7551, 7555-7556, 7558-7561, 7566, 7571-7573,
7577-7580, 7582-7587, 7590-7591, 7595-7596, 7598-7599, 7601-7609,
7611, 7614, 7617, 7619-7623, 7625-7642, 7645, 7647-7650, 7653, 7656,
7658-7659, 7661-7663, 7665-7666, 7669, 7671-7674, 7677-7683,
7685-7689, 7692, 7695-7697, 7699-7700, 7702-7710, 7712-7716, 7718,
7720-7728, 7730-7736, 7739-7742, 7744, 7746, 7748-7752, 7754-7756,
7758, 7760, 7765-7771, 7774, 7776, 7779-7781, 7783-7784, 7786, 7788,
7790-7796, 7798-7803, 7805, 7807, 7811-7813, 7815-7816, 7820-7821,
7825-7826, 7828-7840, 7846-7847, 7850-7852, 7854, 7857, 7859,
7861-7869, 7871-7872, 7874-7881, 7883-7884, 7886, 7888, 7891-7892,
7894-7899, 7901-7902, 7904, 7907, 7910, 7914, 7916-7919, 7921-7925,
7927-7928, 7931-7941, 7943, 7945-7948, 7951, 7953-7955, 7959,
7961-7966, 7968-7972, 7974-7975, 7977, 7979-7981, 7988-7989,
7991-7992, 7994-7997, 7999, 8001-8008, 8011-8015, 8017-8018,
8021-8022, 8024-8025, 8027-8028, 8030-8034, 8037-8038, 8042-8043,
8047, 8049-8059, 8061-8066, 8068-8072, 8074, 8076, 8080-8082,
8084-8086, 8088, 8092-8094, 8096, 8098-8099, 8103, 8105-8111,
8116-8117, 8122, 8124, 8126, 8128, 8133-8137, 8145-8148, 8151-8152,
8156-8158, 8164, 8166, 8171-8174, 8176-8182, 8185, 8187-8192,
8194-8199, 8210-8212, 8215-8216, 8218-8220, 8222-8228, 8233-8237,
8239-8240, 8243-8244, 8246-8252, 8254, 8256, 8258, 8260, 8263-8265,
8268, 8271, 8273-8275, 8279, 8281-8286, 8289, 8291-8295, 8297,
8299-8306, 8308-8309, 8311-8312, 8315-8321, 8323-8326, 8328,
8330-8331, 8333-8336, 8338-8339, 8342-8343, 8345-8346, 8348,
8350-8355, 8357-8360, 8362-8365, 8368, 8371, 8373, 8375-8378,
8380-8382, 8384-8386, 8389-8391, 8393, 8395-8399, 8402, 8405,
8407-8409, 8411, 8413, 8415, 8422, 8426-8430, 8432-8433, 8436,
8440-8442, 8444, 8446, 8448-8449, 8454-8455, 8457, 8461-8462, 8465,
8469, 8473, 8478-8479, 8485-8492, 8495-8497, 8501-8503, 8506,
8510-8512, 8514-8518, 8521, 8524-8525, 8529, 8533-8543, 8547-8548,
8554-8559, 8561-8562, 8566-8570, 8572, 8582, 8584-8585, 8594-8595,
8597-8599, 8602, 8604, 8607-8608, 8610-8611, 8614-8617, 8621,
8626-8628, 8630-8635, 8641-8642, 8647, 8649, 8652, 8654, 8656-8658,
8660-8666, 8668, 8672, 8674-8676, 8678-8679, 8681-8687, 8694,
8698-8699, 8702-8707, 8709, 8713-8714, 8716, 8719-8721, 8723-8725,
8732-8734, 8736, 8738, 8740, 8742, 8744, 8749-8750, 8752-8754, 8759,
8762-8763, 8767, 8769-8770, 8772, 8776-8778, 8780-8783, 8785-8788,
8791-8796, 8798-8800, 8804-8806, 8810-8816, 8818-8819, 8822, 8824,
8827, 8829-8830, 8832-8835, 8837-8838, 8840, 8842-8847, 8850-8855,
8858, 8862, 8866-8867, 8869, 8871-8877, 8879-8881, 8883-8886,
8888-8889, 8891, 8895-8896, 8903, 8907-8909, 8911-8913, 8915,
8917-8921, 8923-8924, 8928-8934, 8936, 8944, 8947-8948, 8950-8953,
8955-8956, 8958-8961, 8964-8965, 8968-8969, 8971, 8973-8982,
8984, 8987-8993, 8995-8998, 9000, 9005-9007, 9015, 9018-9024,
9027-9032, 9034, 9037-9039, 9042, 9044, 9047-9050, 9052-9057,
9061-9063, 9065-9070, 9075, 9077, 9080-9082, 9084-9085, 9087-9091,
9093, 9099-9102, 9105, 9108, 9110-9111, 9113, 9115, 9117-9118, 9124,
9126, 9128, 9130, 9133, 9135-9136, 9138, 9140, 9142, 9145-9148,
9150-9155, 9157, 9167, 9169, 9176-9187, 9195, 9197, 9200, 9202,
9205-9213, 9215, 9217-9221, 9225-9228, 9231, 9239, 9241, 9244-9248,
9250-9258, 9260-9261, 9264, 9266, 9268-9270, 9274-9278, 9281, 9285,
9290, 9292-9294, 9298, 9301, 9303-9306, 9308-9310, 9312-9314,
9316-9318, 9320-9322, 9333-9334, 9336-9340, 9343-9344, 9351-9352,
9361, 9366-9371, 9373, 9376-9377, 9379-9382, 9385, 9387, 9390, 9392,
9396-9397, 9399, 9401, 9403-9404, 9406-9408, 9410, 9412, 9415-9418,
9420-9425, 9428, 9430-9437, 9442, 9451-9453, 9455, 9461, 9463, 9465,
9468-9469, 9472, 9474, 9480, 9482, 9484, 9497-9499, 9501, 9503,
9505-9506, 9508, 9510-9513, 9515-9516, 9518, 9520, 9522-9523,
9526-9530, 9532-9533, 9535-9543, 9549, 9551-9553, 9559-9560, 9562,
9564-9565, 9570-9573, 9575-9579, 9582-9583, 9586-9587, 9594,
9599-9601, 9603, 9605-9607, 9615, 9617-9619, 9621-9625, 9628,
9633-9635, 9638, 9641, 9643-9644, 9646-9650, 9654-9662, 9664-9665,
9667, 9670-9671, 9673, 9676-9677, 9680, 9682-9685, 9687, 9691-9693,
9697, 9699, 9703-9706, 9714, 9716, 9719-9721, 9723, 9727, 9732-9734,
9736, 9738-9740, 9743-9749, 9752-9758, 9762-9763, 9766-9767, 9770,
9772-9774, 9776-9778, 9780, 9785, 9787-9794, 9796, 9800-9802, 9811,
9816-9821, 9823-9829, 9831, 9843, 9848, 9850, 9852-9854, 9857-9858,
9861, 9863, 9866, 9869, 9871-9872, 9874, 9876, 9878, 9882, 9890,
9892, 9894-9895, 9898-9903, 9906-9910, 9912-9916, 9918, 9921,
9923-9928, 9930-9937, 9939, 9941, 9946, 9949-9950, 9952, 9954-9955,
9957, 9960-9961, 9966, 9968, 9970, 9973-9977, 9979, 9985-9987, 9989,
9995, 10001-10002, 10004-10012, 10014, 10016, 10018-10020,
10022-10024, 10027-10034, 10038-10040, 10044-10045, 10050-10066,
10068-10071, 10073, 10075-10076, 10078-10079, 10081-10082,
10086-10089, 10091-10099, 10102-10107, 10110-10121, 10125-10126,
10128-10132, 10134, 10143-10145, 10147, 10152-10157, 10159-10160,
10162-10163, 10165-10169, 10172-10173, 10175-10176, 10180-10183,
10185, 10194, 10196-10197, 10199-10202, 10204-10208, 10211-10213,
10218-10221, 10223-10227, 10232, 10234-10235, 10238, 10240,
10243-10244, 10246, 10248-10254, 10257, 10262-10264, 10266, 10269,
10271-10274, 10276, 10283-10284, 10288, 10291-10295, 10297,
10304-10307, 10309-10310, 10313, 10315-10318, 10320-10329, 10332,
10336, 10338-10339, 10341-10346, 10354, 10357-10360, 10362, 10364,
10367, 10369-10370, 10373-10375, 10377, 10379-10381, 10383-10384,
10387, 10389, 10395-10396, 10399, 10401-10402, 10405-10407, 10409,
10411-10413, 10416-10417, 10422, 10427, 10434-10435, 10437, 10441,
10443-10445, 10447-10450, 10452, 10456, 10459, 10462, 10464-10474,
10476-10477, 10479-10481, 10483, 10486-10487, 10490, 10493-10504,
10507-10512, 10515-10516, 10519, 10522-10523, 10525-10526, 10529,
10531-10539, 10541, 10543-10545, 10548-10550, 10552, 10555-10556,
10558, 10560, 10564, 10568-10574, 10576-10577, 10579-10580, 10582,
10588-10593, 10595, 10598-10601, 10604, 10606-10611, 10618-10619,
10624-10625, 10628, 10631-10632, 10634, 10640, 10642-10646,
10648-10653, 10659-10665, 10667-10669, 10671, 10673-10675,
10677-10680, 10684-10685, 10687-10693, 10697-10698, 10700,
10702-10703, 10705, 10708-10715, 10717, 10720, 10722, 10729-10730,
10732-10733, 10735, 10737-10740, 10743, 10745, 10750, 10753-10754,
10756-10758, 10760-10764, 10768, 10770-10772, 10775-10776, 10782,
10785-10786, 10788, 10791-10794, 10796-10800, 10803, 10806-10809,
10819-10820, 10822-10825, 10829-10831, 10833, 10836-10838,
10840-10843, 10845-10847, 10851-10853, 10856, 10863, 10865-10870,
10872, 10874-10878, 10880, 10885, 10892, 10894-10897, 10899-10910,
10912-10913, 10915-10920, 10930-10931, 10935, 10938-10942,
10944-10945, 10949, 10951, 10954, 10958-10959, 10964, 10967-10975,
10979, 10981-10984, 10987, 10990-10991, 10993-10994, 10996-11002,
11004-11007, 11010-11011, 11014, 11016, 11018, 11020, 11022,
11025-11026, 11028-11030, 11032-11038, 11040-11051, 11055, 11057,
11059-11066, 11068-11070, 11072, 11074, 11076, 11079-11083, 11085,
11087-11091, 11095-11109, 11115-11117, 11120, 11124-11127,
11129-11130, 11133-11140, 11142-11145, 11147, 11151, 11158, 11160,
11162-11163, 11165, 11171-11172, 11175, 11177, 11179, 11181,
11183-11184, 11186-11187, 11189-11191, 11193-11195, 11198, 11201,
11204-11208, 11219, 11221, 11224, 11226-11229, 11231-11233,
11237-11238, 11243, 11245, 11247, 11249-11250, 11252-11255,
11258-11264, 11268-11270, 11272, 11274, 11276-11277, 11279-11280,
11284, 11286-11287, 11289-11290, 11292, 11296, 11298, 11300,
11302-11306, 11308-11314, 11321, 11324-11325, 11330-11332, 11334,
11336-11338, 11340, 11344-11347, 11349, 11351, 11353-11356,
11360-11363, 11365, 11370-11375, 11380-11393, 11395-11396, 11398,
11400-11403, 11408-11409, 11412-11414, 11419, 11421-11425, 11429,
11431, 11435, 11437-11439, 11441, 11444, 11449-11450, 11452-11453,
11455, 11461-11462, 11465-11466, 11468-11469, 11471, 11473,
11475-11477, 11482-11483, 11485-11486, 11488, 11490-11491,
11493-11497, 11501, 11504, 11506-11511, 11516, 11519, 11524,
11527-11528, 11531, 11535, 11537-11539, 11541, 11544-11545, 11557,
11564, 11568-11569, 11571-11572, 11574-11579, 11583-11585,
11588-11589, 11591-11593, 11595, 11597-11600, 11602-11603,
11606-11609, 11612, 11615-11623, 11625-11626, 11628-11633,
11635-11637, 11644, 11646-11648, 11652-11655, 11659-11660,
11666-11670, 11672, 11676-11683, 11685-11686, 11689, 11693-11696,
11698-11700, 11703-11704, 11707, 11709, 11711-11712, 11714-11716,
11718-11723, 11726-11727, 11734, 11738-11740, 11744-11745,
11747-11750, 11752, 11755-11757, 11759, 11761-11763, 11765-11769,
11771-11774, 11776-11784, 11786-11791, 11793-11795, 11797-11801,
11804, 11806, 11808-11812, 11815-11816, 11818-11819, 11821-11823,
11826-11832, 11834-11837, 11839-11841, 11848, 11851-11855,
11862-11863, 11865-11866, 11868, 11870-11871, 11873-11880,
11882-11885, 11888-11893, 11898, 11900-11901, 11903-11905, 11907,
11909-11912, 11915, 11918-11921, 11923, 11926, 11928, 11930, 11933,
11935, 11937, 11940-11945, 11948-11949, 11952-11954, 11956-11964,
11966-11970, 11973-11974, 11976-11981, 11983-11985, 11987-11989,
11991-11994, 11997-12001, 12003-12006, 12008-12009, 12011,
12013-12014, 12016, 12019-12022, 12024-12027, 12029-12031, 12033,
12035-12040, 12042-12043, 12048, 12051-12055, 12058-12061,
12063-12070, 12072-12076, 12086-12090, 12092-12096, 12098-12100,
12105, 12107, 12109-12113, 12116-12120, 12122-12123, 12125,
12127-12128, 12130-12131, 12133, 12137-12139, 12141-12145, 12148,
12151, 12153-12155, 12157-12159, 12161, 12163, 12166-12167,
12170-12171, 12174-12180, 12182-12189, 12193-12194, 12196,
12201-12202, 12206-12210, 12213-12215, 12218-12221, 12223-12225,
12227-12231, 12233, 12237-12238, 12241, 12246-12248, 12250-12252,
12255-12256, 12259, 12262-12264, 12266-12270, 12275, 12277-12278,
12280, 12282, 12290-12296, 12298-12303, 12309, 12311-12314,
12316-12319, 12322, 12325, 12328, 12330, 12332-12348, 12350-12353,
12357-12361, 12363-12364, 12366-12367, 12370, 12372-12374,
12377, 12379, 12382-12391, 12393-12394, 12396-12400, 12402, 12404,
12409-12410, 12412, 12415-12417, 12419-12423, 12425-12427,
12430-12434, 12437-12438, 12440, 12446-12447, 12450-12456,
12459-12463, 12465-12466, 12469-12470, 12472-12473, 12475-12481,
12485-12486, 12491, 12495, 12497-12499, 12501-12503, 12505,
12507-12508, 12510-12512, 12515-12517, 12519-12525, 12527, 12529,
12531-12532, 12535-12557, 12561, 12563-12565, 12567-12568, 12570,
12574-12575, 12577-12578, 12581-12585, 12587-12592, 12594-12595,
12597-12598, 12601, 12603, 12605-12607, 12609, 12611, 12613-12614,
12620-12622, 12624, 12626, 12628, 12630, 12633-12636, 12641-12643,
12645-12649, 12651, 12654-12658, 12660-12684, 12687, 12689-12690,
12692-12697, 12699, 12701, 12703-12710, 12714-12715, 12718-12727,
12729-12732, 12734-12741, 12743-12744, 12747-12755, 12757-12765,
12767-12768, 12771-12778, 12781, 12784-12791, 12795, 12798,
12801-12805, 12807-12809, 12812, 12815, 12817-12820, 12824, 12827,
12830-12831, 12835, 12837-12845, 12847-12848, 12850, 12853-12857,
12860-12863, 12866-12867, 12869-12870, 12872-12874, 12876-12877,
12879, 12881, 12885, 12888-12889, 12893-12896, 12899, 12901,
12904-12905, 12907, 12912-12913, 12915, 12917, 12919-12920,
12922-12926, 12928-12929, 12931-12939, 12941-12944, 12946, 12951,
12955-12956, 12958-12963, 12967-12970, 12972, 12974-12975, 12977,
12980-12982, 12984-12986, 12988-12992, 12995-12996, 12998-12999,
13001, 13003, 13006-13010, 13016-13017, 13023, 13025-13027,
13033-13035, 13037-13038, 13049-13051, 13053-13056, 13058,
13060-13062, 13064-13070, 13072, 13075, 13078, 13080, 13083-13087,
13091-13097, 13101, 13103, 13105, 13108-13110, 13112-13116,
13119-13122, 13126, 13130-13131, 13133-13137, 13139-13147,
13149, 13151, 13154-13155, 13159-13163, 13166-13168, 13170-13171,
13173-13174, 13176-13179, 13182-13186, 13188, 13190-13197,
13199-13200, 13202-13205, 13207-13216, 13218-13227, 13229-13231,
13234-13235, 13237, 13239-13240, 13242-13243, 13245-13247,
13250-13252, 13254, 13262, 13264-13266, 13269-13281, 13283-13284,
13286-13287, 13291, 13294-13298, 13300, 13303, 13305, 13307-13311,
13316, 13318-13319, 13322, 13324-13332, 13334, 13336-13337,
13340-13343, 13345, 13347, 13349-13351, 13355-13358, 13361-13368,
13370-13371, 13373-13374, 13376, 13378, 13380-13386, 13389-13390,
13392, 13394-13398, 13400-13405, 13407, 13409-13415, 13419-13420,
13422-13424, 13428, 13431-13439, 13441, 13443-13444, 13446-13452,
13455-13461, 13463, 13465-13468, 13470-13471, 13474-13475, 13477,
13479, 13482, 13484-13489, 13492-13500, 13504-13505, 13507-13514,
13516-13517, 13519, 13525-13526, 13531, 13533, 13535-13537,
13540-13541, 13543-13544, 13548-13552, 13554, 13556, 13559-13564,
13566-13568, 13573, 13575, 13577-13580, 13582, 13584, 13587-13588,
13591-13593, 13596-13599, 13601-13604, 13608-13610, 13615, 13617,
13619-13622, 13625-13626, 13628-13629, 13633, 13635-13637,
13640-13643, 13647-13648, 13650-13658, 13660-13661, 13664,
13666-13667, 13669-13672, 13674-13675, 13678-13679, 13682-13686,
13689-13690, 13693-13699, 13703, 13712-13714, 13719-13720, 13722,
13724, 13726, 13728, 13730, 13732, 13735-13740, 13742, 13745-13748,
13751-13752, 13754-13761, 13763, 13768-13770, 13772, 13774-13775,
13778, 13781, 13786, 13788, 13790-13793, 13798-13802, 13809-13812,
13816, 13823-13824, 13826-13827, 13829-13830, 13832, 13835-13836,
13838-13842, 13844, 13847, 13849, 13852-13854, 13856, 13860,
13862-13864, 13867, 13872, 13874, 13876-13878, 13881, 13883, 13885,
13889, 13891-13895, 13901-13902, 13905, 13907-13916, 13920,
13922-13923, 13925, 13929, 13933, 13935, 13939-13950, 13953, 13955,
13957-13963, 13966-13969, 13971-13972, 13974, 13976, 13979,
13981-13983, 13985-13989, 13994, 13996-13997, 13999-14000,
14004-14006, 14008-14009, 14012-14017, 14019, 14021, 14024, 14027,
14030-14031, 14033, 14039, 14043, 14046, 14049-14051, 14057-14058,
14061, 14064-14070, 14072-14075, 14079-14080, 14082, 14084-14087,
14089-14090, 14092-14102, 14107-14108, 14112-14113, 14116-14121,
14123-14125, 14127-14130, 14132, 14134, 14141, 14143-14144,
14148-14149, 14152-14157, 14159-14165, 14167-14171, 14173, 14175,
14177-14178, 14182-14184, 14186, 14189, 14191-14194, 14196-14197,
14199, 14203-14204, 14206-14207, 14210, 14213, 14216-14218, 14221,
14223-14224, 14226-14230, 14233, 14235-14237, 14239-14240,
14242-14244, 14246-14247, 14249, 14252-14255, 14257, 14260,
14262-14263, 14266, 14268-14269, 14273, 14279-14285, 14289-14293,
14296, 14298, 14300-14301, 14310, 14322, 14324, 14326, 14332-14333,
14337-14339, 14341-14344, 14346-14347, 14349, 14351, 14353,
14356-14360, 14362, 14364-14367, 14370, 14373-14374, 14380,
14386-14388, 14391, 14393-14394, 14396, 14398-14400, 14403, 14411,
14418, 14423-14424, 14431-14432, 14436-14437, 14441, 14446-14447,
14449-14451, 14455-14457, 14459-14460, 14462-14463, 14465,
14471-14473, 14475-14476, 14478, 14480-14482, 14484, 14487,
14490-14494, 14496, 14498-14499, 14502-14505, 14508-14509, 14513,
14515-14517, 14520-14522, 14524-14526, 14528, 14532-14533, 14536,
14538, 14540-14541, 14543-14544, 14546, 14552, 14555-14558, 14560,
14562, 14564-14570, 14574-14575, 14577, 14580-14582, 14586-14592,
14595, 14599, 14602, 14604-14606, 14608-14612, 14617-14620,
14623-14624, 14626-14627, 14629, 14632-14633, 14635-14636,
14638-14639, 14641-14642, 14644-14651, 14653-14656, 14659,
14661, 14664-14671, 14673, 14675-14676, 14680-14682, 14687,
14689-14690, 14692, 14694-14698, 14701, 14703-14704, 14706-14709,
14711, 14713-14715, 14723, 14727, 14729-14732, 14734, 14737,
14745-14747, 14749-14751, 14753-14755, 14760, 14762-14765,
14767-14768, 14771-14775, 14781-14786, 14788-14792, 14794-14796,
14798-14799, 14801-14807, 14809-14810, 14812, 14814, 14816-14817,
14819-14822, 14827-14828, 14830-14831, 14833-14834, 14837-14839,
14842-14846, 14848-14849, 14851-14853, 14855, 14858-14864, 14867,
14880-14887, 14891, 14894, 14896-14899, 14901-14904, 14907-14909,
14911, 14913, 14915, 14917-14919, 14921-14923, 14925, 14928, 14930,
14935-14936, 14938-14939, 14941, 14948, 14952-14953, 14955-14956,
14960, 14962, 14966-14972, 14974-14975, 14977, 14979-14982, 14985,
14987-14989, 14992, 14994-14997, 15000, 15002-15005, 15008-15010,
15012-15017, 15019-15023, 15027-15028, 15030-15031, 15033, 15035,
15037-15041, 15043-15044, 15046-15048, 15051-15053, 15056,
15058-15060, 15062-15066, 15068-15074, 15076-15077, 15081-15083,
15085, 15087, 15089, 15094, 15098, 15102, 15104-15108, 15110-15116,
15119-15123, 15125-15126, 15128, 15132, 15139-15142, 15144-15145,
15147, 15151-15152, 15155, 15158, 15163-15164, 15166-15168,
15170-15172, 15174-15175, 15177-15178, 15181-15183, 15185-15186,
15188-15190, 15192, 15194, 15197, 15202, 15209, 15215, 15219,
15224-15226, 15228, 15230, 15232-15233, 15235-15238, 15249-15250,
15252-15253, 15255, 15257-15259, 15262, 15266, 15268-15271,
15273-15274, 15277, 15279-15280, 15283-15287, 15289-15290,
15292-15297, 15300, 15302, 15308-15312, 15315, 15319, 15328-15331,
15334, 15338, 15342, 15344, 15346, 15351, 15353-15354, 15360, 15365,
15371, 15374, 15377-15383, 15387, 15390-15391, 15393, 15395,
15398-15400, 15407, 15409, 15411-15416, 15418-15419, 15423-15426,
15428, 15430, 15432-15434, 15437, 15444, 15446, 15448, 15450-15456,
15459-15460, 15463-15464, 15468-15476, 15478-15482, 15484,
15487-15493, 15497-15498, 15504-15507, 15515, 15517, 15519-15521,
15523-15527, 15529-15531, 15533-15534, 15536-15539, 15542, 15545,
15549, 15551, 15555, 15567, 15570-15571, 15573, 15575-15576, 15581,
15583-15584, 15586, 15589-15594, 15598-15599, 15601-15602, 15605,
15609-15611, 15614-15615, 15618-15620, 15626, 15635, 15645, 15648,
15650-15652, 15659, 15661-15666, 15668, 15670-15671, 15674,
15676-15677, 15679-15691, 15694, 15700-15701, 15703-15713,
15715-15716, 15718, 15720-15722, 15727-15730, 15732-15733,
15736-15740, 15742, 15746, 15749-15765, 15768, 15770, 15773-15774,
15777, 15779-15784, 15786-15802, 15804-15809, 15811, 15813-15828,
15830-15835, 15837-15838, 15840-15846, 15848-15858, 15860-15861,
15864, 15866, 15868-15878, 15880, 15882-15886, 15892-15893,
15897-15898, 15900-15902, 15904, 15906-15907, 15910-15923,
15926-15927, 15929-15933, 15935-15936, 15942-15943, 15945-15947,
15949-15950, 15953, 15956-15963, 15965-15966, 15968, 15970-15971,
15973-15974, 15976, 15978-15981, 15983, 15986, 15988-15989,
15991-15995, 15997-15998, 16004-16005, 16007-16012, 16015-16016,
16018-16020, 16023-16032, 16034, 16037-16038, 16044, 16046-16050,
16053, 16057, 16059, 16061, 16063-16064, 16069, 16071-16079,
16082-16083, 16085-16088, 16094-16096, 16098, 16101, 16109-16110,
16112-16115, 16117, 16119-16120, 16127-16129, 16131, 16137,
16139-16141, 16143-16145, 16149, 16151, 16154-16155, 16159-16162,
16166-16174, 16178, 16180-16182, 16185-16189, 16191, 16197-16204,
16206-16209, 16216-16217, 16219, 16223-16227, 16229, 16233-16234,
16236-16239, 16241-16244, 16246-16247, 16249, 16252, 16254, 16257,
16259-16260, 16263-16264, 16266-16269, 16273, 16276, 16279-16284,
16288-16290, 16292, 16295, 16298, 16302-16303, 16305-16309, 16318,
16321, 16327, 16330-16334, 16336-16341, 16343, 16347, 16350,
16352-16353, 16356, 16358-16359, 16363, 16365-16370, 16374, 16376,
16381, 16383, 16385-16392, 16394-16401, 16403-16404, 16407, 16409,
16412-16414, 16416-16423, 16425-16426, 16430-16433, 16435, 16437,
16439-16441, 16444-16447, 16450, 16452-16455, 16457-16459
Single Strand Oligonucleotides (Sense Strand of Target Gene)
SegID range: 16426-45713
SeqIDs w/o G Runs:
16426-16443, 16451-16453, 16455-16465, 16480-16511, 16554-16810,
16824-16869, 16884-16919, 16934-17023, 17037-17389, 17403-17628,
17643-17720, 17734-17741, 17765-17790, 17814-17818, 17843-17880,
17894-17927, 17941-17950, 17964-17998, 18012-18082, 18098,
18114-18140, 18155, 18174-18189, 18216-18220, 18234-18264,
18278-18336, 18350-18354, 18368-18377, 18391-18402, 18421-18428,
18444-18445, 18459-18578, 18592-18628, 18642-18660, 18674-18687,
18702-18703, 18718-18722, 18737-18744, 18758-18770, 18791-18808,
18823-18926, 18956-19009, 19023-19040, 19054-19078, 19092-19133,
19147-19205, 19220-19401, 19415-19417, 19431-19497, 19511-19539,
19553-19555, 19569-19591, 19605-19606, 19620-19846, 19861-20036,
20050-20056, 20084-20099, 20114-20130, 20144-20172, 20186-20261,
20275-20454, 20469-20683, 20697-20848, 20862-21014, 21049-21165,
21180-21242, 21256-21261, 21275-21280, 21294-21361, 21398-21446,
21460-21521, 21535-21603, 21617-21984, 21998-22155, 22174-22182,
22196-22318, 22341-22450, 22464-22465, 22479-22500, 22515-22522,
22548-22598, 22613-22657, 22671-22699, 22713-22723, 22737-22753,
22767-22793, 22816-22817, 22831-22919, 22933-22954, 22980-23032,
23046-23050, 23065-23077, 23098, 23119-23211, 23225-23290,
23305-23318, 23333-23340, 23357-23362, 23389-23425, 23480-23483,
23513-23535, 23557-23602, 23648-23668, 23684-23744, 23765-23780,
23802-23815, 23841-23889, 23903-23920, 23935-23954, 23969-24010,
24033-24053, 24067-24088, 24110-24135, 24149-24169, 24191-24198,
24213-24219, 24235-24242, 24256-24309, 24323-24369, 24389-24511,
24525-24717, 24739-24799, 24814-24913, 24928-24930, 24944-24992,
25006-25072, 25086-25182, 25198-25248, 25262-25274, 25288-25487,
25502-25823, 25838-25932, 25947-26034, 26061-26135, 26149,
26164-26220, 26254-26297, 26311-26322, 26345, 26359-26361,
26375-26392, 26407, 26422-26521, 26535-26544, 26559-26603,
26617-26670, 26694-26793, 26807, 26821-26848, 26862, 26905-26979,
26993-27012, 27043-27072, 27088-27264, 27279-27295, 27309-27325,
27339-27405, 27420-27539, 27554-27573, 27587-27614, 27653-27671,
27697, 27729-27790, 27812-27832, 27858-27897, 27928-27935,
27949-27951, 27966-27976, 27990-27997, 28024-28139, 28153-28272,
28287-28306, 28320-28627, 28641-28650, 28664-28666, 28691-28694,
28708-28794, 28814-29339, 29354-29395, 29423-29515, 29535-29839,
29873-30111, 30125-30439, 30453-30461, 30475-30691, 30715-30731,
30763-30884, 30898-30941, 30955-30996, 31010-31244, 31259-31304,
31318-31352, 31366-31378, 31392-31439, 31453-31501, 31531-31596,
31611-31618, 31632-31750, 31765-31776, 31790-31899, 31913-31923,
31937-31940, 31954-32036, 32050-32066, 32080-32176, 32191-32227,
32241-32250, 32264-32270, 32285-32337, 32351-32359, 32373-32388,
32408-32478, 32503, 32517-32589, 32603-32729, 32744-32795,
32809-32953, 32967-32968, 32993-33318, 33332-33367, 33382-33464,
33478-33507, 33531-33590, 33604-33606, 33633-33639, 33653-33690,
33704-33709, 33723, 33739-33749, 33776, 33797-34293, 34308-34364,
34389-34414, 34430-34457, 34471-34472, 34486-34506, 34531-34562,
34599-34804, 34818-34832, 34876-34881, 34896-34945, 34985-35012,
35047-35053, 35067-35091, 35105-35240, 35255-35263, 35277-35352,
35368-35379, 35393-35498, 35523-35582, 35635-35764, 35779-35901,
35917-36076, 36099-36103, 36125-36130, 36148-36160, 36174-36217,
36231-36242, 36265-36317, 36336-36523, 36550-36605, 36619-36804,
36818-37213, 37260-37264, 37292-37303, 37323-37418, 37437-37482,
37518-37557, 37578-37592, 37617-37619, 37633-37640, 37654-37726,
37740-37800, 37822-37830, 37844-37865, 37880-37945, 37959-37974,
38000-38107, 38121-38138, 38152-38344, 38358-38398, 38412-38530,
38547-38564, 38578-38669, 38683-38700, 38714-38775, 38797-38866,
38892-38902, 38908-38925, 38948-38970, 38993-39015, 39036-39069,
39083-39423, 39437-40260, 40275-40294, 40309-40460, 40474-40493,
40523-40526, 40557-40656, 40670-40672, 40688-40796, 40824-40826,
40847-40861, 40875-40899, 40913-40967, 40982-41097, 41111-41285,
41309-41311, 41326-41450, 41464-41600, 41614-41684, 41698,
41724-41758, 41772-41809, 41824-41882, 41896-41921, 41935-42033,
42047-42172, 42186-42187, 42222-42253, 42278-42448, 42463-42601,
42615-42621, 42635-42711, 42725-42740, 42760-42887, 42901-42965,
42980-42988, 43002-43010, 43024-43030, 43053-43096, 43110-43228,
43242-43243, 43270-43287, 43301-43391, 43405-43505, 43519-43523,
43537-43557, 43571-43622, 43636-43668, 43682-43695, 43716-43722,
43736-43816, 43830-43851, 43865-44058, 44072-44344, 44358-44406,
44420-44445, 44459-44464, 44484-44496, 44516-44547, 44561-44583,
44627-44630, 44644-44738, 44752-44763, 44777-44861, 44878-44925,
44941-45010, 45036-45040, 45074-45201, 45215-45267, 45293-45342,
45355-45378, 45391-45433, 45452-45513, 45524-45665, 45679-45688,
45702-45713
SeqIDs w/o miR Seeds:
16426, 16430-16433, 16435, 16437, 16439-16441, 16444-16447, 16450,
16452-16455, 16457-16459, 16462-16465, 16467-16468, 16470-16472,
16478-16487, 16489, 16492, 16495-16497, 16499-16500, 16504,
16507-16508, 16510-16516, 16524-16529, 16531-16534, 16536, 16541,
16544, 16549, 16552, 16554, 16558, 16560-16565, 16567, 16570-16572,
16574, 16576, 16580, 16582, 16585-16586, 16588-16593, 16595-16597,
16599-16602, 16604-16605, 16607-16611, 16613, 16615-16621, 16623,
16625, 16627-16636, 16638, 16640-16643, 16646, 16649-16650,
16652-16663, 16665-16668, 16670-16671, 16673, 16676-16677,
16679-16682, 16685-16686, 16690, 16692, 16694, 16699-16701, 16703,
16705-16707, 16709-16710, 16712, 16714-16718, 16720-16725, 16729,
16732-16735, 16737-16742, 16747-16753, 16755, 16757-16758,
16760-16776, 16778-16779, 16781-16783, 16785-16789, 16792,
16795-16803, 16806, 16809-16811, 16813-16816, 16818-16820,
16822-16823, 16826-16827, 16830-16832, 16834-16835, 16837-16847,
16851-16852, 16854, 16858-16861, 16863, 16866, 16868-16870,
16872-16874, 16882, 16884-16894, 16896, 16898, 16900-16901,
16905-16907, 16909, 16911-16913, 16915, 16917, 16919, 16922-16923,
16925, 16928, 16930, 16932-16934, 16936-16937, 16940, 16942-16946,
16948, 16953-16954, 16956-16957, 16959-16961, 16964-16966,
16968-16971, 16973, 16975-16984, 16987, 16989-16993, 16998-17003,
17005, 17010-17012, 17014-17015, 17017, 17019-17021, 17023-17025,
17027-17029, 17039, 17041, 17043, 17045, 17048, 17052-17053,
17055-17056, 17058-17059, 17061-17068, 17072, 17075, 17078, 17081,
17084-17087, 17089, 17092, 17094-17097, 17099-17101, 17104,
17107-17108, 17112-17120, 17122, 17124-17125, 17128-17130,
17132-17133, 17139, 17141, 17143-17146, 17149, 17154-17156,
17158-17159, 17161-17162, 17164, 17169-17176, 17179-17182, 17185,
17187-17188, 17190, 17195, 17197-17198, 17200-17210, 17213,
17217-17220, 17222-17227, 17229-17230, 17234-17236, 17238-17239,
17241, 17245-17253, 17255, 17257-17262, 17264-17265, 17267,
17270-17271, 17273-17276, 17279, 17281-17283, 17285-17288, 17292,
17294-17296, 17299, 17301-17309, 17313, 17316-17318, 17320-17321,
17324-17325, 17328, 17333, 17337, 17340-17343, 17347-17350,
17354-17355, 17357, 17359-17362, 17364-17367, 17370, 17372-17374,
17377-17384, 17386-17387, 17391, 17396, 17398, 17402, 17405, 17407,
17410-17412, 17414, 17420-17421, 17424-17428, 17430-17432, 17435,
17439-17447, 17449-17451, 17453, 17455, 17457-17460, 17462-17464,
17467, 17470, 17472-17475, 17478, 17481-17483, 17490, 17492-17493,
17495-17500, 17502-17503, 17505-17506, 17510-17515, 17518-17519,
17522, 17526-17528, 17530, 17532-17535, 17537-17538, 17541, 17543,
17545, 17547-17549, 17558-17559, 17562, 17565, 17567, 17570, 17572,
17577-17581, 17583, 17586-17588, 17590-17593, 17595, 17597-17598,
17600, 17603, 17605-17609, 17611-17613, 17615-17622, 17626, 17628,
17630, 17632-17635, 17642, 17645, 17649-17652, 17657-17660,
17662-17668, 17670-17671, 17673-17674, 17676-17678, 17680,
17683-17684, 17686-17687, 17689-17691, 17698-17709, 17711-17712,
17715-17716, 17718, 17721-17722, 17724-17730, 17732, 17734,
17741-17747, 17750-17751, 17753-17756, 17764-17765, 17767-17770,
17772, 17775-17776, 17779, 17781-17783, 17785, 17790-17794,
17796, 17798-17799, 17807-17808, 17810, 17812, 17816-17819,
17823-17824, 17826, 17829, 17831-17832, 17834, 17837, 17841,
17843-17844, 17846-17853, 17855-17857, 17860-17866, 17870,
17872-17877, 17881-17883, 17886-17887, 17889-17890, 17894-17896,
17898, 17900-17901, 17904, 17906, 17910-17920, 17923-17925,
17927-17929, 17931-17934, 17936, 17941-17944, 17949, 17952-17953,
17955, 17960, 17963-17965, 17967, 17969, 17971-17972, 17974-17975,
17983, 17987, 17994-17998, 18000, 18004, 18007, 18012, 18014-18016,
18021-18027, 18029-18030, 18032, 18036, 18038, 18040-18042,
18044-18046, 18049, 18051, 18058, 18060-18063, 18067-18069,
18071-18072, 18076-18079, 18081, 18083-18085, 18087-18092,
18097-18100, 18102-18108, 18110, 18112, 18114-18116, 18118, 18125,
18129, 18131, 18133-18135, 18139, 18142, 18145-18146, 18150-18151,
18153-18155, 18161-18162, 18166, 18169-18178, 18180, 18182-18183,
18187-18193, 18198, 18201, 18203, 18206-18210, 18214-18219,
18225-18227, 18231, 18233-18234, 18236, 18239-18242, 18245,
18247-18248, 18251-18252, 18255-18256, 18262-18267, 18270, 18273,
18280, 18282-18283, 18286, 18288, 18295, 18297, 18300-18302,
18305-18306, 18310, 18312, 18315, 18317-18319, 18322-18325,
18327-18328, 18330, 18332, 18335-18336, 18338, 18340-18341, 18345,
18350, 18352-18353, 18357-18359, 18363, 18366, 18369, 18371,
18374-18376, 18378-18380, 18383-18384, 18388, 18390-18399,
18401-18402, 18405, 18407-18413, 18416, 18421, 18425-18428,
18434-18435, 18441, 18443-18445, 18447-18449, 18451-18453, 18457,
18459-18461, 18463-18471, 18473-18474, 18478-18480, 18482-18487,
18489-18491, 18493-18494, 18498, 18500, 18503-18504, 18507-18510,
18514-18517, 18521, 18523-18527, 18529, 18531, 18534, 18536-18539,
18541, 18545, 18548-18553, 18557-18561, 18563-18564, 18566-18567,
18569-18572, 18574, 18576-18580, 18582, 18588-18589, 18591,
18593-18601, 18603, 18605-18610, 18612-18615, 18617-18621, 18623,
18625, 18631, 18635-18638, 18640-18642, 18644-18645, 18648-18651,
18653, 18658-18659, 18662-18668, 18673-18674, 18676-18678,
18682-18683, 18685-18693, 18697, 18699, 18702, 18704, 18708,
18710-18712, 18720-18721, 18724-18728, 18736-18739, 18741,
18743-18746, 18748-18751, 18755, 18757-18759, 18761, 18763-18764,
18766, 18770, 18777, 18779, 18781-18786, 18788-18794, 18796-18797,
18801, 18803-18805, 18808, 18812-18813, 18818, 18821, 18823-18824,
18830-18831, 18836, 18838-18839, 18841, 18844-18846, 18848, 18853
, 18856-18857, 18859-18860, 18863-18868, 18873-18878, 18880-18881,
18887, 18890-18893, 18897, 18900-18901, 18903, 18907, 18911-18915,
18917-18918, 18920, 18924, 18929-18930, 18933, 18935, 18943,
18947-18948, 18951-18954, 18958, 18960, 18962, 18966-18970,
18973, 18975, 18977-18978, 18980-18982, 18984-18991, 18993-18995,
18998, 19002-19005, 19008, 19010-19020, 19022, 19024-19025,
19027-19028, 19036-19038, 19045-19046, 19049, 19055-19056,
19061-19062, 19064, 19067, 19071-19073, 19076-19078, 19080, 19083,
19093-19095, 19097, 19099-19102, 19104-19107, 19109, 19113-19114,
19116, 19120, 19123-19126, 19128, 19130-19134, 19137, 19139,
19141-19142, 19147, 19150-19155, 19157-19162, 19164-19169,
19172-19173, 19176-19181, 19183-19186, 19188-19189, 19194-19195,
19204-19210, 19215, 19221, 19226-19232, 19234, 19236, 19240,
19244-19247, 19249-19252, 19255-19257, 19260-19261, 19263-19265,
19271, 19275-19276, 19278-19279, 19282, 19284, 19286, 19289-19290,
19292-19294, 19298, 19303, 19306, 19309-19315, 19317-19318,
19323-19326, 19329-19332, 19336-19337, 19341, 19343-19352, 19354,
19356-19363, 19365, 19368, 19371-19374, 19376-19379, 19381-19382,
19384-19389, 19391, 19396, 19398-19399, 19404-19406, 19414-19415,
19417, 19419, 19421-19424, 19426-19427, 19430-19432, 19434,
19440-19444, 19446, 19448-19449, 19451-19461, 19463, 19468-19472,
19475-19476, 19478, 19480-19481, 19483-19484, 19487, 19490, 19493,
19500-19503, 19506, 19510, 19512, 19514-19515, 19517-19520,
19522-19525, 19527-19529, 19532, 19538, 19541-19542, 19544-19547,
19552-19553, 19556-19558, 19560-19564, 19566-19567, 19569-19570,
19572-19573, 19578-19582, 19584-19585, 19587-19592, 19594-19597,
19599-19600, 19605-19610, 19613-19617, 19619-19621, 19623,
19626-19628, 19630, 19634, 19637-19639, 19641-19643, 19645,
19648, 19651, 19655-19659, 19661-19662, 19664, 19667-19671,
19673-19674, 19678-19679, 19682-19683, 19685-19691, 19695-19703,
19705-19707, 19709-19713, 19716-19717, 19719-19722, 19724-19730,
19732-19740, 19742-19744, 19746, 19750-19752, 19755-19758, 19760,
19762, 19764-19765, 19768-19769, 19771, 19774-19776, 19778-19782,
19785, 19787, 19789-19798, 19800-19807, 19811-19812, 19814-19816,
19818-19820, 19822, 19825, 19827-19828, 19831-19832, 19834-19839,
19842, 19844-19846, 19848-19852, 19858, 19860-19861, 19863-19869,
19871-19878, 19880-19881, 19883-19885, 19890-19891, 19893,
19895-19900, 19903-19905, 19907-19908, 19911-19914, 19916,
19918-19919, 19922-19923, 19925-19928, 19930-19932, 19934-19935,
19937, 19939-19940, 19942, 19944-19946, 19948-19949, 19952-19953,
19957-19958, 19960, 19963, 19965, 19967-19973, 19976-19977, 19983,
19985-19989, 19991, 19993-19994, 19996, 19998, 20000-20002,
20005-20020, 20022, 20024-20025, 20027-20035, 20037, 20039-20041,
20044-20046, 20049, 20051-20055, 20057, 20059-20064, 20071, 20073,
20075-20076, 20078, 20081, 20083-20084, 20086, 20089-20092, 20094,
20096-20097, 20102-20105, 20111-20115, 20121, 20129-20131,
20133-20135, 20137, 20140, 20144-20149, 20151, 20155, 20159,
20162-20164, 20166-20167, 20169, 20171, 20173, 20176, 20180-20181,
20185-20186, 20189, 20191-20197, 20202-20211, 20215, 20218-20219,
20223-20228, 20232, 20236-20245, 20248-20254, 20256-20257,
20259-20267, 20269, 20272, 20274-20275, 20277-20280, 20282,
20285-20286, 20288, 20290, 20293-20300, 20306, 20308, 20310-20312,
20314-20317, 20320-20322, 20324, 20328, 20330-20332, 20334-20346,
20348, 20350-20356, 20358, 20360-20372, 20374-20377, 20379-20380,
20391-20392, 20394-20395, 20398-20399, 20401-20403, 20406-20408,
20410, 20412-20417, 20419-20420, 20422, 20425, 20427-20428, 20430,
20432-20433, 20437-20438, 20440-20444, 20447-20448, 20450-20461,
20467-20472, 20476, 20479-20483, 20488-20491, 20494-20496,
20498-20500, 20504, 20508-20513, 20516, 20518-20519, 20521,
20523-20525, 20527, 20529-20532, 20534, 20537, 20539-20543, 20545,
20551-20555, 20557, 20561-20563, 20566-20572, 20574-20576,
20578-20588, 20590-20593, 20595-20597, 20601-20603, 20605, 20607,
20609-20614, 20617-20618, 20620-20632, 20634, 20637-20638,
20641-20644, 20647-20648, 20652-20654, 20657, 20664, 20667,
20670-20671, 20674, 20680-20686, 20688-20689, 20691-20693, 20695,
20698, 20701-20705, 20707-20708, 20711, 20713, 20716-20719, 20721,
20728, 20732-20734, 20738-20741, 20743-20744, 20746-20748,
20750-20751, 20754, 20757, 20759-20764, 20766, 20768, 20772-20779,
20781-20787, 20789-20790, 20792-20796, 20799, 20802-20803,
20806-20826, 20831-20833, 20835-20837, 20839-20840, 20844-20847,
20849-20850, 20854, 20858, 20860-20862, 20866, 20868, 20870-20876,
20878-20887, 20889-20890, 20894, 20897-20899, 20906-20907,
20910-20913, 20916, 20918-20922, 20925, 20928-20933, 20939-20946,
20948, 20950-20955, 20957-20960, 20962-20966, 20968, 20970,
20973-20974, 20976-20978, 20982-20985, 20987-20989, 20994, 20996,
20998-20999, 21001-21003, 21005-21006, 21008, 21010-21012,
21016-21019, 21021-21022, 21029, 21032, 21035, 21037, 21039, 21041,
21044-21045, 21047-21051, 21053, 21057, 21065, 21067-21069, 21076,
21078-21080, 21084-21085, 21087, 21089-21094, 21096, 21099-21100,
21103-21108, 21110, 21112-21114, 21116-21117, 21121-21126, 21128,
21131-21138, 21140-21142, 21146, 21149-21150, 21152-21154, 21156,
21158-21159, 21163, 21165-21166, 21170, 21175, 21179, 21185-21194,
21196-21199, 21201, 21205-21209, 21211-21212, 21214, 21216-21219,
21221-21227, 21229-21231, 21233-21236, 21238, 21241, 21243,
21248-21251, 21256-21259, 21261, 21263-21269, 21273, 21275-21280,
21284, 21286-21287, 21293-21295, 21297-21298, 21300, 21302-21310,
21313-21314, 21317, 21320-21321, 21325, 21327-21328, 21330,
21332-21333, 21337-21338, 21340-21341, 21344, 21346-21348, 21351,
21354-21358, 21365-21370, 21375-21377, 21379, 21381, 21385, 21387,
21389, 21395, 21397-21403, 21405-21406, 21408-21411, 21418-21422,
21424-21425, 21427-21431, 21433, 21436, 21438-21439, 21444,
21448-21449, 21451-21453, 21455, 21457-21461, 21465-21467,
21471-21473, 21477, 21479, 21482-21484, 21486, 21490, 21492, 21494,
21496-21497, 21499, 21501, 21503-21507, 21511-21514, 21516-21517,
21519, 21522-21525, 21527-21531, 21536, 21541, 21543-21545,
21547-21552, 21556-21557, 21560-21561, 21568-21569, 21571-21578,
21580, 21586, 21590-21593, 21596-21598, 21604-21607, 21609,
21611-21613, 21615-21617, 21619, 21625-21628, 21631, 21633-21634,
21637-21639, 21642-21653, 21655-21657, 21660-21662, 21664-21665,
21670-21671, 21673, 21675, 21678-21680, 21682, 21687-21695, 21697,
21699-21702, 21708-21711, 21713, 21718, 21721, 21723, 21725-21726,
21731, 21733-21738, 21740-21745, 21747, 21750-21752, 21757-21758,
21760, 21762, 21769-21774, 21777-21780, 21783-21785, 21788-21792,
21794-21796, 21798-21800, 21802-21806, 21811-21814, 21816, 21818,
21820-21828, 21830-21837, 21841, 21843, 21845, 21847-21848,
21850-21851, 21853-21855, 21857-21859, 21861, 21863-21868,
21870-21874, 21876-21878, 21880-21881, 21883, 21887-21888,
21890-21897, 21899-21903, 21905, 21907-21909, 21911-21914,
21917-21918, 21921, 21925, 21927, 21929-21934, 21937, 21940-21942,
21944-21945, 21947-21948, 21952, 21954-21956, 21960-21963,
21965-21966, 21968, 21971-21973, 21976, 21978, 21980-21983,
21985, 21987-21988, 21991-21993, 21995, 21997-21998, 22001, 22003,
22005-22007, 22009-22010, 22017, 22020-22021, 22023, 22025-22026,
22029-22031, 22034, 22037, 22039, 22041, 22043, 22046, 22049, 22051,
22053-22063, 22065-22066, 22068-22070, 22073, 22075-22076,
22082-22089, 22091-22093, 22095, 22097-22102, 22105-22106, 22109,
22112-22113, 22116-22117, 22119-22125, 22130, 22132-22133,
22135-22136, 22139-22143, 22153-22157, 22161-22162, 22165-22166,
22168-22171, 22175, 22180-22181, 22185-22186, 22188-22190,
22196-22199, 22201, 22205-22206, 22208, 22210-22215, 22217,
22222-22225, 22227, 22229-22234, 22236-22241, 22243, 22247,
22249-22250, 22252-22255, 22257-22261, 22263-22265, 22267-22269,
22271, 22273, 22275-22278, 22287-22288, 22290-22292, 22296, 22298,
22301, 22303, 22308, 22310, 22313, 22316, 22320, 22323-22324, 22329,
22332, 22339, 22341-22342, 22345-22347, 22349, 22355, 22357-22359,
22361-22363, 22365, 22367-22369, 22373, 22376-22377, 22379-22380,
22384, 22387-22393, 22397-22398, 22401, 22403-22407, 22410,
22413-22414, 22416-22427, 22430-22432, 22434-22437, 22440-22441,
22446, 22448-22450, 22452-22459, 22461, 22469-22474, 22476,
22479-22480, 22482-22485, 22491, 22493-22495, 22500, 22502-22503,
22505-22506, 22508, 22515, 22519-22523, 22525, 22527, 22531-22532,
22541-22542, 22545-22546, 22548-22551, 22553-22554, 22556, 22564,
22566-22569, 22571-22579, 22581, 22583, 22585-22589, 22592-22597,
22601, 22604-22605, 22608-22609, 22612-22615, 22618-22622,
22624-22635, 22637, 22641-22642, 22644-22645, 22647-22650, 22653,
22655, 22658-22660, 22664-22667, 22673, 22680, 22682, 22685-22691,
22694-22698, 22700-22708, 22712, 22719, 22721, 22723-22724, 22730,
22732-22734, 22737, 22742-22744, 22746, 22749, 22751-22752,
22754-22755, 22757, 22759, 22762-22763, 22766, 22768, 22771, 22774,
22777-22778, 22780, 22784-22789, 22792, 22795-22796, 22798-22801,
22804, 22807, 22809, 22811-22813, 22815-22816, 22818-22821,
22823-22825, 22827, 22831-22836, 22838-22841, 22845-22854,
22856-22859, 22861, 22866-22871, 22873, 22875-22879, 22883,
22885-22887, 22891-22894, 22896-22898, 22901-22902, 22904-22906,
22908-22914, 22916-22919, 22923-22927, 22931, 22933, 22935, 22938,
22944-22948, 22950-22952, 22954-22956, 22958-22959, 22961, 22964,
22968-22974, 22976-22977, 22981, 22983-22984, 22989-22991,
22994-22995, 22999-23008, 23010-23011, 23014-23015, 23017-23020,
23022-23025, 23028-23029, 23031-23032, 23035-23038, 23041, 23044,
23046-23047, 23049-23054, 23056-23057, 23065, 23067-23072,
23074-23075, 23077-23079, 23081-23082, 23085, 23092-23093,
23096-23102, 23104, 23111, 23113-23114, 23117, 23119-23123,
23125-23126, 23129-23130, 23134, 23136-23138, 23140, 23142-23146,
23148-23150, 23152-23158, 23160-23166, 23168-23173, 23176-23178,
23180, 23182, 23184-23185, 23187-23188, 23190-23204, 23206-23213,
23216-23218, 23221-23222, 23227-23228, 23232-23235, 23237-23241,
23243, 23245, 23247-23248, 23250-23252, 23254-23256, 23259-23262,
23264, 23268-23270, 23275-23280, 23283-23287, 23291, 23293,
23295-23296, 23298-23299, 23302, 23304-23310, 23312, 23314-23317,
23321-23322, 23324, 23326-23327, 23333, 23335-23337, 23339-23342,
23344-23345, 23347, 23349, 23358-23359, 23362, 23364-23370,
23377-23384, 23386-23389, 23391-23393, 23396-23403, 23405-23406,
23409, 23412, 23414-23415, 23417-23418, 23420-23422, 23424,
23426-23430, 23433, 23435-23436, 23438-23442, 23454-23455,
23457, 23463, 23466, 23469, 23471-23474, 23476-23477, 23483,
23486-23487, 23489, 23491-23492, 23494-23501, 23503-23507,
23512-23514, 23516-23518, 23520, 23522-23523, 23525-23527, 23529,
23531-23533, 23535-23537, 23540, 23544, 23549-23551, 23553-23554,
23557, 23559-23562, 23564, 23566-23567, 23570, 23572-23582, 23584,
23586-23589, 23591-23592, 23597-23605, 23607-23610, 23616-23620,
23622-23624, 23627, 23630-23631, 23633-23635, 23637, 23642,
23644-23645, 23649-23650, 23652-23653, 23656-23657, 23659-23663,
23667-23668, 23670, 23672-23673, 23680, 23684-23685, 23687-23691,
23693-23694, 23696-23697, 23699-23701, 23704-23705, 23707-23715,
23720, 23722-23724, 23726-23727, 23729, 23731-23732, 23734-23735,
23737, 23739-23746, 23748, 23750, 23756-23757, 23759, 23762, 23765,
23768, 23776-23777, 23783, 23785-23788, 23794-23799, 23801-23806,
23808-23809, 23812, 23816, 23818, 23820, 23823-23824, 23827,
23829-23833, 23835-23836, 23839-23849, 23851, 23854-23872,
23876-23878, 23880-23884, 23888-23890, 23892-23893, 23895-23898,
23903, 23905-23911, 23916, 23919-23928, 23936-23938, 23941-23942,
23944-23946, 23948, 23950-23952, 23954-23962, 23965, 23969-23971,
23975, 23978, 23980-23981, 23984, 23986, 23988-23989, 23991,
23993-23996, 23998-24005, 24007, 24011-24013, 24015-24016, 24018,
24021, 24024-24026, 24028, 24034-24035, 24037-24043, 24048, 24050,
24055-24057, 24061, 24063-24064, 24066-24068, 24072-24078, 24080,
24082, 24085-24086, 24088, 24091-24096, 24102, 24106-24110,
24115-24117, 24119-24120, 24122-24125, 24127-24130, 24132, 24135,
24137-24138, 24141-24143, 24145, 24149, 24151-24156, 24158-24162,
24164, 24166-24171, 24176-24177, 24179-24180, 24183-24186,
24193-24194, 24197, 24201, 24204, 24206-24207, 24213-24216,
24218-24219, 24222, 24224-24229, 24238, 24240-24242, 24244-24245,
24247, 24249, 24257, 24260-24265, 24267-24268, 24270-24273,
24281, 24284, 24290-24301, 24303, 24308-24309, 24312, 24316-24319,
24322, 24324, 24326, 24328-24329, 24333, 24336-24342, 24344-24345,
24347-24349, 24353, 24355-24356, 24358-24359, 24361, 24364,
24367-24368, 24375-24376, 24380, 24383, 24387, 24389-24393, 24395,
24398-24402, 24404, 24406, 24408-24410, 24412-24415, 24417-24419,
24421-24423, 24425, 24429-24433, 24436-24437, 24439, 24446, 24451,
24453, 24457, 24459, 24461-24462, 24465-24467, 24469, 24471, 24473,
24475, 24477-24479, 24481-24482, 24484-24488, 24491-24492,
24494-24496, 24498-24500, 24504-24506, 24508-24516, 24518-24524,
24527, 24530, 24533-24537, 24539, 24541-24542, 24544-24549,
24552-24554, 24562-24563, 24565-24568, 24570-24571, 24573-24574,
24576-24577, 24581, 24583, 24585-24599, 24601-24602, 24605,
24609-24619, 24621-24622, 24624-24630, 24633-24642, 24645,
24648-24655, 24657-24658, 24664-24668, 24670-24676, 24678,
24681-24684, 24686, 24690-24692, 24694-24695, 24700-24701, 24704,
24706, 24708, 24710, 24712-24718, 24721-24724, 24726-24728,
24730-24733, 24735, 24738-24739, 24741, 24747-24752, 24755, 24758,
24760-24761, 24763-24765, 24767-24768, 24773, 24775-24776,
24780-24786, 24789-24790, 24792, 24794, 24797-24799, 24801-24803,
24805-24809, 24811, 24814-24815, 24817, 24819-24820, 24823,
24826-24829, 24831-24836, 24838-24841, 24844, 24846, 24849-24852,
24856, 24859-24860, 24862, 24865-24867, 24869, 24873-24875,
24877, 24879-24880, 24886-24888, 24891, 24893-24897, 24899-24900,
24902-24915, 24919, 24922, 24927-24928, 24931-24933, 24937-24938,
24941, 24943, 24945, 24947-24950, 24955-24956, 24958-24960,
24962-24963, 24965-24967, 24969-24971, 24973-24979, 24981,
24983-24985, 24987-24990, 24996-24997, 24999, 25003, 25007-25009,
25011-25013, 25016-25017, 25019-25025, 25033-25035, 25037,
25040-25045, 25047-25053, 25055-25060, 25062-25063, 25065-25066,
25069, 25072, 25075, 25077-25078, 25081, 25087-25089, 25091, 25093,
25095, 25101, 25105-25106, 25108, 25110, 25112, 25114-25118,
25123-25129, 25133-25135, 25137-25140, 25142-25145, 25147,
25149-25150, 25152, 25154-25160, 25163, 25165-25166, 25168,
25171-25176, 25180, 25182-25183, 25185-25186, 25188-25190, 25197,
25199, 25201-25202, 25205, 25208-25209, 25211-25225, 25228-25230,
25232-25233, 25238, 25240-25241, 25243, 25246, 25248-25249,
25251-25252, 25254, 25256, 25259, 25261-25265, 25267, 25269,
25272-25273, 25275, 25278, 25282-25285, 25288, 25290-25291,
25293-25296, 25298-25302, 25304, 25306-25308, 25310-25312,
25314, 25316-25318, 25320, 25322-25324, 25326, 25330, 25335-25340,
25345, 25347-25348, 25350-25352, 25354-25358, 25360-25364,
25367-25370, 25372-25373, 25376, 25378, 25382-25384, 25386-25388,
25391-25392, 25398-25404, 25407-25408, 25410-25415, 25418-25422,
25425-25426, 25428-25429, 25434-25435, 25437, 25440-25442, 25445,
25447-25448, 25450, 25452, 25455, 25457-25459, 25461, 25463-25464,
25469-25470, 25473-25478, 25480-25481, 25483-25486, 25488,
25492-25493, 25495, 25497, 25501, 25503, 25505-25507, 25509-25511,
25514-25519, 25521-25523, 25525, 25528-25532, 25535-25538, 25540,
25543-25547, 25552-25556, 25558, 25561, 25565-25567, 25569-25570,
25572-25579, 25582-25585, 25588-25601, 25603, 25605, 25609-25612,
25615-25619, 25621-25624, 25626-25632, 25634-25635, 25637-25640,
25645-25647, 25649, 25651-25652, 25656-25657, 25663, 25666-25668,
25670-25673, 25675-25678, 25680, 25682-25684, 25686, 25688, 25691,
25695, 25698, 25701-25702, 25704-25705, 25707, 25709-25711, 25713,
25717-25723, 25725, 25727, 25730, 25732-25733, 25735, 25737,
25739-25740, 25742-25744, 25752-25758, 25761-25762, 25765-25766,
25769-25770, 25773-25774, 25776-25782, 25785-25787, 25789, 25791,
25793-25794, 25796, 25798-25799, 25801-25802, 25807, 25809-25817,
25819, 25822, 25828, 25837-25838, 25840-25842, 25845, 25847-25848,
25850-25860, 25862, 25864-25866, 25869-25870, 25873-25878,
25880-25882, 25887, 25889-25891, 25893-25894, 25896-25897,
25899-25901, 25903-25904, 25906, 25908, 25912, 25915-25916,
25918-25921, 25924, 25926-25929, 25931, 25936-25938, 25941, 25943,
25949-25951, 25953-25961, 25963-25964, 25966, 25968, 25977,
25979-25984, 25989-25992, 25995, 25997, 25999, 26002-26008, 26011,
26013, 26017-26024, 26026-26028, 26031-26032, 26034, 26039, 26047,
26051-26055, 26058, 26060, 26063-26065, 26068, 26070-26073,
26076-26080, 26082, 26084-26086, 26088-26093, 26095-26096,
26098-26099, 26101, 26103-26107, 26111, 26114, 26116, 26118-26119,
26122-26126, 26133, 26136, 26138, 26142-26144, 26146-26149,
26152-26155, 26157, 26167-26171, 26173, 26175-26177, 26179, 26181,
26183-26186, 26188, 26190-26191, 26193-26194, 26196-26197,
26201-26204, 26207, 26210, 26213-26214, 26217, 26219-26223, 26225,
26229, 26234-26236, 26239, 26241-26242, 26253-26257, 26260,
26262-26263, 26267-26271, 26273-26274, 26278-26279, 26281,
6285-26287, 26289-26296, 26302-26304, 26306, 26310-26311,
26314-26318, 26320-26322, 26324, 26326, 26328, 26336, 26339, 26341,
26343-26346, 26348-26353, 26355-26356, 26358-26360, 26362, 26368,
26370, 26372, 26374-26375, 26377-26385, 26388, 26392-26393, 26396,
26399, 26401, 26403, 26405-26406, 26408-26413, 26419, 26422-26424,
26426-26427, 26430-26431, 26435-26439, 26441, 26446-26450, 26452,
26455, 26458-26460, 26463-26466, 26468-26470, 26472-26473,
6475-26476, 26478, 26483-26486, 26488, 26490, 26493-26494,
26496-26502, 26506-26510, 26512-26515, 26517, 26519, 26522-26523,
26525-26527, 26529-26531, 26533, 26535, 26537-26538, 26541,
26545-26546, 26548-26551, 26554-26555, 26559-26560, 26562-26564,
26568-26569, 26572-26575, 26577-26581, 26589-26595, 26598-26609,
26611-26614, 26616-26618, 26623-26624, 26627, 26631-26636,
26639-26641, 26643-26650, 26653-26657, 26659-26660, 26662-26665,
26667-26669, 26671, 26673-26678, 26684-26685, 26687, 26690,
26694-26719, 26721-26722, 26724-26731, 26733, 26735-26746,
26748, 26750-26766, 26768, 26770, 26772-26773, 26775, 26778,
26780-26781, 26788-26792, 26797-26799, 26801, 26808-26810,
26813-26814, 26817-26819, 26821, 26824-26825, 26828, 26830-26831,
26836-26837, 26839-26840, 26847-26851, 26853-26857, 26861-26865,
26868-26869, 26880, 26882, 26887, 26889-26891, 26893, 26895-26897,
26901, 26906-26912, 26914-26916, 26918, 26921-26929, 26931,
26933-26935, 26937, 26939-26943, 26946-26947, 26949-26956,
26960-26961, 26964, 26966-26968, 26970-26972, 26975, 26977-26978,
26980, 26982, 26985, 26988, 26994, 27000, 27002-27005, 27008-27012,
27016-27017, 27026-27027, 27029, 27032-27034, 27036-27037, 27044,
27048-27049, 27051-27053, 27055, 27057-27063, 27066-27069, 27071,
27074, 27076, 27090, 27093-27098, 27101, 27103-27105, 27110,
27112-27113, 27115-27116, 27118, 27130-27135, 27137-27138,
27140-27142, 27144-27146, 27148-27154, 27160, 27162, 27164-27165,
27167-27174, 27178-27179, 27181-27182, 27186, 27188-27189,
27192-27195, 27197, 27199-27200, 27203-27204, 27207, 27209, 27212,
27214, 27216, 27220, 27224-27227, 27229-27230, 27232, 27234,
27236-27238, 27240-27243, 27245, 27247, 27253-27254, 27256-27257,
27259, 27262-27264, 27267-27268, 27272, 27277, 27279, 27283,
27285-27289, 27294-27297, 27299-27301, 27303, 27305-27306,
27309-27319, 27322-27323, 27325-27326, 27328-27333, 27335-27337,
27339-27340, 27342-27344, 27346-27351, 27353-27356, 27360-27361,
27364-27366, 27369-27370, 27372, 27374-27375, 27379-27383,
27386-27389, 27391, 27393-27397, 27399-27400, 27402, 27404, 27407,
27410-27412, 27414, 27417, 27420-27424, 27430-27441, 27443, 27445,
27448-27449, 27452-27459, 27462-27463, 27467-27469, 27473-27474,
27476-27477, 27482, 27488-27489, 27492-27496, 27498, 27500,
27502-27506, 27508, 27510, 27513-27514, 27518-27519, 27522-27523,
27525-27526, 27528-27529, 27531-27532, 27534-27535, 27542, 27548,
27555, 27557-27559, 27562-27566, 27569-27572, 27574-27575, 27577,
27579, 27587-27588, 27590-27595, 27597-27599, 27603-27605,
27607-27610, 27616, 27618-27620, 27622-27624, 27626-27628, 27630,
27632, 27635-27638, 27640, 27645, 27652, 27654-27659, 27668-27669,
27672-27675, 27677-27680, 27687, 27689, 27691-27692, 27694,
27697-27698, 27700-27703, 27706, 27709-27710, 27712, 27715,
27719-27723, 27727-27730, 27732-27735, 27738, 27740-27741,
27743-27751, 27753-27756, 27758-27759, 27761-27763, 27765-27766,
27769, 27771, 27773, 27775, 27777, 27779, 27783-27788, 27795-27796,
27798, 27804, 27809-27810, 27812-27813, 27815-27818, 27821, 27823,
27827-27830, 27832-27834, 27836, 27838-27840, 27843, 27847, 27849,
27851, 27853, 27858-27861, 27863, 27866-27867, 27870, 27872,
27875-27877, 27880, 27882-27885, 27887, 27889-27892, 27894-27896,
27898, 27900-27905, 27910, 27912, 27916, 27919, 27922, 27924,
27926-27927, 27932, 27934-27938, 27940-27946, 27948, 27950, 27952,
27954-27956, 27958-27959, 27967-27969, 27971-27976, 27978-27984,
27992-27993, 27995, 27998, 28001, 28003-28006, 28010, 28012, 28017,
28021, 28023-28024, 28027-28028, 28033, 28035, 28038, 28040, 28044,
28054-28056, 28058-28062, 28064-28066, 28068, 28070, 28072-28073,
28075-28077, 28081-28083, 28087-28091, 28096-28098, 28100, 28102,
28104-28106, 28109-28115, 28117, 28120-28121, 28125-28128,
28130-28133, 28135, 28138, 28140, 28144-28149, 28151, 28153-28154,
28161-28173, 28176-28179, 28184-28196, 28201-28202, 28205-28206,
28209-28212, 28214-28223, 28227, 28229-28230, 28233-28238, 28240,
28242, 28247-28248, 28250-28252, 28254-28261, 28264, 28266, 28269,
28271-28278, 28283, 28288, 28290, 28292, 28296-28298, 28304-28308,
28310-28316, 28318, 28320-28321, 28323-28327, 28330, 28332, 28334,
28338, 28342, 28346-28349, 28351-28356, 28358-28374, 28376, 28379,
28381-28384, 28387-28392, 28395-28401, 28404-28408, 28412-28414,
28416-28419, 28423-28433, 28437-28443, 28445-28457, 28461, 28463,
28465-28467, 28469-28473, 28475-28483, 28486, 28488-28491, 28493,
28495-28496, 28498-28500, 28502-28509, 28512-28516, 28518,
28520-28522, 28524-28526, 28529-28530, 28532, 28534, 28538-28542,
28544-28552, 28554, 28556, 28558-28559, 28561, 28563-28564,
28566-28568, 28574-28576, 28578, 28580-28584, 28586-28588, 28590,
28592-28593, 28595-28597, 28599, 28601-28603, 28606, 28608, 28611,
28613-28614, 28616, 28620-28621, 28623-28625, 28627-28630, 28634,
28640, 28642-28647, 28651, 28654-28655, 28657, 28666-28667,
28672-28674, 28680, 28682-28684, 28686, 28688, 28692-28693, 28695,
28698-28699, 28701, 28703, 28709-28711, 28714, 28717-28725, 28727,
28730, 28734-28739, 28741-28744, 28746-28750, 28752, 28754,
28756-28757, 28760-28767, 28771-28772, 28774, 28776, 28778-28780,
28782-28783, 28786-28788, 28791, 28793-28797, 28802-28805, 28809,
28812, 28817, 28819-28821, 28823-28824, 28826, 28829-28841, 28843,
28845-28846, 28851-28853, 28856-28864, 28866-28871, 28874,
28877-28878, 28880-28886, 28888, 28890, 28894, 28896, 28898-28909,
28911, 28913-28915, 28917, 28919-28920, 28922-28923, 28925-28926,
28928, 28930-28931, 28934-28935, 28938, 28941-28944, 28947-28949,
28951-28956, 28958, 28962-28966, 28969-28976, 28978-28981, 28983,
28986-28989, 28992, 28997-28998, 29000, 29002-29003, 29005, 29009,
29011, 29019-29021, 29023, 29025-29026, 29028-29029, 29031-29032,
29034-29036, 29039, 29041-29042, 29045, 29048-29050, 29053-29054,
29056-29062, 29065-29071, 29073-29076, 29079, 29081, 29083-29084,
29087, 29091-29097, 29099-29100, 29106, 29109, 29113, 29116-29118,
29120-29122, 29125-29126, 29129-29130, 29132-29133, 29135, 29137,
29139-29142, 29144-29147, 29149-29151, 29155, 29157-29160, 29162,
29164-29166, 29168, 29171, 29173-29175, 29177-29178, 29180-29186,
29189-29192, 29194-29195, 29197, 29200, 29202, 29206, 29208,
29210-29211, 29214-29218, 29224, 29226, 29229-29231, 29233-29236,
29238-29242, 29244, 29246-29248, 29250, 29252-29261, 29264-29277,
29280, 29282-29295, 29297, 29302-29305, 29307, 29309, 29312, 29314,
29316-29319, 29321-29323, 29325-29327, 29336-29337, 29339-29340,
29344-29349, 29352, 29354-29355, 29357, 29359-29360, 29362-29369,
29373-29378, 29380, 29383-29386, 29389-29391, 29396, 29399, 29401,
29408, 29424-29427, 29429, 29432-29433, 29435, 29437-29439,
29441-29442, 29444-29446, 29448-29456, 29463-29469, 29479, 29481,
29483, 29485-29491, 29494-29498, 29501, 29503-29504, 29508-29509,
29511-29514, 29516-29517, 29519, 29521-29523, 29533, 29535-29536,
29538-29543, 29546, 29549, 29551-29554, 29557-29563, 29565-29566,
29568-29570, 29572, 29575-29578, 29581, 29583-29588, 29593-29608,
29612-29624, 29626, 29628-29629, 29631-29634, 29636-29639, 29642,
29644-29645, 29647, 29650, 29652-29658, 29660-29661, 29663-29664,
29666, 29672, 29674-29676, 29678-29680, 29682, 29685, 29687-29691,
29694-29704, 29707-29716, 29718, 29720, 29722, 29724-29727,
29730-29732, 29735-29736, 29739, 29741, 29745, 29747-29748,
29750-29767, 29772-29776, 29778-29781, 29784-29785, 29787-29788,
29790, 29792, 29794-29809, 29811-29812, 29814-29819, 29823,
29827-29828, 29831-29832, 29837, 29840, 29845-29848, 29851,
29854-29857, 29862, 29864-29866, 29872-29873, 29875, 29877-29884,
29886-29893, 29895-29896, 29898, 29900, 29902, 29904, 29906, 29908,
29911-29912, 29914-29919, 29921-29922, 29924, 29927, 29929,
29931-29934, 29936-29939, 29941-29945, 29947-29949, 29951-29956,
29958-29963, 29965-29966, 29968-29969, 29972, 29974-29976,
29978-29983, 29986-29988, 29990-29997, 29999-30000, 30002-30005,
30007-30009, 30011-30013, 30015, 30018, 30020-30021, 30023-30024,
30026-30027, 30029-30030, 30032, 30034-30035, 30037-30042,
30045-30051, 30053, 30055-30058, 30060, 30062-30066, 30069-30071,
30073-30077, 30079-30087, 30089-30090, 30092, 30094-30095,
30097-30098, 30101-30105, 30107-30109, 30111-30117, 30119,
30121-30122, 30126, 30128, 30130, 30132-30138, 30142-30152,
30155-30161, 30163-30167, 30169-30170, 30174, 30176-30179,
30181-30191, 30195-30196, 30198, 30200-30201, 30203, 30205-30208,
30211-30218, 30222-30225, 30228-30230, 30233-30237, 30239-30244,
30246, 30248-30255, 30257-30258, 30260-30263, 30265, 30268-30277,
30279-30280, 30283-30287, 30289, 30291, 30293-30294, 30296,
30298-30300, 30302-30304, 30306-30314, 30316-30317, 30319-30321,
30325, 30327, 30331, 30334, 30336, 30338, 30341-30347, 30349-30353,
30355, 30358, 30360-30361, 30363-30365, 30368, 30370-30371,
30373-30376, 30378-30387, 30389-30393, 30395-30397, 30400,
30404-30409, 30411-30415, 30417-30422, 30424-30425, 30428-30430,
30432-30433, 30436, 30439-30440, 30442-30443, 30449, 30451-30454,
30456-30457, 30459, 30461-30462, 30465-30466, 30469, 30473-30476,
30478, 30481, 30483-30495, 30499-30502, 30504-30513, 30520-30526,
30528, 30530, 30533-30535, 30538-30539, 30541-30545, 30547, 30549,
30551-30552, 30554-30557, 30561-30563, 30565-30568, 30572-30574,
30576, 30578, 30580, 30582-30583, 30585, 30587, 30590-30598,
30601-30605, 30609-30615, 30617, 30621-30624, 30626-30627, 30629,
30632-30633, 30639-30641, 30643, 30646, 30648, 30650-30654,
30656-30659, 30667-30669, 30671-30672, 30676-30678, 30680-30683,
30685-30688, 30691-30692, 30694, 30696-30698, 30702, 30705-30709,
30711-30712, 30714-30718, 30720, 30723-30727, 30730-30732, 30737,
30740, 30742, 30749, 30751, 30753, 30755-30757, 30761-30767,
30773-30775, 30777-30781, 30783-30794, 30796, 30801-30803, 30805,
30807-30809, 30811, 30813, 30815, 30818-30823, 30825-30827,
30829-30834, 30838-30842, 30845, 30848, 30850-30853, 30855-30856,
30858, 30860-30862, 30864, 30866-30869, 30872-30873, 30877-30878,
30883-30884, 30887-30891, 30894-30898, 30901-30907, 30909,
30911-30912, 30914, 30916, 30919, 30926-30927, 30930-30931,
30934-30938, 30942-30945, 30947-30948, 30954-30958, 30961-30962,
30964-30966, 30968, 30970, 30972-30973, 30975-30979, 30981,
30983-30985, 30987-30989, 30992-30996, 30999-31000, 31002-31006,
31009, 31011-31012, 31017, 31019-31020, 31023-31024, 31027,
31030-31036, 31039-31042, 31044-31045, 31048-31049, 31055, 31057,
31059-31066, 31068, 31071-31072, 31074, 31077, 31082, 31084, 31086,
31089-31095, 31097, 31099-31102, 31104, 31107-31108, 31111-31116,
31119-31120, 31123, 31127, 31129, 31131-31132, 31136-31137, 31140,
31142, 31145-31151, 31153-31154, 31156-31159, 31161, 31163-31164,
31167-31171, 31174-31176, 31181-31182, 31184-31193, 31195-31196,
31200, 31202, 31205, 31209-31213, 31216-31218, 31220-31221, 31223,
31225, 31228-31229, 31231-31234, 31238-31239, 31242-31243, 31250,
31253, 31257-31264, 31266-31269, 31271, 31274, 31277-31278,
31283-31301, 31305-31311, 31314, 31326, 31328-31329, 31331-31333,
31335-31339, 31343-31344, 31346, 31348-31349, 31356-31359, 31362,
31364, 31366-31371, 31373, 31376-31378, 31380-31382, 31385,
31392-31393, 31395-31401, 31403, 31405-31410, 31413, 31415-31417,
31419, 31421-31427, 31429-31432, 31435, 31437, 31440, 31444-31447,
31456, 31460-31462, 31464, 31466-31469, 31471, 31473-31477, 31479,
31485-31486, 31488, 31490-31494, 31496-31499, 31503-31504,
31506-31508, 31519, 31522-31523, 31530-31533, 31535-31536,
31539-31540, 31542, 31547-31548, 31552, 31556-31559, 31561,
31565-31574, 31576-31578, 31580-31581, 31583, 31586-31590,
31592-31598, 31601, 31608, 31611-31617, 31621, 31623-31624, 31626,
31632-31635, 31638, 31641-31642, 31644, 31646-31648, 31651-31659,
31665, 31667-31669, 31671-31672, 31674-31676, 31678-31687, 31690,
31696, 31698-31701, 31703, 31705, 31707, 31709-31711, 31715-31716,
31718, 31721-31722, 31724-31726, 31728, 31730, 31733, 31735-31736,
31739, 31741-31746, 31749-31750, 31752-31755, 31757-31758, 31762,
31764, 31766-31768, 31771-31773, 31777-31779, 31781, 31784-31786,
31791, 31794-31802, 31804, 31806, 31809, 31811, 31814-31816,
31820-31825, 31827-31832, 31836-31839, 31841-31844, 31848-31849,
31851, 31854-31860, 31863, 31865, 31868, 31870-31873, 31876,
31882-31884, 31888-31889, 31893, 31900, 31902-31906, 31908, 31910,
31912-31917, 31919-31920, 31923, 31930-31932, 31935, 31940, 31942,
31947-31949, 31959, 31962-31967, 31969, 31973, 31975, 31977-31979,
31981, 31985-31986, 31992-31995, 31997-31998, 32003-32004,
32007-32008, 32010-32011, 32013-32016, 32021-32022, 32025-32030,
32032, 32034-32037, 32040-32042, 32047-32049, 32051-32052,
32055-32056, 32058-32059, 32062-32067, 32071-32073, 32075,
32079-32084, 32086-32088, 32090-32091, 32093, 32095-32099, 32102,
32104-32107, 32109-32115, 32117-32118, 32120-32121, 32123-32130,
32132, 32134-32136, 32138-32139, 32142, 32144-32149, 32154-32158,
32160, 32162, 32165-32171, 32173-32176, 32178, 32180-32183,
32186-32187, 32189, 32192-32193, 32196-32201, 32203-32204, 32210,
32215-32217, 32219-32221, 32223, 32225, 32227-32228, 32233,
32237-32240, 32242, 32244, 32246-32248, 32250-32251, 32253-32257,
32260, 32263, 32265-32271, 32273, 32275, 32277-32279, 32284-32285,
32287-32289, 32291-32292, 32297-32298, 32301, 32303, 32305,
32308-32315, 32317-32320, 32322-32323, 32329-32330, 32332,
32334-32340, 32343, 32352-32354, 32359-32360, 32366-32368, 32373,
32376-32377, 32381-32382, 32384-32392, 32394, 32396, 32405,
32407-32409, 32415, 32418, 32423-32424, 32427, 32429, 32431-32441,
32444, 32446-32449, 32451-32452, 32454-32459, 32461-32465,
32467-32468, 32470-32473, 32478-32479, 32481, 32484-32485,
32489-32490, 32492-32497, 32503, 32505, 32507, 32511-32512,
32517-32518, 32521-32523, 32526-32533, 32535-32538, 32540-32541,
32543-32550, 32553, 32555-32559, 32561-32566, 32568-32571,
32573-32576, 32584-32586, 32589-32590, 32592-32596, 32598, 32600,
32602-32605, 32607-32609, 32611-32612, 32616, 32626-32630, 32632,
32634-32637, 32640-32642, 32646-32647, 32649-32653, 32655-32660,
32663-32665, 32668, 32670, 32672, 32675-32676, 32681-32684, 32690,
32694-32711, 32717-32719, 32723-32725, 32728-32729, 32731,
32733-32738, 32742, 32744-32746, 32748-32754, 32757, 32765, 32767,
32769, 32775-32778, 32780-32782, 32784-32788, 32790-32791, 32795,
32797, 32800-32801, 32804-32805, 32808, 32813-32815, 32820-32821,
32827-32836, 32838, 32840-32846, 32848, 32850, 32852-32859,
32862-32864, 32868-32875, 32877, 32880, 32882-32883, 32885,
32887-32890, 32895-32900, 32903-32904, 32906-32908, 32910-32916,
32919, 32925, 32928-32929, 32931, 32936-32938, 32941-32946,
32949-32950, 32954, 32956, 32963, 32965, 32967, 32970, 32972-32973,
32976, 32981, 32984-32988, 32991, 32994-32995, 32998, 33000-33001,
33003, 33010, 33012-33013, 33015-33016, 33018-33019, 33022-33023,
33025, 33028-33032, 33035-33050, 33052-33057, 33059, 33065-33066,
33068-33076, 33080, 33082-33086, 33088, 33090, 33092-33096,
33099-33101, 33104-33106, 33108, 33114, 33118, 33120-33121, 33124,
33132-33133, 33135-33139, 33142-33157, 33159, 33161-33167,
33169-33174, 33176-33178, 33180, 33182, 33184-33187, 33191, 33195,
33198-33201, 33204-33209, 33211, 33213, 33217, 33219-33221,
33225-33229, 33231-33232, 33235-33238, 33243, 33245-33249, 33254,
33256-33261, 33264, 33271-33272, 33274, 33279-33280, 33282-33283,
33285-33286, 33293-33294, 33296-33298, 33300-33303, 33305-33307,
33309-33310, 33312-33313, 33315, 33317, 33319-33325, 33328-33329,
33332, 33335-33338, 33344-33346, 33348, 33350-33356, 33362-33364,
33366, 33368, 33370, 33373-33377, 33380, 33383, 33389, 33392, 33394,
33396, 33398, 33400, 33403, 33405-33406, 33408, 33410-33415, 33418,
33420-33421, 33423-33427, 33429-33431, 33434, 33436-33439, 33441,
33444, 33446-33448, 33450-33451, 33453, 33455, 33458, 33461, 33463,
33465, 33467, 33469-33472, 33474, 33478-33480, 33482-33484,
33486-33487, 33489, 33491-33493, 33496-33499, 33501-33503, 33505,
33512, 33514-33515, 33520, 33522-33523, 33528, 33531, 33534, 33536,
33538, 33542-33543, 33546, 33548-33549, 33551, 33553, 33555-33556,
33558-33559, 33561, 33564, 33566-33572, 33574-33576, 33580-33584,
33586, 33593, 33595-33597, 33602, 33610-33611, 33613-33614, 33622,
33627, 33630, 33633-33634, 33636, 33638-33641, 33643-33644, 33646,
33648, 33654, 33660, 33666-33667, 33669-33672, 33674-33675,
33680-33682, 33684-33688, 33690-33693, 33697-33699, 33703-33704,
33706, 33709, 33715, 33718, 33720, 33723-33726, 33728, 33733, 33735,
33738-33743, 33745, 33747-33748, 33753-33756, 33762-33765,
33767-33770, 33774-33776, 33782, 33784-33785, 33793, 33798,
33801-33802, 33804, 33806-33808, 33814, 33816-33817, 33819-33823,
33825-33826, 33828-33834, 33836-33837, 33839, 33842-33843,
33845, 33847-33850, 33852, 33854-33855, 33858-33860, 33862,
33865-33866, 33869, 33872, 33875-33879, 33881-33882, 33884,
33890-33894, 33896, 33898, 33901-33902, 33908-33911, 33914-33917,
33919-33923, 33925, 33927-33928, 33931, 33933, 33935-33942, 33944,
33946-33947, 33949-33950, 33954, 33957-33961, 33964, 33966-33967,
33969, 33971, 33973-33977, 33979, 33981-33988, 33990-33991,
33994-33995, 33997-33998, 34000, 34002-34017, 34019-34021,
34023-34027, 34029-34032, 34034, 34036-34048, 34050-34051,
34053-34055, 34057, 34063, 34066-34074, 34076, 34079, 34081-34083,
34085-34091, 34093-34094, 34097-34098, 34100-34105, 34107,
34109-34117, 34120-34121, 34124, 34126-34129, 34131, 34135-34136,
34138, 34142-34145, 34149-34153, 34155-34157, 34159-34166, 34168,
34170-34172, 34174-34178, 34184, 34187-34188, 34191-34192, 34195,
34197, 34199-34201, 34204, 34212, 34214, 34216-34217, 34219,
34221-34222, 34230, 34232-34236, 34238-34239, 34241-34243,
34245-34247, 34249-34250, 34252-34253, 34258-34259, 34263, 34267,
34270, 34273-34284, 34286-34288, 34290-34291, 34293, 34295,
34297-34298, 34301-34302, 34306, 34310, 34312-34313, 34316-34321,
34324, 34327-34328, 34330-34335, 34338, 34341-34347, 34350,
34352-34353, 34355-34356, 34358-34365, 34370, 34374-34377, 34384,
34389-34390, 34392, 34395-34396, 34399, 34401-34403, 34405-34406,
34408-34414, 34416, 34419, 34421, 34423, 34431-34435, 34437,
34439-34440, 34443, 34445, 34447, 34453, 34456-34458, 34460-34461,
34463, 34471, 34474, 34477-34481, 34483, 34486, 34488, 34490-34492,
34495, 34497, 34499-34500, 34505-34509, 34511-34515, 34521, 34523,
34527, 34530, 34533, 34535, 34537-34543, 34545-34546, 34551-34554,
34556, 34558, 34560, 34563-34564, 34566-34568, 34570, 34575-34576,
34578, 34580-34583, 34587, 34589, 34591, 34593-34594, 34596-34603,
34605-34607, 34609-34612, 34618-34627, 34631, 34634, 34636-34641,
34643-34644, 34648, 34650, 34652-34655, 34657, 34659-34661,
34663-34664, 34666-34670, 34673-34675, 34677-34685, 34687-34692,
34696-34697, 34699-34700, 34702-34704, 34706-34712, 34724-34725,
34727-34729, 34731-34745, 34750, 34754, 34756-34758, 34762, 34766,
34769, 34771-34772, 34775-34776, 34782-34783, 34787-34790, 34792,
34799-34806, 34809, 34811, 34813, 34818, 34821-34823, 34825,
34827-34831, 34833-34835, 34837, 34840, 34845, 34848, 34850, 34852,
34855, 34858, 34863-34866, 34868, 34870, 34874-34881, 34883-34887,
34889, 34893, 34897-34899, 34901-34902, 34904-34905, 34908,
34911-34913, 34915, 34917-34926, 34928-34930, 34933-34936, 34940,
34945, 34948, 34959, 34962, 34965, 34971, 34976-34977, 34982-34986,
34992-34994, 34999-35011, 35014, 35017-35021, 35024, 35026-35027,
35029, 35045-35048, 35052, 35054, 35058-35061, 35063-35065, 35068,
35072, 35074-35075, 35077-35079, 35081, 35083-35086, 35088-35089,
35091, 35094, 35098, 35106-35110, 35113-35114, 35118, 35120,
35129-35130, 35132, 35134, 35136, 35138-35139, 35143, 35145-35146,
35148-35156, 35158-35162, 35168-35170, 35172, 35174, 35176,
35185-35190, 35192, 35194-35195, 35199-35202, 35204-35207,
35209-35211, 35213-35215, 35218-35219, 35221-35225, 35227-35228,
35232, 35234-35236, 35238, 35242-35243, 35248-35249, 35252,
35259-35262, 35265, 35267-35268, 35270-35272, 35276, 35279, 35282,
35285-35286, 35288-35289, 35291, 35293-35312, 35314-35317,
35320-35328, 35330, 35334-35335, 35337, 35339-35341, 35343,
35345-35347, 35349-35361, 35369-35371, 35375-35384, 35392,
35394-35396, 35404, 35406-35411, 35413-35418, 35420-35422, 35425,
35427, 35434, 35436-35438, 35441-35443, 35447, 35450-35452, 35454,
35456, 35458-35463, 35467-35469, 35473, 35475-35482, 35484,
35486-35488, 35490-35497, 35499, 35501, 35503, 35505-35506, 35508,
35511, 35514-35516, 35522, 35527-35529, 35533-35535, 35537,
35542-35543, 35545-35546, 35548, 35550, 35554-35555, 35559-35564,
35566-35569, 35575-35576, 35578-35581, 35584-35587, 35589,
35592-35593, 35603, 35612, 35616, 35619-35621, 35623, 35629, 35632,
35634-35636, 35638-35639, 35641-35643, 35646-35647, 35649-35651,
35653-35655, 35657-35660, 35662, 35666-35667, 35669, 35671-35672,
35674-35677, 35679, 35681-35684, 35688-35692, 35694-35699, 35701,
35703, 35710-35712, 35717-35719, 35721-35722, 35724, 35728,
35731-35732, 35734-35737, 35739-35744, 35748-35751, 35755-35760,
35762-35769, 35772-35773, 35778-35780, 35785-35788, 35794-35796,
35798, 35802, 35804-35805, 35809, 35812, 35814-35815, 35817, 35819,
35821-35823, 35827-35828, 35831-35837, 35839-35841, 35843,
35845-35846, 35848-35850, 35853-35854, 35856-35857, 35859,
35861-35862, 35864-35866, 35870-35871, 35874, 35876-35878, 35884,
35886-35890, 35892, 35895, 35901-35904, 35906-35907, 35913,
35915-35919, 35922-35923, 35925-35932, 35934, 35936, 35938-35940,
35942, 35946-35947, 35949-35951, 35953, 35955, 35957-35958, 35960,
35963-35964, 35966-35968, 35970-35971, 35976-35980, 35982-35984,
35986-35988, 35991-35998, 36003, 36006, 36010, 36012-36013, 36016,
36018-36022, 36024, 36027-36029, 36031-36034, 36037-36041, 36045,
36047, 36050-36052, 36055-36058, 36060-36062, 36064, 36066, 36068,
36073, 36077-36080, 36082, 36089-36091, 36093, 36097-36098, 36102,
36105, 36109, 36115, 36118-36119, 36121, 36124-36130, 36132, 36136,
36139-36141, 36145, 36148, 36150-36151, 36153, 36157, 36159-36161,
36164, 36166, 36168, 36175-36176, 36178-36179, 36182, 36184,
36186-36189, 36191-36200, 36202-36209, 36214-36216, 36220-36226,
36228, 36230-36232, 36234-36236, 36240, 36242-36243, 36252, 36255,
36257, 36259, 36266-36273, 36277-36278, 36281-36283, 36285-36287,
36289, 36292, 36294-36295, 36299, 36301-36302, 36310, 36312-36313,
36315-36317, 36324-36326, 36333, 36339-36345, 36350, 36353-36357,
36359-36361, 36364, 36366-36368, 36370-36379, 36381-36382, 36386,
36388-36395, 36397, 36400-36401, 36403-36405, 36407, 36409, 36411,
36413-36414, 36416, 36419-36420, 36422, 36425, 36427, 36430-36433,
36437, 36439-36440, 36442, 36447-36449, 36452, 36454-36455,
36461-36472, 36474-36475, 36480, 36482-36483, 36485-36487, 36490,
36492, 36499, 36504, 36511-36512, 36514, 36516-36520, 36527-36529,
36531, 36536, 36538-36541, 36543-36549, 36552-36554, 36557-36561,
36564, 36566-36572, 36574-36575, 36577-36580, 36583-36587,
36589-36592, 36595-36596, 36598-36602, 36611, 36613, 36615,
36618-36619, 36624, 36626-36627, 36629-36631, 36633-36635, 36639,
36642, 36647-36648, 36650-36652, 36654, 36660-36668, 36670-36671,
36673, 36677, 36680-36685, 36687, 36689-36697, 36700, 36702,
36705-36707, 36709-36714, 36716, 36718, 36720-36727, 36729-36731,
36734-36738, 36740-36743, 36745-36746, 36748, 36750, 36755, 36757,
36761-36763, 36765-36766, 36769-36772, 36774, 36779-36784, 36787,
36789, 36792-36794, 36796, 36804, 36806-36807, 36810, 36812, 36816,
36818-36823, 36826, 36828, 36830-36831, 36837, 36843-36846, 36848,
36851, 36853, 36855, 36857-36862, 36865, 36867, 36869-36874,
36876-36879, 36881-36887, 36890-36891, 36893-36898, 36905,
36907-36908, 36910, 36915-36920, 36922-36924, 36927-36930, 36934,
36936-36937, 36939-36949, 36951, 36954-36956, 36958-36963, 36965,
36967-36977, 36980-36982, 36984-36991, 36995, 36997, 36999, 37001,
37003, 37006-37010, 37014-37015, 37017-37018, 37021-37022, 37025,
37028-37029, 37031-37034, 37040-37044, 37046-37047, 37049-37051,
37053-37054, 37056, 37061, 37064-37065, 37067, 37074-37075, 37077,
37079-37082, 37085-37091, 37093-37097, 37099-37112, 37114,
37116-37117, 37120, 37122-37123, 37132-37136, 37138-37141, 37145,
37147-37156, 37160-37163, 37169-37170, 37174, 37177, 37181,
37183-37184, 37187, 37189-37190, 37192-37194, 37196-37198,
37201-37203, 37206-37211, 37213-37216, 37218, 37228, 37230-37231,
37234, 37236, 37240-37244, 37246, 37250-37255, 37258, 37263, 37265,
37270-37273, 37275, 37278, 37280, 37285, 37294, 37298, 37300, 37303,
37305-37306, 37308-37310, 37312-37313, 37317, 37325-37326,
37328-37331, 37334, 37337, 37339-37340, 37342-37344, 37346-37348,
37350, 37355, 37358-37359, 37362-37363, 37365-37367, 37369-37370,
37373, 37377-37379, 37381, 37386, 37388, 37390, 37392-37393,
37396-37399, 37405, 37407-37408, 37412-37413, 37415, 37417,
37422, 37428, 37435-37441, 37443-37444, 37446, 37448, 37451,
37453-37457, 37459-37460, 37463-37464, 37466, 37468, 37473-37474,
37482-37485, 37488, 37494-37495, 37497, 37499, 37501-37503,
37505-37507, 37509-37510, 37512, 37515-37516, 37518-37519, 37521,
37524-37525, 37529-37531, 37535-37542, 37549-37553, 37555-37556,
37558, 37561-37563, 37570-37573, 37580-37582, 37585-37587, 37591,
37594-37595, 37599-37600, 37603-37606, 37608, 37610, 37617,
37619-37625, 37630-37631, 37633-37635, 37638, 37642-37643, 37650,
37653, 37658, 37661, 37665, 37667-37669, 37671-37672, 37674,
37678-37680, 37683, 37685-37686, 37688, 37691, 37693-37694, 37696,
37703, 37706-37707, 37709, 37717-37720, 37722, 37726, 37728, 37737,
37740, 37745-37748, 37751-37754, 37756-37761, 37763-37769, 37771,
37773, 37775, 37777-37779, 37785-37786, 37788, 37790-37792,
37794-37795, 37798-37799, 37802, 37806, 37808, 37825-37826, 37829,
37831, 37833-37834, 37836, 37843-37849, 37851-37852, 37855, 37857,
37859-37864, 37868-37870, 37872-37874, 37877, 37879, 37884-37886,
37888-37889, 37891-37893, 37895-37908, 37915-37923, 37925,
37927-37929, 37931-37932, 37934, 37936, 37938, 37940-37942,
37946-37948, 37950-37952, 37958-37965, 37967-37971, 37977-37980,
37982, 37988-37992, 37994, 37997, 38001-38013, 38015, 38017, 38019,
38021-38022, 38025, 38027-38034, 38036-38037, 38039-38047, 38052,
38055, 38057-38062, 38065-38067, 38069, 38071, 38074-38077,
38084-38089, 38091, 38095-38096, 38099, 38102-38103, 38107-38110,
38112, 38114, 38123, 38126, 38129, 38133-38134, 38137-38138,
38140, 38142-38144, 38153, 38156, 38158-38160, 38162-38163,
38167-38168, 38170, 38173-38174, 38176, 38179-38181, 38184, 38187,
38192-38194, 38196, 38198-38199, 38203, 38205, 38207-38210,
38212-38214, 38216, 38218-38219, 38221-38235, 38238, 38240-38242,
38246, 38249-38252, 38254-38256, 38258, 38260-38262, 38264,
38268-38269, 38271-38272, 38276, 38278-38280, 38282-38283, 38286,
38288, 38290, 38292, 38294-38295, 38297, 38301-38302, 38304-38307,
38310, 38313-38315, 38317, 38321-38322, 38324, 38334, 38336, 38340,
38342, 38345-38347, 38349-38350, 38357, 38361-38362, 38364-38365,
38369-38370, 38374-38375, 38377-38378, 38380, 38382-38384,
38386-38387, 38391, 38393-38398, 38400, 38403-38411, 38414-38421,
38423-38424, 38426-38431, 38433-38437, 38439-38442, 38445, 38448,
38454, 38459, 38464-38465, 38468-38471, 38474, 38477-38478,
38480-38484, 38487, 38490-38492, 38495-38503, 38505, 38507-38515,
38517-38518, 38521-38523, 38526-38531, 38534, 38537, 38540, 38543,
38547-38548, 38550-38552, 38554-38556, 38559-38562, 38565-38567,
38570-38571, 38574, 38579-38582, 38584-38585, 38590, 38592,
38594-38601, 38605-38609, 38612-38613, 38616, 38618, 38625-38634,
38636-38642, 38644, 38648, 38651-38658, 38660-38663, 38665-38668,
38670-38673, 38675, 38677, 38681, 38684, 38686-38687, 38689-38690,
38692, 38695, 38699-38700, 38702-38705, 38707-38709, 38713-38716,
38719-38727, 38729-38730, 38732-38734, 38736-38741, 38743, 38745,
38747, 38749, 38751-38752, 38754, 38759, 38761-38762, 38766-38767,
38769-38772, 38776-38777, 38779, 38786-38788, 38790-38791, 38794,
38798-38801, 38804-38812, 38814, 38816-38821, 38824, 38826-38827,
38832-38835, 38837, 38839-38852, 38855-38860, 38863, 38865,
38869-38874, 38879-38881, 38884-38890, 38892, 38894, 38897-38898,
38902-38904, 38906, 38909, 38912, 38915, 38917, 38919, 38921-38925,
38927, 38929-38932, 38940, 38944, 38946-38947, 38949-38950,
38954-38955, 38957-38960, 38962-38963, 38965, 38968, 38970,
38975-38976, 38983-38986, 38994-38995, 39000, 39002-39005,
39015, 39017-39021, 39023-39024, 39037, 39039-39042, 39044,
39046-39050, 39052, 39055-39056, 39059-39061, 39068, 39071-39072,
39074, 39078-39079, 39082, 39086, 39088-39090, 39092-39097,
39100-39102, 39106-39109, 39112-39114, 39116-39122, 39124,
39126-39128, 39132-39133, 39135-39138, 39140-39141, 39143-39145,
39147, 39150-39152, 39154-39158, 39160, 39162, 39164, 39166-39172,
39175-39178, 39180-39182, 39184-39185, 39187-39190, 39192-39193,
39195, 39199-39201, 39204-39208, 39210-39212, 39219, 39221-39222,
39224-39231, 39234-39235, 39237, 39244-39248, 39250-39252,
39257-39260, 39262-39266, 39268-39269, 39271, 39279, 39281-39283,
39285-39286, 39288, 39290-39292, 39297-39307, 39310-39311,
39314-39315, 39318-39319, 39322, 39324-39328, 39330-39334,
39336-39340, 39342-39343, 39345-39347, 39349-39352, 39354,
39357-39360, 39362-39365, 39368, 39370-39372, 39374-39384,
39386-39387, 39389, 39396, 39398-39403, 39405-39409, 39412,
39414-39424, 39427-39430, 39432, 39434-39435, 39438, 39440-39441,
39443-39446, 39448-39450, 39452-39453, 39455, 39457-39458, 39460,
39462-39463, 39465-39470, 39472, 39474-39492, 39494-39501,
39503-39504, 39506, 39508-39509, 39511-39515, 39517-39520, 39523,
39527-39528, 39530, 39532-39535, 39537-39539, 39541, 39543-39550,
39552-39560, 39562, 39564, 39566, 39569-39570, 39572-39573,
39575-39576, 39578, 39580-39581, 39583-39590, 39592-39593, 39596,
39599-39605, 39607, 39611, 39614-39615, 39618-39620, 39624,
39627-39631, 39634-39636, 39638, 39640, 39644-39647, 39649-39652,
39655, 39659, 39661, 39663-39664, 39666-39668, 39670-39671, 39673,
39676, 39680-39682, 39684-39688, 39693-39699, 39701-39706, 39708,
39710, 39715, 39717, 39719-39722, 39724-39727, 39729, 39732-39734,
39736-39738, 39740-39743, 39745-39746, 39748-39749, 39752-39753,
39755, 39757-39758, 39763-39764, 39769-39770, 39772-39786,
39788-39791, 39794, 39796, 39798-39803, 39805-39815, 39822,
39824-39825, 39829, 39831-39832, 39839-39840, 39844-39846, 39849,
39851-39854, 39856-39859, 39861-39863, 39865-39866, 39868-39870,
39873-39874, 39876, 39880, 39882, 39886-39896, 39898-39899, 39901,
39903-39908, 39910, 39912-39913, 39916, 39918, 39922, 39924,
39926-39931, 39934-39937, 39944-39949, 39952, 39954-39957,
39963-39968, 39971-39973, 39978, 39980-39981, 39984-39987, 39989,
39992-39995, 40002-40008, 40010-40015, 40020-40028, 40031-40034,
40037-40039, 40041-40049, 40051-40052, 40054-40055, 40057-40061,
40066-40069, 40074-40078, 40080-40082, 40085, 40087, 40089-40091,
40094-40095, 40097, 40099-40100, 40102-40103, 40105, 40108-40117,
40119-40121, 40124-40132, 40134-40136, 40138-40139, 40141-40147,
40150, 40153-40162, 40164-40166, 40168-40170, 40173-40177,
40179-40200, 40202, 40204-40207, 40209-40215, 40218-40220,
40222-40224, 40227, 40229-40234, 40237, 40239, 40241, 40243-40247,
40250-40251, 40254-40255, 40257-40260, 40263-40264, 40266-40267,
40269-40272, 40274-40279, 40283, 40286-40289, 40291-40297,
40299-40303, 40309-40310, 40313-40314, 40317-40322, 40325-40326,
40328, 40330-40336, 40338, 40343-40344, 40346, 40349, 40353-40355,
40358, 40362-40364, 40366-40371, 40373-40374, 40376-40379,
40381-40388, 40391-40392, 40395-40397, 40399-40405, 40407-40409,
40411, 40416-40417, 40419-40422, 40424, 40426, 40428-40429,
40433-40434, 40443, 40445-40446, 40448, 40450-40452, 40456,
40458-40460, 40462, 40464-40465, 40468-40469, 40474-40476,
40478-40480, 40487, 40489-40492, 40494-40496, 40498, 40501-40502,
40504, 40507, 40510, 40513-40514, 40517, 40523-40524, 40526, 40530,
40532-40535, 40543-40545, 40547, 40550, 40553, 40555, 40557-40562,
40564-40567, 40570, 40572, 40575, 40577-40578, 40580, 40582, 40584,
40586-40592, 40594, 40596, 40599-40600, 40605-40608, 40610-40611,
40613, 40616, 40618-40621, 40623-40624, 40627-40631, 40633-40636,
40638, 40641, 40646, 40648, 40650-40651, 40653, 40658-40660, 40662,
40664-40665, 40667, 40670, 40673-40678, 40688-40689, 40695-40697,
40699, 40703, 40705, 40707, 40709, 40712-40714, 40716-40718,
40720-40723, 40726-40727, 40729, 40731-40732, 40734, 40738-40739,
40745, 40748-40753, 40755, 40757-40760, 40766, 40770, 40773-40778,
40781-40782, 40784-40785, 40787, 40789, 40797, 40799-40806, 40808,
40811-40813, 40816, 40818, 40826, 40829, 40831, 40833-40834, 40844,
40848-40850, 40861, 40864-40870, 40872, 40875-40880, 40882, 40885,
40888-40890, 40895, 40899-40900, 40912, 40916, 40918, 40921-40926,
40929, 40931, 40934-40936, 40938-40939, 40941-40943, 40945-40948,
40951-40953, 40955, 40957-40960, 40962-40964, 40966, 40969, 40974,
40976, 40979, 40981-40982, 40985, 40987-40988, 40993, 40995-40997,
41004, 41006-41008, 41010-41011, 41013-41015, 41017, 41021-41022,
41024, 41026-41033, 41037-41038, 41041, 41043-41048, 41051, 41057,
41060-41062, 41064, 41067-41071, 41074, 41079-41084, 41087,
41089-41095, 41098-41101, 41103-41105, 41115, 41119-41120,
41122-41125, 41127, 41130, 41133, 41136-41140, 41142-41144,
41146-41147, 41149-41154, 41156-41157, 41159, 41161-41162, 41165,
41167-41170, 41172, 41174-41178, 41181-41183, 41188-41191,
41194-41199, 41203-41206, 41209-41210, 41212, 41217, 41219, 41221,
41223-41225, 41227, 41229-41230, 41233, 41235, 41237, 41240-41246,
41251, 41253, 41255-41256, 41258-41261, 41264-41269, 41274-41275,
41277-41284, 41288-41292, 41299-41304, 41307-41309, 41311-41313,
41316-41322, 41326, 41328-41332, 41334, 41337, 41339-41340,
41342-41348, 41350-41354, 41357, 41359-41363, 41365-41366,
41368-41369, 41373-41375, 41378-41393, 41396, 41398, 41400,
41402-41403, 41405, 41407-41414, 41416-41418, 41420, 41422-41423,
41425-41427, 41430-41438, 41440-41444, 41446-41449, 41453,
41455-41456, 41459-41461, 41463, 41466, 41468-41469, 41471,
41474-41475, 41482, 41485, 41487-41490, 41493-41495, 41497,
41499-41504, 41507, 41510-41511, 41514-41522, 41525-41526,
41528-41529, 41531, 41536-41537, 41540, 41542-41543, 41545, 41547,
41549-41552, 41558-41559, 41564-41565, 41567-41568, 41570-41571,
41573-41578, 41580-41582, 41594-41595, 41597-41601, 41604-41607,
41609, 41613-41617, 41621, 41624-41627, 41629-41631, 41634,
41636-41641, 41643-41644, 41646-41649, 41651, 41655-41658, 41660,
41662-41664, 41667-41668, 41670-41671, 41673, 41678-41689,
41691-41694, 41696-41703, 41707, 41709, 41713-41714, 41718,
41720-41726, 41729-41733, 41743-41744, 41749, 41751-41753, 41755,
41759, 41762-41767, 41772, 41778-41780, 41782, 41785-41790,
41796-41799, 41801, 41804, 41807, 41809, 41811, 41814, 41822,
41824-41825, 41827-41831, 41833-41837, 41839-41841, 41843-41845,
41847, 41851-41855, 41857, 41859, 41861-41862, 41864, 41866-41867,
41873, 41876-41879, 41886, 41890-41891, 41895, 41897, 41899-41901,
41903, 41906-41910, 41912-41913, 41915, 41920-41926, 41931,
41934-41936, 41939-41944, 41946-41947, 41949-41950, 41952-41953,
41955-41958, 41962, 41964-41968, 41971-41974, 41977-41980,
41982-41986, 41989-41991, 41993-41996, 42001-42002, 42005-42007,
42009-42010, 42013, 42017, 42022, 42027-42031, 42033-42034, 42038,
42046, 42048, 42050, 42054-42059, 42062-42063, 42065-42067,
42074-42077, 42079-42083, 42085, 42091-42095, 42097-42099, 42102,
42105-42110, 42112, 42114, 42117-42118, 42122-42128, 42134, 42137,
42143-42145, 42147-42153, 42155, 42157, 42159-42161, 42164-42165,
42167-42168, 42171, 42173-42176, 42188, 42192, 42196, 42213, 42215,
42222, 42228-42229, 42232, 42234, 42236-42237, 42242-42243, 42245,
42247-42251, 42253, 42257-42260, 42262, 42274, 42277, 42279, 42282,
42284-42285, 42287-42290, 42292-42293, 42295, 42299-42309,
42311-42316, 42319-42323, 42325, 42331-42340, 42342-42343,
42348-42349, 42352-42357, 42359-42361, 42363-42364, 42367, 42370,
42376-42377, 42379, 42381, 42384, 42386-42395, 42397-42400,
42402-42408, 42410, 42412-42416, 42421, 42426-42433, 42435, 42440,
42442, 42444, 42449, 42451-42452, 42455-42456, 42461-42462, 42466,
42472, 42474, 42477-42479, 42481-42482, 42484-42485, 42487-42488,
42494-42496, 42498-42499, 42502-42503, 42507, 42513-42516, 42521,
42524-42525, 42527-42528, 42530-42534, 42536, 42538-42540,
42552-42563, 42567, 42570-42572, 42574-42576, 42579-42586,
42590-42591, 42594, 42597, 42599, 42601-42604, 42606, 42608-42610,
42612, 42615-42617, 42619-42620, 42624-42625, 42630, 42635,
42640-42643, 42646-42648, 42652-42654, 42657-42659, 42661,
42666-42668, 42670-42671, 42673, 42678-42683, 42686-42687, 42689,
42691-42695, 42697, 42699, 42702-42705, 42707, 42709, 42711-42712,
42715, 42721, 42724-42725, 42731, 42733, 42735-42736, 42739-42749,
42753, 42756-42757, 42760, 42765-42769, 42773, 42775-42777,
42779-42780, 42783-42784, 42786-42787, 42789-42790, 42800,
42802-42809, 42811-42813, 42816-42817, 42820-42821, 42823-42824,
42826-42830, 42833, 42836, 42839-42840, 42842-42843, 42845,
42847-42850, 42853-42854, 42856-42860, 42862, 42864-42879, 42881,
42885-42886, 42888-42891, 42893-42895, 42899, 42902-42910,
42914-42915, 42917, 42919, 42921-42926, 42928, 42931, 42933-42936,
42938-42939, 42941, 42943-42944, 42947-42953, 42955, 42957,
42959-42962, 42964, 42969, 42971, 42980, 42986, 42989, 42995,
42997-42998, 43000-43001, 43003, 43005-43006, 43008-43009,
43011-43015, 43018-43019, 43021, 43024, 43026, 43029, 43034,
43037-43038, 43040-43041, 43043-43044, 43047-43048, 43055-43056,
43058, 43061, 43063-43064, 43066-43068, 43070, 43073-43075,
43077-43078, 43080, 43084-43085, 43088, 43092, 43094-43096,
43098, 43100, 43102, 43106-43107, 43110-43111, 43113-43115,
43117, 43123-43130, 43132-43134, 43138-43139, 43141, 43143,
43145-43148, 43150-43155, 43157, 43159, 43161, 43164-43166, 43173,
43179-43180, 43184-43189, 43194-43197, 43202, 43205, 43207-43208,
43211, 43215-43217, 43219-43222, 43224, 43227, 43229, 43231-43232,
43234-43236, 43238, 43241-43242, 43244, 43247, 43249-43256,
43259-43261, 43267, 43269, 43274-43275, 43280-43281, 43288-43291,
43298-43299, 43301-43304, 43307, 43310-43313, 43315, 43317, 43321,
43325-43329, 43333, 43335, 43338-43346, 43350-43352, 43354-43357,
43360-43362, 43366-43371, 43376-43383, 43385-43387, 43389,
43392-43393, 43395, 43397, 43400, 43402, 43408-43410, 43414-43416,
43418-43421, 43423-43424, 43426, 43430, 43432-43433, 43435-43436,
43441, 43444-43445, 43449, 43454-43462, 43466-43467, 43469, 43475,
43478-43480, 43482, 43484-43488, 43492, 43494, 43496, 43500,
43503-43504, 43506, 43510-43513, 43516, 43519-43520, 43522,
43524-43532, 43534, 43536, 43540, 43555-43561, 43563-43564, 43570,
43575-43581, 43583, 43585, 43587-43588, 43591-43592, 43594-43596,
43600-43601, 43603-43607, 43610-43612, 43614-43619, 43621-43624,
43628, 43631, 43636, 43639, 43652, 43655-43657, 43659-43660, 43664,
43669-43676, 43679-43684, 43686-43687, 43690-43693, 43695,
43697-43698, 43700, 43703, 43711-43712, 43714-43716, 43718-43722,
43724-43726, 43738, 43740-43744, 43747-43749, 43752-43754, 43757,
43760, 43763, 43765-43766, 43768-43770, 43772-43775, 43778,
43781-43782, 43786-43791, 43794, 43796-43797, 43801, 43803-43804,
43806, 43810-43811, 43813, 43815, 43817, 43821-43822, 43824-43825,
43827, 43834-43843, 43845-43847, 43849, 43851-43854, 43856,
43858-43859, 43864, 43869-43872, 43874, 43876, 43879, 43881-43887,
43891-43894, 43897-43904, 43907, 43909, 43912, 43914, 43919,
43921-43923, 43927-43929, 43932-43933, 43935-43936, 43939-43941,
43943-43946, 43951-43953, 43956, 43959-43964, 43967-43969, 43971,
43973, 43975-43978, 43983, 43987, 43991-43993, 44000, 44002-44016,
44019-44020, 44024-44027, 44029-44031, 44033, 44036, 44038,
44040-44048, 44052-44054, 44056-44058, 44061-44063, 44065,
44067-44068, 44070-44074, 44076-44078, 44081-44091, 44093,
44095-44098, 44101, 44103, 44105-44113, 44115, 44118, 44120-44123,
44126-44129, 44131-44132, 44135-44137, 44139-44151, 44153-44155,
44157, 44163-44166, 44168-44173, 44179-44180, 44182-44186,
44188-44189, 44193-44196, 44198-44200, 44202-44206, 44209-44211,
44213, 44218-44219, 44221-44222, 44229, 44233, 44237-44243, 44245,
44251, 44253, 44255-44259, 44261-44266, 44270-44273, 44277-44280,
44282-44283, 44290-44291, 44298-44307, 44310-44313, 44317, 44320,
44322-44323, 44325-44326, 44329-44335, 44337-44338, 44340,
44344-44351, 44354, 44357-44366, 44370-44373, 44376, 44378-44384,
44386-44387, 44389-44390, 44392-44395, 44397-44400, 44406,
44408-44412, 44414, 44417, 44419-44424, 44426, 44429-44430, 44432,
44437-44438, 44441, 44443-44444, 44446-44450, 44462, 44467-44469,
44471-44472, 44474, 44476, 44483, 44485-44487, 44491-44492,
44496-44502, 44505-44506, 44509, 44514-44518, 44520-44522, 44524,
44526-44527, 44530, 44532, 44535, 44539, 44541, 44543-44544,
44546-44549, 44551-44552, 44554-44558, 44562-44564, 44569-44570,
44572-44574, 44578-44580, 44582, 44589-44590, 44596, 44603,
44605-44607, 44609, 44611, 44613, 44616-44621, 44626, 44629, 44635,
44649, 44651, 44653-44658, 44663-44670, 44672-44677, 44684-44687,
44689, 44692-44693, 44696-44699, 44702-44703, 44706-44710,
44713, 44715, 44717-44718, 44720-44728, 44731-44733, 44736,
44739, 44741-44745, 44748, 44751-44753, 44759, 44761-44762, 44764,
44766-44767, 44770-44772, 44774, 44776-44778, 44786-44790,
44792-44793, 44798-44799, 44801, 44806, 44808, 44813-44815,
44817-44820, 44822, 44824-44825, 44827, 44829, 44831, 44834,
44836-44839, 44841-44842, 44844-44850, 44854, 44856-44857,
44859-44861, 44870, 44875, 44879-44880, 44882, 44886-44892,
44894-44895, 44899-44902, 44906, 44908-44910, 44912, 44914-44917,
44922-44924, 44926, 44932, 44934, 44937, 44941, 44944-44945,
44947-44950, 44953, 44955-44956, 44960-44961, 44963-44965, 44967,
44969, 44971-44972, 44975, 44977, 44980-44982, 44984-44985,
44987-44994, 44999, 45001-45002, 45004-45005, 45007-45019, 45021,
45025, 45027-45029, 45032, 45034-45036, 45039-45041, 45044-45045,
45047, 45049, 45054, 45056, 45060, 45063-45064, 45069, 45071,
45073-45077, 45081-45082, 45084, 45086, 45092-45093, 45095, 45098,
45101-45103, 45105, 45107, 45109, 45113, 45115-45118, 45120-45121,
45123, 45125, 45127-45130, 45136-45140, 45142-45144, 45146-45148,
45150-45152, 45154-45158, 45161-45162, 45164, 45166, 45168-45172,
45174, 45176, 45179, 45186-45188, 45190-45192, 45201-45206,
45208-45209, 45211-45212, 45218-45220, 45229-45230, 45232,
45234-45236, 45238, 45240-45241, 45245-45248, 45253, 45255, 45261,
45268, 45270-45272, 45275, 45277-45278, 45280-45281, 45285, 45290,
45292, 45294-45298, 45300, 45303, 45305-45306, 45308-45310,
45312-45314, 45316-45318, 45320-45322, 45329-45332, 45334-45336,
45339, 45347, 45350, 45352-45353, 45355-45359, 45361-45362, 45364,
45367, 45376, 45381, 45383, 45388-45389, 45391-45395, 45398-45399,
45402-45404, 45406-45409, 45411-45417, 45420-45426, 45431-45434,
45439-45442, 45444, 45448-45449, 45451, 45455, 45460-45465,
45467-45468, 45470-45476, 45482, 45485, 45488, 45494, 45496-45498,
45502, 45504, 45506, 45509, 45513, 45515-45516, 45521-45524,
45527-45529, 45533, 45535, 45537-45541, 45543-45545, 45547-45550,
45553-45570, 45572-45575, 45577-45589, 45592-45595, 45597, 45600,
45602-45604, 45606-45610, 45612, 45615-45616, 45618-45621, 45624,
45627, 45632-45633, 45636-45637, 45639, 45642, 45644, 45646-45648,
45651, 45653-45655, 45658-45660, 45662, 45665, 45667, 45669,
45671-45675, 45678-45681, 45685-45687, 45689-45695, 45697-45698,
45701-45704, 45706, 45709-45713

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A single stranded oligonucleotide having a sequence 5′-X-Y-Z, wherein X is any nucleotide, Y is a nucleotide sequence of 6 nucleotides in length that is not a seed sequence of a human microRNA, and Z is a nucleotide sequence of 1-23 nucleotides in length, wherein the single stranded oligonucleotide is complementary with at least 8 consecutive nucleotides of a PRC2-associated region of a FOXP3 gene.

2. The single stranded oligonucleotide of claim 1, wherein the oligonucleotide does not comprise three or more consecutive guanosine nucleotides.

3. The single stranded oligonucleotide of claim 1, wherein the oligonucleotide does not comprise four or more consecutive guanosine nucleotides.

4. The single stranded oligonucleotide of claim 1, wherein the oligonucleotide is 8 to 30 nucleotides in length.

5. The single stranded oligonucleotide of claim 1, wherein the oligonucleotide is 8 to 10 nucleotides in length and all but 1, 2, or 3 of the nucleotides of the complementary sequence of the PRC2-associated region are cytosine or guano sine nucleotides.

6. The single stranded oligonucleotide of claim 1, wherein at least one nucleotide of the oligonucleotide is a nucleotide analogue.

7. The single stranded oligonucleotide of claim 6, wherein the at least one nucleotide analogue results in an increase in Tm of the oligonucleotide in a range of 1 to 5° C. compared with an oligonucleotide that does not have the at least one nucleotide analogue.

8. The single stranded oligonucleotide of claim 1, wherein at least one nucleotide of the oligonucleotide comprises a 2′ O-methyl.

9. The single stranded oligonucleotide of claim 1, wherein each nucleotide of the oligonucleotide comprises a 2′ O-methyl.

10. The single stranded oligonucleotide of claim 1, wherein the oligonucleotide comprises at least one ribonucleotide, at least one deoxyribonucleotide, or at least one bridged nucleotide.

11. The single strand oligonucleotide of claim 10, wherein the bridged nucleotide is a LNA nucleotide, a cEt nucleotide or a ENA modified nucleotide.

12. The single stranded oligonucleotide of claim 1, wherein each nucleotide of the oligonucleotide is a LNA nucleotide.

13. The single stranded oligonucleotide of claim 1, wherein the nucleotides of the oligonucleotide comprise alternating deoxyribonucleotides and 2′-fluoro-deoxyribonucleotides.

14. The single stranded oligonucleotide of claim 1, wherein the nucleotides of the oligonucleotide comprise alternating deoxyribonucleotides and 2′-O-methyl nucleotides.

15. The single stranded oligonucleotide of claim 1, wherein the nucleotides of the oligonucleotide comprise alternating deoxyribonucleotides and ENA nucleotide analogues.

16. The single stranded oligonucleotide of claim 1, wherein the nucleotides of the oligonucleotide comprise alternating deoxyribonucleotides and LNA nucleotides.

17. The single stranded oligonucleotide of claim 13, wherein the 5′ nucleotide of the oligonucleotide is a deoxyribonucleotide.

18. The single stranded oligonucleotide of claim 1, wherein the nucleotides of the oligonucleotide comprise alternating LNA nucleotides and 2′-O-methyl nucleotides.

19. The single stranded oligonucleotide of claim 18, wherein the 5′ nucleotide of the oligonucleotide is a LNA nucleotide.

20. The single stranded oligonucleotide of claim 1, wherein the nucleotides of the oligonucleotide comprise deoxyribonucleotides flanked by at least one LNA nucleotide on each of the 5′ and 3′ ends of the deoxyribonucleotides.

21. The single stranded oligonucleotide of claim 1, further comprising phosphorothioate internucleotide linkages between at least two nucleotides.

22. The single stranded oligonucleotide of claim 21, further comprising phosphorothioate internucleotide linkages between all nucleotides.

23. The single stranded oligonucleotide of claim 1, wherein the nucleotide at the 3′ position of the oligonucleotide has a 3′ hydroxyl group.

24. The single stranded oligonucleotide of claim 1, wherein the nucleotide at the 3′ position of the oligonucleotide has a 3′ thiophosphate.

25. The single stranded oligonucleotide of claim 1, further comprising a biotin moiety conjugated to the 5′ nucleotide.

26. A single stranded oligonucleotide comprising a region of complementarity that is complementary with at least 8 consecutive nucleotides of a PRC2-associated region of a FOXP3 gene, wherein the oligonucleotide has at least one of:
a) a sequence that is 5′X-Y-Z, wherein X is any nucleotide and wherein X is anchored at the 5′ end of the oligonucleotide, Y is a nucleotide sequence of 6 nucleotides in length that is not a human seed sequence of a microRNA, and Z is a nucleotide sequence of 1 to 23 nucleotides in length;
b) a sequence that does not comprise three or more consecutive guanosine nucleotides;
c) a sequence that has less than a threshold level of sequence identity with every sequence of nucleotides, of equivalent length to the second nucleotide sequence, that are between 50 kilobases upstream of a 5′-end of an off-target gene and 50 kilobases downstream of a 3′-end of the off-target gene;
d) a sequence that is complementary to a PRC2-associated region that encodes an RNA that forms a secondary structure comprising at least two single stranded loops; and/or
e) a sequence that has greater than 60% G-C content.

27. The single stranded oligonucleotide of claim 26, wherein the oligonucleotide has the sequence 5′X-Y-Z and wherein the oligonucleotide is 8-50 nucleotides in length.

28. The single stranded oligonucleotide of claim 1, wherein the single stranded oligonucleotide, when delivered to a cell, is capable of increasing the level of CTLA4, GITR, and/or IL-10 expression in the cell.

29. The single stranded oligonucleotide of claim 28, wherein the cell is a T cell.

30. The single stranded oligonucleotide of claim 1, wherein the single stranded oligonucleotide, when delivered to a population of T cells, is capable of increasing the number of CD4+CD25+FOXP3+ T cells in the population of T cells.

31. A composition comprising a single stranded oligonucleotide of claim 1 and a carrier.

32. A composition comprising a single stranded oligonucleotide of claim 1 in a buffered solution.

33. The composition of claim 32, wherein the oligonucleotide is conjugated to the carrier.

34. The composition of claim 33, wherein the carrier is a peptide.

35. The composition of claim 33, wherein the carrier is a steroid.

36. A pharmaceutical composition comprising a composition of claim 31 and a pharmaceutically acceptable carrier.

37. A kit comprising a container housing the composition of claim 31.

38. A method of increasing expression of FOXP3 in a cell, the method comprising delivering the single stranded oligonucleotide of claim 1 into the cell.

39. The method of claim 38, wherein delivery of the single stranded oligonucleotide into the cell results in a level of expression of FOXP3 that is at least 50% greater than a level of expression of FOXP3 in a control cell that does not comprise the single stranded oligonucleotide.

40. The method of claim 38, wherein delivery of the single stranded oligonucleotide into the cell results in an increased level of CTLA4, GITR, and/or IL-10 expression compared to an appropriate control cell that does not comprise the singled stranded oligonucleotide.

41. The method of claim 40, wherein delivery of the single stranded oligonucleotide into the cell results in a level of expression of CTLA4, GITR, and/or IL-10 that is at least 30% greater than a level of expression of CTLA4, GITR, and/or IL-10 in a control cell that does not comprise the single stranded oligonucleotide.

42. The method of claim 38, wherein the cell is a T cell.

43. A method increasing levels of FOXP3 in a subject, the method comprising administering the single stranded oligonucleotide of claim 1 to the subject.

44. The method of claim 43, wherein administration of the single stranded oligonucleotide to the subject results in an increased level of CTLA4, GITR, and/or IL-10 expression the subject compared to an appropriate control subject who has not been administered the single stranded oligonucleotide.

45. The method of claim 44, wherein administration of the single stranded oligonucleotide to the subject results in a level of expression of CTLA4, GITR, and/or IL-10 that is at least 30% greater than a level of CTLA4, GITR, and/or IL-10 in the appropriate control subject who has not been administered the single stranded oligonucleotide.

46. The method of claim 43, wherein administration of the single stranded oligonucleotide to the subject results in an increased level of CTLA4, GITR, and/or IL-10 in a T cell of the subject compared to a T cell in the control subject who has not been administered the single stranded oligonucleotide.

47. The method of claim 46, wherein administration of the single stranded oligonucleotide to the subject results in a level of expression of CTLA4, GITR, and/or IL-10 in the T cell of the subject that is at least 30% greater than a level of CTLA4, GITR, and/or IL-10 in the T cell in the control subject who has not been administered the single stranded oligonucleotide.

48. The method of claim 43, wherein administration of the of the single stranded oligonucleotide to the subject results in an increased number of CD4+CD25+FOXP3+ T cells in the subject compared to a control subject who has not been administered the single stranded oligonucleotide.

49. The method of claim 48, wherein administration of the single stranded oligonucleotide to the subject results in a number of CD4+CD25+FOXP3+ T cells in the subject that is at least 30% greater than a number of CD4+CD25+FOXP3+ T cells in the control subject who has not been administered the single stranded oligonucleotide.

50. A method of treating a condition or disease associated with decreased levels of FOXP3 in a subject, the method comprising administering the single stranded oligonucleotide of claim 1 to the subject.

51. The method of claim 50, wherein the condition or disease is associated with aberrant immune cell activation.

52. A method of increasing expression of FOXP3 in a cell, the method comprising delivering an oligonucleotide having a region of complementarity that is complementary with at least 8 consecutive nucleotides of a EZH1 mRNA or EZH2 mRNA to the cell.

53. The method of claim 52, wherein the oligonucleotide is 8 to 30 nucleotides in length.

54. The method of claim 52, wherein at least one nucleotide of the oligonucleotide is a nucleotide analogue.

55. The method of claim 52, wherein the oligonucleotide comprises a gapmer.

56. The method of claim 55, wherein the gapmer comprises a central region of at least 4 DNA nucleotides flanked one both sides by at least two nucleotide analogues.

57. The method of claim 55, wherein the at least two nucleotide analogues comprise at least one LNA or at least one 2′-O modified ribonucleotide.
